(19) United States
(12) Patent Application Publication
(10) Pub. No.: US 2025/0149121 A1
(43) Pub. Date: May 8, 2025

(54) DETECTION OF PEPTIDE STRUCTURES FOR DIAGNOSING AND TREATING SEPSIS AND COVID

(71) Applicant: VENN BIOSCIENCES CORPORATION, South San Francisco, CA (US)

(72) Inventors: Chad Eagle PICKERING, South San Francisco, CA (US); Gege XU, South San Francisco, CA (US); Hector Han-Li HUANG, South San Francisco, CA (US); Xin CONG, South San Francisco, CA (US)

(21) Appl. No.: 18/681,704

(22) PCT Filed: Aug. 5, 2022
(86) PCT No.: PCT/US2022/074637

§ 371 Date: Feb. 6, 2024

Related U.S. Application Data
(60) Provisional application No. 63/230,695, filed on Aug. 7, 2021.
(60) Provisional application No. 63/230,696, filed on Aug. 7, 2021.

(51) Int. Cl. G16B 40/20; G16B 15/00; G16B 40/30; G16H 10/40; G16H 50/20

(52) U.S. Cl. CPC G16B 40/20; G16B 15/00; G16B 40/30; G16H 10/40; G16H 50/20

(57) ABSTRACT

Embodiments disclosed herein generally relate to technologies for evaluating a biological sample obtained from a subject with respect to a sepsis state or coronavirus disease (COVID). Some methods relating to the technologies can include receiving peptide structure data corresponding to the biological sample obtained from the subject, identifying a peptide structure profile for the biological sample using the peptide structure data, and computing a disease indicator using the peptide structure profile and a model. The disease indicator can indicate whether the biological sample is positive for the sepsis state. The disease indicator can indicate whether the biological sample is positive for COVID. The peptide structure profile can comprise quantification data for a set of peptide structures associated with the sepsis state. The peptide structure profile can include peptides that are glycosylated, aglycosylated, or both. An additional step in the method can comprise generating at least one of a diagnosis output or a treatment output based on the disease indicator.

### CROSS-REFERENCE TO RELATED APPLICATIONS

This application is a Non-Provisional application claiming the benefit under 35 U.S.C. 119(e) of U.S. Provisional Application No. 63/230,696, filed on Aug. 7, 2021, titled “Detection of Peptide Structures for Diagnosing and Treating Sepsis,” and U.S. Provisional Application No. 63/230,695, filed on Aug. 7, 2021, titled “Detection of Peptide Structures for Diagnosing and Treating Covid,” the disclosures of which are incorporated herein by reference in their entirety.

### FIELD

The present disclosure generally relates to methods and systems for analyzing peptide structures for diagnosing and/or treating a disease state. More particularly, the present disclosure relates to analyzing quantification data for a set of peptide structures detected in a biological sample obtained from a subject for use in diagnosing and/or treating the subject, the set of peptide structures being associated with sepsis and/or COVID.

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes.

Glycoprotein analysis is fraught with challenges on several levels. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a need for improved analytical methods that involve site-specific analysis of glycoproteins to obtain information about protein glycosylation patterns, which can in turn provide quantitative information that can be used to identify disease processes. The present disclosure addresses this and other needs by combining site-specific glycoprotein analysis with machine learning and advanced mass spectrometry instrumentation to quantitatively analyze peptide structures that are indicative of specific disease states, including, but not limited to, sepsis.

Coronavirus-related diseases have demonstrated that they have the potential to be both virulent and easily transmissible, as evidenced by the common cold which is typically caused by a coronavirus and the Coronavirus disease 2019 (COVID-19) caused by the severe acute respiratory syndrome coronavirus 2 (SARS-CoV-2). However, coronaviruses may affect different persons in different ways, with certain persons experiencing more severe symptoms than others. For example, while some people experience only mild symptoms upon contracting COVID-19, others experience severe symptoms (e.g., fever, chills, nausea, vomiting, diarrhea, trouble breathing, pain and/or pressure in the chest, confusion, blue-colored lips, etc.). Persons with underlying health conditions can be more susceptible to the severe symptoms of COVID-19 and may be at risk for death. Still others who have COVID-19 are asymptomatic. In certain cases, a person having COVID-19 may experience symptoms that present similarly to another type of disease or condition. Thus, it may be desirable to have methods and systems capable of distinguishing between these different states.

Sepsis is a leading cause of death worldwide, resulting in millions of deaths globally each year. Sepsis occurs in 1-2% of all hospitalizations. Because patients often have multiple disease states simultaneously, sepsis-related deaths are likely underestimated. One of the most common methods of diagnosing sepsis involves observing a patient for symptoms relating to systematic inflammatory response syndrome (SIRS). But this methodology may not always provide an accurate diagnosis. For example, a patient with sepsis may experience symptoms that present similar to another type of disease or condition. In certain instances, a patient with sepsis may present similarly to a patient with symptomatic coronavirus disease (COVID), such as COVID-19. Thus, it may be desirable to have methods and systems capable of diagnosing sepsis and/or distinguishing between patients who have sepsis and those that do not.

### SUMMARY

Aspects of the disclosure comprise a method of determining whether a biological sample corresponds to a sepsis state. In some embodiments, the method can comprise receiving peptide structure data corresponding to the biological sample obtained from a subject. In some embodiments, the method can comprise inputting quantification data identified from the peptide structure data for a set of peptide structures into a supervised machine learning model, wherein the set of peptide structures includes at least one peptide structure identified in Table 1. In some embodiments, the method can comprise analyzing the quantification data using the supervised machine learning model to generate a disease indicator that classifies the biological sample as corresponding to a cluster of a plurality of clusters that has a least distance to the biological sample, the plurality of clusters corresponding a plurality of states that includes the sepsis state. In some embodiments, the method can comprise generating a diagnosis output based on the disease indicator.

Aspects of the disclosure comprise a method of identifying a sepsis-specific set of peptide structures for use in diagnosing a sepsis state, the method comprising. In some embodiments, the method can comprise receiving quantification data for a panel of peptide structures for a plurality of subjects diagnosed with a plurality of states, the plurality of states including the sepsis state. In some embodiments, the method can comprise comparing quantification metrics corresponding to the panel of peptide structures for a first portion of the plurality of subjects diagnosed with the sepsis state to that of each portion of a set of other portions of the plurality of subjects to generate a set of comparisons using a differential expression analysis. In some embodiments, the method can comprise selecting a portion of the panel of peptide structures having a false discovery rate below 0.05 across the set of comparisons as a set of peptide structures to be associated with the sepsis state. In some embodiments, the set of peptide structures includes at least one glycopeptide structure defined by a peptide sequence and a glycan structure linked to the peptide sequence at a linking site of the peptide sequence. In some embodiments, the method can comprise analyzing the quantification data for the set of peptide structures for the plurality of subjects using an unsupervised machine learning model to cluster the plurality of subjects. In some embodiments, the method can comprise training a supervised machine learning model to determine whether a biological sample obtained from a subject corresponds to the sepsis state or another state of the plurality of states.

Aspects of the disclosure include a method of evaluating a biological sample obtained from a subject with respect to a sepsis state. In some embodiments, the method can comprise receiving peptide structure data corresponding to the biological sample obtained from the subject. In some embodiments, the method can comprise identifying a peptide structure profile for the biological sample using the peptide structure data, the peptide structure profile comprising quantification data for a set of peptide structures associated with the sepsis state. In some embodiments, the set of peptide structures includes at least two peptide structures from a selected group of peptide structures identified in Table 1. In some embodiments, at least two peptide structures in the selected group of peptide structures includes a glycopeptide structure defined by a peptide sequence and a glycan structure linked to a linking site of the peptide sequence. In some embodiments, the method can comprise computing a disease indicator using the peptide structure profile and a model, wherein the disease indicator indicates whether the biological sample is positive for the sepsis state. In some embodiments, the method can comprise generating at least one of a diagnosis output or a treatment output based on the disease indicator.

Aspects of the disclosure can comprise a method of evaluating a biological sample obtained from a subject with respect to a symptomatic disease state corresponding to a coronavirus disease (COVID). In some embodiments, the method can comprise receiving peptide structure data corresponding to the biological sample obtained from the subject. In some embodiments, the method can comprise identifying a peptide structure profile for the biological sample using the peptide structure data, the peptide structure profile comprising quantification data for a set of peptide structures associated with the symptomatic disease state. In some embodiments, the set of peptide structures includes at least two peptide structures from a selected group of peptide structures identified in Table 1-1. In some embodiments, at least two peptide structures in the selected group of peptide structures includes a glycopeptide structure defined by a peptide sequence and a glycan structure linked to a linking site of the peptide sequence, although in some embodiments one or more peptide structures is an aglycosylated peptide. In some embodiments, the method comprises computing a disease indicator using the peptide structure profile and a model, wherein the disease indicator indicates whether the biological sample is positive for the symptomatic disease state. In some embodiments, the method can comprise generating at least one of a diagnosis output or a treatment output based on the disease indicator.

Aspects of the disclosure can comprise a method for analyzing a set of peptide structures in a sample from a patient. In some embodiments, the method can comprise obtaining the sample from the patient. In some embodiments, the method can comprise preparing the sample to form a prepared sample comprising a set of peptide structures. In some embodiments, the method can comprise inputting the prepared sample into a reaction monitoring mass spectrometry system to detect a set of product ions associated with each peptide structure of the set of peptide structures, the set of peptide structures comprising at least one of. In some embodiments, the peptide structures can be associated with the product and precursor ions listed in Table 2 and having a range of ±0.5, ±0.8, or ±1.0.

Aspects of the disclosure can comprise a method for analyzing a set of peptide structures in a sample from a patient. In some embodiments, the method can comprise obtaining the sample from the patient. In some embodiments, the method can comprise preparing the sample to form a prepared sample comprising a set of peptide structures. In some embodiments, the method can comprise inputting the prepared sample into a reaction monitoring mass spectrometry system to detect a set of product ions associated with each peptide structure of the set of peptide structures, the set of peptide structures comprising at least one of the peptide structures shown in Table 1 with a corresponding product ion and precursor ion shown in Table 4-1, each including a range of ±0.5, ±0.8, or ±1.0.

Aspects of the disclosure can comprise a composition comprising a peptide structure or a product ion, wherein the peptide structure or product ion comprises an amino acid sequence having at least 90% sequence identity to any one of SEQ ID NOS: 24-54, corresponding to peptide structures PS-1 to PS-46 in Table 1 and the product ion is selected as one from a group consisting of product ions identified in Table 2 including product ions falling within an identified m/z range.

Aspects of the disclosure can comprise a composition comprising a peptide structure or a product ion, wherein the peptide structure or product ion comprises an amino acid sequence having at least 90% sequence identity to any one of SEQ ID NOS: 84-116, corresponding to peptide structures PS-1 to PS-46 in Table 1-1 and the product ion is selected as one from a group consisting of product ions identified in Table 2-1 including product ions falling within an identified m/z range.

Aspects of the disclosure comprise a composition comprising a glycopeptide structure selected as one from a group of glycopeptide structures consisting of any of the glycopeptide structures identified in Table 1 and the glycopeptide structures can be linked to the listed glycans shown in Table 5.

Aspects of the disclosure comprise a composition comprising a glycopeptide structure selected as one from a group of glycopeptide structures consisting of any of the glycopeptide structures identified in Table 1-1 and the glycopeptide structures can be linked to the listed glycans shown in Table 5-1.

Aspects of the disclosure comprise a composition comprising a peptide structure selected as one from a group of aglycosylated peptide structures listed in Table 1.

Aspects of the disclosure comprise a composition comprising a peptide structure selected as one from a group of aglycosylated peptide structures listed in Table 1-1.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

### BRIEF DESCRIPTION OF THE DRAWINGS

The present disclosure is described in conjunction with the appended figures:

FIG. 1 is a schematic diagram of an exemplary workflow 100 for the detection of peptide structures associated with a disease state for use in diagnosis and/or treatment in accordance with one or more embodiments.

FIGS. 2A and 2B. FIG. 2A is a schematic diagram of preparation workflow 200 in accordance with one or more embodiments. FIG. 2B is a schematic diagram of data acquisition 124 in accordance with one or more embodiments.

FIG. 3 is a block diagram of an analysis system 300 in accordance with one or more embodiments.

FIG. 4 is a block diagram of a computer system in accordance with various embodiments.

FIG. 5 is a flowchart of a process for evaluating a biological sample obtained from a subject with respect to a sepsis state in accordance with one or more embodiments.

FIG. 6 is a flowchart of a process for determining whether a biological sample corresponds to a sepsis state in accordance with various embodiments.

FIG. 7 is a flowchart of a process for identifying a sepsis-specific set of peptide structures for use in diagnosing a sepsis state of the coronavirus disease in accordance with one or more embodiments.

FIG. 8 is a flowchart of a process for treating a subject for a sepsis state in accordance with one or more embodiments.

FIG. 9 is a table that provides information about subjects involved in an exemplary experiment, including, number, sample type, gender, and median age, where available in accordance with one or more embodiments.

FIG. 10 is a plot that show differential expression (FDR<0.05) data for the 115 samples that has been clustered in accordance with one or more embodiments.

FIG. 11 is an illustration of a heat map depicting the 46 peptide structures, also identified in Table 1, in accordance with one or more embodiments.

FIG. 12 is a plot that shows a k-means clustering graph using markers differentially expressed between sepsis and other groups in accordance with one or more embodiments.

FIG. 13 is a flowchart of a process for evaluating a biological sample obtained from a subject with respect to a symptomatic disease state of COVID in accordance with one or more embodiments.

FIG. 14 is a flowchart of a process for determining whether a biological sample corresponds to a symptomatic disease state of a coronavirus disease (COVID) in accordance with various embodiments.

FIG. 15 is a flowchart of a process for identifying a coronavirus disease (COVID)-specific set of peptide structures for use in diagnosing a symptomatic disease state of the coronavirus disease in accordance with one or more embodiments.

FIG. 16 is a flowchart of a process for diagnosing a symptomatic disease state of a coronavirus disease (COVID) in accordance with one or more embodiments.

FIG. 17 is a flowchart of a process for treating a subject for a coronavirus disease state in accordance with one or more embodiments.

FIG. 18 is a table that provides information about subjects involved in an exemplary experiment, including, number, sample type, gender, and median age, where available in accordance with one or more embodiments.

FIG. 19 is a plot showing a principal component analysis via a singular value decomposition of the centered and scaled data matrix of all 115 patient samples and all peptide structures (e.g., glycosylated and aglycosylated) of the panel in accordance with one or more embodiments.

FIG. 20 is an illustration of a heat map depicting the peptide structures, also identified in Table 2-2 in accordance with one or more embodiments.

FIG. 21 is a plot showing a k-means clustering graph using markers differentially expressed between symptomatic COVID and all other groups in accordance with one or more embodiments.

FIG. 22 is an illustration of a 5-fold cross validated LASSO regression model classifying COVID versus other patients in accordance with one or more embodiments.

### DETAILED DESCRIPTION

### I. Overview

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general for several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and to more accurately diagnose certain disease conditions, such as sepsis, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information about a disease state. This information can be used to distinguish the disease state from other states, diagnose a subject as having or not having the disease state, determine a likelihood that a subject has the disease state, or a combination thereof.

Accordingly, the embodiments described herein provide various methods and systems for analyzing proteins in subjects and, in particular, glycoproteins. In one or more embodiments, a machine learning model is trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject having a particular disease state. Further, certain peptide structures that are associated with a particular disease state (e.g., a sepsis state) may be more relevant to that disease state than other peptide structures that are also associated with that disease state.

Analyzing the abundance of peptide sequences and glycosylated peptide sequences in a biological sample may provide a more accurate way in which to distinguish a disease state of interest, such as a sepsis state, from other states (e.g., the common cold, a healthy state, sepsis, a symptomatic disease state of a coronavirus disease (COVID), a symptomatic disease state of COVID, etc.). This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

The description below provides exemplary implementations of the methods and systems described herein for the research, diagnosis, and/or treatment (e.g., designing, planning, and/or manufacturing of a treatment) of a sepsis state. The sepsis state may be a general state of sepsis regardless of whether mild or severe, a severe sepsis state, a moderate sepsis state, a mild sepsis state, or some other sepsis state. Descriptions and examples of various terms, as used herein, are provided in Section II below.

### II. Exemplary Descriptions of Terms

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “amino acid,” as used herein, generally refers to any organic compound that includes an amino group (e.g. —NH2), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation can relate to techniques used to release glycans.

The term “asymptomatic,” as used herein, generally refers to a subject displaying no signs of a disease state such as loss of function, however, may test as a carrier for a disease (e.g. biomarkers for a disease state may be present). The term asymptomatic can comprise the term “pre-symptomatic” which can mean a subject may not display signs of a disease state, but signs may later develop.

The term “linking site” or “glycosylation site” as used herein generally refers to the location where a sugar molecule of a glycan or glycan structure is directly bound (e.g. covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g. ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g. to diagnose a health state, an asymptomatic state, or a symptomatic state).

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refer to breaking apart a polymer (e.g. cutting a polypeptide at a cut site). Proteins may be digested in preparation for mass spectrometry using trypsin digestion protocols. Proteins may be digested using other proteases in preparation for mass spectrometry if access is limited to cleavage sites.

The terms “immune checkpoint inhibitor therapeutic” and “immune checkpoint inhibitor drug,” as used herein, generally refer to drugs or therapeutics that can target immune checkpoint molecules (e.g. molecules on immune cells that need to be activated (or inactivated) to start an immune response). Non-limiting examples of immune checkpoint inhibitor therapeutics can include pembrolizumab, nivolumab, and cemiplimab.

The term “common cold,” as used herein, generally refers to any virus. Common colds can include any viruses associated with corona virus. Common colds can include any viruses associated with sepsis.

The terms “coronavirus disease” and “COVID” are used interchangeably and generally refer to a group of related RNA viruses that can cause disease states in mammals and birds. Non-limiting examples of coronavirus diseases can include SARS, MERS, and COVID-19.

The term “disease state” as used herein, generally refers to a condition that negatively affects the structure or function of an organism. Non-limiting examples of causes of disease states may include pathogens, immune system dysfunctions, cell damage caused by aging, cell damage caused by other factors (e.g. trauma and cancer). Disease states can include any state of a disease whether symptomatic or asymptomatic. Disease states can cause minor, moderate, or severe disruptions in structure or function of an organism (e.g. a subject).

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refer to a peptide or polypeptide comprising at least one glycan residue. In various embodiments, glycopeptides comprise carbohydrate moieties (e.g. one or more glycans) covalently attached to a side chain (i.e. R group) of an amino acid residue.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins, include but are not limited to apolipoprotein C-III (APOC3), alpha-1-antichymotrypsin (AACT), afamin (AFAM), alpha-1-acid glycoprotein 1 & 2 (AGP12), apolipoprotein B-100 (APOB), apolipoprotein D (APOD), complement C1s subcomponent (C1S), calpain-3 (CAN3), clusterin (CLUS), complement component C8AChain (CO8A), alpha-2-HS-glycoprotein (FETUA), haptoglobin (HPT), immunoglobulin heavy constant gamma 1 (IgG1), immunoglobulin J chain (IgJ), plasma kallikrein (KLKB1), serum paraoxonase/arylesterase 1 (PON1), prothrombin (THRB), serotransferrin (TRFE), protein unc-13 homologA (UN13A), and zinc-alpha-2-glycoprotein (ZA2G). A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.”

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction can relate to techniques used to release glycans.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sepsis,” as used herein, generally refers to a subject's response to infection that can cause injury to its own tissues or organs. Sepsis can be caused by a variety of different organisms, including, but not limited to bacteria, viruses, and fungi. In various embodiments, the presence of two or more of the following can indicate that a subject has sepsis: abnormal body fever, heart rate, respiratory rate, or blood gas, and white blood cell count.

The term “sequence,” as used herein, generally refers to a biological sequence including one-dimensional monomers that can be assembled to generate a polymer. Non-limiting examples of sequences include nucleotide sequences (e.g. ssDNA, dsDNA, and RNA), amino acid sequences (e.g. proteins, peptides, and polypeptides), and carbohydrates (e.g. compounds including Cₘ (H₂O)ₙ).

The terms “severe acute respiratory syndrome coronavirus 2” and “SARS-CoV-2,” as used herein, generally refers to the virus that causes coronavirus disease (e.g. COVID-19). SARS-CoV-2 can be a virus of the species severe acute respiratory syndrome-related coronavirus (SARSr-CoV).

The term “coronavirus” generally refers to virus in a group of related RNA viruses that cause diseases in, for example, mammals and birds. In humans and birds, coronaviruses can cause respiratory tract infections. A coronavirus may be comprised of ssRNA molecules enclosed within an envelope embedded with protein molecules. The viral envelope may be a lipid bilayer in which spike structural proteins are anchored. Generally, spike proteins are used for interaction with host cells; however, other proteins may play this role in certain coronavirus variants. The earliest reports of coronavirus in animals occurred sometime in the first half of the twentieth century. The most common symptoms included respiratory infection leading to gasping and trouble breathing. The existence of human-related coronaviruses was discovered in the 1960s with various other coronaviruses having since been identified. The other coronaviruses include, for example, but are not limited to, SARS-CoV identified in 2003, HCoV NL63 identified in 2003, identified in HCoV HKU1, MERS-CoV identified in 2013, and SARS-CoV-2 identified in 2019.

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. A subject can include a microorganism or microbe (e.g., bacteria, fungi, archaea, viruses).

The term “symptomatic,” as used herein, generally refers to a subject showing signs of a disease state. Symptoms can include anything abnormal or not commonly experienced by a subject. Symptoms or signs can include observational data indicative or a disturbance in a subject's homeostasis. Symptoms or signs can be detected during a physical examination. Symptoms can be used to arrive at a diagnosis in a subject. Specific, non-limiting examples of symptoms can include fever, chills, cough, shortness of breath or difficulty breathing, fatigue, muscle or body aches, headache, new loss of taste or smell, sore throat, congestion or runny nose, nausea or vomiting, diarrhea, trouble breathing, persistent pain or pressure in the chest, new confusion, inability to wake or stay awake, pale, gray, or blue-colored skin, lips, or nail beds, depending on skin tone, pneumonia, or painful urination with a kidney infection.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

### III. Overview of Exemplary Workflow

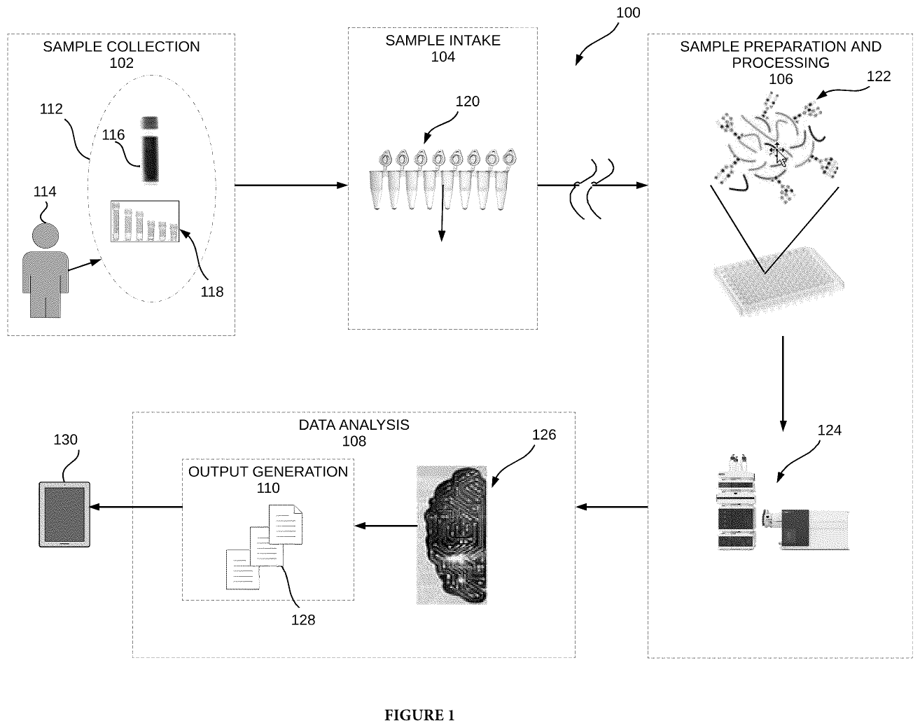

FIG. 1 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g. ssDNA, dsDNA, RNA), organelles, amino acids, peptides, proteins, carbohydrates, glycoproteins, or any combination thereof.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for the research, diagnosis, and/or treatment of a sepsis state (e.g., a sepsis condition).

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), or combination thereof. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of, for example, sepsis.

### IV. Detection and Quantification of Peptide Structures

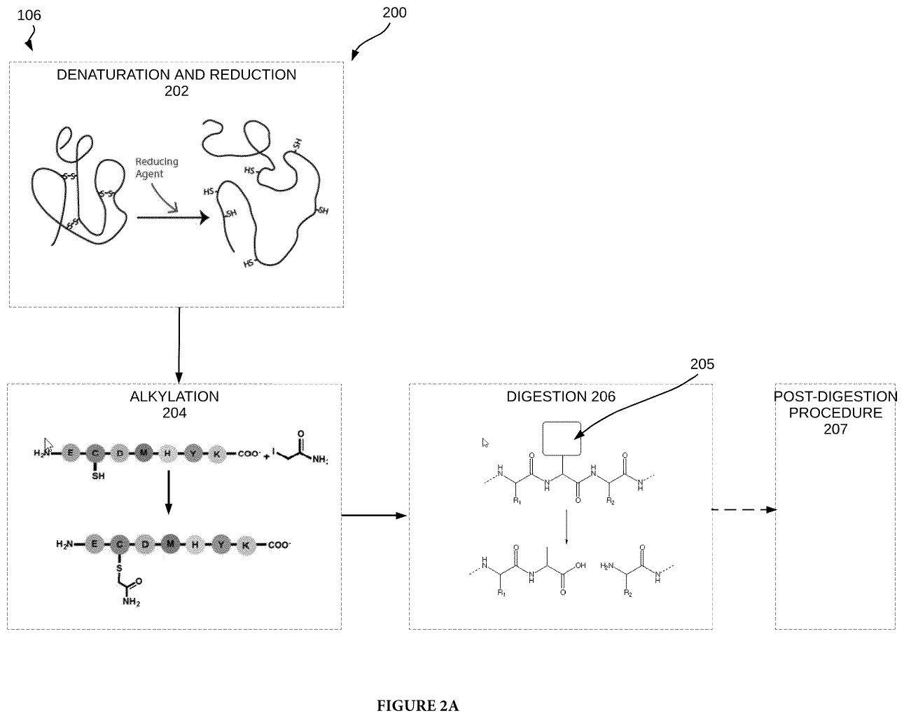

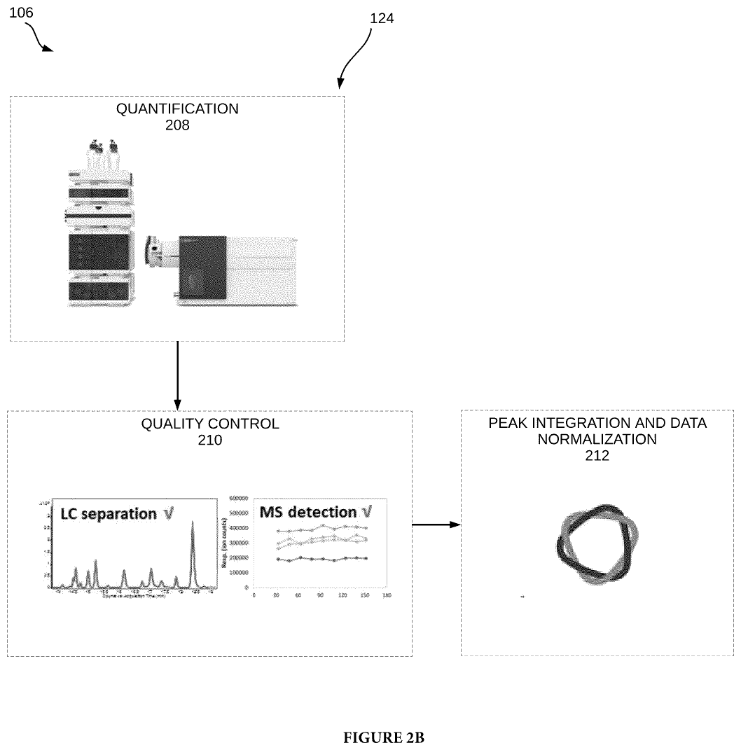

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry. In various embodiments, preparation workflow 200 may include denaturation and reduction 202, alkylation 204, and digestion 206.

In general, polymers, such as proteins, in their native form, can fold to include secondary, tertiary, and/or other higher order structures. Such higher order structures may functionalize proteins to complete tasks (e.g. enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g. peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, a Triple Quadrupole LC/MS™ includes example instruments suited for identification and targeted quantification 208. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and their absolute or relative quantities assessed.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g. using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996, the disclosures of which are incorporated by reference herein in their entireties.

### V. Peptide Structure Data Analysis

### V.A. Exemplary System for Peptide Structure Data Analysis

### V.A.1. Analysis System for Peptide Structure Data Analysis

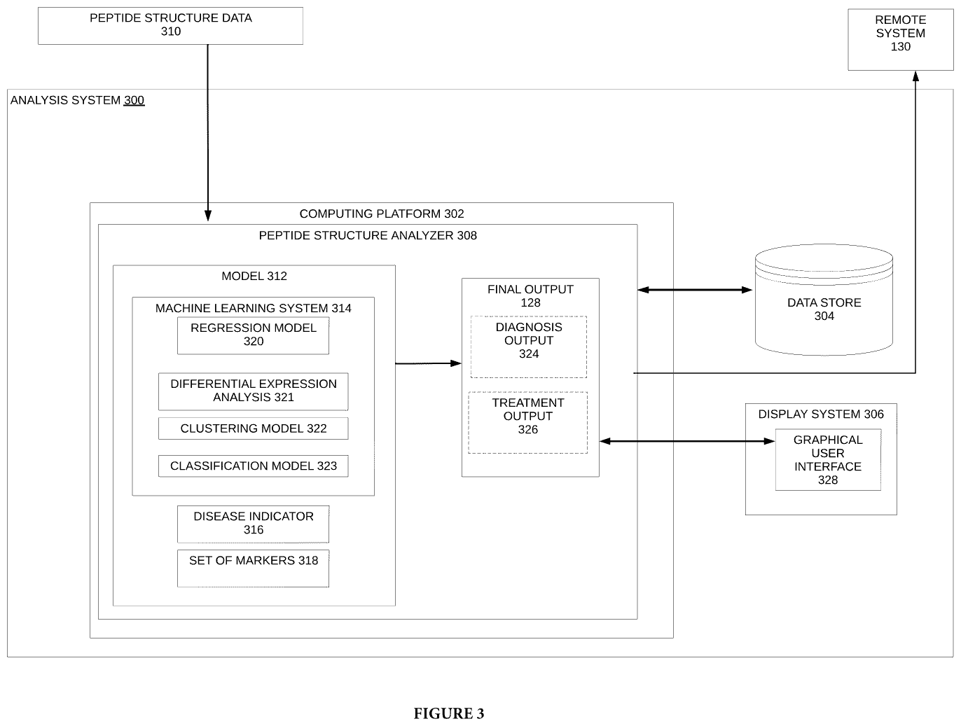

FIG. 3 is a block diagram of an analysis system 300 in accordance with one or more embodiments. Analysis system 300 can be used to both detect and analyze various peptide structures that have been associated with sepsis and/or COVID. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In one or more embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure analyzer 308 includes model 312 that is configured to receive peptide structure data 310 for processing. Model 312 may be implemented in any of a number of different ways. Model 312 may be implemented using any number of models, functions, equations, algorithms, and/or other mathematical techniques.

In one or more embodiments, model 312 includes machine learning system 314, which may itself be comprised of any number of machine learning models and/or algorithms. For example, machine learning system 314 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 312 includes a machine learning model 314 that comprises any number of or combination of the models or algorithms described above.

In various embodiments, model 312 analyzes peptide structure data 310 to generate disease indicator 316 that indicates whether the biological sample is positive for the sepsis state based on set of peptide structures 318 identified as being associated with the sepsis state. In some embodiments, model 312 analyzes peptide structure data 310 to generate disease indicator 316 that indicates whether the biological sample is positive for the symptomatic disease state based on set of peptide structures 318 identified as being associated with the symptomatic disease state. Peptide structure data 310 may comprise a set of quantification metrics for each peptide structure of a plurality of peptide structures. A quantification metric for a peptide structure may be selected from one of a relative quantity, an adjusted quantity, and a normalized quantity. In some cases, a quantification metric for a peptide structure is selected from one of a relative concentration, an adjusted concentration, and a normalized concentration. In this manner, peptide structure data 310 may provide abundance information about the plurality of peptide structures with respect to biological sample 112.

Disease indicator 316 may take various forms. In one or more embodiments, disease indicator 316 takes the form of a classification of biological sample 112 as corresponding to a particular state (e.g., a sepsis state, another disease state, or another state). Another disease state may be, for example, but is not limited to a symptomatic disease state of COVID. In some embodiments, disease indicator 316 may include a likelihood that the subject is positive for the sepsis state.

In some embodiments, a peptide structure of set of peptide structures 318 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 318 comprises an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the sepsis state based on training of model 312. In one or more embodiments, set of peptide structures 318 includes at least one, at least three, or at least five of the peptide structures identified in Table 1 below in Section V.B.3. The number of peptide structures selected from Table 1 for inclusion in set of peptide structures 318 may be based on, for example, a desired level of accuracy. In one or more embodiments, set of peptide structures 318 includes at least one, at least three, or at least five of the peptide structures identified in Table 1-1 below in Section IX.A. The number of peptide structures selected from Table 1-1 for inclusion in set of peptide structures 318 may be based on, for example, a desired level of accuracy.

In various embodiments, machine learning model 314 takes the form of regression model 320. Regression model 320 may be, for example, a penalized multivariable regression model that is trained to identify set of peptide structures 318 from a plurality of (or panel of) peptide structures identified in various subjects. Regression model 320 may be trained to identify weight coefficients for peptide structures and those peptide structures having non-zero weights or weight coefficients above a selected threshold (e.g., absolute weight coefficient above 0.1, 0.2, 0.3, etc.) may be selected for inclusion in set of peptide structures 318.

In other embodiments, model 312 takes the form of k-means clustering model 322 that uses a k-means clustering algorithm and/or unsupervised learning to generate disease indicator 316. In these examples, model 312 may be used to analyze peptide structure data 310 to generate disease indicator 316 in the form of a score that is based on at least one of a set of peptide structures 318. Model 312 may compute a distance of the score to each centroid of a plurality of centroids for a plurality of states. The plurality of states may include, for example, the sepsis state as well as at least one other state. The other state or states may include, for example, at least one of a healthy state, common cold state, an asymptomatic disease state of a coronavirus disease (COVID), a symptomatic disease state of COVID, or another disease state. In one or more embodiments, the referenced COVID is the Coronavirus disease-2019 (COVID-2019) caused by the severe acute respiratory syndrome coronavirus 2 (SARS-CoV-2).

Peptide structure analyzer 308 may generate final output 128 based on disease indicator 316 output by model 312. In other embodiments, final output 128 may be an output generated by model 312.

In some embodiments, final output 128 includes disease indicator 316. In other embodiments, final output 128 includes diagnosis output 324 and/or treatment output 326. Diagnosis output 324 may include, for example, a determination of whether the subject is positive for the sepsis state based on disease indicator 316. Treatment output 326 may include, for example, at least one of an identification of a therapeutic to treat the subject, a design for the therapeutic, or a treatment plan for administering the therapeutic. In some embodiments, the therapeutic is an immune checkpoint inhibitor. In various embodiments, treatment output 326 includes a dosage for the therapeutic to be used in treating the subject. This dosage may be computed based on, for example, the disease indicator.

Final output 128 may be sent to remote system 130 for processing in some examples. In other embodiments, final output 128 may be displayed on graphical user interface 328 in display system 306 for viewing by a human operator. The human operator may use final output 128 to diagnose and/or treat subject when final output 128 indicates the subject is positive for the sepsis state.

### V.A.2. Computer Implemented System

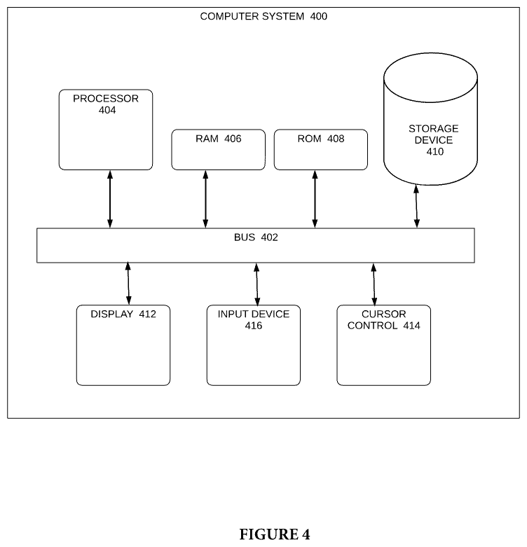

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### V.B. Exemplary Methodology for Peptide Structure Data Analysis

### V.B.1. Machine Learning-Based Analysis

As described above, analysis of peptide structure data, such as peptide structure data 310 in FIG. 3, may be performed using various machine learning-based techniques and/or algorithms. For example, without limitation, peptide structure data may be analyzed using a machine learning system (e.g., machine learning system 314 in FIG. 3) that comprises one or more supervised machine learning models, one or more unsupervised machine learning models, or a combination thereof. The machine learning system may include, for example, at least one of a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a multivariable regression model, a penalized multivariable regression model, some other type of supervised machine learning model, some other type of unsupervised machine learning model, or a combination thereof.

### V.B.2. Non-Machine Learning-Based Analysis

In some embodiments, non-machine learning-based techniques and/or algorithms may be used to analyze peptide structure data, such as peptide structure data 310 in FIG. 3. Such techniques and/or algorithms may include, for example, without limitation, one or more mathematical models, equations, and/or functions, one or more statistical models, one or more deterministic algorithms, or a combination thereof.

### V.B.3. General Methodology

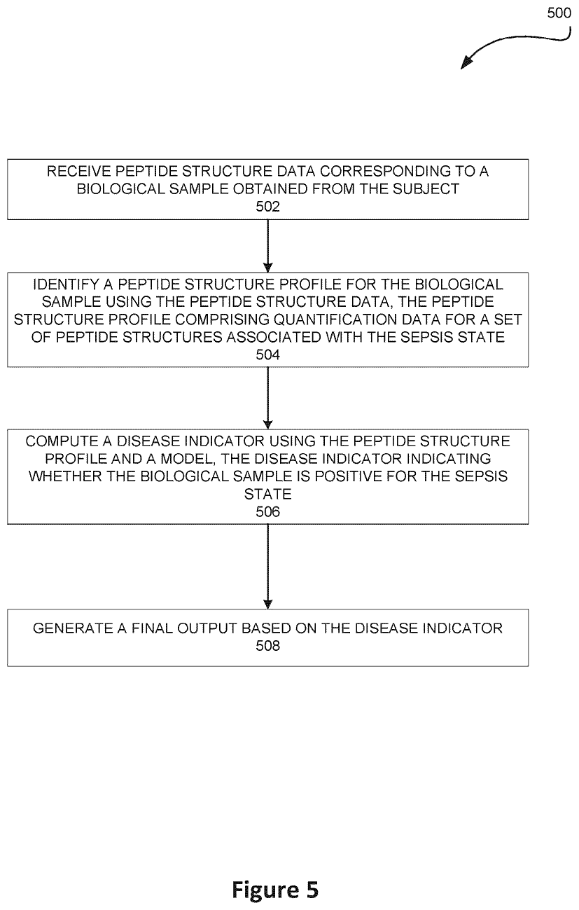

FIG. 5 is a flowchart of a process for evaluating a biological sample obtained from a subject with respect to a sepsis state in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate at least one of a diagnosis output or a treatment output for the subject.

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample.

Step 504 includes identifying a peptide structure profile for the biological sample using the peptide structure data, the peptide structure profile comprising quantification data for a set of peptide structures associated with the sepsis state. In step 504, the set of peptide structures includes at least two peptide structures from a selected group of peptide structures identified in Table 1 below. The selected group of peptide structures may be, for example, a portion of the peptide structure identified in Table 1. At least two peptide structures in the selected group of peptide structures includes a glycopeptide structure defined by a peptide sequence and a glycan structure linked to a linking site of the peptide sequence.

Step 506 includes computing a disease indicator using the peptide structure profile and a model, the disease indicator indicating whether the biological sample is positive for the sepsis state. Step 506 may be performed using model 312 in FIG. 3. For example, step 506 may be performed using machine learning system 314 in FIG. 3. In various embodiments, the disease indicator comprises a classification of the biological sample as belonging to a cluster that corresponds to a sepsis state or another cluster corresponding to another state (e.g., a healthy state, a symptomatic disease state of COVID, an asymptomatic disease state of COVID, a common cold state, some other state, or a combination thereof). In some embodiments, the disease indicator includes an identification of the cluster, an identification of the state corresponding to the cluster, or both.

Step 508 includes generating a final output based on the disease indicator. The final output may include, for example, without limitation, at least one of a diagnosis output or a treatment output. The diagnosis output may include the disease indicator or a diagnosis made based on the disease indicator. The treatment output may include, for example, at least one of an identification of a treatment to treat the subject, a design for the treatment, a manufacturing plan for the treatment, or a treatment plan for administering the treatment.

TABLE 1
Peptide Structures associated with Sepsis

| | | | | Linking | Linking | | |
|---|---|---|---|---|---|---|---|
| | Peptide | (Protein) | (Peptide) | Site Pos. | Site Pos. | Glycan | Mono- |
| PS-ID | Structure (PS) | SEQ ID | SEQ ID | in Protein | in Peptide | Structure | isotopic |
| NO. | NAME | No: | No: | Sequence | Sequence | GL NO. | Mass (Da) |
| PS-1 | AGP12-72MC_7601 | 1 | 24 | 72 | 15 | 7601 | 5538.39 |
| PS-2 | AGP12-72MC_7602 | 1 | 24 | 72 | 15 | 7602 | 5829.49 |
| PS-3 | CLUS-86_6503 | 2 | 25 | 86 | 5 | 6503 | 3807.44 |
| PS-4 | APOD-98_5412 | 3 | 26 | 98 | 16 | 5412 | 4603.94 |
| PS-5 | VTNC-86_5421 | 4 | 27 | 86 | 2 | 5402 | 4584.93 |
| PS-6 | AACT-127_5401 | 5 | 28 | 127 | 3 | 5401 | 4125.73 |
| PS-7 | AACT-127_6502 | 5 | 28 | 127 | 3 | 6502 | 4781.95 |
| PS-8 | THRB-143_5402 | 6 | 29 | 143 | 8 | 5402 | 4915.03 |
| PS-9 | VTNC-169_5301 | 4 | 30 | 169 | 1 | 5301 | 2621.06 |
| PS-10 | HPT-184_6411 | 7 | 31 | 184 | 6 | 6411 | 4900.17 |
| PS-11 | CO8B-243_6610 | 8 | 32 | 243 | 11 | 6610 | 4231.67 |
| PS-12 | HPT-241_7602 | 7 | 33 | 241 | 6 | 7602 | 4729.04 |
| PS-13 | HEMO-240/246_5402 | 9 | 34 | 240 | 1 or 7 | 5402 | 4055.56 |
| PS-14 | ITIH1-285_5402/5421 | 10 | 35 | 285 | 19 | 5402 | 5133.19 |
| PS-15 | ITIH1-285_5511 | 10 | 35 | 285 | 19 | 5511 | 5191.23 |
| PS-16 | ITIH1-285_5402 | 10 | 35 | 285 | 19 | 5402 | 5133.19 |
| PS-17 | A1AT-271MC_5402 | 11 | 36 | 271 | 4 | 5402 | 5744.60 |
| PS-18 | AACT-271_6513 | 5 | 37 | 271 | 4 | 6513 | 4758.93 |
| PS-19 | CLUS-291_5402 | 2 | 38 | 291 | 2 | 5402 | 3148.20 |
| PS-20 | CLUS-291_5402 | 2 | 38 | 291 | 2 | 5402 | 3148.20 |
| PS-21 | IGG1-180_3410 | 12 | 39 | 180 | 5 | 3410 | 2633.04 |
| PS-22 | IGG1-180_3510 | 12 | 39 | 180 | 5 | 3510 | 2836.12 |
| PS-23 | IGG2-176_4310 | 13 | 39 | 176 | 5 | 4310 | 2560.02 |
| PS-24 | IGG2-176_4400 | 13 | 39 | 176 | 5 | 4400 | 2617.04 |
| PS-25 | IGG2-176_4410 | 13 | 39 | 176 | 5 | 4410 | 2763.10 |
| PS-26 | IGG2-176_4411 | 13 | 39 | 176 | 5 | 4411 | 3054.20 |
| PS-27 | IGG2-176_4511 | 13 | 39 | 176 | 5 | 4511 | 3257.28 |
| PS-28 | IGG2-176_5410 | 13 | 39 | 176 | 5 | 5410 | 2925.15 |
| PS-29 | IGG2-176_5411 | 13 | 39 | 176 | 5 | 5411 | 3216.25 |
| PS-30 | IGG2-176_5510 | 13 | 39 | 176 | 5 | 5510 | 3128.23 |
| PS-31 | FETUA-346_1101 | 14 | 40 | 346 | 6 | 1101 | 2671.29 |
| PS-32 | FETUA-346_1102 | 14 | 40 | 346 | 6 | 1102 | 2962.39 |
| PS-33 | KLKB1-396_5402 | 15 | 41 | 396 | 6 | 5402 | 4561.95 |
| PS-34 | KLKB1-453_5402 | 15 | 42 | 453 | 7 | 5402 | 3640.56 |
| PS-35 | KLKB1-494_5401 | 15 | 43 | 494 | 6 | 5401 | 4159.85 |
| PS-36 | ITIH1-588_5402 | 10 | 44 | 588 | 2 | 5402 | 5034.18 |
| PS-37 | CO2-621_6301 | 16 | 45 | 621 | 10 | 6301 | 3326.42 |
| PS-38 | KNG1-628_1102 | 17 | 46 | 628 | 8 | 1102 | 2280.98 |
| PS-39 | ITIH1-653_1101 | 10 | 47 | 653 | 12 | 1101 | 2813.31 |
| PS-40 | A2MG-991_5402 | 18 | 48 | 991 | 46 | 5402 | 8432.83 |
| PS-41 | UN13A-1005_7420 | 19 | 49 | 1005 | 4 | 7420 | 4791.91 |
| PS-42 | QUANTPEP-CLUS- | 2 | 50 | N/A | N/A | N/A | 1392.69 |
| | ASSIIDELFQDR | | | | | | |
| PS-43 | QUANTPEP-APOA1- | 20 | 51 | N/A | N/A | N/A | 1234.68 |
| | DLATVYVDVLK | | | | | | |
| PS-44 | QUANTPEP-A2GL- | 21 | 52 | N/A | N/A | N/A | 1178.67 |
| | DLLLPQPDLR | | | | | | |
| PS-45 | QUANTPEP-TTR- | 22 | 53 | N/A | N/A | N/A | 2454.14 |
| | TSESGELHGLTTEEEFVEGIYK | | | | | | |
| PS-46 | QUANTPEP-SEPP1- | 23 | 54 | N/A | N/A | N/A | 831.47 |
| | VSLATVDK | | | | | | |

### V.C. Classification Model for Distinguishing Between Patients Suffering from Sepsis Versus Other Conditions

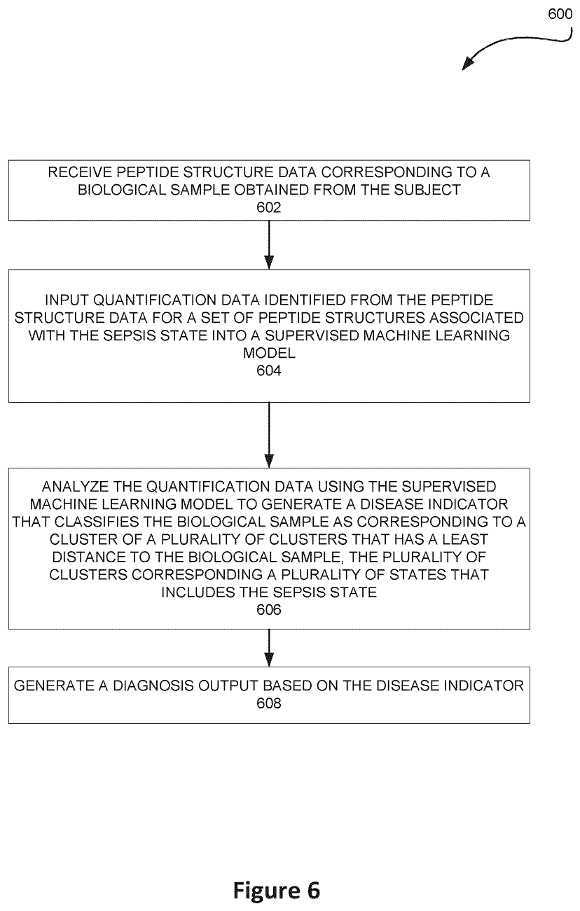

FIG. 6 is a flowchart of a process for determining whether a biological sample corresponds to a sepsis state in accordance with various embodiments. Process 600 may be implemented using, for example, at least a portion of workflow 100 as described in FIGS. 1, 2A, and 2B and/or analysis system 300 as described in FIG. 3. In some embodiments, process 600 may be one example of an implementation for process 500 in FIG. 5.

Process 600 may be one example of using a supervised machine learning model as a classifier for determining whether a given peptide structure profile of a biological sample obtained from a subject corresponds to one of a plurality of states (e.g., a sepsis state or another state). The peptide structure profile may include quantification data for a set of peptide structures that have been previously identified as being associated with the sepsis state.

Step 602 includes receiving peptide structure data corresponding to the biological sample obtained from a subject. The peptide structure data may have been generated using a reaction monitoring mass spectrometry system. For example, the peptide structure may have been generated using multiple reaction monitoring mass spectrometry (MRM-MS).

Step 604 includes inputting quantification data identified from the peptide structure data for a set of peptide structures associated with the sepsis state into a supervised machine learning model. The quantification data for a peptide structure of the set of peptide structures may include at least one of a relative quantity, an adjusted quantity, a normalized quantity, a relative concentration, an adjusted concentration, a normalized concentration, or some other type of quantification metric.

The set of peptide structures includes at least one peptide structure selected from a group of peptide structures identified in Table 1 above. The set of peptide structures may include at least one peptide structure that comprises a glycopeptide structure defined by a peptide sequence and a glycan structure linked to the peptide sequence at a linking site of the peptide sequence, as identified in Table 1, with the peptide sequence being one of SEQ ID NOS: 24-49 as defined in Table 3. The set of peptide structures may include at least one peptide structure that comprises an aglycosylated peptide structure defined by a peptide sequence, as identified in Table 1, with the peptide sequence being one of SEQ ID NOS: 50-54 as defined in Table 3.

Step 606 includes analyzing the quantification data using the supervised machine learning model to generate a disease indicator that classifies the biological sample as corresponding to a cluster of a plurality of clusters that has a least distance to the biological sample, the plurality of clusters corresponding a plurality of states that includes the sepsis state. The supervised machine learning model may include, for example, at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

The plurality of clusters may have been determined using, for example, an unsupervised machine learning model. For example, the supervised machine learning model may have been trained using training data generated from the unsupervised machine learning model. The training data may include a plurality of peptide structure profiles for a plurality of subjects and may identify a corresponding state of the plurality of states for each peptide structure profile of the plurality of peptide structure profiles. The unsupervised machine learning model may be trained to cluster the plurality of peptide structure profiles into the plurality of clusters. In various embodiments, the unsupervised machine learning model includes a k-means clustering model.

A peptide structure profile of the plurality of peptide structure profiles for a corresponding subject of the plurality of subjects may be selected based on a differential expression analysis of quantification metrics for a panel of peptide structures for the plurality of subjects. For example, the set of peptide structures included in the peptide structure profile may be determined based on the differential expression analysis. The differential expression analysis may be used to compare the quantification metrics corresponding to the panel of peptide structures for a first portion of the plurality of subjects diagnosed with the sepsis state to that of each portion of a set of other portions of the plurality of subjects to generate a set of comparisons. A portion of the panel of peptide structures having a false discovery rate below 0.05 across the set of comparisons may be selected as the set of peptide structures.

In some embodiments, the comparison of the quantification metrics may include comparing the quantification metrics corresponding to the panel of peptide structures for the first portion of the plurality of subjects diagnosed with the sepsis state to that of a second portion of the plurality of subjects diagnosed with a symptomatic disease state of COVID to generate a first comparison of the set of comparisons. The comparing may further include comparing quantification metrics corresponding to the panel of peptide structures for the first portion of the plurality of subjects diagnosed with the sepsis state to that of at least one of: a third portion of the plurality of subjects diagnosed with a common cold state to generate a second comparison of the set of comparisons; a fourth portion of the plurality of subjects diagnosed with a healthy state to generate a third comparison of the set of comparisons; and a fifth portion of the plurality of subjects diagnosed with an asymptomatic state of a coronavirus disease to generate a fourth comparison of the set of comparisons.

Step 608 includes generating a diagnosis output based on the disease indicator. The diagnosis output may be used to diagnose the patient with a high level of accuracy.

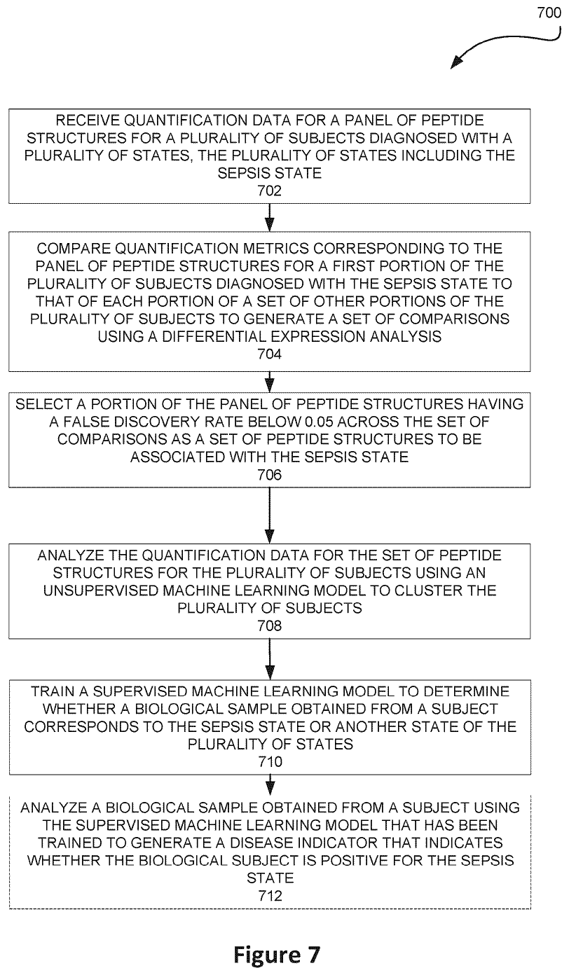

FIG. 7 is a flowchart of a process for identifying a sepsis-specific set of peptide structures for use in diagnosing a sepsis state of the coronavirus disease in accordance with one or more embodiments. Process 700 may be implemented using, for example, at least a portion of workflow 100 as described in FIGS. 1, 2A, and 2B and/or analysis system 300 as described in FIG. 3. In some embodiments, process 700 may be one example of an implementation for process 500 in FIG. 5.

Step 702 includes receiving quantification data for a panel of peptide structures for a plurality of subjects diagnosed with a plurality of states, the plurality of states including the sepsis state of the coronavirus disease.

Step 704 includes comparing quantification metrics corresponding to the panel of peptide structures for a first portion of the plurality of subjects diagnosed with the sepsis state to that of each portion of a set of other portions of the plurality of subjects to generate a set of comparisons using a differential expression analysis.

Step 706 includes selecting a portion of the panel of peptide structures having a false discovery rate below 0.05 across the set of comparisons as a set of peptide structures to be associated with the coronavirus disease. The set of peptide structures includes at least one glycopeptide structure defined by a peptide sequence and a glycan structure linked to the peptide sequence at a linking site of the peptide sequence.

Step 708 includes analyzing the quantification data for the set of peptide structures for the plurality of subjects using an unsupervised machine learning model to cluster the plurality of subjects.

Step 710 includes training a supervised machine learning model to determine whether a biological sample obtained from a subject corresponds to the sepsis state or another state of the plurality of states.

Process 700 may further include step 712. Step 712 includes analyzing a biological sample obtained from a subject using the supervised machine learning model that has been trained to generate a disease indicator that indicates whether the biological subject is positive for the sepsis state. The disease indicator may classify the biological sample as corresponding to a cluster of a plurality of clusters that has a least distance to the biological sample, the plurality of clusters corresponding a plurality of states that includes the sepsis state. The plurality of states may further include, for example, at least one of a sepsis state, an other state, an asepsis state, a healthy state, or a common cold state.

### VI. Exemplary Applications for Peptide Structure Analysis

### VI.A. Diagnosing Sepsis

The exemplary methodologies described in Section V may be used to diagnose a subject suffering from a sepsis state (or symptomatic COVID). The embodiments described herein may enable faster and more accurate diagnosis of a sepsis state. Being able to more quickly and accurately diagnose a subject (or patient) suffering from sepsis may enable treating the subject more quickly, which may lead to a more desirable treatment outcome for the subject. Further, being able to more quickly and accurately diagnose a subject (or patient) suffering from sepsis may be particularly useful in a hospital setting to help reduce hospitalization times and/or sepsis mortality.

### VI.B. Treating Sepsis

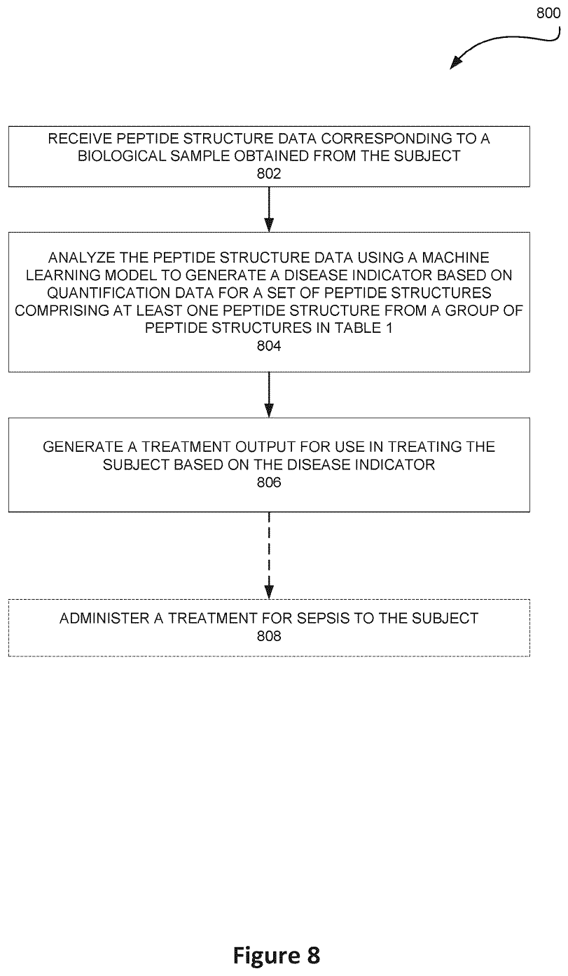

FIG. 8 is a flowchart of a process for treating a subject for a sepsis state in accordance with one or more embodiments. Process 800 may be implemented using at least a portion of workflow 100 as described FIGS. 1, 2A, and/or 2B and/or analysis system 300 as described in FIG. 3.

Step 802 includes receiving peptide structure data corresponding to a biological sample obtained from the subject.

Step 804 includes analyzing the peptide structure data using a machine learning model to generate a disease indicator based on quantification data for a set of peptide structures comprising at least one peptide structure from a group of peptide structures in Table 1. Step 804 may be implemented in various ways. For example, step 804 may be implemented using at least a portion of process 600 in FIG. 6, process 700 in FIG. 7, or process 800 in FIG. 8 as described above.

Step 806 includes generating a treatment output for use in treating the subject based on the disease indicator. The treatment output may include, for example, at least one of an identification of a therapeutic to treat the subject, a design for the therapeutic, or a treatment plan for administering the therapeutic. The therapeutic may include, for example, without limitation, one or more broad-spectrum antibiotics, one or more targeted antibiotics that are targeted against one or more particular types of bacteria, a vasopressor, or a combination thereof.

In some embodiments, process 800 further includes step 808. Step 808 includes administering a treatment for sepsis to the subject. Step 808 may include, for example, administering a therapeutic dosage of a therapeutic for sepsis to the subject. In some embodiments, the therapeutic may be an antibiotic treatment.

### VII. Peptide Structure and Product Ion Compositions, Kits and Reagents

Aspects of the disclosure include compositions comprising one or more of the peptide structures listed in Table 1. In some embodiments, a composition comprises a plurality of the peptide structures listed in Table 1. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 of the peptide structures listed in Table 1. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ NOs: 24-45, listed in Table 1.

Aspects of the disclosure include compositions comprising one or more precursor ions having a defined charge and/or defined mass-to-charge (m/z) ratio, as listed in Table 2. Aspects of the disclosure include compositions comprising one or more product ions having a defined mass-to-charge (m/z) ratio, which product ions are produced by converting a peptide structure described herein (e.g., a peptide structure listed in Table 1) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1). In some embodiments, a composition comprises a set of the product ions listed in Table 1, having an m/z ratio selected from the list provided for each peptide structure in Table 1.

In some embodiments, a composition comprises at least one of peptide structures PS-1 to PS-46 identified in Table 1.

In some embodiments, a composition comprises a peptide structure or a product ion. The peptide structure or product ion comprises an amino acid sequence having at least 90% sequence identity to any one of SEQ ID NOS: 24-54, as identified in Table 3, corresponding to peptide structures PS-1 to PS-46 in Table 1. The product ion is selected as one from a group consisting of product ions identified in Table 2 including product ions falling within an identified m/z range of the m/z ratio identified in Table 2 and characterized as having a precursor ion having an m/z ratio within an identified m/z range of the m/z ratio identified in Table 2. A first range for the product ion m/z ratio may be ±0.5. A first range for the product ion m/z ratio may be ±0.8. A third range for the product ion m/z ratio may be ±1.0. A first range for precursor ion m/z ratio may be ±1.0; a second range for the precursor ion m/z ratio may be (±1.5). Thus, the composition may include a product ion having an m/z ratio that falls within at least one of the first range (±0.5), the second range (±0.8), or the third range (±1.0) of the product ion m/z ratio identified in Table 2, and characterized as having a precursor ion having an m/z ratio that falls within at least one of a first range (±1.0) or a second range (±1.5) of the precursor ion m/z ratio identified in Table 2.

TABLE 2
Peptide Structures used with K-means Clustering Model

| | | | | | | | Second | Second |
|---|---|---|---|---|---|---|---|---|
| PS-ID | RT | Collision | Precursor | Precursor | Product | Product | Product | Product |
| NO. | (min) | Energy | M/Z | Charge | m/z | Charge | m/z | Charge |
| PS-1 | 35.45 | 30 | 1386.1 | 4 | 366.1 | 1 | N/A | N/A |
| PS-2 | 36.99 | 29 | 1167.3 | 5 | 366.1 | 1 | 1550.3 | 2 |
| PS-3 | 6.24 | 38 | 1270.2 | 3 | 366.1 | 1 | N/A | N/A |
| PS-4 | 17.95 | 35 | 1152.5 | 4 | 274.1 | 1 | N/A | N/A |
| PS-5 | 14.48 | 35 | 918.4 | 5 | 366.1 | 1 | 1292.7 | 2 |
| PS-6 | 24.22 | 20 | 1032.9 | 4 | 366.1 | 1 | 1208.6 | 2 |
| PS-7 | 24.67 | 25 | 1197 | 4 | 366.1 | 1 | 1208.6 | 2 |
| PS-8 | 14.56 | 22 | 984.4 | 5 | 274.1 | 1 | 1457.7 | 2 |
| PS-9 | 17.97 | 28 | 874.7 | 3 | 366.1 | 1 | N/A | N/A |
| PS-10 | 24.29 | 30 | 1225.8 | 4 | 366.1 | 1 | N/A | N/A |
| PS-11 | 11.13 | 33 | 1073.4 | 4 | 366.1 | 1 | N/A | N/A |
| PS-12 | 21.48 | 29 | 1183.5 | 4 | 366.1 | 1 | N/A | N/A |
| PS-13 | 5.24 | 30 | 1015.2 | 4 | 366.1 | 1 | 685.6 | 3 |
| PS-14 | 30.52 | 38 | 1284.6 | 4 | 366.1 | 1 | N/A | N/A |
| PS-15 | 30.49 | 32 | 1039.1 | 5 | 366.1 | 1 | N/A | N/A |
| PS-16 | 28.47 | 30 | 1285.1 | 4 | 366.1 | 1 | N/A | N/A |
| PS-17 | 42.81 | 28 | 1150.3 | 5 | 1249.3 | 3 | 366.1 | 1 |
| PS-18 | 22.63 | 30 | 1191.2 | 4 | 366.1 | 1 | 978.5 | 2 |
| PS-19 | 3.09 | 33 | 1050.4 | 3 | 366.1 | 1 | N/A | N/A |
| PS-20 | 3.1 | 26 | 788.3 | 4 | 366.1 | 1 | N/A | N/A |
| PS-21 | 5.67 | 21 | 878.8 | 3 | 204.1 | 1 | 1392.6 | 1 |
| PS-22 | 5.88 | 23 | 946.5 | 3 | 204.1 | 1 | 1392.6 | 1 |
| PS-23 | 10.52 | 35 | 1281.5 | 2 | 366.1 | 1 | 1360.6 | 1 |
| PS-24 | 10.61 | 21 | 873.4 | 3 | 204.1 | 1 | 1360.6 | 1 |
| PS-25 | 10.51 | 22 | 922.1 | 3 | 204.1 | 1 | 1360.6 | 1 |
| PS-26 | 11.3 | 30 | 764.8 | 4 | 204.1 | 1 | 1360.6 | 1 |
| PS-27 | 11.11 | 35 | 1087.1 | 3 | 204.1 | 1 | N/A | N/A |
| PS-28 | 10.44 | 24 | 976.1 | 3 | 366.1 | 1 | N/A | N/A |
| PS-29 | 11.13 | 26 | 1073.1 | 3 | 366.1 | 1 | N/A | N/A |
| PS-30 | 10.52 | 25 | 1043.8 | 3 | 366.1 | 1 | N/A | N/A |
| PS-31 | 17.14 | 25 | 891.8 | 3 | 1009 | 2 | 274.1 | 1 |
| PS-32 | 17.22 | 20 | 988.8 | 3 | 274.1 | 1 | 1009 | 2 |
| PS-33 | 34.96 | 35 | 1142 | 4 | 204.1 | 1 | N/A | N/A |
| PS-34 | 26.91 | 20 | 911.4 | 4 | 274.1 | 1 | 820.4 | 2 |
| PS-35 | 20.85 | 32 | 1041 | 4 | 366.1 | 1 | N/A | N/A |
| PS-36 | 45.24 | 30 | 1008.2 | 5 | 366.1 | 1 | N/A | N/A |
| PS-37 | 16.64 | 34 | 1109.8 | 3 | 366.1 | 1 | 829.4 | 2 |
| PS-38 | 11.08 | 20 | 761.7 | 3 | 274.1 | 1 | N/A | N/A |
| PS-39 | 19.42 | 22 | 939.1 | 3 | 274.1 | 1 | N/A | N/A |
| PS-40 | 39.53 | 24 | 1206.3 | 7 | 366.1 | 1 | N/A | N/A |
| PS-41 | 25.73 | 36 | 1199.2 | 4 | 366.1 | 1 | N/A | N/A |
| PS-42 | 29.99 | 20 | 697.4 | 2 | 565.3 | 1 | 922.4 | 1 |
| PS-43 | 26.57 | 17 | 618.3 | 2 | 736.4 | 1 | 936.5 | 1 |
| PS-44 | 22.53 | 15 | 590.3 | 2 | 342.2 | 1 | 725.4 | 1 |
| PS-45 | 24.63 | 25 | 819.1 | 3 | 609.3 | 1 | 855.5 | 1 |
| PS-46 | 10.39 | 10 | 416.7 | 2 | 646.4 | 1 | 733.4 | 1 |

Table 3 defines the peptide sequences for SEQ ID NOS: 24-54 from Table 1. Table 3 further identifies a corresponding protein SEQ ID NO for each peptide sequence. Each peptide sequence in Table 3 is defined as an amino acid sequence.

TABLE 3
Peptide SEQ ID NOS

| | | Corres- |
|---|---|---|
| SEQ | | ponding |
| ID | | Protein |
| NO: | Peptide Sequence | SEQ ID NO: |
| 24 | SVQEIQATFFYFTPNKTEDTIFLR | 1 |
| 25 | EDALNETR | 2 |
| 26 | ADGTVNQIEGEATPVNLTEPAK | 3 |
| 27 | NNATVHEQVGGPSLTSDLQAQSK | 4 |
| 28 | TLNQSSDELQLSMGNAMFVK | 5 |
| 29 | YPHKPEINSTTHPGADLQENFCR | 6 |
| 30 | NGSLFAFR | 4 |
| 31 | MVSHHNLTTGATLINEQWLLTTAK | 7 |
| 32 | EYESYSDFERNVTEK | 8 |
| 33 | VVLHPNYSQVDIGLIK | 7 |
| 34 | NGTGHGNSTHHGPEYMR | 9 |
| 35 | ICDLLVANNHFAHFFAPQNLTNMNK | 10 |
| 36 | YLGNATAIFFLPDEGKLQHLENELTHDIITK | 11 |
| 37 | YTGNASALFILPDQDK | 5 |
| 38 | HNSTGCLR | 2 |
| 39 | EEQYNSTYR | 12 |
| 40 | TVVQPSVGAAAGPVVPPCPGR | 14 |
| 41 | IVGGTNSSWGEWPWQVSLQVK | 15 |
| 42 | IYSGILNLSDITK | 15 |
| 43 | LQAPLNYTEFQKPICLPSK | 15 |
| 44 | ANLSSQALQMSLDYGFVTPLTSMSIR | 10 |
| 45 | QSVPAHFVALNGSK | 16 |
| 46 | SVSEINPTTQMK | 17 |
| 47 | TFVLSALQPSPTHSSSNTQR | 10 |
| 48 | ASVSVLGDILGSAMQNTQNLLQMPYGCGEQNM | 18 |
| | VLFAPNIYVLDYLNETQQLTPEIK | |
| 49 | ACLNSTYEYIFNNCHELYSR | 19 |
| 50 | ASSIIDELFQDR | 2 |
| 51 | DLATVYVDVLK | 20 |
| 52 | DLLLPQPDLR | 21 |
| 53 | TSESGELHGLTTEEEFVEGIYK | 22 |
| 54 | VSLATVDK | 23 |

Table 4 identifies the proteins of SEQ ID NOS: 1-23 from Table 1. Table 4 identifies a corresponding protein abbreviation and protein name for each of protein SEQ ID NOS: 1-23. Further, Table 4 identifies a corresponding Uniprot TD for each of protein SEQ ID NOS: 1-23.

TABLE 4
Protein SEQ ID NOS

| SEQ ID | Protein | | |
|---|---|---|---|
| NO: | Abbreviation | Protein Name | Uniprot ID |
| 1 | AGP12 | Alpha-1-acid glycoprotein 1&2 | P02763 & |
| | | | P19652 |
| 2 | CLUS | Clusterin | P10909 |
| 3 | APOD | Apolipoprotein D | P05090 |
| 4 | VTNC | Vitronectin | P04004 |
| 5 | AACT | Alpha-1-antichymotrypsin | P01011 |
| 6 | THRB | Prothrombin | P00734 |
| 7 | HPT | Haptoglobin | P00738 |
| 8 | CO8B | ComplementComponentC8BChain | P07358 |
| 9 | HEMO | Hemopexin | P02790 |
| 10 | ITIH1 | Inter-alpha-trypsin inhibitor | P19827 |
| | | heavy chain H1 | |
| 11 | A1AT | Alpha-1-antitrypsin | P01009 |
| 12 | IGG1 | Immunoglobulin heavy | P01857 |
| | | constant gamma 1 | |
| 13 | IGG2 | Immunoglobulin heavy | P01859 |
| | | constant gamma 2 | |
| 14 | FETUA | Alpha-2-HS-glycoprotein | P02765 |
| 15 | KLKB1 | Plasma Kallikrein | P03952 |
| 16 | CO2 | Complement C2 | P06681 |
| 17 | KNG1 | Kininogen-1 | P01042 |
| 18 | A2MG | Alpha-2-macroglobulin | P01023 |
| 19 | UN13A | Protein unc-13Homolog A | Q9UPW8 |
| 20 | APOA1 | Apolipoprotein A-I | P02647 |
| 21 | A2GL | Leucine-richAlpha-2-glycoprotein | P02750 |
| 22 | TTR | Transthyretin | P02766 |
| 23 | SEPP1 | Selenoprotein P | P49908 |

Table 5 identifies and defines the glycan structures from Table 1. Table 5 identifies a graphical representation of the structure and a coded representation of the composition for each glycan structure included in Table 1. As used herein, the 4-digit GL NO. is a designation that represents the number of hexoses, the number of HexNAcs, the number of Fucoses, and the number of Neuraminic Acids.

TABLE 5
Glycan Structure GL NOS: Structure and Composition

| Glycan Structure | | |
|---|---|---|
| GL NO. | Structure | Composition |
| 1101 | 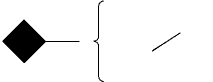 | Hex(1)HexNAc(1)Fuc(0)NeuAc(1) |
| 1102 | 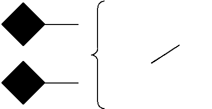 | Hex(1)HexNAc(1)Fuc(0)NeuAc(2) |
| 3410 | 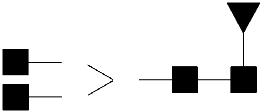 | Hex(3)HexNAc(4)Fuc(1)NeuAc(0) |
| 3510 | 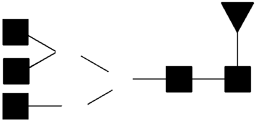 | Hex(3)HexNAc(5)Fuc(1)NeuAc(0) |
| | 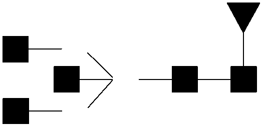 | |
| 4310 | 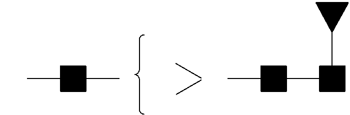 | Hex(4)HexNAc(3)Fuc(1)NeuAc(0) |
| 4400 | 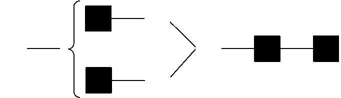 | Hex(4)HexNAc(4)Fuc(0)NeuAc(0) |
| 4410 | 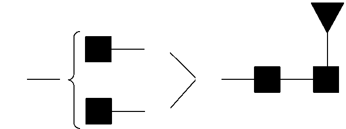 | Hex(4)HexNAc(4)Fuc(1)NeuAc(0) |
| 4411 | 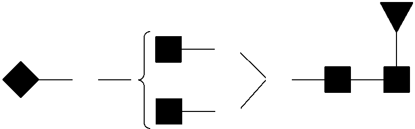 | Hex(4)HexNAc(4)Fuc(1)NeuAc(1) |
| 4511 | 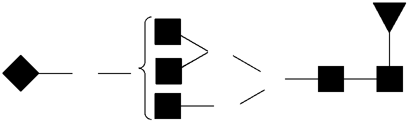 | Hex(4)HexNAc(5)Fuc(1)NeuAc(1) |
| 5301 | 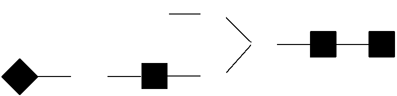 | Hex(5)HexNAc(3)Fuc(0)NeuAc(1) |
| 5401 | 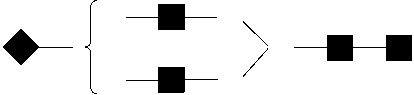 | Hex(5)HexNAc(4)Fuc(0)NeuAc(1) |
| 5402 | 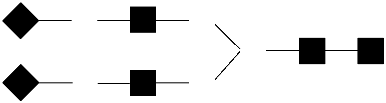 | Hex(5)HexNAc(4)Fuc(0)NeuAc(2) |
| 5410 | 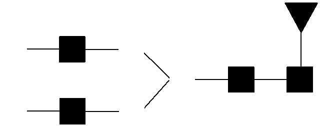 | Hex(5)HexNAc(4)Fuc(1)NeuAc(0) |
| 5411 | 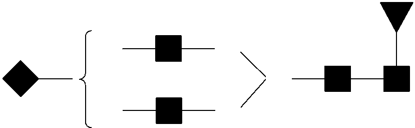 | Hex(5)HexNAc(4)Fuc(1)NeuAc(1) |
| 5412 | 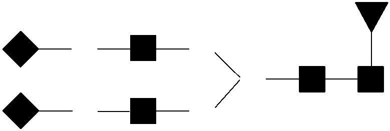 | Hex(5)HexNAc(4)Fuc(1)NeuAc(2) |
| 5421 | 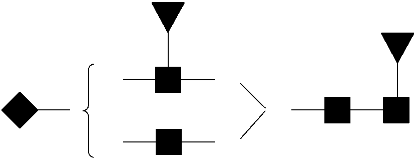 | Hex(5)HexNAc(4)Fuc(2)NeuAc(1) |
| 5510 | 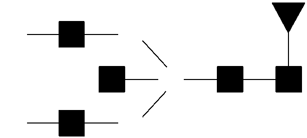 | Hex(5)HexNAc(5)Fuc(1)NeuAc(0) |
| 5511 | 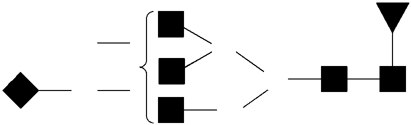 | Hex(5)HexNAc(5)Fuc(1)NeuAc(1) |
| | 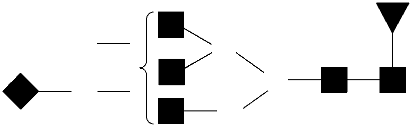 | |
| 6301 | 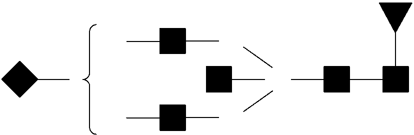 | Hex(6)HexNAc(3)Fuc(0)NeuAc(1) |
| | 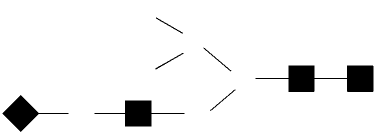 | |
| 6411 | 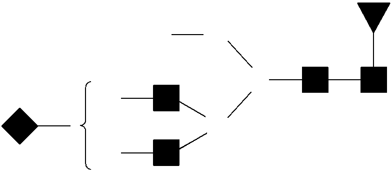 | Hex(6)HexNAc(4)Fuc(1)NeuAc(1) |
| 6502 | 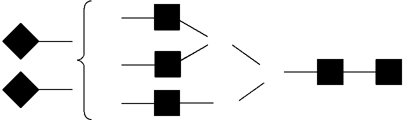 | Hex(6)HexNAc(5)Fuc(0)NeuAc(2) |
| 6503 | 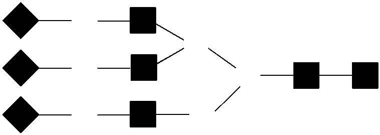 | Hex(6)HexNAc(5)Fuc(0)NeuAc(3) |
| 6513 | 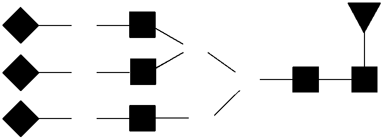 | Hex(6)HexNAc(5)Fuc(1)NeuAc(3) |
| 6610 | 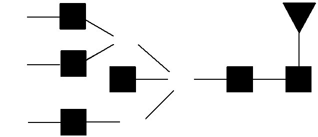 | Hex(6)HexNAc(6)Fuc(1)NeuAc(0) |
| 7420 | 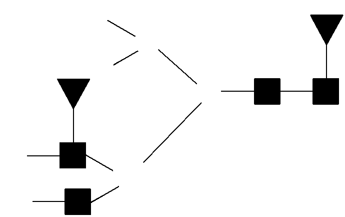 | Hex(7)HexNAc(4)Fuc(2)NeuAc(0) |
| 7601 | 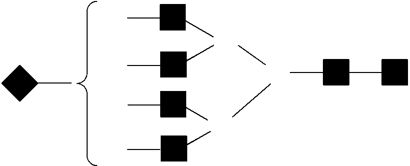 | Hex(7)HexNAc(6)Fuc(0)NeuAc(1) |
| 7602 | 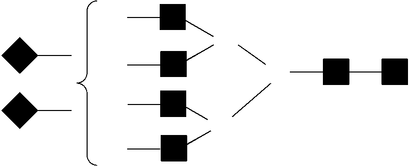 | Hex(7)HexNAc(6)Fuc(0)NeuAc(2) |

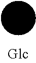

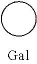

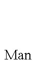

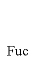

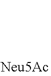

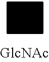

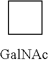

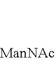

[symbol]

Xyl

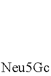

[symbol]

GlcN

[symbol]

GalN

[symbol]

ManN

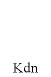

[symbol]

GlcA

[symbol]

GalA

[symbol]

ManA

[symbol]

IdoA

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating various disease conditions, including, without limitation, sepsis. A transition includes a precursor ion and at least one product ion grouping. As reviewed herein, the peptide structures in Table 1, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, sepsis.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 2 or an m/z ratio within an identified m/z ratio as provided in Table 2. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VIII. Representative Experimental Results

Objective: The objective of the exemplary experiment was to determine whether a set of peptide structures could be identified that could be used for accurate differentiation of patients suffering from symptomatic COVID, patients suffering from sepsis, and patients with other conditions including a healthy state, an asymptomatic disease state of COVID, and the common cold.

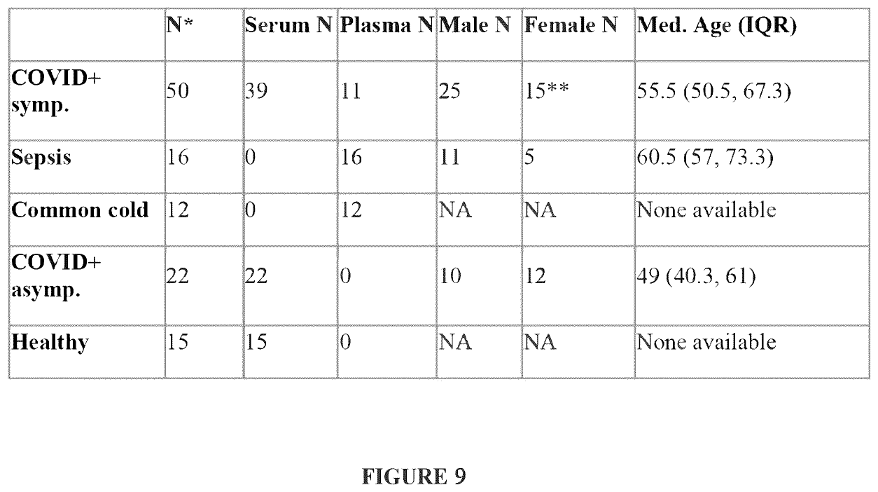

Methodology: In the exemplary experiment, 115 samples were collected from various sources, stored at −80 C, and processed through a targeted MRM panel, and analyzed. The 115 samples were collected for: 50 (39 serum, 11 plasma) patients who tested positive via PCR with severe/symptomatic COVID-19 infection, 22 serum samples from individuals who did not experience any symptoms of COVID-19 but whose serology tests were confirmed positive after infection, 16 plasma samples from patients who presented with sepsis (8 mild, 8 severe), 12 plasma samples from patients who had a common cold caused by a non-COVID-19 coronavirus at the time of blood draw, and 15 serum samples who were classified as historic healthy controls. FIG. 9 is a table that provides information about the subjects involved in this exemplary experiment, including, number, sample type, gender, and median age, where available in accordance with one or more embodiments.

A panel of 597 peptide structures were considered for analysis, consisting of 531 glycopeptide structures and 66 aglycosylated peptide structures. In order to compare two groups, differential expression analysis was performed. In particular, a linear regression was performed on a marker-by-marker basis with group membership serving as the sole binary independent variable. Because each of the 597 peptide structures was being compared simultaneously, corrections were made to achieve a FDR (false discovery rate) of less than 0.05 merits significance. A peptide structure achieving statistical significance implied that the mean normalized abundance between the two groups was significantly different. Overlapping sets of statistically significant peptide structures between sets of groups were then assessed. For example, differential expression analysis was performed to compare sepsis samples with the samples for each of the four other groups separately (four pairings).

Results: Of the panel of 597 peptide structures, 46 peptide structures (which may be referred to as markers or peptide structure markers) appeared in all four sets of the statistically significant peptide structures, where symptomatic COVID-19 was the primary comparison group. These 46 peptide structures formed a “sepsis-specific” signal.

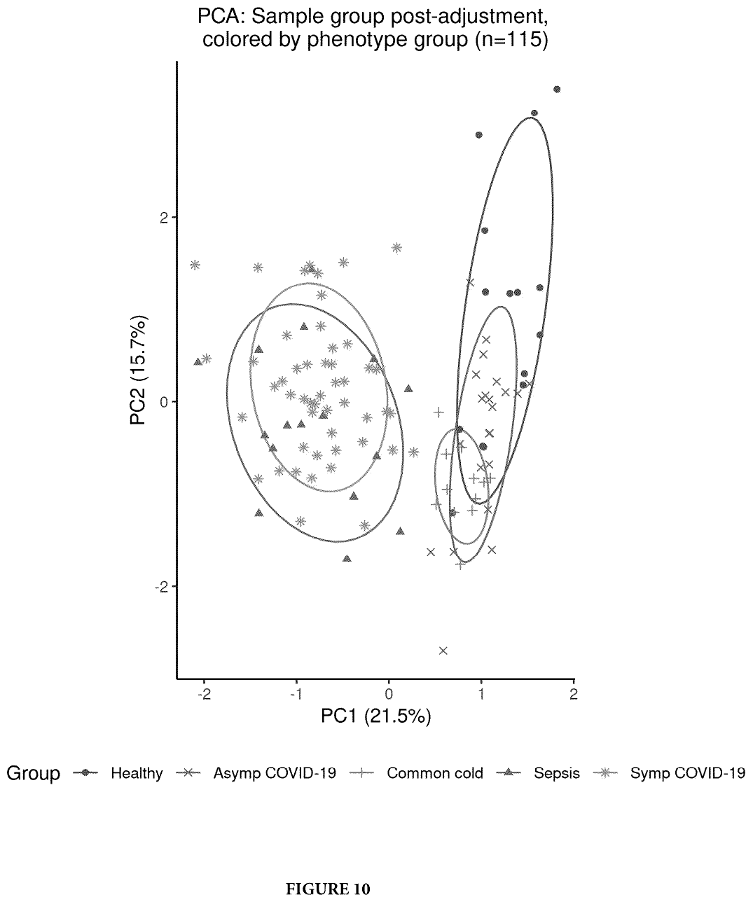

FIG. 10 is a plot showing a principal component analysis via a singular value decomposition of the centered and scaled data matrix of all 115 patient samples and all peptide structures (e.g., glycosylated and aglycosylated) of the panel. The vast degree of separation between groups, as shown in the plot supports the claim that there are multiple significant peptide structures (e.g., markers) between symptomatic COVID and other disease states.

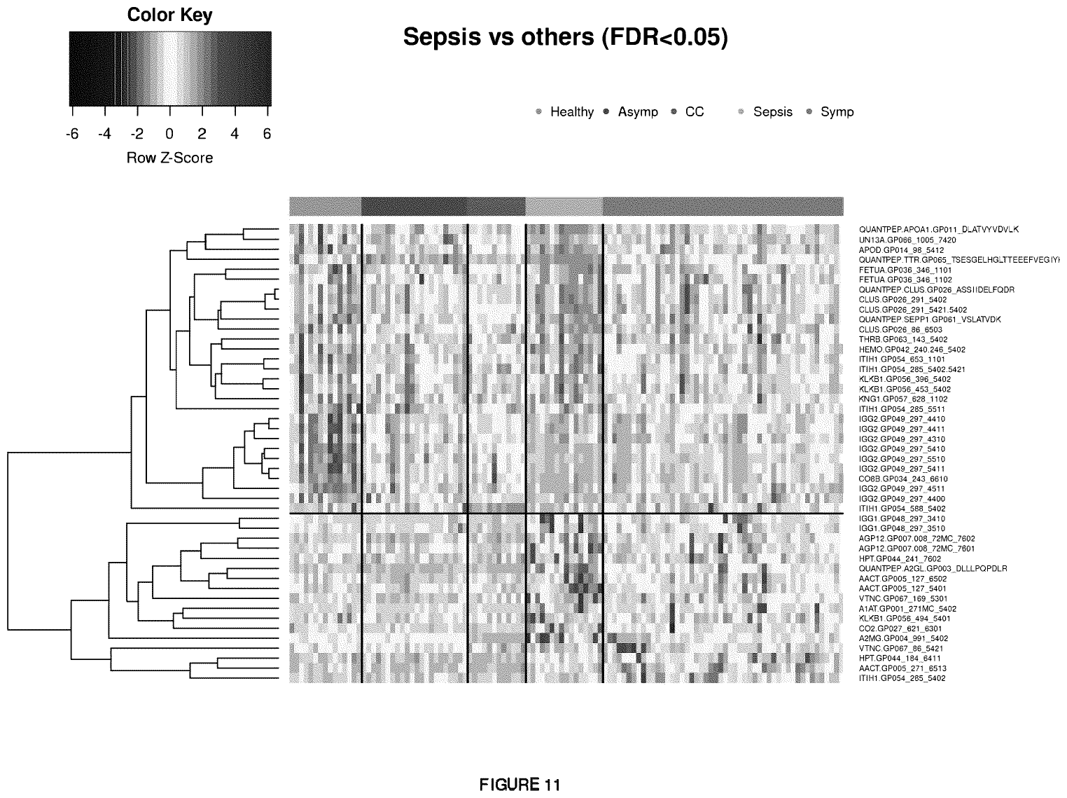

FIG. 11 is an illustration of a heat map depicting the 46 peptide structures, also identified in Table 1, in accordance with one or more embodiments. As indicated in FIG. 11, the 46 peptide structures are significantly differentially expressed between the sepsis state and other states, which include a healthy state, a common cold state, an asymptomatic disease state of COVID, or a symptomatic disease state of COVID.

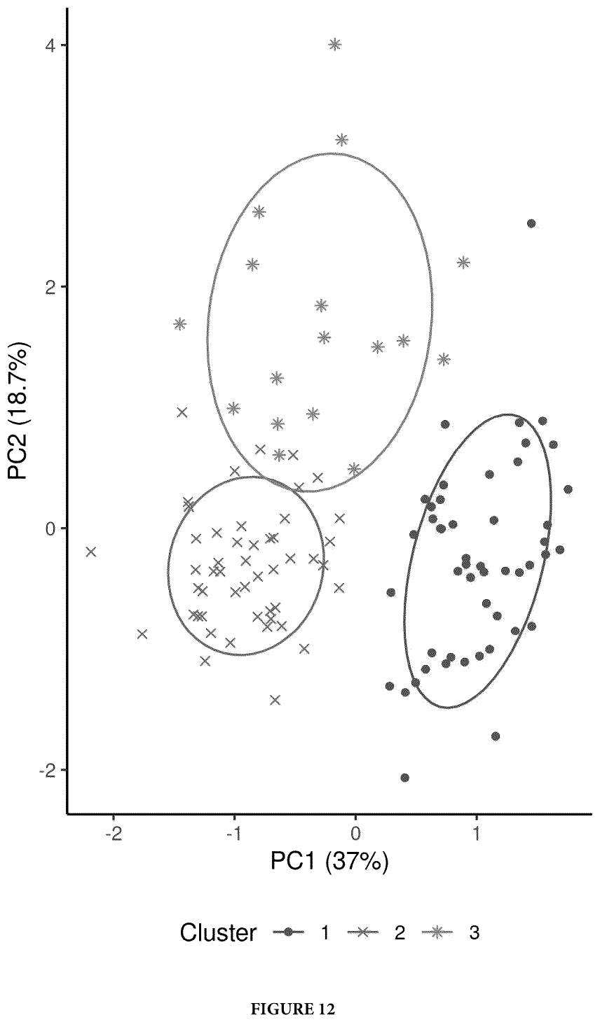

Validation: A k-means clustering model was used to determine whether the 46 peptide structures could accurately differentiate between sepsis and two other states: the symptomatic disease state of COVID and an “other” state. FIG. 12 is a plot that shows a k-means clustering graph using markers differentially expressed between sepsis and other groups in accordance with one or more embodiments. As indicated in FIG. 12, 88% of sepsis subjects are allocated to cluster 2, which is the cluster corresponding to the sepsis state.

### IX. Exemplary Methodology for Peptide Structure Data Analysis

### IX.A. Exemplary Methodology for Peptide Structure Data Analysis

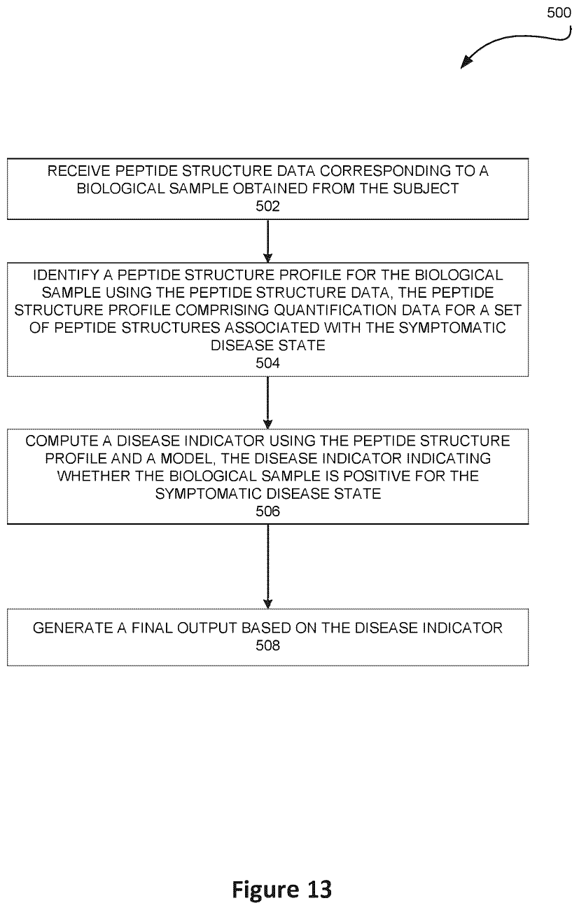

FIG. 13 is a flowchart of a process for evaluating a biological sample obtained from a subject with respect to a symptomatic disease state of COVID in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate at least one of a diagnosis output or a treatment output for the subject.

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample.

Step 504 includes identifying a peptide structure profile for the biological sample using the peptide structure data, the peptide structure profile comprising quantification data for a set of peptide structures associated with the symptomatic disease state. In step 504, the set of peptide structures includes at least two peptide structures from a selected group of peptide structures identified in Table 1-1 below. The selected group of peptide structures may be, for example, a portion of the peptide structure identified in Table 1-1. At least two peptide structures in the selected group of peptide structures includes a glycopeptide structure defined by a peptide sequence and a glycan structure linked to a linking site of the peptide sequence.

Step 506 includes computing a disease indicator using the peptide structure profile and a model, the disease indicator indicating whether the biological sample is positive for the symptomatic disease state. In various embodiments, the disease indicator comprises a probability that the biological sample is positive for the symptomatic disease state and the supervised machine learning model is configured to generate an output that identifies the biological sample as either positive for the symptomatic disease state when the disease indicator is greater than a selected threshold or negative for the symptomatic disease state when the disease indicator is not greater than the selected threshold.

In various embodiments, the disease indicator comprises a classification of the biological sample as belonging to a cluster that corresponds to the symptomatic disease state of COVID or another cluster corresponding to another state (e.g., a healthy state, an asymptomatic disease state of COVID, a common cold state, a sepsis state, some other state, or a combination thereof). In some embodiments, the disease indicator includes an identification of the cluster, an identification of the state corresponding to the cluster, or both.

Step 508 includes generating a final output based on the disease indicator. The final output may include, for example, without limitation, at least one of a diagnosis output or a treatment output. The diagnosis output may include the disease indicator, or a diagnosis made based on the disease indicator. The treatment output may include, for example, at least one of an identification of a treatment to treat the subject, a design for the treatment, a manufacturing plan for the treatment, or a treatment plan for administering the treatment.

TABLE 1-1
Peptide Structures associated with Symptomatic COVID

| | | | | | Linking | Linking | |
|---|---|---|---|---|---|---|---|
| | Peptide | (Protein) | (Peptide) | Mono- | Site Pos. | Site Pos. | Glycan |
| PS-ID | Structure (PS) | SEQ ID | SEQ ID | isotopic | in Protein | in Peptide | Structure |
| NO. | NAME | NO. | NO. | mass (Da) | Sequence | Sequence | GL NO. |
| PS-1 | ANGT-47_5401 | 55 | 84 | 4447.95 | 47 | 12 | 5401 |
| PS-2 | HEMO-64_5401 | 56 | 85 | 4440.74 | 64 | 15 | 5401 |
| PS-3 | AGP12-72_6503 | 57 | 86 | 4779.95 | 72 | 15 | 6503 |
| PS-4 | AGP12-72_6513 | 57 | 86 | 4926.00 | 72 | 15 | 6513 |
| PS-5 | AGP12-72MC_7602 | 57 | 87 | 5829.49 | 72 | 15 | 7602 |
| PS-6 | CFAI-103_5402 | 58 | 88 | 3515.36 | 103 | 4 | 5402 |
| PS-7 | ZA2G-112_5420 | 59 | 89 | 3979.65 | 112 | 10 | 5420 |
| PS-8 | CERU-138_5412 | 60 | 90 | 4242.66 | 138 | 10 | 5412 |
| PS-9 | VTNC-169_5301 | 61 | 91 | 2621.06 | 169 | 1 | 5301 |
| PS-10 | HPT-184_6411 | 62 | 92 | 4900.17 | 184 | 6 | 6411 |
| PS-11 | HPT-241_7602 | 62 | 93 | 4729.04 | 241 | 6 | 7602 |
| PS-12 | IC1-253_5412 | 63 | 94 | 4450.92 | 253 | 4 | 5412 |
| PS-13 | IC1-253_6513 | 63 | 94 | 5107.14 | 253 | 4 | 6513 |
| PS-14 | APOH-253_5401 | 64 | 95 | 3163.24 | 253 | 3 | 5401 |
| PS-15 | ITIH1-285_5402/5421 | 65 | 96 | 5133.19 | 285 | 19 | 5402 |
| PS-16 | ITIH1-285_5402 | 65 | 96 | 5133.19 | 285 | 19 | 5402 |
| PS-17 | A1AT-271_5402 | 66 | 97 | 3959.66 | 271 | 4 | 5402 |
| PS-18 | A1AT-271_5412 | 66 | 97 | 4105.72 | 271 | 4 | 5412 |
| PS-19 | A1AT-271_6503 | 66 | 97 | 4615.89 | 271 | 4 | 6503 |
| PS-20 | AACT-271_6513 | 67 | 98 | 4758.93 | 271 | 4 | 6513 |
| PS-21 | IGG1-180_3410 | 68 | 99 | 2633.04 | 180 | 5 | 3410 |
| PS-22 | IGG2-176_5410 | 69 | 99 | 2925.15 | 176 | 5 | 5410 |
| PS-23 | IGG2-176_5510 | 69 | 99 | 3128.23 | 176 | 5 | 5510 |
| PS-24 | CAN3-366_6503 | 70 | 100 | 4677.79 | 366 | 9 | 6503 |
| PS-25 | CFAH-529_5402 | 71 | 101 | 6822.70 | 529 | 2 | 5402 |
| PS-26 | ITIH1-653_1101 | 65 | 102 | 2813.31 | 653 | 12 | 1101 |
| PS-27 | CERU-762_6523 | 60 | 103 | 5174.11 | 762 | 9 | 6523 |
| PS-28 | CFAH-882_5420/5401 | 71 | 104 | 3933.66 | 882 | 15 | 5401 |
| PS-29 | APOB-983_5401 | 72 | 105 | 5463.24 | 983 | 16 | 5401 |
| PS-30 | UN13A-1005_7420 | 73 | 106 | 4791.91 | 1005 | 4 | 7420 |
| PS-31 | APOA1- | 74 | 107 | 1234.68 | N/A | N/A | N/A |
| | DLATVYVDVLK | | | | | | |
| PS-32 | A2GL-DLLLPQPDLR | 75 | 108 | 1178.67 | N/A | N/A | N/A |
| PS-33 | TTR- | 76 | 109 | 2454.14 | N/A | N/A | N/A |
| | TSESGELHGLTTEEEFVEGIYK | | | | | | |
| PS-34 | KNG1-YFIDFVAR | 77 | 110 | 1029.53 | N/A | N/A | N/A |
| PS-35 | AGP1-93_7614 | 78 | 111 | 5578.17 | 93 | 7 | 7614 |
| PS-36 | AACT-271_6503 | 67 | 98 | 4612.87 | 271 | 4 | 6503 |
| PS-37 | THRB-416MC_5401 | 79 | 112 | 5084.28 | 416 | 17 | 5401 |
| PS-38 | HPT-241_5411 | 62 | 93 | 3853.74 | 241 | 6 | 5411 |
| PS-39 | AACT-271_6502 | 67 | 98 | 4321.78 | 271 | 4 | 6502 |
| PS-40 | A2MG-991_5402 | 80 | 113 | 8432.83 | 991 | 46 | 5402 |
| PS-41 | A1AT-271MC_5412 | 66 | 97 | 5890.66 | 271 | 4 | 5412 |
| PS-42 | PON1-YVYIAELLAHK | 81 | 114 | 1318.73 | N/A | N/A | N/A |
| PS-43 | HRG-125_5420/5401 | 82 | 115 | 3927.64 | 125 | 5 | 5401 |
| PS-44 | CFAH-882_5411 | 71 | 104 | 4079.71 | 882 | 15 | 5411 |
| PS-45 | ANT3- | 83 | 116 | 1527.74 | N/A | N/A | N/A |
| | FATTFYQHLADSK | | | | | | |

### IX.A.1. Example 1: Classification Model for Distinguishing Between Patients Suffering from Symptomatic COVID Versus Other Conditions

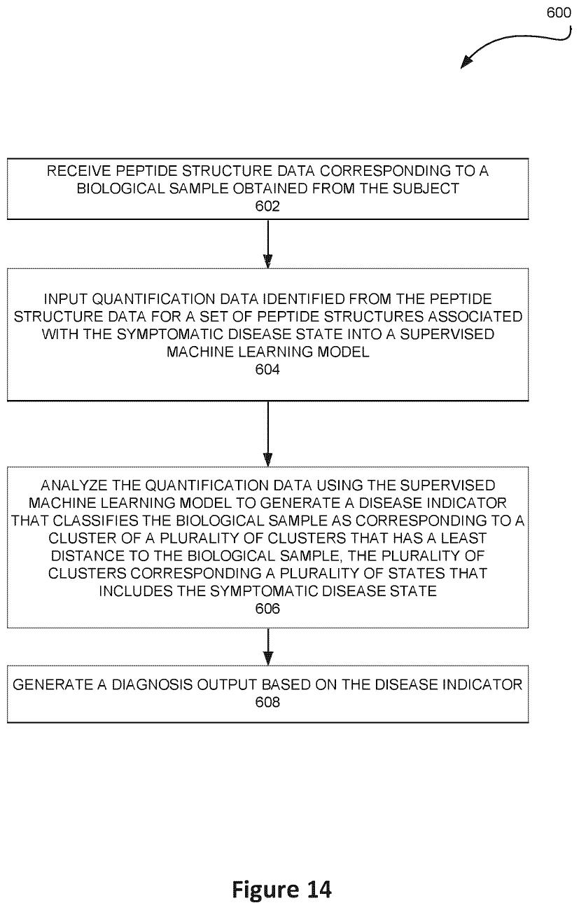

FIG. 14 is a flowchart of a process for determining whether a biological sample corresponds to a symptomatic disease state of a coronavirus disease (COVID) in accordance with various embodiments. Process 600 may be implemented using, for example, at least a portion of workflow 100 as described in FIGS. 1, 2A, and 2B and/or analysis system 300 as described in FIG. 3. In some embodiments, process 600 may be one example of an implementation for process 500 in FIG. 13.

Process 600 may be one example of using a supervised machine learning model as a classifier for determining whether a given peptide structure profile of a biological sample obtained from a subject corresponds to one of a plurality of states (e.g., a symptomatic disease state of COVID or another state). The peptide structure profile may include quantification data for a set of peptide structures that have been previously identified as being associated with the symptomatic disease state of COVID.

Step 602 includes receiving peptide structure data corresponding to the biological sample obtained from a subject. The peptide structure data may have been generated using a reaction monitoring mass spectrometry system. For example, the peptide structure may have been generated using multiple reaction monitoring mass spectrometry (MIRM-MS).

Step 604 includes inputting quantification data identified from the peptide structure data for a set of peptide structures associated with the symptomatic disease state into a supervised machine learning model. The quantification data for a peptide structure of the set of peptide structures may include at least one of a relative quantity, an adjusted quantity, a normalized quantity, a relative concentration, an adjusted concentration, a normalized concentration, or some other type of quantification metric.

The set of peptide structures includes at least one peptide structure selected from a group of peptide structures identified in Table 2-1 below. The set of peptide structures may include at least one peptide structure that comprises a glycopeptide structure defined by a peptide sequence and a glycan structure linked to the peptide sequence at a linking site of the peptide sequence, as identified in Table 1-1, with the peptide sequence being one of SEQ ID NOS: 84-106 as defined in Table 5-1. The set of peptide structures may include at least one peptide structure that comprises an aglycosylated peptide structure defined by a peptide sequence, as identified in Table 1-1, with the peptide sequence being one of SEQ ID NOS: 107-110 as defined in Table 5-1.

Step 606 includes analyzing the quantification data using the supervised machine learning model to generate a disease indicator that classifies the biological sample as corresponding to a cluster of a plurality of clusters that has a least distance to the biological sample, the plurality of clusters corresponding a plurality of states that includes the symptomatic disease state. The supervised machine learning model may include, for example, at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

The plurality of clusters may have been determined using, for example, an unsupervised machine learning model. For example, the supervised machine learning model may have been trained using training data generated from the unsupervised machine learning model. The training data may include a plurality of peptide structure profiles for a plurality of subjects and may identify a corresponding state of the plurality of states for each peptide structure profile of the plurality of peptide structure profiles. The unsupervised machine learning model may be trained to cluster the plurality of peptide structure profiles into the plurality of clusters. In various embodiments, the unsupervised machine learning model includes a k-means clustering model.

A peptide structure profile of the plurality of peptide structure profiles for a corresponding subject of the plurality of subjects may be selected based on a differential expression analysis of quantification metrics for a panel of peptide structures for the plurality of subjects. For example, the set of peptide structures included in the peptide structure profile may be determined based on the differential expression analysis. The differential expression analysis may be used to compare the quantification metrics corresponding to the panel of peptide structures for a first portion of the plurality of subjects diagnosed with the symptomatic disease state of COVID to that of each portion of a set of other portions of the plurality of subjects to generate a set of comparisons. A portion of the panel of peptide structures having a false discovery rate below 0.05 across the set of comparisons may be selected as the set of peptide structures.

In some embodiments, the comparison of the quantification metrics may include comparing the quantification metrics corresponding to the panel of peptide structures for the first portion of the plurality of subjects diagnosed with the symptomatic disease state of COVID to that of a second portion of the plurality of subjects diagnosed with a sepsis state to generate a first comparison of the set of comparisons. The comparing may further include comparing quantification metrics corresponding to the panel of peptide structures for the first portion of the plurality of subjects diagnosed with the symptomatic disease state of COVID to that of at least one of: a third portion of the plurality of subjects diagnosed with a common cold state to generate a second comparison of the set of comparisons; a fourth portion of the plurality of subjects diagnosed with a healthy state to generate a third comparison of the set of comparisons; and a fifth portion of the plurality of subjects diagnosed with an asymptomatic state of COVID to generate a fourth comparison of the set of comparisons.

Step 608 includes generating a diagnosis output based on the disease indicator. The diagnosis output may be used to diagnose the patient with a high level of accuracy.

TABLE 2-1
Peptide Structures used with K-Means Clustering Model

| | | | | | Linking | Linking | |
|---|---|---|---|---|---|---|---|
| | Peptide | (Protein) | (Peptide) | Mono- | Site Pos. | Site Pos. | Glycan |
| PS-ID | Structure (PS) | SEQ ID | SEQ ID | isotopic | in Protein | in Peptide | Structure |
| NO. | NAME | NO. | NO. | mass (Da) | Sequence | Sequence | GL NO. |
| PS-1 | ANGT-47_5401 | 55 | 84 | 4447.95 | 47 | 12 | 5401 |
| PS-2 | HEMO-64_5401 | 56 | 85 | 4440.74 | 64 | 15 | 5401 |
| PS-3 | AGP12-72_6503 | 57 | 86 | 4779.95 | 72 | 15 | 6503 |
| PS-4 | AGP12-72_6513 | 57 | 86 | 4926.00 | 72 | 15 | 6513 |
| PS-5 | AGP12-72MC_7602 | 57 | 87 | 5829.49 | 72 | 15 | 7602 |
| PS-6 | CFAI-103_5402 | 58 | 88 | 3515.36 | 103 | 4 | 5402 |
| PS-7 | ZA2G-112_5420 | 59 | 89 | 3979.65 | 112 | 10 | 5420 |
| PS-8 | CERU-138_5412 | 60 | 90 | 4242.66 | 138 | 10 | 5412 |
| PS-9 | VTNC-169_5301 | 61 | 91 | 2621.06 | 169 | 1 | 5301 |
| PS-10 | HPT-184_6411 | 62 | 92 | 4900.17 | 184 | 6 | 6411 |
| PS-11 | HPT-241_7602 | 62 | 93 | 4729.04 | 241 | 6 | 7602 |
| PS-12 | IC1-253_5412 | 63 | 94 | 4450.92 | 253 | 4 | 5412 |
| PS-13 | IC1-253_6513 | 63 | 94 | 5107.14 | 253 | 4 | 6513 |
| PS-14 | APOH-253_5401 | 64 | 95 | 3163.24 | 253 | 3 | 5401 |
| PS-15 | ITIH1-285_5402/5421 | 65 | 96 | 5133.19 | 285 | 19 | 5402 |
| PS-16 | ITIH1-285_5402 | 65 | 96 | 5133.19 | 285 | 19 | 5402 |
| PS-17 | A1AT-271_5402 | 66 | 97 | 3959.66 | 271 | 4 | 5402 |
| PS-18 | A1AT-271_5412 | 66 | 97 | 4105.72 | 271 | 4 | 5412 |
| PS-19 | A1AT-271_6503 | 66 | 97 | 4615.89 | 271 | 4 | 6503 |
| PS-20 | AACT-271_6513 | 67 | 98 | 4758.93 | 271 | 4 | 6513 |
| PS-21 | IGG1-180_3410 | 68 | 99 | 2633.04 | 180 | 5 | 3410 |
| PS-22 | IGG2-176_5410 | 69 | 99 | 2925.15 | 176 | 5 | 5410 |
| PS-23 | IGG2-176_5510 | 69 | 99 | 3128.23 | 176 | 5 | 5510 |
| PS-24 | CAN3-366_6503 | 70 | 100 | 4677.79 | 366 | 9 | 6503 |
| PS-25 | CFAH-529_5402 | 17 | 101 | 6822.70 | 529 | 2 | 5402 |
| PS-26 | ITIH1-653_1101 | 65 | 102 | 2813.31 | 653 | 12 | 1101 |
| PS-27 | CERU-762_6523 | 60 | 103 | 5174.11 | 762 | 9 | 6523 |
| PS-28 | CFAH-882_5420/5401 | 71 | 104 | 3933.66 | 882 | 15 | 5401 |
| PS-29 | APOB-983_5401 | 72 | 105 | 5463.24 | 983 | 16 | 5401 |
| PS-30 | UN13A-1005_7420 | 73 | 106 | 4791.91 | 1005 | 4 | 7420 |
| PS-31 | APOA1- | 74 | 107 | 1234.68 | N/A | N/A | N/A |
| | DLATVYVDVLK | | | | | | |
| PS-32 | A2GL-DLLLPQPDLR | 75 | 108 | 1178.67 | N/A | N/A | N/A |
| PS-33 | TTR- | 76 | 109 | 2454.14 | N/A | N/A | N/A |
| | TSESGELHGLTTEEEFVEGIYK | | | | | | |
| PS-34 | KNG1-YFIDFVAR | 77 | 110 | 1029.53 | N/A | N/A | N/A |

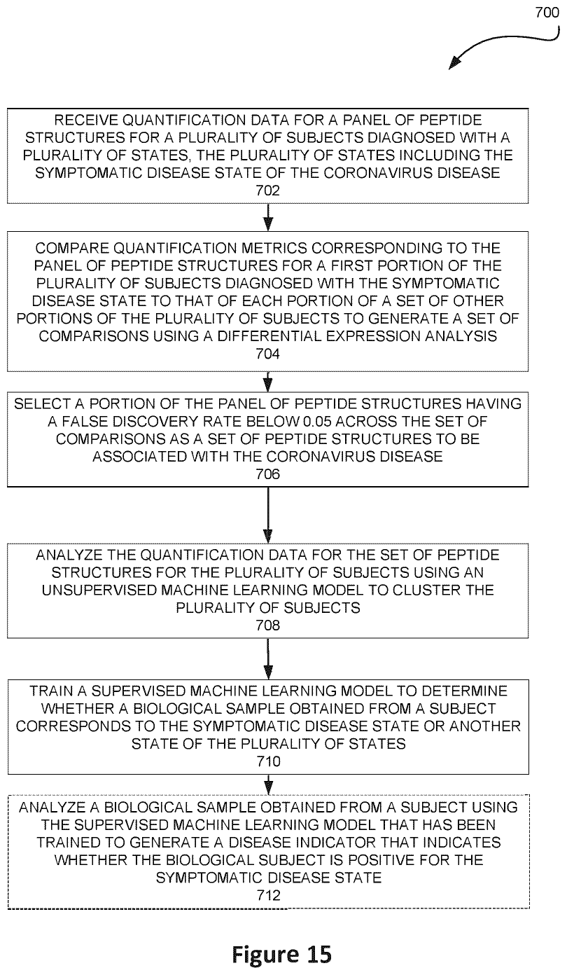

FIG. 15 is a flowchart of a process for identifying a coronavirus disease (COVID)-specific set of peptide structures for use in diagnosing a symptomatic disease state of the coronavirus disease in accordance with one or more embodiments. Process 700 may be implemented using, for example, at least a portion of workflow 100 as described in FIGS. 1, 2A, and 2B and/or analysis system 300 as described in FIG. 3. In some embodiments, process 700 may be one example of an implementation for process 500 in FIG. 13.

Step 702 includes receiving quantification data for a panel of peptide structures for a plurality of subjects diagnosed with a plurality of states, the plurality of states including the symptomatic disease state of the coronavirus disease.

Step 704 includes comparing quantification metrics corresponding to the panel of peptide structures for a first portion of the plurality of subjects diagnosed with the symptomatic disease state to that of each portion of a set of other portions of the plurality of subjects to generate a set of comparisons using a differential expression analysis.

Step 706 includes selecting a portion of the panel of peptide structures having a false discovery rate below 0.05 across the set of comparisons as a set of peptide structures to be associated with the coronavirus disease. The set of peptide structures includes at least one glycopeptide structure defined by a peptide sequence and a glycan structure linked to the peptide sequence at a linking site of the peptide sequence.

Step 708 includes analyzing the quantification data for the set of peptide structures for the plurality of subjects using an unsupervised machine learning model to cluster the plurality of subjects. The unsupervised machine learning model may include, for example, a k-means clustering model.

Step 710 includes training a supervised machine learning model to determine whether a biological sample obtained from a subject corresponds to the symptomatic disease state or another state of the plurality of states. The supervised machine learning model may include, for example, at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm Process 700 may further include step 712. Step 712 includes analyzing a biological sample obtained from a subject using the supervised machine learning model that has been trained to generate a disease indicator that indicates whether the biological subject is positive for the symptomatic disease state. The disease indicator may classify the biological sample as corresponding to a cluster of a plurality of clusters that has a least distance to the biological sample, the plurality of clusters corresponding a plurality of states that includes the symptomatic disease state. The plurality of states may further include, for example, at least one of a sepsis state, an other state, an asymptomatic disease state of COVID, a healthy state, or a common cold state.

### IX.A.2. Example 2: Regression Model to Determine Likelihood that Patient Suffering from Symptomatic COVID

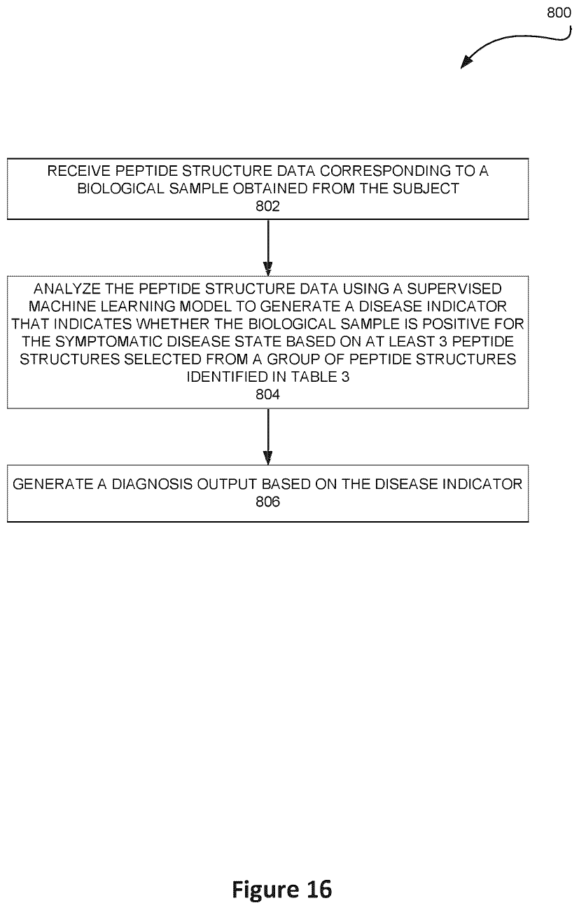

FIG. 16 is a flowchart of a process for diagnosing a symptomatic disease state of a coronavirus disease (COVID) in accordance with one or more embodiments. Process 800 may be implemented using, for example, at least a portion of workflow 100 as described in FIGS. 1, 2A, and 2B and/or analysis system 300 as described in FIG. 3. In some embodiments, process 800 may be one example of an implementation for process 500 in FIG. 13.

Step 802 includes receiving peptide structure data corresponding to a biological sample obtained from a subject. The peptide structure data may have been generated from a prepared sample using, for example, multiple reaction monitoring mass spectrometry (MRM-MS). The peptide structure data may include quantification data for each peptide structure of a panel of peptide structures. The quantification data for a peptide structure of the plurality of peptide structures may include at least one of a relative quantity, an adjusted quantity, a normalized quantity, a relative concentration, an adjusted concentration, a normalized concentration, or another quantification metric.

Step 804 includes analyzing the peptide structure data using a supervised machine learning model to generate a disease indicator that indicates whether the biological sample is positive for the symptomatic disease state based on at least 3 peptide structures selected from a group of peptide structures identified in Table 3-1. The group of peptide structures in Table 3-1 (below) comprises a group of peptide structures associated with the symptomatic disease state. The group of peptide structures is listed in Table 3-1 with respect to relative significance to the disease indicator.

The disease indicator that is generated may include, for example, at least one of a probability that the subject is positive for the symptomatic disease state, an odds that the subject is positive for the symptomatic disease state, a logarithm of the odds that the subject is positive for the symptomatic disease state, or a classification of the biological sample as either positive or negative for the symptomatic disease state.

In one or more embodiments, step 804 may be implemented using a regression model. In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures indicating the relative significance of the corresponding peptide structure to the disease indicator. In some embodiments, step 804 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In one or more embodiments, the disease indicator comprises a probability that the biological sample is positive for the symptomatic disease state and the supervised machine learning model is configured to generate an output that identifies the biological sample as either positive for the symptomatic disease state when the disease indicator is greater than a selected threshold or negative for the symptomatic disease state when the disease indicator is not greater than the selected threshold. The selected threshold may be, for example, a value selected as either equal to or above 0.5. In some cases, the selected threshold may be a value within ±0.02 of 0.525.

In various embodiments, the supervised machine learning model is trained to determine weight coefficients for a panel of peptide structures such that a first portion of the weight coefficients for a first portion of the panel of peptide structures are non-zero and a second portion of the weight coefficients for a second portion of the panel of peptide structures are zero, with the first portion of the panel of peptide structures forming the group of peptide structures identified in Table 3-1. In some embodiments, the supervised machine learning model is trained using training data that comprises a plurality of peptide structure profiles for a plurality of subjects and a corresponding state of a plurality of states for each peptide structure profile of the plurality of peptide structure profiles. The plurality of subjects may include, for example, a first portion diagnosed with the symptomatic disease state and at least one of: a second portion having a healthy state; a third portion diagnosed with a common cold state; a fourth portion diagnosed with an asymptomatic disease state of the coronavirus disease (COVID); or a fifth portion diagnosed with a sepsis state.

Step 806 includes generating a diagnosis output based on the disease indicator.

TABLE 3-1
Peptide Structure Markers for Regression Model for COVID

| | | | | | Linking | Linking | |
|---|---|---|---|---|---|---|---|
| | Peptide | (Protein) | (Peptide) | Mono- | Site Pos. | Site Pos. | Glycan |
| PS-ID | Structure (PS) | SEQ ID | SEQ ID | isotopic | in Protein | in Peptide | Structure |
| NO. | NAME | NO. | NO. | mass (Da) | Sequence | Sequence | GL NO. |
| PS-35 | AGP1-93_7614 | 78 | 111 | 5578.17 | 93 | 7 | 7614 |
| PS-36 | AACT-271_6503 | 67 | 98 | 4612.87 | 271 | 4 | 6503 |
| PS-10 | HPT-184_6411 | 62 | 92 | 4900.17 | 184 | 6 | 6411 |
| PS-37 | THRB-416MC_5401 | 79 | 112 | 5084.28 | 416 | 17 | 5401 |
| PS-38 | HPT-241_5411 | 62 | 93 | 3853.74 | 241 | 6 | 5411 |
| PS-39 | AACT-271_6502 | 67 | 98 | 4321.78 | 271 | 4 | 6502 |
| PS-13 | IC1-253_6513 | 63 | 94 | 5107.14 | 253 | 4 | 6513 |
| PS-40 | A2MG-991_5402 | 80 | 113 | 8432.83 | 991 | 46 | 5402 |
| PS-41 | A1AT-271MC_5412 | 66 | 97 | 5890.66 | 271 | 4 | 5412 |
| PS-42 | PON1-YVYIAELLAHK | 81 | 114 | 1318.73 | N/A | N/A | N/A |
| PS-1 | ANGT-47_5401 | 55 | 84 | 4447.95 | 47 | 12 | 5401 |
| PS-19 | A1AT-271_6503 | 66 | 97 | 4615.89 | 271 | 4 | 6503 |
| PS-43 | HRG-125_5420/5401 | 82 | 115 | 3927.64 | 125 | 5 | 5401 |
| PS-28 | CFAH-882_5420/5401 | 71 | 104 | 3933.66 | 882 | 15 | 5401 |
| PS-44 | CFAH-882_5411 | 71 | 104 | 4079.71 | 882 | 15 | 5411 |
| PS-45 | ANT3- | 83 | 116 | 1527.74 | N/A | N/A | N/A |
| | FATTFYQHLADSK | | | | | | |
| PS-16 | ITIH1-285_5402 | 65 | 96 | 5133.19 | 285 | 19 | 5402 |
| PS-2 | HEMO-64_5401 | 56 | 85 | 4440.74 | 64 | 15 | 5401 |

### X. Exemplary Applications for Peptide Structure Analysis

### X.A. Diagnosing COVID

The exemplary methodologies described in Section V may be used to diagnose a subject suffering from a symptomatic disease state of COVID (or symptomatic COVID). This diagnosis may be used to determine a method of treatment for a subject. The embodiments described herein may enable faster and more accurate diagnosis of the symptomatic disease state of COVID. Being able to more quickly and accurately diagnose a subject (or patient) suffering from symptomatic COVID may enable treating the subject more quickly, which may lead to a more desirable treatment outcome for the subject. Further, being able to more quickly and accurately diagnose a subject (or patient) suffering from symptomatic COVID may be particularly useful in a hospital setting to help reduce hospitalization times and/or sepsis or other COVID-related mortality.

### X.B. Treating COVID

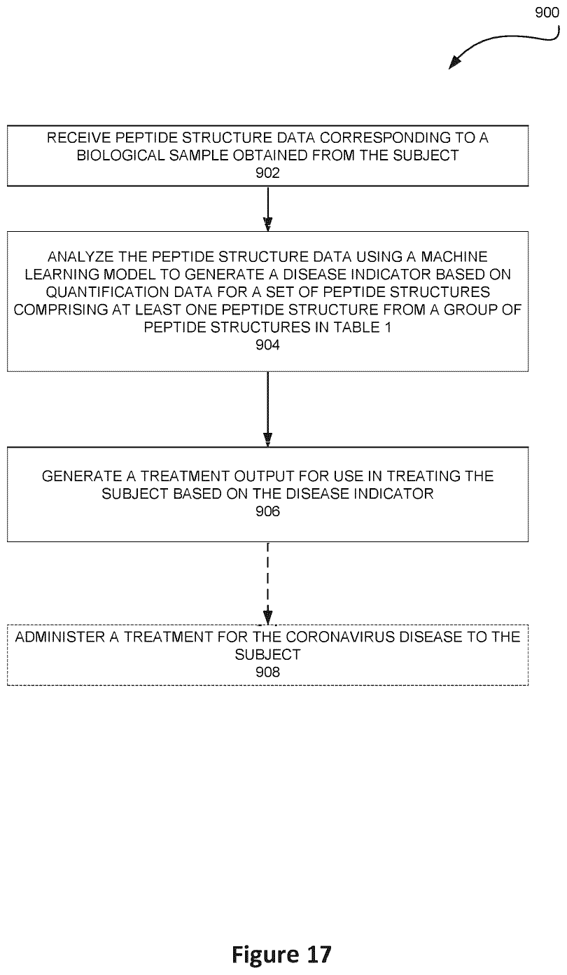

FIG. 17 is a flowchart of a process for treating a subject for a symptomatic disease state of COVID in accordance with one or more embodiments. Process 900 may be implemented using at least a portion of workflow 100 as described FIGS. 1, 2A, and/or 2B and/or analysis system 300 as described in FIG. 3.

Step 902 includes receiving peptide structure data corresponding to a biological sample obtained from the subject.

Step 904 includes analyzing the peptide structure data using a machine learning model to generate a disease indicator based on quantification data for a set of peptide structures comprising at least one peptide structure from a group of peptide structures in Table 1-1. Step 904 may be implemented in various ways. For example, step 904 may be implemented using at least a portion of process 600 in FIG. 14, process 700 in FIG. 15, or process 800 in FIG. 16 as described above.

Step 906 includes generating a treatment output for use in treating the subject based on the disease indicator. The treatment output may include, for example, at least one of an identification of a therapeutic to treat the subject, a design for the therapeutic, or a treatment plan for administering the therapeutic. The therapeutic may include, for example, without limitation, an immune checkpoint inhibitor and/or an anti-coronavirus therapeutic (e.g., an antibody that binds to and neutralizes a coronavirus). In some embodiments, step 806 may further include determining a dosage for a therapeutic to treat the subject based on at least one of the diagnosis output or the disease indicator.

In some embodiments, process 900 further includes step 908. Step 908 includes administering a treatment for the coronavirus disease to the subject. Step 908 may include, for example, administering a therapeutic dosage of a therapeutic for the coronavirus disease to the subject. In some embodiments, the therapeutic may be selected from the group consisting of remdesivir, baricitinib, tocilizumab, favipiravir, merimepodib, a monoclonal antibody therapeutic, Nirmatrelvir with Ritonavi, Molnupiravir, an immune checkpoint inhibitor, Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/MIN128), FK-506 (Tacrolimus), Ternatin 4 (DA3), Tigecycline, Tomivosertib (eFT-508), Verdinexor, WDB002, Zotatifin (eFT226), or a cell therapy, such as immune cells (T cells, NK cells, etc.) of any kind comprising an engineered antigen receptor (chimeric antigen receptor and/or T cell receptor) directed at one or more antigens of the coronavirus (including the spike protein, membrane protein, envelope protein, nucleocapsid protein, Nsp2, Nsp3, Nsp4, Nsp6, Nsp7, Nsp8, Nsp9, Nsp10, Nsp11, 3C-like proteinase, leader protein, ORF7b, 2′-O-ribose methyltransferase, endoRNAse, 3′-to-5′ exonuclease, helicase, RNA-dependent RNA polymerase, orf1a polyprotein, ORF10 protein, ORF8 protein, ORF7a protein, ORF6 protein, ORF3a, and/or orf1ab polyprotein). Thus, treatment may include at least one of an antiviral therapeutic, an anti-inflammatory therapeutic, or an immune-based therapeutic.

### XI. Peptide Structure and Product Ion Compositions, Kits and Reagents

Aspects of the disclosure include compositions comprising one or more of the peptide structures listed in Table 1-1. In some embodiments, a composition comprises a plurality of the peptide structures listed in Table 1-1. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of the peptide structures listed in Table 1-1. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ NOs: 30-62, listed in Table 1-1.

Aspects of the disclosure include compositions comprising one or more precursor ions having a defined charge and/or defined mass-to-charge (m/z) ratio, as listed in Table 4-1. Aspects of the disclosure include compositions comprising one or more product ions having a defined mass-to-charge (m/z) ratio, which product ions are produced by converting a peptide structure described herein (e.g., a peptide structure listed in Table 1-1) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1-1). In some embodiments, a composition comprises a set of the product ions listed in Table 1-1, having an m/z ratio selected from the list provided for each peptide structure in Table 1-1 or Table 2-1.

In some embodiments, a composition comprises at least one of peptide structures PS-1 to PS-45 identified in Table 1-1.

In some embodiments, a composition comprises a peptide structure or a product ion. The peptide structure or product ion comprises an amino acid sequence having at least 90% sequence identity to any one of SEQ ID NOS: 84-116, as identified in Table 5-1, corresponding to peptide structures PS-1 to PS-45 in Table 1-1.

In some embodiments, the product ion is selected as one from a group consisting of product ions identified in Table 2-1, including product ions falling within an identified m/z range of the m/z ratio identified in Table 2-1 and characterized as having a precursor ion having an m/z ratio within an identified m/z range of the m/z ratio identified in Table 4-1. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 4-1, and characterized as having a precursor ion having an m/z ratio that falls within at least one of a first range (±1.0) or a second range (±1.5) of the precursor ion m/z ratio identified in Table 4-1.

TABLE 4-1
Peptide Structures and m/z Ratios

| | | | | | 1st | 1st | 2nd | 2nd |
|---|---|---|---|---|---|---|---|---|
| PS-ID | RT | Collision | Precursor | Precursor | Product | Product | Product | Product |
| NO. | (min) | Energy | m/z | Charge | m/z | Charge | m/z | Charge |
| PS-1 | 17.61 | 15 | 891 | 5 | 366.1 | 1 | 913.5 | 3 |
| PS-2 | 35.4 | 30 | 1111.7 | 4 | 204.1 | 1 | N/A | N/A |
| PS-3 | 29.11 | 30 | 1196.5 | 4 | 1062.5 | 2 | 366.1 | 1 |
| PS-4 | 29 | 30 | 1233 | 4 | 1062.5 | 2 | 366.1 | 1 |
| PS-5 | 36.99 | 29 | 1167.3 | 5 | 366.1 | 1 | 1550.3 | 2 |
| PS-6 | 8.35 | 30 | 1173.1 | 3 | 366.1 | 1 | 757.8 | 2 |
| PS-7 | 19.37 | 39 | 1327.6 | 3 | 366.1 | 1 | N/A | N/A |
| PS-8 | 13.04 | 33 | 1061.9 | 4 | 366.1 | 1 | 1048.5 | 2 |
| PS-9 | 17.97 | 28 | 874.7 | 3 | 366.1 | 1 | N/A | N/A |
| PS-10 | 24.29 | 30 | 1225.8 | 4 | 366.1 | 1 | N/A | N/A |
| PS-11 | 21.48 | 29 | 1183.5 | 4 | 366.1 | 1 | N/A | N/A |
| PS-12 | 26.44 | 30 | 1114.2 | 4 | 204.1 | 1 | 1152.6 | 2 |
| PS-13 | 26.63 | 40 | 1278.3 | 4 | 204.1 | 1 | 1152.6 | 2 |
| PS-14 | 14.42 | 33 | 1055.8 | 3 | 1453.6 | 1 | 366.1 | 1 |
| PS-15 | 30.52 | 38 | 1284.6 | 4 | 366.1 | 1 | N/A | N/A |
| PS-16 | 28.47 | 30 | 1285.1 | 4 | 366.1 | 1 | N/A | N/A |
| PS-17 | 30.08 | 24 | 991.2 | 4 | 366.1 | 1 | 980.5 | 2 |
| PS-18 | 30.02 | 25 | 1027.7 | 4 | 366.1 | 1 | 980 | 2 |
| PS-19 | 30.4 | 30 | 1155 | 4 | 274.1 | 1 | 366.1 | 1 |
| PS-20 | 22.63 | 30 | 1191.2 | 4 | 366.1 | 1 | 978.5 | 2 |
| PS-21 | 5.67 | 21 | 878.8 | 3 | 204.1 | 1 | 1392.6 | 1 |
| PS-22 | 10.44 | 24 | 976.1 | 3 | 366.1 | 1 | N/A | N/A |
| PS-23 | 10.52 | 25 | 1043.8 | 3 | 366.1 | 1 | N/A | N/A |
| PS-24 | 25.73 | 36 | 1199.5 | 4 | 366.1 | 1 | N/A | N/A |
| PS-25 | 28.7 | 30 | 1366.3 | 5 | 366.1 | 1 | 1206.3 | 4 |
| PS-26 | 19.42 | 22 | 939.1 | 3 | 274.1 | 1 | N/A | N/A |
| PS-27 | 15.62 | 25 | 1295 | 4 | 274.1 | 1 | N/A | N/A |
| PS-28 | 12.04 | 31 | 984.7 | 4 | 366.1 | 1 | N/A | N/A |
| PS-29 | 24.35 | 25 | 1367.6 | 4 | 366.1 | 1 | N/A | N/A |
| PS-30 | 25.73 | 36 | 1199.2 | 4 | 366.1 | 1 | N/A | N/A |
| PS-31 | 26.57 | 17 | 618.3 | 2 | 736.4 | 1 | 936.5 | 1 |
| PS-32 | 22.53 | 15 | 590.3 | 2 | 342.2 | 1 | 725.4 | 1 |
| PS-33 | 24.63 | 25 | 819.1 | 3 | 609.3 | 1 | 855.5 | 1 |
| PS-34 | 23.34 | 14 | 515.8 | 2 | 607.3 | 1 | 720.4 | 1 |
| PS-35 | 17.6 | 27 | 1116.9 | 5 | 366.1 | 1 | 1060 | 2 |
| PS-36 | 22.69 | 30 | 1154.7 | 4 | 274.1 | 1 | 978.5 | 2 |
| PS-37 | 35.8 | 25 | 1272.6 | 4 | 366.1 | 1 | N/A | N/A |
| PS-38 | 20.38 | 30 | 1285.6 | 3 | 366.1 | 1 | 999.5 | 2 |
| PS-39 | 19.71 | 35 | 1441.6 | 3 | 366.1 | 1 | 978.5 | 2 |
| PS-40 | 39.53 | 24 | 1206.3 | 7 | 366.1 | 1 | N/A | N/A |
| PS-41 | 42.55 | 29 | 1179.1 | 5 | 366.1 | 1 | 1248.6 | 3 |
| PS-42 | 23.89 | 19 | 660.4 | 2 | 529.3 | 2 | 781.5 | 1 |
| PS-43 | 20.18 | 39 | 1310.6 | 3 | 366.1 | 1 | N/A | N/A |
| PS-44 | 11.95 | 32 | 1021.4 | 4 | 366.1 | 1 | N/A | N/A |
| PS-45 | 17.35 | 28 | 764.9 | 2 | 234.1 | 1 | 961.5 | 1 |

Table 5-1 defines the peptide sequences for SEQ ID NOS: 84-116 from Table 1-1. Table 5-1 further identifies a corresponding protein SEQ TD NO for each peptide sequence. Each peptide sequence in Table 3-1 is defined as an amino acid sequence.

TABLE 5-1
Peptide SEQ ID NOS

| | | Corres- |
|---|---|---|
| SEQ | | ponding |
| ID | | Protein |
| NO: | Peptide Sequence | SEQ ID NO: |
| 84 | VYIHPFHLVIHNESTCEQLAK | 55 |
| 85 | CSDGWSFDATTLDDNGTMLFFK | 56 |
| 86 | SVQEIQATFFYFTPNK | 57 |
| 87 | SVQEIQATFFYFTPNKTEDTIFLR | 57 |
| 88 | FLNNGTCTAEGK | 58 |
| 89 | DIVEYYNDSNGSHVLQGR | 59 |
| 90 | EHEGAIYPDNTTDFQR | 60 |
| 91 | NGSLFAFR | 61 |
| 92 | MVSHHNLTTGATLINEQWLLTTAK | 62 |
| 93 | VVLHPNYSQVDIGLIK | 62 |
| 94 | VLSNNSDANLELINTWVAK | 63 |
| 95 | LGNWSAMPSCK | 64 |
| 96 | ICDLLVANNHFAHFFAPQNLTNMNK | 65 |
| 97 | YLGNATAIFFLPDEGK | 66 |
| 98 | YTGNASALFILPDQDK | 67 |
| 99 | EEQYNSTYR | 68 |
| 100 | NPWGQVEWNGSWSDR | 70 |
| 101 | LNDTLDYECHDGYESNTGSTTGSIVCGYNGWSD | 71 |
| | LPICYER | |
| 102 | TFVLSALQPSPTHSSSNTQR | 65 |
| 103 | ELHHLQEQNVSNAFLDK | 60 |
| 104 | IPCSQPPQIEHGTINSSR | 71 |
| 105 | QVFPGLNYCTSGAYSNASSTDSASYYPLTGDTR | 72 |
| 106 | ACLNSTYEYIFNNCHELYSR | 73 |
| 107 | DLATVYVDVLK | 74 |
| 108 | DLLLPQPDLR | 75 |
| 109 | TSESGELHGLTTEEEFVEGIYK | 76 |
| 110 | YFIDFVAR | 77 |
| 111 | QDQCIYNTTYLNVQR | 78 |
| 112 | WVLTAAHCLLYPPWDKNFTENDLLVR | 79 |
| 113 | ASVSVLGDILGSAMQNTQNLLQMPYGCGEQNMV | 26 |
| | LFAPNIYVLDYLNETQQLTPEIK | |
| 114 | YVYIAELLAHK | 27 |
| 115 | VIDFNCTTSSVSSALANTK | 28 |
| 116 | FATTFYQHLADSK | 29 |

Table 6-1 identifies the proteins of SEQ TD NOS: 55-83 from Table 1-1. Table 6-1 identifies a corresponding protein abbreviation and protein name for each of protein SEQ ID NOS: 55-83. Further, Table 6-1 identifies a corresponding Uniprot TD for each of protein SEQ ID NOS: 55-83.

TABLE 6-1
Protein SEQ ID NOS

| SEQ | Protein | | Uniprot |
|---|---|---|---|
| ID NO. | Abbreviation | Protein Name | ID |
| 55 | ANGT | Angiotensinogen | P01019 |
| 56 | HEMO | Hemopexin | P02790 |
| 57 | AGP12 | Alpha-1-acid | P02763& |
| | | glycoprotein 1&2 | P19652 |
| 58 | CFAI | Complement Factor | P05156 |
| 59 | ZA2G | Zinc-alpha-2-glycoprotein | P25311 |
| 60 | CERU | Ceruloplasmin | P00450 |
| 61 | VTNC | Vitronectin | P04004 |
| 62 | HPT | Haptoglobin | P00738 |
| 63 | IC1 | Plasma protease C1 inhibitor | P05155 |
| 64 | APOH | Beta-2-glycoprotein 1 | P02749 |
| 65 | ITIH1 | Inter-alpha-trypsin inhibitor | P19827 |
| | | heavy chain H1 | |
| 66 | A1AT | Alpha-1-antitrypsin | P01009 |
| 67 | AACT | Alpha-1-antichymotrypsin | P01011 |
| 68 | IGG1 | Immunoglobulin heavy | P01857 |
| | | constant gamma 1 | |
| 69 | IGG2 | Immunoglobulin heavy | P01859 |
| | | constant gamma 2 | |
| 70 | CAN3 | Calpain-3 | P20807 |
| 71 | CFAH | Complement Factor H | P08603 |
| 72 | APOB | Apolipoprotein B-100 | P04114 |
| 73 | UN13A | Protein unc-13 Homolog A | Q9UPW8 |
| 74 | APOA1 | Apolipoprotein A-I | P02647 |
| 75 | A2GL | Leucine-richAlpha-2- | P02750 |
| | | glycoprotein | |
| 76 | TTR | Transthyretin | P02766 |
| 77 | KNG1 | Kininogen-1 | P01042 |
| 78 | AGP1 | Alpha-1-acid glycoprotein 1 | P02763 |
| 79 | THRB | Prothrombin | P00734 |
| 80 | A2MG | Alpha-2-macroglobulin | P01023 |
| 81 | PON1 | Serum paraoxonase/ | P27169 |
| | | arylesterase 1 | |
| 82 | HRG | Histidine-rich Glycoprotein | P04196 |
| 83 | ANT3 | Antithrombin-III | P01008 |

Table 7-1 identifies and defines the glycan structures from Table 1-1. Table 7-1 identifies a graphical representation of the structure and a coded representation of the composition for each glycan structure included in Table 1-1. As used herein, the 4-digit GL NO. is a designation that represents the number of hexoses, the number of HexNAcs, the number of Fucoses, and the number of Neuraminic Acids.

TABLE 7-1
Glycan Structure GL NOS: Structure and Composition

| Glycan | | |
|---|---|---|
| Structure | | |
| GL NO. | Structure | Composition |
| 1101 | 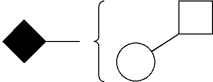 | Hex(1)HexNAc (1)Fuc(0)NeuAc(1) |
| 3410 | 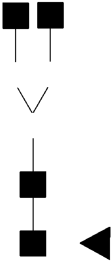 | Hex(3)HexNAc (4)Fuc(1)NeuAc(0) |
| 5301 | 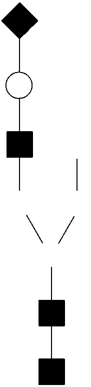 | Hex(5)HexNAc (3)Fuc(0)NeuAc(1) |
| 5401 | 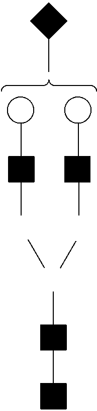 | Hex(5)HexNAc (4)Fuc(0)NeuAc(1) |
| 5402 | 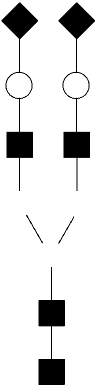 | Hex(5)HexNAc (4)Fuc(0)NeuAc(2) |
| 5410 | 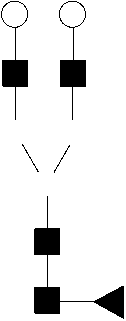 | Hex(5)HexNAc (4)Fuc(1)NeuAc(0) |
| 5411 | 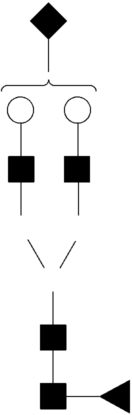 | Hex(5)HexNAc (4)Fuc(1)NeuAc(1) |
| 5412 | 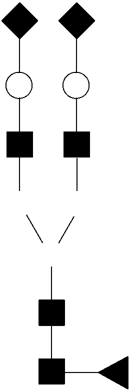 | Hex(5)HexNAc (4)Fuc(1)NeuAc(2) |
| 5420 | 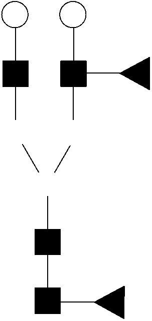 | Hex(5)HexNAc (4)Fuc(2)NeuAc(0) |
| 5510 | 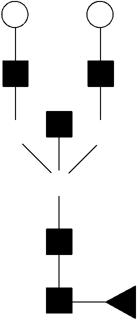 | Hex(5)HexNAc (5)Fuc(1)NeuAc(0) |
| 6411 | 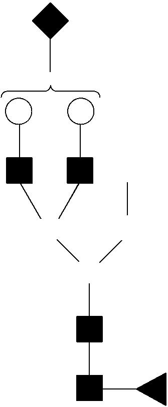 | Hex(6)HexNAc (4)Fuc(1)NeuAc(1) |
| 6502 | 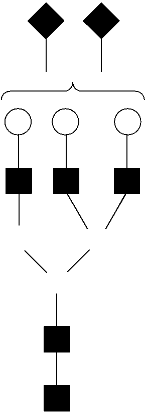 | Hex(6)HexNAc (5)Fuc(0)NeuAc(2) |
| 6503 | 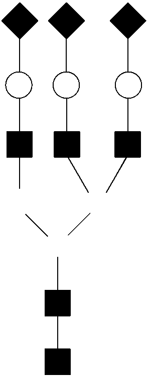 | Hex(6)HexNAc (5)Fuc(0)NeuAc(3) |
| 6513 | 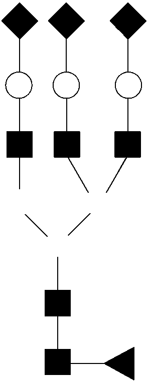 | Hex(6)HexNAc (5)Fuc(1)NeuAc(3) |
| 6523 | 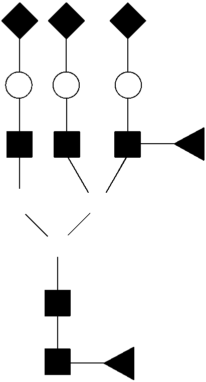 | Hex(6)HexNAc (5)Fuc(2)NeuAc(3) |
| 7420 | 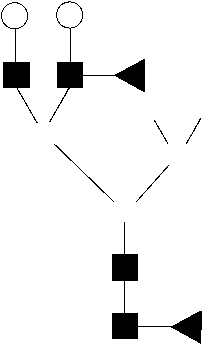 | Hex(7)HexNAc (4)Fuc(2)NeuAc(0) |
| 7602 | 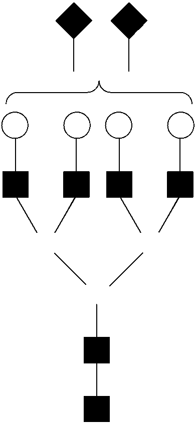 | Hex(7)HexNAc (6)Fuc(0)NeuAc(2) |
| 7614 | 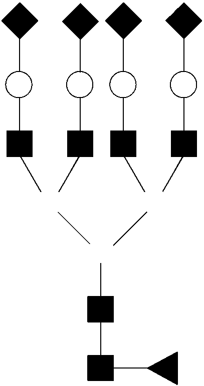 | Hex(7)HexNAc (6)Fuc(1)NeuAc(4) |

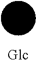

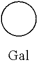

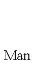

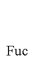

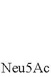

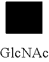

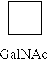

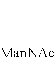

[symbol]

Xyl

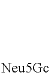

[symbol]

Glcn

[symbol]

Galn

[symbol]

ManN

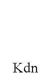

[symbol]

GlcA

[symbol]

GalA

[symbol]

ManA

[symbol]

IdoA

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating various disease conditions, including, without limitation, COVID (e.g., COVID-19). A transition includes a precursor ion and at least one product ion grouping. As reviewed herein, the peptide structures in Table 1-1, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, COVID-19.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 4-1 or an m/z ratio within an identified m/z ratio as provided in Table 4-1. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### XII. Embodiments of Treatment and Diagnosis

In various embodiments, the disclosure provides systems, methods, and compositions for the treatment or diagnosis of a coronavirus, including at least SARS-CoV-2. Provided herein are examples of one or more peptide structures that are related to the presence of SARS-CoV-2 in a subject, or the susceptibility of a subject to SARS-CoV-2, such as susceptibility greater than the general population, or to a subject with an increased risk of having symptoms of COVID. In at least some cases, the peptide structures are related to an individual having a likelihood of having one or more COVID symptoms of any kind upon infection with a coronavirus. The disclosure provides systems, methods, and compositions that identify a subject in need of treatment or prevention of SARS-CoV-2 infection based on the presence of at least one peptide structure encompassed herein, such as one or more peptide structures in Table 2-1, as one example. In some embodiments, the peptide structures act as markers for symptomatic COVID that are accurate regardless of the status of one or more characteristics of the individual: biological sex, sample source, sample collection, diabetes status, obese status, immunocompromised state, age, etc.

In some embodiments, a healthy subject is subject to systems or methods of the disclosure in order to determine whether or not the subject is susceptible to being symptomatic of COVID instead of asymptomatic. In cases wherein a sample from the subject indicates the presence of one or more peptide structures as disclosed herein, the subject may alter their behavior, e.g., upon onset of one or more COVID symptoms or upon exposure to an infected individual. The subject may begin COVID treatment, isolate from others, etc. The subject may be able to delay the onset and/or reduce the severity of one or more symptoms of COVID and/or may be able to reduce the chance of mortality. In some cases, a sample from a healthy subject may indicate that no peptide structures as disclosed herein are present in the sample, and the individual may expect to be asymptomatic upon infection with SARS-CoV-2. In such cases, upon exposure to an infected person, the subject may take precautions to avoid spread of the virus.

In some embodiments of the disclosure, there is identification of a symptomatic disease state in a subject that is suspected of having SARS-CoV-2 infection yet in which onset (or detectable onset) of one or more symptoms of the infection has not yet occurred. The subject may or may not know that they were exposed to an infected individual and/or environment.

The embodiments concern classifying biological samples, measuring for one or more certain markers from a biological sample, assaying for one or more certain markers from a biological sample, determining the presence of one or more certain markers from a biological sample, and so forth. The embodiments of the disclosure utilize models that accurately identify that an individual may become symptomatic of COVID upon exposure to SARS-CoV-2, whether from an infected individual and/or in an environment (such as a public place, in a vehicle, on a plane, in a workplace, etc.).

In various embodiments, there are methods of treating a subject for COVID upon measuring one or more peptide structures in a sample of any kind from the subject. The individual may or may not exhibit one or more symptoms of COVID. The individual may or may not have known about exposure to an infected individual and/or environment. In various embodiments, there are method of diagnosing a subject for COVID upon measuring one or more peptide structures in a sample of any kind from the subject. The individual may or may not exhibit one or more symptoms of COVID. The individual may or may not have known about exposure to an infected individual and/or environment.

Any individual subject to methods encompassed herein may or may not have long COVID (which may also be known as long-haul COVID, post-acute COVID-19, post-acute sequelae of SARS CoV-2 infection (PASC), long-term effects of COVID, and chronic COVID). The individual may have one or more symptoms that can last more than 3 or 4 weeks or even months after infection.

Individuals subject to the methods of the disclosure may not have one or more symptoms of COVID but may need a sample processed to measure for the one or more peptide structures encompassed herein because of a need to manage their actions in the event of the presence of one or more peptide structures in the sample, such as prior to travel of any kind, prior to contact with an immunocompromised subject, prior to contact with the elderly, prior to contact with a cancer patient, prior to exposure to large groups of people, and so forth.

Embodiments of the disclosure include methods that distinguish a symptomatic disease state from a common cold state, an asymptomatic disease state, a sepsis state, and/or a healthy state in a subject, comprising the step of detecting or measuring in a sample from the subject (or a processed output from the sample) one or more peptide structures encompassed herein. The subject may or may not have one or more symptoms associated with COVID. The subject may have one or more symptoms common between COVID and the common cold. The subject may have one or more symptoms common between COVID and sepsis.

Embodiments of the disclosure provide methods of diagnosing a coronavirus disease (COVID) in a subject, comprising the step of identifying one or more peptide structures identified in Table 2-1 from a sample from the subject. Any sample for any method may comprise blood, plasma, serum, urine, saliva, mucosal excretions, sputum, stool, nasal mucus, phlegm, and/or tears. The method may comprise the step of identifying that occurs once or multiple times, and in some cases one or more symptoms becomes undetectable between multiple identifying steps.

Embodiments of the disclosure include methods of identifying or managing an at-risk subject for a coronavirus disease (COVID), the method comprising measuring whether a biological sample obtained from the subject evidences COVID using part or all of any method encompassed herein, and subjecting the subject to one or more medical tests or procedures, and/or subjecting the subject to one or more preventatives or therapies in response to the identification of the symptomatic disease state. The subject may have one or more COVID symptoms at the time of measuring and/or at the time of obtaining the sample. The subject may be asymptomatic at the time of measuring and/or at the time of obtaining the sample.

Embodiments of the disclosure include methods of identifying a subject suitable for, or in need of, COVID prevention or treatment, the method comprising the step of measuring from a biological sample taken from the subject for the presence of one or a combination of peptide structures identified in Table 2-1, wherein their detection indicates that the subject should have COVID prevention or treatment. The subject may have one or more COVID symptoms at the time of measuring and/or at the time of obtaining the sample, or the subject may be asymptomatic at the time of measuring and/or at the time of obtaining the sample.

Embodiments of the disclosure include methods of predicting whether a subject will be symptomatic upon coronavirus infection, comprising the step of measuring from a biological sample taken from the subject for the presence of one or a combination of peptide structures identified in Table 2-1.

### XIII. Representative Experimental Results

### XIII.A. Samples Used for Experiments

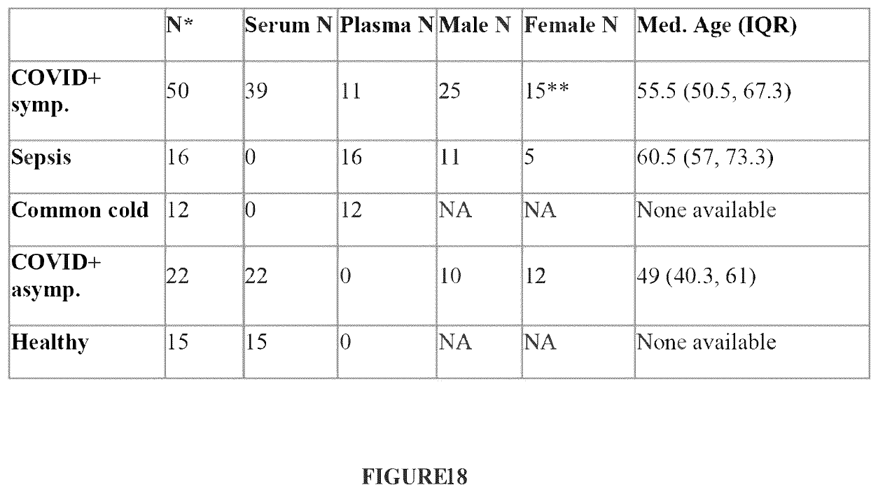

The samples used Example Experiments 1 and 2 described below included 115 samples collected from various sources, stored at −80 C, and processed through a targeted MRM panel, and analyzed. The 115 samples were collected for: 50 (39 serum, 11 plasma) patients who tested positive via PCR with severe/symptomatic COVID-19 infection, 22 serum samples from individuals who did not experience any symptoms of COVID-19 but whose serology tests were confirmed positive after infection, 16 plasma samples from patients who presented with sepsis (8 mild, 8 severe), 12 plasma samples from patients who had a common cold caused by a non-COVID-19 coronavirus at the time of blood draw, and 15 serum samples who were classified as historic healthy controls. FIG. 18 is a table that provides information about the subjects involved in these example experiments, including, number, sample type, gender, and median age, where available.

### XIII.B. Example Experiment 1

Objective: The objective was to determine whether a set of peptide structures could be identified that could be used for accurate differentiation of patients suffering from symptomatic COVID (in particular COVID-19), patients suffering from sepsis, and patients with other conditions including a healthy state, an asymptomatic disease state of COVID, and the common cold.

Methodology: A panel of 597 peptide structures for the various samples were considered for analysis, consisting of 531 glycopeptide structures and 66 aglycosylated peptide structures. In order to compare two groups (e.g., two portions of subjects having different states), differential expression analysis was performed. In particular, a linear regression was performed on a marker-by-marker basis with group membership serving as the sole binary independent variable. Because each of the 597 peptide structures was being compared simultaneously, corrections were made to achieve a FDR (false discovery rate) of less than 0.05 merits significance. A peptide structure achieving statistical significance implied that the mean normalized abundance between the two groups was significantly different. Overlapping sets of statistically significant peptide structures between sets of groups were then assessed. For example, differential expression analysis was performed to compare symptomatic COVID samples with the samples for each of the four other groups separately (four pairings).

Results: Of the panel of 597 peptide structures, 34 peptide structures (which may be referred to as markers or peptide structure markers) appeared in all four sets of the statistically significant peptide structures, where symptomatic COVID-19 was the primary comparison group. These 34 peptide structures formed a “symptomatic-COVID-specific” signal.

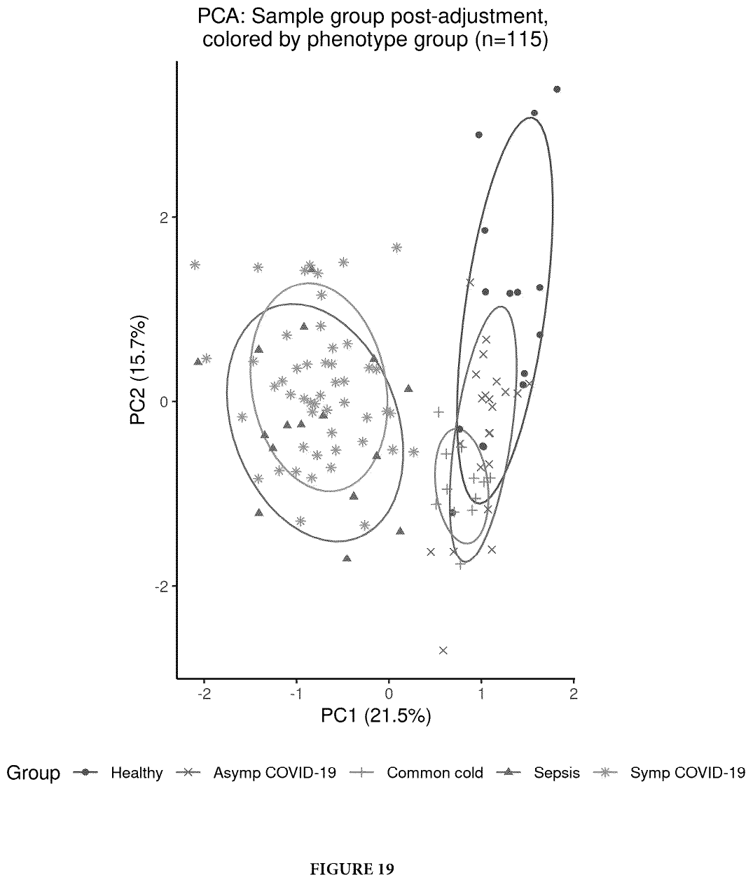

FIG. 19 is a plot showing a principal component analysis via a singular value decomposition of the centered and scaled data matrix of all 115 patient samples and all peptide structures (e.g., glycosylated and aglycosylated) of the panel. The vast degree of separation between groups, as shown in the plot, supports the claim that there are multiple significant peptide structures (e.g., markers) between symptomatic COVID and other disease states.

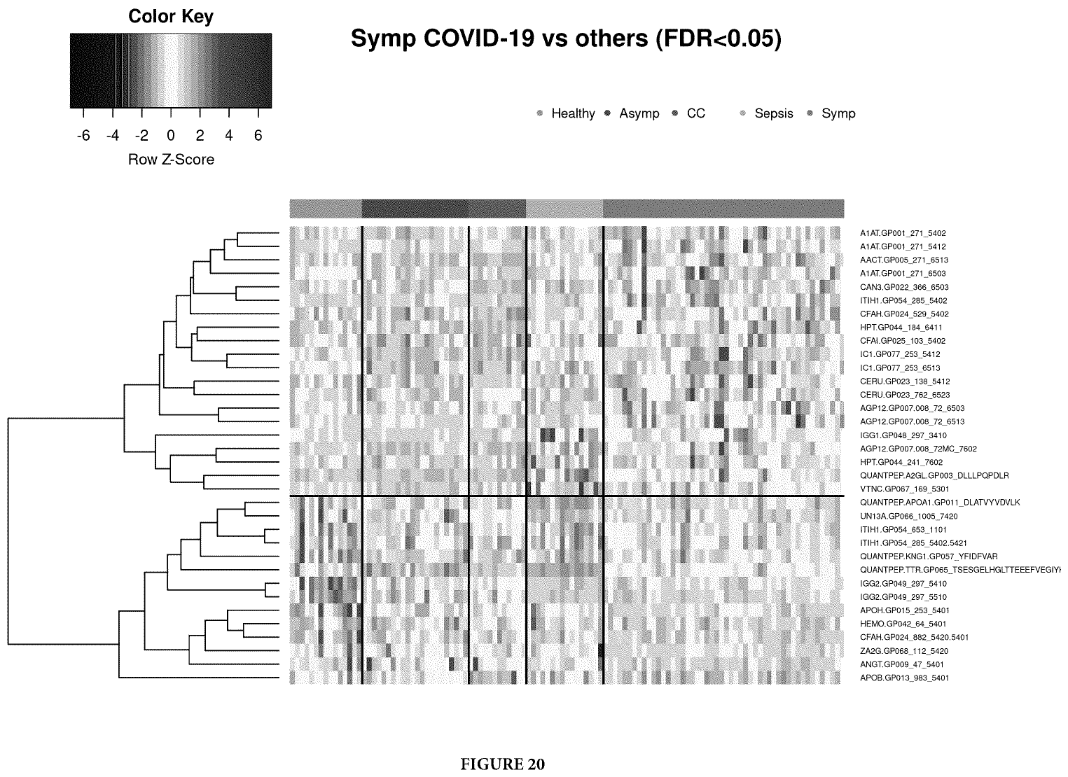

FIG. 20 is an illustration of a heat map depicting the peptide structures, also identified in Table 2-1 in accordance with one or more embodiments. As indicated in FIG. 20, 34 biomarkers are significantly differentially expressed between the symptomatic disease state of COVID (symptomatic COVID) and all other states. The other states include a healthy state, a common cold state, an asymptomatic disease state of COVID, and a sepsis state.

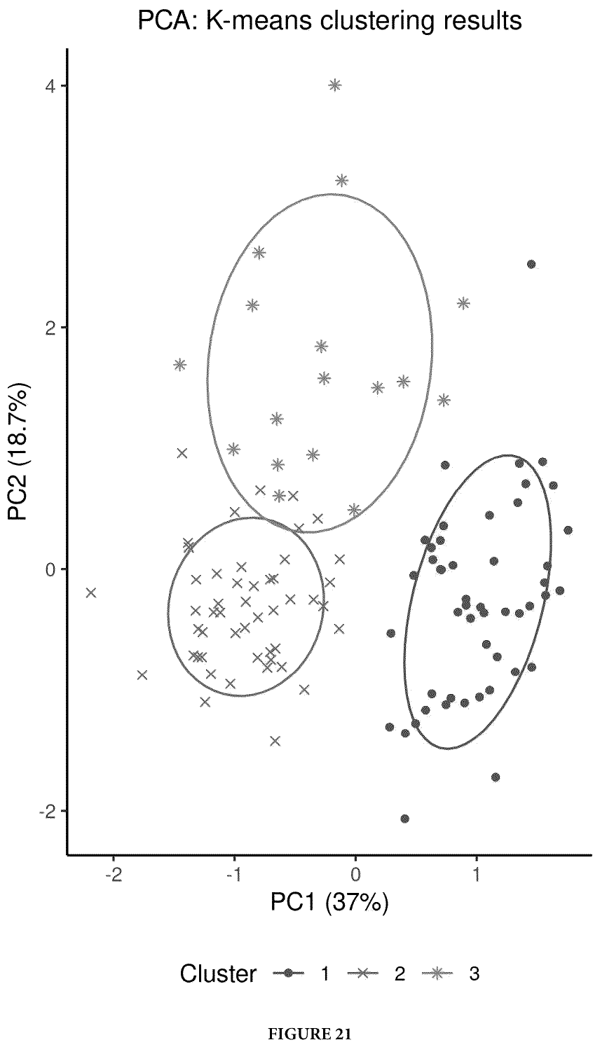

Validation: A k-means clustering model was used to determine whether the 34 peptide structures could accurately differentiate between the symptomatic disease state of COVID and two other states: the sepsis state and an “other” state (this “other” state comprising a combination of samples corresponding to the healthy state, the common cold state, and the asymptomatic disease state of COVID). FIG. 21 is a plot showing a k-means clustering graph using markers differentially expressed between symptomatic COVID and all other groups in accordance with one or more embodiments. As indicated in FIG. 21, 94% of symptomatic COVID-19 subjects are allocated to cluster 3, which is the cluster corresponding to the symptomatic disease state of COVID.

### XIII.C. Example Experiment 2

Objective: The objective was to determine whether a set of peptide structures forming a peptide structure profile for a subject could be analyzed and used to determine a likelihood that the subject is suffering from symptomatic COVID and in particular, symptomatic COVID-19.

Methodology: A panel of 597 peptide structures for the various samples were considered for analysis. Of the 115 samples, 75% (n=86) were used for training and 25% (n=29) were held for testing. A penalized multivariable regression model was used to consider all 597 markers on the panel to build a model using 5-fold repeated cross-validation to optimize hyperparameters. The coefficients of many of these 597 peptide structures shrank to 0 as the model was fit. The final model is mathematically expressed using non-zero weights from the training. The mathematical expression:

$$log it ( p ) = {β}_{0} + {\sum }_{i = 1}^{m} {β}_{i} {X}_{i}$$

where p is the predicted probability of symptomatic COVID-19 for a particular patient, βᵢ is the coefficient of the i-th marker, Xᵢ is the normalized abundance (concentration) for that patient's i-th marker, β₀ is the intercept term, and m is the total number of markers.

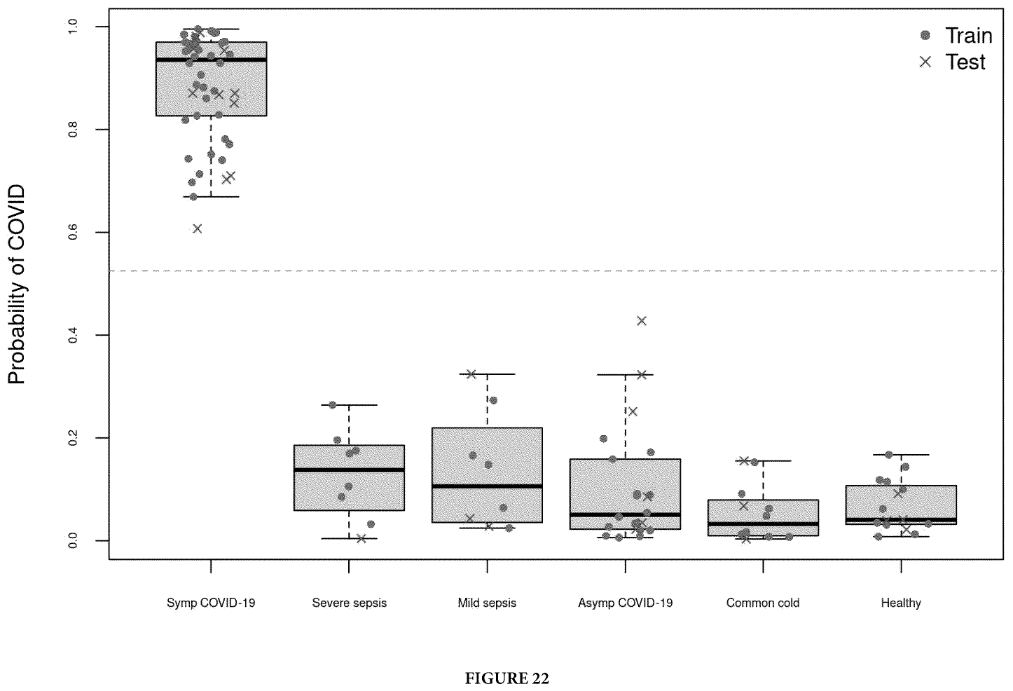

Results and Validation: The final model was tested on the 25% testing set. Assessing performance metrics such as accuracy, sensitivity, specificity, and AUC, the final model was able to accurately diagnose with 100% accuracy. The lambda hyperparameter pertaining to the amount of regularization was tuned to 0.0351, and the probability threshold used to classify patients as symptomatic COVID-19 or not was 0.524754. If the predicted probability was larger than 0.524754, the patient was classified as symptomatic COVID-19, and not symptomatic COVID-19 if otherwise. FIG. 22 is an illustration of a 5-fold cross validated LASSO regression model classifying COVID versus other patients in accordance with one or more embodiments. As indicated in FIG. 22, using the set of peptide structures results in diagnosis with an accuracy level of 100%.

### XIV. Recitations of Various Embodiments of the Present Disclosure

Embodiment 1: A method of determining whether a biological sample corresponds to a sepsis state, the method comprising: receiving peptide structure data corresponding to the biological sample obtained from a subject; inputting quantification data identified from the peptide structure data for a set of peptide structures into a supervised machine learning model, wherein the set of peptide structures includes at least one peptide structure identified in Table 1; analyzing the quantification data using the supervised machine learning model to generate a disease indicator that classifies the biological sample as corresponding to a cluster of a plurality of clusters that has a least distance to the biological sample, the plurality of clusters corresponding a plurality of states that includes the sepsis state; and generating a diagnosis output based on the disease indicator.

Embodiment 2: The method of embodiment 1, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 24-49 as defined in Table 3.

Embodiment 3: The method of embodiment 1 or embodiment 2, wherein the at least one peptide structure comprises an aglycosylated peptide structure defined by a peptide sequence, as identified in Table 1, with the peptide sequence being one of SEQ ID NOS: 50-54 as defined in Table 3.

Embodiment 4: The method of any one of embodiments 1-3, wherein the supervised machine learning model comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

Embodiment 5: The method of any one of embodiments 1-4, further comprising: training the supervised machine learning model using training data generated from an unsupervised machine learning model, wherein the training data comprises a plurality of peptide structure profiles for a plurality of subjects and identifies a corresponding state of the plurality of states for each peptide structure profile of the plurality of peptide structure profiles; and wherein the unsupervised machine learning model is trained to cluster the plurality of peptide structure profiles into the plurality of clusters.

Embodiment 6: The method of any one of embodiments 1-4, wherein the unsupervised machine learning model is a k-means clustering model.

Embodiment 7: The method of any one of embodiments 1-6, wherein a peptide structure profile of the plurality of peptide structure profiles for a corresponding subject of the plurality of subjects is selected based on a differential expression analysis of quantification metrics for a panel of peptide structures for the plurality of subjects.

Embodiment 8: The method of embodiment 7, further comprising: comparing, using the differential expression analysis, the quantification metrics corresponding to the panel of peptide structures for a first portion of the plurality of subjects diagnosed with the sepsis state to that of each portion of a set of other portions of the plurality of subjects to generate a set of comparisons; and selecting a portion of the panel of peptide structures having a false discovery rate below 0.05 across the set of comparisons as the set of peptide structures.

Embodiment 9: The method of embodiment 8, wherein the comparing comprises: comparing the quantification metrics corresponding to the panel of peptide structures for the first portion of the plurality of subjects diagnosed with the sepsis state to that of a second portion of the plurality of subjects diagnosed with a symptomatic disease state of a coronavirus disease to generate a first comparison of the set of comparisons.

Embodiment 10: The method of embodiment 9, wherein the comparing further comprises: comparing the quantification metrics corresponding to the panel of peptide structures for the first portion of the plurality of subjects diagnosed with the sepsis state to that of at least one of: a third portion of the plurality of subjects diagnosed with a common cold state to generate a second comparison of the set of comparisons; a fourth portion of the plurality of subjects diagnosed with a healthy state to generate a third comparison of the set of comparisons; a fifth portion of the plurality of subjects diagnosed with an asymptomatic state of the coronavirus disease to generate a fourth comparison of the set of comparisons.

Embodiment 11: The method of any one of embodiments 1-10, wherein the quantification data for a peptide structure of the set of peptide structures comprises at least one of a relative quantity, an adjusted quantity, a normalized quantity, a relative concentration, an adjusted concentration, or a normalized concentration.

Embodiment 12: The method of any one of embodiments 1-11, wherein the peptide structure data is generated using multiple reaction monitoring mass spectrometry (MRM-MS).

Embodiment 13: The method of any one of embodiments 1-12, wherein the sepsis state is selected from a group consisting of a mild sepsis state, a moderate sepsis state, or a severe sepsis state.

Embodiment 14: The method of any one of embodiments 1-13, further comprising: creating a sample from the biological sample; and preparing the sample using reduction, alkylation, and enzymatic digestion to form a prepared sample that includes a set of peptide structures.

Embodiment 15: The method of any one of embodiments 1-14, further comprising: generating the peptide structure data from the prepared sample using multiple reaction monitoring mass spectrometry (MRM-MS).

Embodiment 16: The method of any one of embodiments 1-15, wherein generating the diagnosis output comprises: generating a report that includes a diagnosis that the subject is positive for the sepsis state in response to the disease indicator classifying the biological sample as corresponding to the sepsis state.

Embodiment 17: The method of any one of embodiments 1-16, wherein the biological sample comprises at least one of a whole blood sample, a plasma sample, or a serum sample.

Embodiment 18: The method of any one of embodiments 1-17, further comprising: generating a treatment output based on at least one of the diagnosis output or the disease indicator.

Embodiment 19: The method of embodiment 18, wherein the treatment output comprises at least one of an identification of a treatment to treat the subject, a design for the treatment, a manufacturing plan for the treatment, or a treatment plan for administering the treatment.

Embodiment 20: The method of embodiment 18 or embodiment 19, wherein the treatment comprises at least one antibiotic.

Embodiment 21: The method of any one of embodiments 18-20, wherein the treatment comprises at least one of a broad-spectrum antibiotic, a targeted antibiotic, or a vasopressor.

Embodiment 22: The method of any one of embodiments 18-21, wherein generating the treatment output comprises: determining a dosage for a therapeutic to treat the subject based on at least one of the diagnosis output or the disease indicator.

Embodiment 23: The method of any one of embodiments 18-22, wherein the diagnosis output identifies that the biological sample is positive for the sepsis state and further comprising: administering a therapeutic dosage of a therapeutic for the sepsis state to the subject, the therapeutic comprising one or more antibiotics.

Embodiment 24: A method of identifying a sepsis-specific set of peptide structures for use in diagnosing a sepsis state, the method comprising: receiving quantification data for a panel of peptide structures for a plurality of subjects diagnosed with a plurality of states, the plurality of states including the sepsis state; comparing quantification metrics corresponding to the panel of peptide structures for a first portion of the plurality of subjects diagnosed with the sepsis state to that of each portion of a set of other portions of the plurality of subjects to generate a set of comparisons using a differential expression analysis; selecting a portion of the panel of peptide structures having a false discovery rate below 0.05 across the set of comparisons as a set of peptide structures to be associated with the sepsis state, wherein the set of peptide structures includes at least one glycopeptide structure defined by a peptide sequence and a glycan structure linked to the peptide sequence at a linking site of the peptide sequence; analyzing the quantification data for the set of peptide structures for the plurality of subjects using an unsupervised machine learning model to cluster the plurality of subjects; and training a supervised machine learning model to determine whether a biological sample obtained from a subject corresponds to the sepsis state or another state of the plurality of states.

Embodiment 25: The method of embodiment 24, further comprising: analyzing the biological sample obtained from the subject using the supervised machine learning model that has been trained to generate a disease indicator that indicates whether the biological subject is positive for the sepsis state.

Embodiment 26: The method of embodiment 24 or embodiment 25, wherein the plurality of states further includes at least one of a common cold state, a healthy state, a symptomatic disease state of a coronavirus disease (COVID), or an asymptomatic disease state of the coronavirus disease.

Embodiment 27: The method of any one of embodiments 24-26, wherein the sepsis state is either a mild sepsis state or a severe sepsis state.

Embodiment 28: The method of any one of embodiments 24-27, wherein the unsupervised machine learning model comprises a k-means clustering model and wherein the supervised machine learning model comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

Embodiment 29: The method of any one of embodiments 24-28, wherein the set of peptide structures includes at least three peptide structures identified in Table 1.

Embodiment 30: A method of evaluating a biological sample obtained from a subject with respect to a sepsis state, the method comprising: receiving peptide structure data corresponding to the biological sample obtained from the subject; identifying a peptide structure profile for the biological sample using the peptide structure data, the peptide structure profile comprising quantification data for a set of peptide structures associated with the sepsis state, wherein the set of peptide structures includes at least two peptide structures from a selected group of peptide structures identified in Table 1; and wherein at least two peptide structures in the selected group of peptide structures includes a glycopeptide structure defined by a peptide sequence and a glycan structure linked to a linking site of the peptide sequence; computing a disease indicator using the peptide structure profile and a model, wherein the disease indicator indicates whether the biological sample is positive for the sepsis state; and generating at least one of a diagnosis output or a treatment output based on the disease indicator.

Embodiment 31: The method of embodiment 30, wherein the model includes a supervised machine learning model that comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

Embodiment 32: The method of embodiment 30 or embodiment 31, wherein the model comprises a supervised machine learning model trained using an output of an unsupervised machine learning model that is trained to cluster a plurality of peptide structure profiles for a plurality of subjects according to a plurality of states, the plurality of states including the sepsis state.

Embodiment 33: The method of any one of embodiments 30-32, wherein the treatment output comprises at least one of an identification of a treatment to treat the subject, a design for the treatment, a manufacturing plan for the treatment, or a treatment plan for administering the treatment.

Embodiment 34: The method of any one of embodiments 30-33, further comprising: administering a therapeutic dosage of a therapeutic for the sepsis state to the subject based on the at least one of the diagnosis output or the treatment output, the therapeutic comprising one or more antibiotics.

Embodiment 35: A method of designing a treatment for a sepsis state in a subject, the method comprising: designing a therapeutic for treating the subject in response to identifying the subject as being positive for the sepsis state using the method of any one of embodiments 1-22, 24-29, or 30-33.

Embodiment 36: A method of planning a treatment for a sepsis state in a subject, the method comprising: generating a treatment plan for treating the subject in response to identifying the subject as being positive for the sepsis state using the method of any one of embodiments 1-22, 24-29, or 30-33.

Embodiment 37: A method of manufacturing a treatment for a sepsis state in a subject, the method comprising: manufacturing a therapeutic for treating the subject in response to identifying the subject as being positive for the sepsis state using the method of any one of embodiments 1-22, 24-29, or 30-33.

Embodiment 38: A method of treating a sepsis state in a subject, the method comprising: administering to the subject a therapeutic to treat the subject based on identifying the subject as being positive for the sepsis state using the method of any one of embodiments 1-22, 24-29, or 30-33.

Embodiment 39: A method of treating a sepsis state in a subject, the method comprising: selecting a therapeutic to treat the subject based on determining that the subject is responsive to the therapeutic using the method of any of embodiments 1-22, 24-29, or 30-33; and administering the selected therapeutic to the subject.

Embodiment 40: A method for analyzing a set of peptide structures in a sample from a patient, the method comprising: (a) obtaining the sample from the patient; (b) preparing the sample to form a prepared sample comprising a set of peptide structures; (c) inputting the prepared sample into a reaction monitoring mass spectrometry system to detect a set of product ions associated with each peptide structure of the set of peptide structures, the set of peptide structures comprising at least one of: a first peptide structure associated with the set of product ions that includes a product ion having a mass-to-charge (m/z) ratio within a range selected from a group consisting of 366.1±0.5, ±0.8, and ±1.0 and that is characterized as having a precursor ion having an m/z ratio of 1386.1 within a range selected from a group consisting of ±1.0 and ±1.5; a second peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1167.3 within a range selected from a group consisting of ±1.0 and ±1.5; a third peptide structure associated with the corresponding set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1270.2 within a range selected from a group consisting of ±1.0 and ±1.5; a fourth peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 1152.5 within a range selected from a group consisting of ±1.0 and ±1.5; a fifth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 918.4 within a range selected from a group consisting of ±1.0 and ±1.5; a sixth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1032.9 within a range selected from a group consisting of ±1.0 and ±1.5; a seventh peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1197 within a range selected from a group consisting of ±1.0 and ±1.5; an eighth peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 984.4 within a range selected from a group consisting of ±1.0 and ±1.5; a ninth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 874.7 within a range selected from a group consisting of ±1.0 and ±1.5; a tenth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1225.8 within a range selected from a group consisting of ±1.0 and ±1.5; an 11^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1073.4 within a range selected from a group consisting of ±1.0 and ±1.5; a 12^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1183.5 within a range selected from a group consisting of ±1.0 and ±1.5; a 13^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1015.2 within a range selected from a group consisting of ±1.0 and ±1.5; a 14^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1284.6 within a range selected from a group consisting of ±1.0 and ±1.5; a 15^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1039.1 within a range selected from a group consisting of ±1.0 and ±1.5; a 16^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1285.1 within a range selected from a group consisting of ±1.0 and ±1.5; a 17^{th} peptide structure associated with the set of product ions that includes a product ion having a mass-to-charge (m/z) ratio that is within a range selected from a group consisting of 1249.3±0.5, ±0.8, and ±1.0 and that is characterized as having a precursor ion having an m/z ratio of 1150.3 within a range selected from a group consisting of ±1.0 and ±1.5; an 18^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1191.2 within a range selected from a group consisting of ±1.0 and ±1.5; a 19^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1050.4 within a range selected from a group consisting of ±1.0 and ±1.5; a 20^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 788.3 within a range selected from a group consisting of ±1.0 and ±1.5; a 21^{st} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 878.8 within a range selected from a group consisting of ±1.0 and ±1.5; a 22^{nd} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 946.5 within a range selected from a group consisting of ±1.0 and ±1.5; a 23^{rd} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1281.5 within a range selected from a group consisting of ±1.0 and ±1.5; a 24^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 873.4 within a range selected from a group consisting of ±1.0 and ±1.5; a 25^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 922.1 within a range selected from a group consisting of ±1.0 and ±1.5; a 26^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 764.8 within a range selected from a group consisting of ±1.0 and ±1.5; a 27^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 1087.1 within a range selected from a group consisting of ±1.0 and ±1.5; a 28^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 976.1 within a range selected from a group consisting of ±1.0 and ±1.5; a 29^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1073.1 within a range selected from a group consisting of ±1.0 and ±1.5; a 30^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1043.8 within a range selected from a group consisting of ±1.0 and ±1.5; a 31^{st} peptide structure associated with the set of product ions that includes a product ion having a mass-to-charge (m/z) ratio that is within a range selected from a group consisting of 1009±0.5, ±0.8, and ±1.0 and that is characterized as having a precursor ion having an m/z ratio of 891.8 within a range selected from a group consisting of ±1.0 and ±1.5; a 32^{nd} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 988.8 within a range selected from a group consisting of ±1.0 and ±1.5; a 33^{rd} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 1142 within a range selected from a group consisting of ±1.0 and ±1.5; a 34^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 911.4 within a range selected from a group consisting of ±1.0 and ±1.5; a 35^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1041 within a range selected from a group consisting of ±1.0 and ±1.5; a 36^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1008.2 within a range selected from a group consisting of ±1.0 and ±1.5; a 37^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1109.8 within a range selected from a group consisting of ±1.0 and ±1.5; a 38^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 761.7 within a range selected from a group consisting of ±1.0 and ±1.5; a 39^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 939.1 within a range selected from a group consisting of ±1.0 and ±1.5; a 40^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1206.3 within a range selected from a group consisting of ±1.0 and ±1.5; a 41^{st} structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1199.2 within a range selected from a group consisting of ±1.0 and ±1.5; a 42^{nd} peptide structure associated with the set of product ions that includes a product ion having a mass-to-charge (m/z) ratio that is within a range selected from a group consisting of 565.1±0.5, ±0.8, and ±1.0 and that is characterized as having a precursor ion having an m/z ratio of 697.4 within a range selected from a group consisting of ±1.0 and ±1.5; a 43^{rd} peptide structure associated with the set of product ions that includes a product ion having a mass-to-charge (m/z) ratio that is within a range selected from a group consisting of 736.4±0.5, ±0.8, and ±1.0 and that is characterized as having a precursor ion having an m/z ratio of 618.3 within a range selected from a group consisting of ±1.0 and ±1.5; a 44^{th} peptide structure associated with the set of product ions that includes a product ion having a mass-to-charge (m/z) ratio that is within a range selected from a group consisting of 342.2±0.5, ±0.8, and ±1.0 and that is characterized as having a precursor ion having an m/z ratio of 590.3 within a range selected from a group consisting of ±1.0 and ±1.5; a 45^{th} peptide structure associated with the set of product ions that includes a product ion having a mass-to-charge (m/z) ratio that is within a range selected from a group consisting of 342.2±0.5, ±0.8, and ±1.0 and that is characterized as having a precursor ion having an m/z ratio of 590.3 within a range selected from a group consisting of ±1.0 and ±1.5; and a 46^{th} peptide structure associated with the set of product ions that includes a product ion having a mass-to-charge (m/z) ratio that is within a range selected from a group consisting of 646.4±0.5, ±0.8, and ±1.0 and that is characterized as having a precursor ion having an m/z ratio of 416.7 within a range selected from a group consisting of ±1.0 and ±1.5; and (d) generating quantification data for the set of product ions using the reaction monitoring mass spectrometry system.

Embodiment 41: The method of embodiment 40, further comprising: generating a diagnosis output using the quantification data and a model that has been trained using at least one of supervised or unsupervised machine learning.

Embodiment 42: The method of embodiment 40 or embodiment 41, wherein the reaction monitoring mass spectrometry system uses or at least one of multiple reaction monitoring mass spectrometry (MRM-MS) or selected reaction monitoring mass spectrometry (SRM-MS) to detect the set of product ions and generate the quantification data.

Embodiment 43: The method of any one of embodiments 40-42, wherein the sample comprises a plasma sample.

Embodiment 44: The method of any one of embodiments 40-43, wherein the sample comprises a serum sample.

Embodiment 45: The method of any one of embodiments 40-44, wherein preparing the sample comprises at least one of: denaturing one or more proteins in the sample using heat to form one or more denatured proteins; reducing the one or more denatured proteins in the sample using a reducing agent to form one or more reduced proteins; alkylating the one or more proteins in the sample using an alkylating agent to prevent reformation of disulfide bonds in the one or more reduced proteins to form one or more alkylated proteins; or digesting the one or more alkylated proteins in the sample using a proteolysis catalyst to form the prepared sample comprising the set of peptide structures.

Embodiment 46: A composition comprising at least one of peptide structures PS-1 to PS-46 identified in Table 1.

Embodiment 47: A composition comprising a peptide structure or a product ion, wherein: the peptide structure or product ion comprises an amino acid sequence having at least 90% sequence identity to any one of SEQ ID NOS: 24-54, corresponding to peptide structures PS-1 to PS-46 in Table 1; and the product ion is selected as one from a group consisting of product ions identified in Table 2 including product ions falling within an identified m/z range.

Embodiment 48: A composition comprising a glycopeptide structure selected as one from a group of glycopeptide structures consisting of: wherein: a first glycopeptide structure having a monoisotopic mass of 5538.39 and comprising: the amino acid sequence of SEQ ID NO: 24; and glycan structure GL NO. 7601 linked to the 15^{th} residue of SEQ ID NO: 24; a second glycopeptide structure having a monoisotopic mass of 5829.49 and comprising: the amino acid sequence of SEQ ID NO: 24; and glycan structure GL NO. 7602 linked to the 15^{th} residue of SEQ ID NO: 24; a third glycopeptide structure having a monoisotopic mass of 3807.44 and comprising: the amino acid sequence of SEQ ID NO: 25; and glycan structure GL NO. 6503 linked to the 5^{th} residue of SEQ ID NO: 25; a fourth glycopeptide structure having a monoisotopic mass of 4603.94 and comprising: the amino acid sequence of SEQ ID NO: 26; and glycan structure GL NO. 5412 linked to the 16^{th} residue of SEQ ID NO: 26; a fifth glycopeptide structure having a monoisotopic mass of 4584.93 and comprising: the amino acid sequence of SEQ ID NO: 27; and glycan structure GL NO. 5421 linked to the 2^{nd} residue of SEQ ID NO: 27; a sixth glycopeptide structure having a monoisotopic mass of 4125.73 and comprising: the amino acid sequence of SEQ ID NO: 28; and glycan structure GL NO. 5401 linked to the 3^{rd} residue of SEQ ID NO: 28; a seventh glycopeptide structure having a monoisotopic mass of 4781.95 and comprising: the amino acid sequence of SEQ ID NO: 28; and glycan structure GL NO. 6502 linked to the 3^{rd} residue of SEQ ID NO: 28; an eighth glycopeptide structure having a monoisotopic mass of 4915.03 and comprising: the amino acid sequence of SEQ ID NO: 29; and glycan structure GL NO. 5402 linked to the 8^{th} residue of SEQ ID NO: 29; a ninth glycopeptide structure having a monoisotopic mass of 2621.06 and comprising: the amino acid sequence of SEQ ID NO: 30; and glycan structure GL NO. 5301 linked to the 1st residue of SEQ ID NO: 30; a tenth glycopeptide structure having a monoisotopic mass of 4900.17 and comprising: the amino acid sequence of SEQ ID NO: 31; and glycan structure GL NO. 6411 linked to the 6^{th} residue of SEQ ID NO: 31; an 11^{th} glycopeptide structure having a monoisotopic mass of 4231.67 and comprising: the amino acid sequence of SEQ ID NO: 32; and glycan structure GL NO. 6610 linked to the 11^{th} residue of SEQ ID NO: 32; a 12^{th} glycopeptide structure having a monoisotopic mass of 4729.04 and comprising: the amino acid sequence of SEQ ID NO: 33; and glycan structure GL NO. 7602 linked to the 6^{th} residue of SEQ ID NO: 33; a 13^{th} glycopeptide structure having a monoisotopic mass of 4055.56 and comprising: the amino acid sequence of SEQ ID NO: 34; and glycan structure GL NO. 5402 linked to the 1^{st} or 7^{th} residue of SEQ ID NO: 34; a 14^{th} glycopeptide structure having a monoisotopic mass of 5133.19 and comprising: the amino acid sequence of SEQ ID NO: 34; and glycan structure GL NO. 5402 linked to the 7^{th} residue of SEQ ID NO: 34; a 15^{th} glycopeptide structure having a monoisotopic mass of 5191.23 and comprising: comprising the amino acid sequence of SEQ ID NO: 35; and glycan structure GL NO. 5402 linked to the 19^{th} residue of SEQ ID NO: 35; a 16^{th} glycopeptide structure having a monoisotopic mass of 5133.19 and comprising: comprising the amino acid sequence of SEQ ID NO: 35; and glycan structure GL NO. 5511 linked to the 19^{th} residue of SEQ ID NO: 35; a 17^{th} glycopeptide structure having a monoisotopic mass of 5744.60 and comprising: the amino acid sequence of SEQ ID NO: 35; and glycan structure GL NO. 5402 linked to the 19^{th} residue of SEQ ID NO: 35; a 18^{th} glycopeptide structure having a monoisotopic mass of 4758.93 and comprising: the amino acid sequence of SEQ ID NO: 36; and glycan structure GL NO. 5402 linked to the 4^{th} residue of SEQ ID NO: 36; a 19^{th} glycopeptide structure having a monoisotopic mass of 3148.20 and comprising: the amino acid sequence of SEQ ID NO: 37; and glycan structure GL NO. 6513 linked to the 4^{th} residue of SEQ ID NO: 37; a 20^{th} glycopeptide structure having a monoisotopic mass of 3148.20 and comprising: the amino acid sequence of SEQ ID NO: 38; and glycan structure GL NO. 5402 linked to the 2^{nd} residue of SEQ ID NO: 38; a 21^{st} glycopeptide structure having a monoisotopic mass of 2633.04 and comprising: the amino acid sequence of SEQ ID NO: 39; and glycan structure GL NO. 3410 linked to the 5^{th} residue of SEQ ID NO: 39; a 22^{nd} glycopeptide structure having a monoisotopic mass of 2836.12 and comprising: the amino acid sequence of SEQ ID NO: 39; and glycan structure GL NO. 3510 linked to the 5^{th} residue of SEQ ID NO: 39; a 23^{rd} glycopeptide structure having a monoisotopic mass of 2560.02 and comprising: the amino acid sequence of SEQ ID NO: 39; and glycan structure GL NO. 4310 linked to the 5^{th} residue of SEQ ID NO: 39; a 24^{th} glycopeptide structure having a monoisotopic mass of 2617.04 and comprising: the amino acid sequence of SEQ ID NO: 39; and glycan structure GL NO. 4400 linked to the 5^{th} residue of SEQ ID NO: 39; a 25^{th} glycopeptide structure having a monoisotopic mass of 2763.10 and comprising: the amino acid sequence of SEQ ID NO: 39; and glycan structure GL NO. 4410 linked to the 5^{th} residue of SEQ ID NO: 39; a 26^{th} glycopeptide structure having a monoisotopic mass of 3054.20 and comprising: the amino acid sequence of SEQ ID NO: 39; and glycan structure GL NO. 4411 linked to the 5^{th} residue of SEQ ID NO: 39; a 27^{h} glycopeptide structure having a monoisotopic mass of 3257.28 and comprising: the amino acid sequence of SEQ ID NO: 39; and glycan structure GL NO. 4511 linked to the 5^{th} residue of SEQ ID NO: 39; a 28^{th} glycopeptide structure having a monoisotopic mass of 2925.15 and comprising: the amino acid sequence of SEQ ID NO: 39; and glycan structure GL NO. 5410 linked to the 5^{th} residue of SEQ ID NO: 39; a 29^{th} glycopeptide structure having a monoisotopic mass of 3216.25 and comprising: the amino acid sequence of SEQ ID NO: 39; and glycan structure GL NO. 5411 linked to the 5^{th} residue of SEQ ID NO: 39; a 30^{th} glycopeptide structure having a monoisotopic mass of 3128.23 and comprising: the amino acid sequence of SEQ ID NO: 39; and glycan structure GL NO. 5510 linked to the 5^{th} residue of SEQ ID NO: 39; a 31^{st} glycopeptide structure having a monoisotopic mass of 2671.29 and comprising: the amino acid sequence of SEQ ID NO: 40; and glycan structure GL NO. 1101 linked to the 6^{th} residue of SEQ ID NO: 40; a 32^{nd} glycopeptide structure having a monoisotopic mass of 2962.39 and comprising: the amino acid sequence of SEQ ID NO: 40; and glycan structure GL NO. 1102 linked to the 6^{th} residue of SEQ ID NO: 40; a 33^{rd} glycopeptide structure having a monoisotopic mass of 4561.95 and comprising: the amino acid sequence of SEQ ID NO: 41; and glycan structure GL NO. 5402 linked to the 6^{th} residue of SEQ ID NO: 41; a 34^{th} glycopeptide structure having a monoisotopic mass of 3640.56 and comprising: the amino acid sequence of SEQ ID NO: 42; and glycan structure GL NO. 5402 linked to the 7^{th} residue of SEQ ID NO: 42; a 35^{th} glycopeptide structure having a monoisotopic mass of 4159.85 and comprising: the amino acid sequence of SEQ ID NO: 43; and glycan structure GL NO. 5401 linked to the 6^{th} residue of SEQ ID NO: 43; a 36^{th} glycopeptide structure having a monoisotopic mass of 5034.18 and comprising: the amino acid sequence of SEQ ID NO: 44; and glycan structure GL NO. 5402 linked to the 2^{nd} residue of SEQ ID NO: 44; a 37^{th} glycopeptide structure having a monoisotopic mass of 3326.42 and comprising: the amino acid sequence of SEQ ID NO: 45; and glycan structure GL NO. 6301 linked to the 10^{th} residue of SEQ ID NO: 45; a 38^{th} glycopeptide structure having a monoisotopic mass of 2280.98 and comprising: the amino acid sequence of SEQ ID NO: 46; and glycan structure GL NO. 1102 linked to the 8^{th} residue of SEQ ID NO: 46; a 39^{th} glycopeptide structure having a monoisotopic mass of 2813.31 and comprising: the amino acid sequence of SEQ ID NO: 47; and glycan structure GL NO. 1101 linked to the 12^{th} residue of SEQ ID NO: 47; a 40^{th} glycopeptide structure having a monoisotopic mass of 8432.83 and comprising: the amino acid sequence of SEQ ID NO: 48; and glycan structure GL NO. 5402 linked to the 46^{th} residue of SEQ ID NO: 48; a 41^{st} glycopeptide structure having a monoisotopic mass of 4791.91 and comprising: the amino acid sequence of SEQ ID NO: 49; and glycan structure GL NO. 7420 linked to the 4^{th} residue of SEQ ID NO: 49; wherein: the glycan structure GL NO. 1101 comprises:

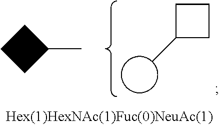

the glycan structure GL NO. 1102 comprises:

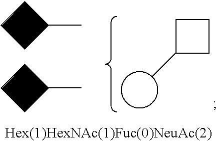

the glycan structure GL NO. 3410 comprises:

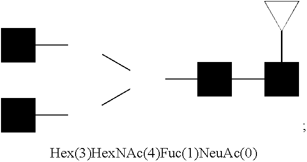

the glycan structure GL NO. 3510 comprises:

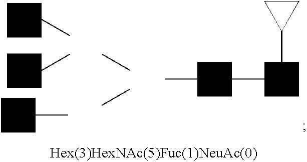

the glycan structure GL NO. 3510 comprises:

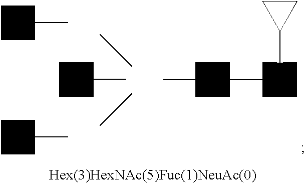

the glycan structure GL NO. 4310 comprises:

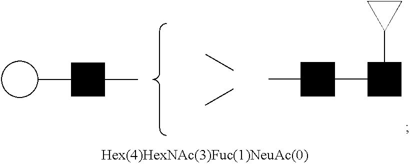

the glycan structure GL NO. 4400 comprises:

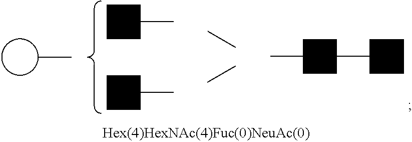

the glycan structure GL NO. 4410 comprises:

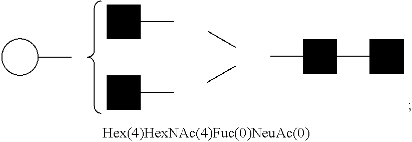

the glycan structure GL NO. 4411 comprises:

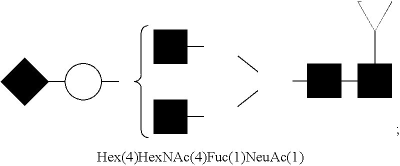

the glycan structure GL NO. 4511 comprises:

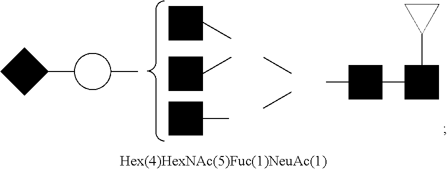

the glycan structure GL NO. 5301 comprises:

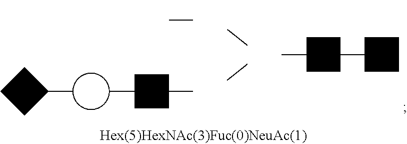

the glycan structure GL NO. 5401 comprises:

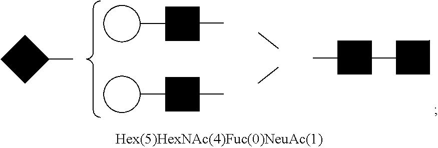

the glycan structure GL NO. 5402 comprises:

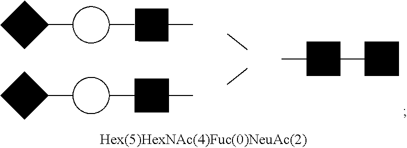

the glycan structure GL NO. 5410 comprises:

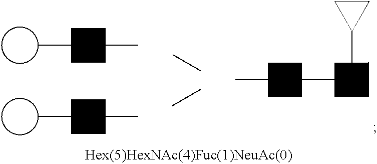

the glycan structure GL NO. 5411 comprises:

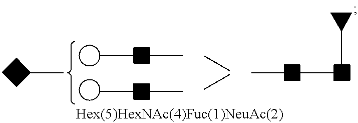

the glycan structure GL NO. 5412 comprises:

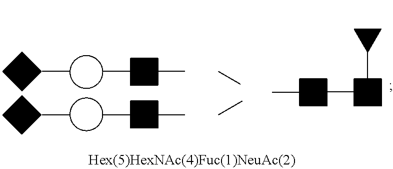

the glycan structure GL NO. 5421 comprises:

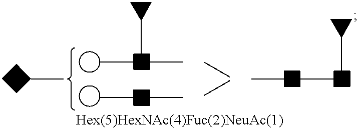

the glycan structure GL NO. 5510 comprises:

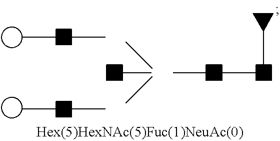

the glycan structure GL NO. 5511 comprises:

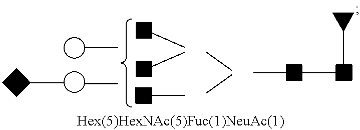

the glycan structure GL NO. 5511 comprises:

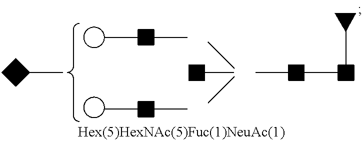

the glycan structure GL NO. 6301 comprises:

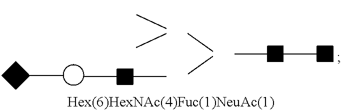

the glycan structure GL NO. 6411 comprises:

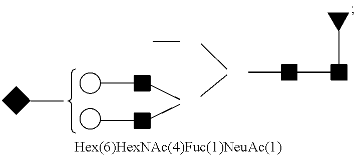

the glycan structure GL NO. 6502 comprises:

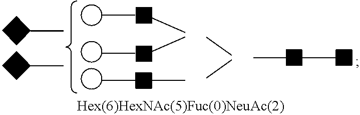

the glycan structure GL NO. 6503 comprises:

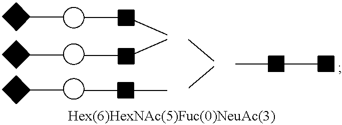

the glycan structure GL NO. 6513 comprises:

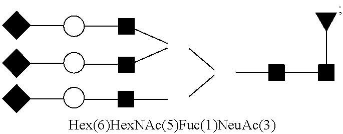

the glycan structure GL NO. 6610 comprises:

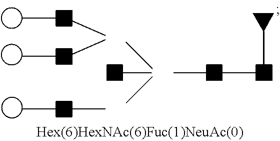

the glycan structure GL NO. 7420 comprises:

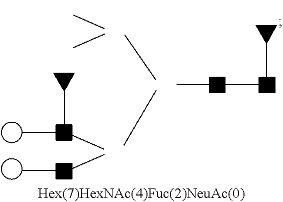

the glycan structure GL NO. 7602 comprises:

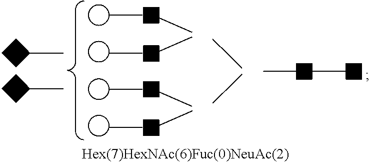

and the glycan structure GL NO. 7601 comprises:

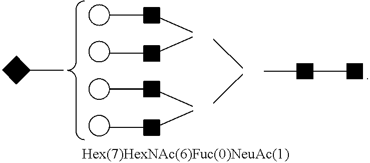

Embodiment 49: The composition of embodiment 48, wherein: the first glycopeptide structure has a precursor ion having a charge of 4; the second glycopeptide structure has a precursor ion having a charge of 5; the third glycopeptide structure has a precursor ion having a charge of 3; the fourth glycopeptide structure has a precursor ion having a charge of 4; the fifth glycopeptide structure has a precursor ion having a charge of 5; the sixth glycopeptide structure has a precursor ion having a charge of 4; the seventh glycopeptide structure has a precursor ion having a charge of 4; the eighth glycopeptide structure has a precursor ion having a charge of 5; the ninth glycopeptide structure has a precursor ion having a charge of 3; the tenth glycopeptide structure has a precursor ion having a charge of 4; the 11^{th} glycopeptide structure has a precursor ion having a charge of 4; the 12^{th} glycopeptide structure has a precursor ion having a charge of 4; the 13^{th} glycopeptide structure has a precursor ion having a charge of 4; the 14^{th} glycopeptide structure has a precursor ion having a charge of 4; the 15^{th} glycopeptide structure has a precursor ion having a charge of 5; the 16^{th} glycopeptide structure has a precursor ion having a charge of 4; the 17^{th} glycopeptide structure has a precursor ion having a charge of 5; the 18^{th} glycopeptide structure has a precursor ion having a charge of 4; the 19^{th} glycopeptide structure has a precursor ion having a charge of 3; the 20^{th} glycopeptide structure has a precursor ion having a charge of 4; the 21^{st} glycopeptide structure has a precursor ion having a charge of 3; the 22^{nd} glycopeptide structure has a precursor ion having a charge of 3; the 23^{rd} glycopeptide structure has a precursor ion having a charge of 2; the 24^{th} glycopeptide structure has a precursor ion having a charge of 3; the 25^{th} glycopeptide structure has a precursor ion having a charge of 3; the 26^{th} glycopeptide structure has a precursor ion having a charge of 4; the 27^{th} glycopeptide structure has a precursor ion having a charge of 3; the 28^{th} glycopeptide structure has a precursor ion having a charge of 3; the 29^{th} glycopeptide structure has a precursor ion having a charge of 3; the 30^{th} glycopeptide structure has a precursor ion having a charge of 3; the 31^{st} glycopeptide structure has a precursor ion having a charge of 3; the 32^{nd} glycopeptide structure has a precursor ion having a charge of 3; the 33^{rd} glycopeptide structure has a precursor ion having a charge of 4; the 34^{th} glycopeptide structure has a precursor ion having a charge of 4; the 35^{th} glycopeptide structure has a precursor ion having a charge of 4; the 36^{th} glycopeptide structure has a precursor ion having a charge of 5; the 37^{th} glycopeptide structure has a precursor ion having a charge of 3; the 38^{th} glycopeptide structure has a precursor ion having a charge of 3; the 39^{th} glycopeptide structure has a precursor ion having a charge of 3; the 40^{th} glycopeptide structure has a precursor ion having a charge of 7; and the 41^{st} glycopeptide structure has a precursor ion having a charge of 4.

Embodiment 50: The composition of embodiment 48 or embodiment 49, wherein: the first glycopeptide structure has a product ion having a mass-to-charge (m/z) ratio within a range selected from a group consisting of 366.1±0.5, ±0.8, and ±1.0; the second glycopeptide structure has a product ion having a mass-to-charge (m/z) ratio that is within a range selected from a group consisting of 366.1±0.5, ±0.8, and ±1.0; the third glycopeptide structure associated with the corresponding set of product ions that includes a product ion having a mass-to-charge (m/z) ratio that is within a range selected from a group consisting of 366.1±0.5, ±0.8, and ±1.0; the fourth glycopeptide structure has a product ion having a mass-to-charge (m/z) ratio that is within a range selected from a group consisting of 274.1±0.5, ±0.8, and ±1.0; the fifth glycopeptide structure has a product ion having a mass-to-charge (m/z) ratio that is within a range selected from a group consisting of 366.1±0.5, ±0.8, and ±1.0; the sixth glycopeptide structure has a product ion having a mass-to-charge (m/z) ratio that is within a range selected from a group consisting of 366.1±0.5, ±0.8, and ±1.0; the seventh glycopeptide structure has a product ion having a mass-to-charge (m/z) ratio that is within a range selected from a group consisting of 366.1±0.5, 0.8, and ±1.0; the eighth glycopeptide structure has a product ion having a mass-to-charge (m/z) ratio that is within a range selected from a group consisting of 274.1±0.5, ±0.8, and ±1.0; the ninth glycopeptide structure has a product ion having a mass-to-charge (m/z) ratio that is within a range selected from a group consisting of 366.1±0.5, ±0.8, and ±1.0; the tenth glycopeptide structure has a product ion having a mass-to-charge (m/z) ratio that is within a range selected from a group consisting of 366.1±0.5, ±0.8, and ±1.0; the 11^{th} glycopeptide structure has a product ion having a mass-to-charge (m/z) ratio that is within a range selected from a group consisting of 366.1±0.5, ±0.8, and ±1.0; the 12^{th} glycopeptide structure has a product ion having a mass-to-charge (m/z) ratio that is within a range selected from a group consisting of 366.1±0.5, ±0.8, and ±1.0; the 13^{th} glycopeptide structure has a product ion having a mass-to-charge (m/z) ratio that is within a range selected from a group consisting of 366.1±0.5, ±0.8, and ±1.0; the 14^{th} glycopeptide structure has a product ion having a mass-to-charge (m/z) ratio that is within a range selected from a group consisting of 366.1±0.5, ±0.8, and ±1.0; the 15^{th} glycopeptide structure has a product ion having a mass-to-charge (m/z) ratio that is within a range selected from a group consisting of 366.1±0.5, ±0.8, and ±1.0; the 16^{th} glycopeptide structure has a product ion having a mass-to-charge (m/z) ratio that is within a range selected from a group consisting of 366.1±0.5, ±0.8, and ±1.0; the 17^{th} glycopeptide structure has a product ion having a mass-to-charge (m/z) ratio that is within a range selected from a group consisting of 1249.3±0.5, ±0.8, and ±1.0; the 18^{th} glycopeptide structure has a product ion having a mass-to-charge (m/z) ratio that is within a range selected from a group consisting of 366.1±0.5, ±0.8, and ±1.0; the 19^{th} glycopeptide structure has a product ion having a mass-to-charge (m/z) ratio that is within a range selected from a group consisting of 366.1±0.5, ±0.8, and ±1.0; the 20^{th} glycopeptide structure has a product ion having a mass-to-charge (m/z) ratio that is within a range selected from a group consisting of 366.1±0.5, ±0.8, and ±1.0; the 21^{st} glycopeptide structure has a product ion having a mass-to-charge (m/z) ratio that is within a range selected from a group consisting of 204.1±0.5, ±0.8, and ±1.0; the 22^{nd} glycopeptide structure has a product ion having a mass-to-charge (m/z) ratio that is within a range selected from a group consisting of 204.1±0.5, ±0.8, and ±1.0; the 23^{rd} glycopeptide structure has a product ion having a mass-to-charge (m/z) ratio that is within a range selected from a group consisting of 366.1±0.5, ±0.8, and ±1.0; the 24^{th} glycopeptide structure has a product ion having a mass-to-charge (m/z) ratio that is within a range selected from a group consisting of 204.1±0.5, ±0.8, and ±1.0; the 25^{th} glycopeptide structure has a product ion having a mass-to-charge (m/z) ratio that is within a range selected from a group consisting of 204.1±0.5, ±0.8, and ±1.0; the 26^{th} glycopeptide structure has a product ion having a mass-to-charge (m/z) ratio that is within a range selected from a group consisting of 204.1±0.5, ±0.8, and ±1.0; the 27^{th} glycopeptide structure has a product ion having a mass-to-charge (m/z) ratio that is within a range selected from a group consisting of 204.1±0.5, ±0.8, and ±1.0; the 28^{th} glycopeptide structure has a product ion having a mass-to-charge (m/z) ratio that is within a range selected from a group consisting of 366.1±0.5, ±0.8, and ±1.0; the 29^{th} glycopeptide structure has a product ion having a mass-to-charge (m/z) ratio that is within a range selected from a group consisting of 366.1±0.5, ±0.8, and ±1.0; the 30^{th} glycopeptide structure has a product ion having a mass-to-charge (m/z) ratio that is within a range selected from a group consisting of 366.1±0.5, ±0.8, and ±1.0; the 31^{st} glycopeptide structure has a product ion having a mass-to-charge (m/z) ratio that is within a range selected from a group consisting of 1009±0.5, ±0.8, and ±1.0; the 32^{nd} glycopeptide structure has a product ion having a mass-to-charge (m/z) ratio that is within a range selected from a group consisting of 274.1±0.5, ±0.8, and ±1.0; the 33^{rd} glycopeptide structure has a product ion having a mass-to-charge (m/z) ratio that is within a range selected from a group consisting of 204.1±0.5, ±0.8, and ±1.0; the 34^{th} glycopeptide structure has a product ion having a mass-to-charge (m/z) ratio that is within a range selected from a group consisting of 274.1±0.5, ±0.8, and ±1.0; the 35^{th} glycopeptide structure has a product ion having a mass-to-charge (m/z) ratio that is within a range selected from a group consisting of 366.1±0.5, ±0.8, and ±1.0; the 36^{th} glycopeptide structure has a product ion having a mass-to-charge (m/z) ratio that is within a range selected from a group consisting of 366.1±0.5, ±0.8, and ±1.0; the 37^{th} glycopeptide structure has a product ion having a mass-to-charge (m/z) ratio that is within a range selected from a group consisting of 366.1±0.5, ±0.8, and ±1.0; the 38^{th} glycopeptide structure has a product ion having a mass-to-charge (m/z) ratio that is within a range selected from a group consisting of 274.1±0.5, ±0.8, and ±1.0; the 39^{th} glycopeptide structure has a product ion having a mass-to-charge (m/z) ratio that is within a range selected from a group consisting of 274.1±0.5, ±0.8, and ±1.0; the 40^{th} glycopeptide structure has a product ion having a mass-to-charge (m/z) ratio that is within a range selected from a group consisting of 366.1±0.5, ±0.8, and ±1.0; and the 41^{st} glycopeptide structure has a product ion having a mass-to-charge (m/z) ratio that is within a range selected from a group consisting of 366.1±0.5, ±0.8, and ±1.0.

Embodiment 51: The composition of any one of embodiments 48-50, wherein: the first glycopeptide structure has a precursor ion having an m/z ratio of 1386.1 within a range selected from a group consisting of ±1.0 and ±1.5; the second glycopeptide structure has a precursor ion having an m/z ratio of 1167.3 within a range selected from a group consisting of ±1.0 and ±1.5; the third glycopeptide structure has a precursor ion having an m/z ratio of 1270.2 within a range selected from a group consisting of ±1.0 and ±1.5; the fourth glycopeptide structure has a precursor ion having an m/z ratio of 1152.5 within a range selected from a group consisting of ±1.0 and ±1.5; the fifth glycopeptide structure has a precursor ion having an m/z ratio of 918.4 within a range selected from a group consisting of ±1.0 and ±1.5; the sixth glycopeptide structure has a precursor ion having an m/z ratio of 1032.9 within a range selected from a group consisting of ±1.0 and ±1.5; the seventh glycopeptide structure has a precursor ion having an m/z ratio of 1197 within a range selected from a group consisting of ±1.0 and ±1.5; the eighth glycopeptide structure has a precursor ion having an m/z ratio of 984.4 within a range selected from a group consisting of ±1.0 and ±1.5; the ninth glycopeptide structure has a precursor ion having an m/z ratio of 874.7 within a range selected from a group consisting of ±1.0 and ±1.5; the tenth glycopeptide structure has a precursor ion having an m/z ratio of 1225.8 within a range selected from a group consisting of ±1.0 and ±1.5; the 11^{th} glycopeptide structure has a precursor ion having an m/z ratio of 1073.4 within a range selected from a group consisting of ±1.0 and ±1.5; the 12^{th} glycopeptide structure has a precursor ion having an m/z ratio of 1183.5 within a range selected from a group consisting of ±1.0 and ±1.5; the 13^{th} glycopeptide structure has a precursor ion having an m/z ratio of 1015.2 within a range selected from a group consisting of ±1.0 and ±1.5; the 14^{th} glycopeptide structure has a precursor ion having an m/z ratio of 1284.6 within a range selected from a group consisting of ±1.0 and ±1.5; the 15^{th} glycopeptide structure has a precursor ion having an m/z ratio of 1039.1 within a range selected from a group consisting of ±1.0 and ±1.5; the 16^{th} glycopeptide structure has a precursor ion having an m/z ratio of 1285.1 within a range selected from a group consisting of ±1.0 and ±1.5; the 17^{th} glycopeptide structure has a precursor ion having an m/z ratio of 1150.3 within a range selected from a group consisting of ±1.0 and ±1.5; the 18^{th} glycopeptide structure has a precursor ion having an m/z ratio of 1191.2 within a range selected from a group consisting of ±1.0 and ±1.5; the 19^{th} glycopeptide structure has a precursor ion having an m/z ratio of 1050.4 within a range selected from a group consisting of ±1.0 and ±1.5; the 20^{th} glycopeptide structure has a precursor ion having an m/z ratio of 788.3 within a range selected from a group consisting of ±1.0 and ±1.5; the 21^{st} glycopeptide structure has a precursor ion having an m/z ratio of 878.8 within a range selected from a group consisting of ±1.0 and ±1.5; the 22^{nd} glycopeptide structure has a precursor ion having an m/z ratio of 946.5 within a range selected from a group consisting of ±1.0 and ±1.5; the 23^{rd} glycopeptide structure has a precursor ion having an m/z ratio of 1281.5 within a range selected from a group consisting of ±1.0 and ±1.5; the 24^{th} glycopeptide structure has a precursor ion having an m/z ratio of 873.4 within a range selected from a group consisting of ±1.0 and ±1.5; the 25^{th} glycopeptide structure has a precursor ion having an m/z ratio of 922.1 within a range selected from a group consisting of ±1.0 and ±1.5; the 26^{th} glycopeptide structure has a precursor ion having an m/z ratio of 764.8 within a range selected from a group consisting of ±1.0 and ±1.5; the 27^{th} glycopeptide structure has a precursor ion having an m/z ratio of 1087.1 within a range selected from a group consisting of ±1.0 and ±1.5; the 28^{th} glycopeptide structure has a precursor ion having an m/z ratio of 976.1 within a range selected from a group consisting of ±1.0 and ±1.5; the 29^{th} glycopeptide structure has a precursor ion having an m/z ratio of 1073.1 within a range selected from a group consisting of ±1.0 and ±1.5; the 30^{th} glycopeptide structure has a precursor ion having an m/z ratio of 1043.8 within a range selected from a group consisting of ±1.0 and ±1.5; the 31^{st} glycopeptide structure has a precursor ion having an m/z ratio of 891.8 within a range selected from a group consisting of ±1.0 and ±1.5; the 32^{nd} glycopeptide structure has a precursor ion having an m/z ratio of 988.8 within a range selected from a group consisting of ±1.0 and ±1.5; the 33^{rd} glycopeptide structure has a precursor ion having an m/z ratio of 1142 within a range selected from a group consisting of ±1.0 and ±1.5; the 34^{th} glycopeptide structure has a precursor ion having an m/z ratio of 911.4 within a range selected from a group consisting of ±1.0 and ±1.5; the 35^{th} glycopeptide structure has a precursor ion having an m/z ratio of 1041 within a range selected from a group consisting of ±1.0 and ±1.5; the 36^{th} glycopeptide structure has a precursor ion having an m/z ratio of 1008.2 within a range selected from a group consisting of ±1.0 and ±1.5; the 37^{th} glycopeptide structure has a precursor ion having an m/z ratio of 1109.8 within a range selected from a group consisting of ±1.0 and ±1.5; the 38^{th} glycopeptide structure has a precursor ion having an m/z ratio of 761.7 within a range selected from a group consisting of ±1.0 and ±1.5; the 39^{th} glycopeptide structure has a precursor ion having an m/z ratio of 939.1 within a range selected from a group consisting of ±1.0 and ±1.5; the 40^{th} glycopeptide structure has a precursor ion having an m/z ratio of 1206.3 within a range selected from a group consisting of ±1.0 and ±1.5; and the 41^{st} structure has a precursor ion having an m/z ratio of 1199.2 within a range selected from a group consisting of ±1.0 and ±1.5.

Embodiment 52: A composition comprising a peptide structure selected as one from a group of aglycosylated peptide structures consisting of: a first peptide structure having a monoisotopic mass of 1392.69 and comprising the amino acid sequence of SEQ ID NO: 50; a second peptide structure having a monoisotopic mass of 1234.68 and comprising the amino acid sequence of SEQ ID NO: 51; a third peptide structure having a monoisotopic mass of 1178.67 and comprising the amino acid sequence of SEQ ID NO: 52; a fourth peptide structure having a monoisotopic mass of 2454.14 and comprising the amino acid sequence of SEQ ID NO: 53; and a fifth peptide structure having a monoisotopic mass of 831.47 and comprising the amino acid sequence of SEQ ID NO: 54.

Embodiment 53: The composition of embodiment 52, wherein: the first peptide structure has a precursor ion having a charge of 2; the second peptide structure has a precursor ion having a charge of 2; the third peptide structure has a precursor ion having a charge of 2; the fourth peptide structure has a precursor ion having a charge of 3; and the fifth peptide structure has a precursor ion having a charge of 2.

Embodiment 54: The composition of any one of embodiments 52-53, wherein: the first peptide structure has a precursor ion m/z ratio within a range selected from a group consisting of 697.4+/−1.0, and +/−1.5; the second peptide structure has a precursor ion m/z ratio within a range selected from a group consisting of 618.3+/−1.0, and +/−1.5; the third peptide structure has a precursor ion m/z ratio within a range selected from a group consisting of 590.3+/−1.0, and +/−1.5; the fourth peptide structure has a precursor ion m/z ratio within a range selected from a group consisting of 819.1+/−1.0, and +/−1.5; and the fifth peptide structure has a precursor ion m/z ratio within a range selected from a group consisting of 416.7+/−1.0, and +/−1.5.

Embodiment 55: The composition of any one of embodiments 52-54, wherein: the first peptide structure has a product ion m/z ratio within a range selected from a group consisting of 565.3+/−0.5, +/−0.8, and +/−1.0; the second peptide structure has a product ion m/z ratio within a range selected from a group consisting of 736.4+/−0.5, +/−0.8, and +/−1.0; the third peptide structure has a product ion m/z ratio within a range selected from a group consisting of 342.2+/−0.5, +/−0.8, and +/−1.0; the fourth peptide structure has a product ion m/z ratio within a range selected from a group consisting of 609.3+/−0.5, +/−0.8, and +/−1.0; and the fifth peptide structure has a product ion m/z ratio within a range selected from a group consisting of 646.4+/−0.5, +/−0.8, and +/−1.0.

Embodiment 56: A kit comprising at least one agent for quantifying at least one peptide structure identified in Table 1 to carry out the method of any one of embodiments 1-45.

Embodiment 57: A kit comprising at least one of a glycopeptide standard, a buffer, or a set of peptide sequences to carry out the method of any one of embodiments 1-45, a peptide sequence of the set of peptide sequences identified by a corresponding one of SEQ ID NOS: 24-54, defined in Table 3.

Embodiment 58: A system comprising: one or more data processors; and a non-transitory computer readable storage medium containing instructions which, when executed on the one or more data processors, cause the one or more data processors to perform part or all of any one of embodiments 1-22, 24-29, or 30-33.

Embodiment 59: A computer-program product tangibly embodied in a non-transitory machine-readable storage medium, including instructions configured to cause one or more data processors to perform part or all of any one of embodiments 1-22, 24-29, or 30-33.

Embodiment 60: A kit comprising at least one of a glycopeptide standard, a buffer, or a set of peptide sequences to carry out the method of any one of embodiments 1-48, a peptide sequence of the set of peptide sequences identified by a corresponding one of SEQ ID NOS: 24-54, defined in Table 3.

Embodiment 61: A system comprising: one or more data processors; and a non-transitory computer readable storage medium containing instructions which, when executed on the one or more data processors, cause the one or more data processors to perform part or all of any one of embodiments 1-23, 25-30, or 31-35.

Embodiment 62: A computer-program product tangibly embodied in a non-transitory machine-readable storage medium, including instructions configured to cause one or more data processors to perform part or all of any one of embodiments 1-23, 25-30, or 31-35.

Embodiment 63. A method of determining whether a biological sample corresponds to a symptomatic disease state of a coronavirus disease (COVID), the method comprising: inputting quantification data identified from peptide structure data for a set of peptide structures into a supervised machine learning model, wherein the set of peptide structures includes at least one peptide structure identified in Table 2-1; analyzing the quantification data using the supervised machine learning model to generate a disease indicator that classifies the biological sample as corresponding to a cluster of a plurality of clusters that has a least distance to the biological sample, the plurality of clusters corresponding a plurality of states that includes the symptomatic disease state; and generating a diagnosis output based on the disease indicator.

Embodiment 64. The method of embodiment 63, further comprising receiving peptide structure data corresponding to the biological sample obtained from a subject.

Embodiment 65. The method of embodiment 63 or 64, wherein the at least one peptide structure comprises a glycopeptide structure defined by a peptide sequence and a glycan structure linked to the peptide sequence at a linking site of the peptide sequence, as identified in Table 2-1, with the peptide sequence being one of SEQ ID NOS: 30-5284-116 as defined in Table 5-1.

Embodiment 66. The method of any one of embodiments 63-65, wherein the at least one peptide structure comprises an aglycosylated peptide structure defined by a peptide sequence, as identified in Table 2-1, with the peptide sequence being one of SEQ ID NOS: 107-110 as defined in Table 5-1.

Embodiment 67. The method of any one of embodiments 63-66, wherein the supervised machine learning model comprises a Support Vector Machine (SVM) classifier.

Embodiment 68. The method of any one of embodiments 63-67, wherein the supervised machine learning model comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

Embodiment 69. The method of any one of embodiments 63-68, further comprising: training the supervised machine learning model using training data generated from an unsupervised machine learning model, wherein the training data comprises a plurality of peptide structure profiles for a plurality of subjects and identifies a corresponding state of the plurality of states for each peptide structure profile of the plurality of peptide structure profiles; and wherein the unsupervised machine learning model is trained to cluster the plurality of peptide structure profiles into the plurality of clusters.

Embodiment 70. The method of embodiment 69, wherein the unsupervised machine learning model is a k-means clustering model.

Embodiment 71. The method of embodiment 69 or embodiment 70, wherein a peptide structure profile of the plurality of peptide structure profiles for a corresponding subject of the plurality of subjects is selected based on a differential expression analysis of quantification metrics for a panel of peptide structures for the plurality of subjects.

Embodiment 72. The method of embodiment 71, further comprising: comparing, using the differential expression analysis, the quantification metrics corresponding to the panel of peptide structures for a first portion of the plurality of subjects diagnosed with the symptomatic disease state to that of each portion of a set of other portions of the plurality of subjects to generate a set of comparisons; and selecting a portion of the panel of peptide structures having a false discovery rate below 0.05 across the set of comparisons as the set of peptide structures.

Embodiment 73. The method of embodiment 72, wherein the comparing comprises: comparing the quantification metrics corresponding to the panel of peptide structures for the first portion of the plurality of subjects diagnosed with the symptomatic disease state to that of a second portion of the plurality of subjects diagnosed with a sepsis state to generate a first comparison of the set of comparisons.

Embodiment 74. The method of embodiment 73, wherein the comparing further comprises: comparing the quantification metrics corresponding to the panel of peptide structures for the first portion of the plurality of subjects diagnosed with the symptomatic disease state to that of at least one of: a third portion of the plurality of subjects diagnosed with a common cold state to generate a second comparison of the set of comparisons; a third portion of the plurality of subjects diagnosed with an asymptomatic disease state of the coronavirus disease to generate a third comparison of the set of comparisons; or a fourth portion of the plurality of subjects diagnosed with a healthy state to generate a fourth comparison of the set of comparisons.

Embodiment 75. The method of any one of embodiments 63-74, wherein the quantification data for a peptide structure of the set of peptide structures comprises at least one of a relative quantity, an adjusted quantity, a normalized quantity, a relative concentration, an adjusted concentration, or a normalized concentration.

Embodiment 76. The method of any one of embodiments 63-75, wherein the peptide structure data is generated using multiple reaction monitoring mass spectrometry (MRM-MS).

Embodiment 77. The method of any one of embodiments 63-76, further comprising: creating a sample from the biological sample; and preparing the sample using reduction, alkylation, and enzymatic digestion to form a prepared sample that includes a set of peptide structures.

Embodiment 78. The method of any one of embodiments 63-77, further comprising: generating the peptide structure data from the prepared sample using multiple reaction monitoring mass spectrometry (MRM-MS).

Embodiment 79. The method of any one of embodiments 63-78, wherein generating the diagnosis output comprises: generating a report that includes a diagnosis that the subject is positive for the symptomatic disease state in response to the disease indicator classifying the biological sample as corresponding to the symptomatic disease state of the coronavirus disease.

Embodiment 80. The method of any one of embodiments 63-79, wherein the coronavirus disease is caused by severe acute respiratory syndrome coronavirus 2 (SARS-CoV-2).

Embodiment 81. The method of any one of embodiments 63-80, further comprising: generating a treatment output based on at least one of the diagnosis output or the disease indicator.

Embodiment 82. The method of embodiment 81, wherein the treatment output comprises at least one of an identification of a treatment to treat the subject, a design for the treatment, a manufacturing plan for the treatment, or a treatment plan for administering the treatment.

Embodiment 83. The method of embodiment 81 or embodiment 82, wherein the treatment comprises at least one of an antiviral therapeutic, an anti-inflammatory therapeutic, or an immune-based therapeutic.

Embodiment 84. The method of any one of embodiments 81-83, wherein the treatment comprises at least one of remdesivir, baricitinib, tocilizumab, favipiravir, merimepodib, a monoclonal antibody therapeutic, Nirmatrelvir with Ritonavi, Molnupiravir, and/or an immune checkpoint inhibitor.

Embodiment 85. The method of any one of embodiments 81-84, wherein generating the treatment output comprises: determining a dosage for a therapeutic to treat the subject based on at least one of the diagnosis output or the disease indicator.

Embodiment 86. The method of any one of embodiments 63-85, wherein the diagnosis output identifies that the biological sample is positive for the symptomatic disease state and further comprising: administering a therapeutic dosage of a therapeutic for the coronavirus disease to the subject, the therapeutic being selected from the group consisting of remdesivir, baricitinib, tocilizumab, favipiravir, merimepodib, a monoclonal antibody treatment, Nirmatrelvir with Ritonavi, Molnupiravir, and/or an immune checkpoint inhibitor.

Embodiment 87. A method of identifying a coronavirus disease (COVID)-specific set of peptide structures for use in diagnosing a symptomatic disease state of the coronavirus disease, the method comprising: receiving quantification data for a panel of peptide structures for a plurality of subjects diagnosed with a plurality of states, the plurality of states including the symptomatic disease state of the coronavirus disease; comparing quantification metrics corresponding to the panel of peptide structures for a first portion of the plurality of subjects diagnosed with the symptomatic disease state to that of each portion of a set of other portions of the plurality of subjects to generate a set of comparisons using a differential expression analysis; selecting a portion of the panel of peptide structures having a false discovery rate below 0.05 across the set of comparisons as a set of peptide structures to be associated with the coronavirus disease, wherein the set of peptide structures includes at least one glycopeptide structure defined by a peptide sequence and a glycan structure linked to the peptide sequence at a linking site of the peptide sequence; analyzing the quantification data for the set of peptide structures for the plurality of subjects using an unsupervised machine learning model to cluster the plurality of subjects; and training a supervised machine learning model to determine whether a biological sample obtained from a subject corresponds to the symptomatic disease state or another state of the plurality of states.

Embodiment 88. The method of embodiment 87, further comprising: analyzing the biological sample obtained from the subject using the supervised machine learning model that has been trained to generate a disease indicator that indicates whether the biological subject is positive for the symptomatic disease state.

Embodiment 89. The method of embodiment 87 or embodiment 88, wherein the plurality of states further includes at least one of a sepsis state, a common cold state, a healthy state, or an asymptomatic disease state of the coronavirus disease.

Embodiment 90. The method of any one of embodiments 87-89, wherein the coronavirus disease is caused by severe acute respiratory syndrome coronavirus 2 (SARS-CoV-2).

Embodiment 91. The method of any one of embodiments 87-90, wherein the unsupervised machine learning model comprises a k-means clustering model and wherein the supervised machine learning model comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

Embodiment 92. The method of any one of embodiments 87-91, wherein the set of peptide structures includes at least three peptide structures identified in Table 2-1.

Embodiment 93. A method of diagnosing a symptomatic disease state of a coronavirus disease (COVID), the method comprising: analyzing peptide structure data using a supervised machine learning model to generate a disease indicator that indicates whether a biological sample is positive for the symptomatic disease state based on at least 3 peptide structures selected from a group of peptide structures identified in Table 3-1, wherein the group of peptide structures in Table 3-1 comprises a group of peptide structures associated with the symptomatic disease state; and wherein the group of peptide structures is listed in Table 3-1 with respect to relative significance to the disease indicator; and generating a diagnosis output based on the disease indicator.

Embodiment 94. The method of embodiment 93, further comprising receiving peptide structure data corresponding to a biological sample obtained from a subject.

Embodiment 95. The method of embodiment 93 or 94, wherein a peptide structure of the at least 3 peptide structures comprises a glycopeptide structure defined by a peptide sequence and a glycan structure linked to the peptide sequence at a linking site of the peptide sequence, as identified in Table 3-1, with the peptide sequence, which is one of SEQ ID NOS: 3084, 3185, 3892, 4094, 4296, 4397, 50 104 and 57-62111-116, being defined in Table 5-1.

Embodiment 96. The method of any of embodiments 93-95, wherein a peptide structure of the at least 3 peptide structures comprises an aglycosylated peptide structure defined by a peptide sequence, as identified in Table 3-1, with the peptide sequence, which is one of SEQ ID NOS: 53-56107-110, being defined in Table 5-1.

Embodiment 97. The method of any one of embodiments 93-96, wherein the at least 3 peptide structures includes 16 glycopeptide structures and 2 aglycosylated peptide structures.

Embodiment 98. The method of any one of embodiments 93-97, wherein the supervised machine learning model comprises a regression model.

Embodiment 99. The method of any one of embodiments 93-98, wherein the supervised machine learning model comprises a penalized multivariable regression model.

Embodiment 100. The method of any one of embodiments 93-99, wherein the peptide structure data comprises quantification data for each peptide structure of a panel of peptide structures, the panel of peptide structures including the at least 3 peptide structures.

Embodiment 101. The method of embodiment 100, wherein the quantification data for a peptide structure of the plurality of peptide structures comprises at least one of a relative quantity, an adjusted quantity, a normalized quantity, a relative concentration, an adjusted concentration, or a normalized concentration.

Embodiment 102. The method of any one of embodiments 93-101, wherein the disease indicator comprises at least one of a probability that the subject is positive for the symptomatic disease state, an odds that the subject is positive for the symptomatic disease state, a logarithm of the odds that the subject is positive for the symptomatic disease state, or a classification of the biological sample as either positive or negative for the symptomatic disease state.

Embodiment 103. The method of embodiment 93, wherein generating the diagnosis output comprises: generating a report that includes a diagnosis that the subject is positive for the symptomatic disease state in response to a determination that the disease indicator is above a selected threshold.

Embodiment 104. The method of embodiment 103, wherein the selected threshold comprises at least one a probability threshold selected as a value between a range from 0.50 to 0.95 or a logit threshold selected as a value either equal to or above 0.0.

Embodiment 105. The method of any one of embodiments 93-104, wherein analyzing the peptide structure data comprises: computing the disease indicator using a weight coefficient associated with each peptide structure of the at least 3 peptide structures, the weight coefficient of a corresponding peptide structure of the at least 3 peptide structures indicating the relative significance of the corresponding peptide structure to the disease indicator.

Embodiment 106. The method of any one of embodiments 93-105, wherein analyzing the peptide structure data comprises: computing a peptide structure profile for the biological sample that identifies a weighted value for each peptide structure of the at least 3 peptide structures, wherein the weighted value for a peptide structure of the at least 3 peptide structures is a product of a quantification metric for the peptide structure identified from the peptide structure data and a weight coefficient for the peptide structure; and computing the disease indicator using the peptide structure profile.

Embodiment 107. The method of any one of embodiments 93-106, wherein the disease indicator comprises a probability that the biological sample is positive for the symptomatic disease state and wherein the supervised machine learning model is configured to generate an output that identifies the biological sample as either positive for the symptomatic disease state when the disease indicator is greater than a selected threshold or negative for the symptomatic disease state when the disease indicator is not greater than the selected threshold.

Embodiment 108. The method of embodiment 107, wherein the selected threshold is a value selected as either equal to or above 0.5.

Embodiment 109. The method of embodiment 107, wherein the selected threshold is a value within ±0.02 of 0.525.

Embodiment 110. The method of any one of embodiments 93-109, wherein: the supervised machine learning model is trained to determine weight coefficients for a panel of peptide structures such that a first portion of the weight coefficients for a first portion of the panel of peptide structures are non-zero and a second portion of the weight coefficients for a second portion of the panel of peptide structures are zero; and the first portion of the panel of peptide structures forms the group of peptide structures identified in Table 3-1.

Embodiment 111. The method of embodiment 110, further comprising: training the supervised machine learning model using training data that comprises a plurality of peptide structure profiles for a plurality of subjects and a corresponding state of a plurality of states for each peptide structure profile of the plurality of peptide structure profiles, wherein the plurality of subjects includes a first portion diagnosed with the symptomatic disease state and at least one of: a second portion having a healthy state; a third portion diagnosed with a common cold state; a fourth portion diagnosed with an asymptomatic disease state of the coronavirus disease (COVID); or a fifth portion diagnosed with a sepsis state.

Embodiment 112. The method of any one of embodiments 93-111, wherein the symptomatic disease state is one of a plurality of symptomatic disease states for the coronavirus (COVID), the plurality of symptomatic disease states corresponding to varying levels of severity.

Embodiment 113. The method of any one of embodiments 93-112, further comprising: creating a sample from the biological sample; and preparing the sample using reduction, alkylation, and enzymatic digestion to form a prepared sample that includes a set of peptide structures.

Embodiment 114. The method of embodiment 113, further comprising: generating the peptide structure data from the prepared sample using multiple reaction monitoring mass spectrometry (MRM-MS).

Embodiment 115. The method of any one of embodiments 93-114, wherein the coronavirus disease (COVID) is caused by severe acute respiratory syndrome coronavirus 2 (SARS-CoV-2).

Embodiment 116. The method of any one of embodiments 93-115, further comprising: generating a treatment output based on at least one of the diagnosis output or the disease indicator.

Embodiment 117. The method of embodiment 116, wherein the treatment output comprises at least one of an identification of a treatment to treat the subject, a design for the treatment, a manufacturing plan for the treatment, or a treatment plan for administering the treatment.

Embodiment 118. The method of embodiment 116 or embodiment 117, wherein the treatment comprises at least one of an antiviral therapeutic, an anti-inflammatory therapeutic, or an immune-based therapeutic.

Embodiment 119. The method of embodiment 116 or embodiment 117, wherein the treatment comprises at least one of remdesivir, baricitinib, tocilizumab, favipiravir, merimepodib, a monoclonal antibody therapeutic, Nirmatrelvir with Ritonavi, Molnupiravir, and/or an immune checkpoint inhibitor.

Embodiment 120. The method of any one of embodiments 116-119, wherein generating the treatment output comprises: determining a dosage for a therapeutic to treat the subject based on at least one of the diagnosis output or the disease indicator.

Embodiment 121. The method of any one of embodiments 93-120, wherein the diagnosis output identifies that the biological sample is positive for the symptomatic disease state and further comprising: administering a therapeutic dosage of a therapeutic for the coronavirus disease to the subject, the therapeutic being selected from the group consisting of remdesivir, baricitinib, tocilizumab, a monoclonal antibody treatment, Nirmatrelvir with Ritonavi, Molnupiravir, and/or an immune checkpoint inhibitor.

Embodiment 122. A method of evaluating a biological sample obtained from a subject with respect to a symptomatic disease state corresponding to a coronavirus disease (COVID), the method comprising: identifying a peptide structure profile for the biological sample using peptide structure data, the peptide structure profile comprising quantification data for a set of peptide structures associated with the symptomatic disease state, wherein the set of peptide structures includes at least two peptide structures from a selected group of peptide structures identified in Table 1-1; and wherein at least two peptide structures in the selected group of peptide structures includes a glycopeptide structure defined by a peptide sequence and a glycan structure linked to a linking site of the peptide sequence; computing a disease indicator using the peptide structure profile and a model, wherein the disease indicator indicates whether the biological sample is positive for the symptomatic disease state; and generating at least one of a diagnosis output or a treatment output based on the disease indicator.

Embodiment 123. The method of embodiment 122, further comprising receiving peptide structure data corresponding to the biological sample obtained from the subject.

Embodiment 124. The method of embodiment 122 or 123, wherein the model is a machine learning model and computing the disease indicator comprises: computing the disease indicator using the machine learning model, the machine learning model including a set of weight coefficients that corresponds to the set of peptide structures, respectively, wherein the disease indicator comprises at least one of a probability that the subject is positive for the symptomatic disease state, an odds that the subject is positive for the symptomatic disease state, a logarithm of the odds that the subject is positive for the symptomatic disease state, or a classification of the biological sample as either positive or negative for the symptomatic disease state.

Embodiment 125. The method of any of embodiments 122-124, wherein the model comprises a supervised machine learning model trained using an output of an unsupervised machine learning model that is trained to cluster a plurality of peptide structure profiles for a plurality of subjects according to a plurality of states, the plurality of states including the symptomatic disease state.

Embodiment 126. The method of any one of embodiments 122-125, wherein the treatment output comprises at least one of an identification of a therapeutic to treat the subject, a design for the therapeutic, or a treatment plan for administering the therapeutic.

Embodiment 127. The method of any one of embodiments 122-126, further comprising: administering a therapeutic dosage of a therapeutic for the coronavirus disease to the subject based on the at least one of the diagnosis output or the treatment output, the therapeutic being selected from the group consisting of remdesivir, baricitinib, tocilizumab, a monoclonal antibody treatment, an immune checkpoint inhibitor, Nirmatrelvir with Ritonavi, Molnupiravir, and a combination thereof.

Embodiment 128. A method of designing a treatment for a symptomatic disease state of a coronavirus disease (COVID) in a subject, the method comprising: designing a therapeutic for treating the subject in response to identifying the subject as being positive for the symptomatic disease state using the method of any one of embodiments 63-85, 87-92, 93-120, or 122-126.

Embodiment 129. A method of planning a treatment for a symptomatic disease state of a coronavirus disease (COVID) in a subject, the method comprising: generating a treatment plan for treating the subject in response to identifying the subject as being positive for the symptomatic disease state using the method of any one of embodiments 63-85, 87-92, 93-120, or 122-126.

Embodiment 130. A method of manufacturing a treatment for a symptomatic disease state of a coronavirus disease (COVID) in a subject, the method comprising: manufacturing a therapeutic for treating the subject in response to identifying the subject as being positive for the symptomatic disease state using the method of any one of embodiments 63-85, 87-92, 93-120, or 122-126.

Embodiment 131. A method of treating a symptomatic disease state of a coronavirus disease (COVID) in a subject, the method comprising: administering to the subject a therapeutic to treat the subject based on identifying the subject as being positive for the symptomatic disease state using the method of any one of embodiments 63-85, 87-92, 93-120, or 122-126.

Embodiment 132. A method of treating a symptomatic disease state of a coronavirus disease (COVID) in a subject, the method comprising: selecting a therapeutic to treat the subject based on determining that the subject is responsive to the therapeutic using the method of any of embodiments 63-85, 87-92, 93-120, or 122-126; and administering the selected therapeutic to the subject.

Embodiment 133. A method for analyzing a set of peptide structures in a sample from a patient, the method comprising: (a) preparing a patient sample to form a prepared sample comprising a set of peptide structures; (b) inputting the prepared sample into a reaction monitoring mass spectrometry system to detect a set of product ions associated with each peptide structure of the set of peptide structures, the set of peptide structures comprising at least one of: a first peptide structure associated with the set of product ions that includes a product ion having a mass-to-charge (m/z) ratio within a range selected from a group consisting of 366.1±0.5, ±0.8, and ±1.0 and that is characterized as having a precursor ion having an m/z ratio within a range selected from a group consisting of 891±1.0 and ±1.5; a second peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio within a range selected from a group consisting of 1111.7±1.0 and ±1.5; a third peptide structure associated with the corresponding set of product ions that includes a product ion having a mass-to-charge (m/z) ratio that is within a range selected from a group consisting of 1062.5±0.5, ±0.8, and ±1.0 and that is characterized as having a precursor ion having an m/z ratio within a range selected from a group consisting of 1196.5±1.0 and ±1.5; a fourth peptide structure associated with the set of product ions that includes a product ion having a mass-to-charge (m/z) ratio that is within a range selected from a group consisting of 1062.5±0.5, ±0.8, and ±1.0 and that is characterized as having a precursor ion having an m/z ratio within a range selected from a group consisting of 1233±1.0 and ±1.5; a fifth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1167.3±1.0 and ±1.5; a sixth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1173.1±1.0 and ±1.5; a seventh peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1327.6±1.0 and ±1.5; an eighth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1061.9±1.0 and ±1.5; a ninth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 874.7±1.0 and ±1.5; a tenth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1225.8±1.0 and ±1.5; an 11th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1183.5±1.0 and ±1.5; a 12th peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio within a range selected from a group consisting of 1114.2±1.0 and ±1.5; a 13th peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio within a range selected from a group consisting of 1278.3±1.0 and ±1.5; a 14th peptide structure associated with the set of product ions that includes a product ion having a mass-to-charge (m/z) ratio that is within a range selected from a group consisting of 1453.6±0.5, ±0.8, and ±1.0 and that is characterized as having a precursor ion having an m/z ratio within a range selected from a group consisting of 1055.8±1.0 and ±1.5; a 15th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1284.6±1.0 and ±1.5; a 16th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1285.1±1.0 and ±1.5; a 17th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 991.2±1.0 and ±1.5; an 18th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1027.7±1.0 and ±1.5; a 19th peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio within a range selected from a group consisting of 1155±1.0 and ±1.5; a 20th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1191.2±1.0 and ±1.5; a 21st peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio within a range selected from a group consisting of 878.8±1.0 and ±1.5; a 22nd peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 976.1±1.0 and ±1.5; a 23rd peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1043.8±1.0 and ±1.5; a 24th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1199.5±1.0 and ±1.5; a 25th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1366.3±1.0 and ±1.5; a 26th peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio within a range selected from a group consisting of 939.1±1.0 and ±1.5; a 27th peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio within a range selected from a group consisting of 1295±1.0 and ±1.5; a 28th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 984.7±1.0 and ±1.5; a 29th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1367.6±1.0 and ±1.5; a 30th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1199.2±1.0 and ±1.5; a 31st peptide structure associated with the set of product ions that includes a product ion having a mass-to-charge (m/z) ratio that is within a range selected from a group consisting of 736.4±0.5, ±0.8, and ±1.0 and that is characterized as having a precursor ion having an m/z ratio within a range selected from a group consisting of 618.3±1.0 and ±1.5; a 32nd peptide structure associated with the set of product ions that includes a product ion having a mass-to-charge (m/z) ratio that is within a range selected from a group consisting of 342.2±0.5, ±0.8, and ±1.0 and that is characterized as having a precursor ion having an m/z ratio within a range selected from a group consisting of 590.3±1.0 and ±1.5; a 33rd peptide structure associated with the set of product ions that includes a product ion having a mass-to-charge (m/z) ratio that is within a range selected from a group consisting of 609.3±0.5, ±0.8, and ±1.0 and that is characterized as having a precursor ion having an m/z ratio within a range selected from a group consisting of 819.1±1.0 and ±1.5; a 34th peptide structure associated with the set of product ions that includes a product ion having a mass-to-charge (m/z) ratio that is within a range selected from a group consisting of 607.3±0.5, ±0.8, and ±1.0 and that is characterized as having a precursor ion having an m/z ratio within a range selected from a group consisting of 515.8±1.0 and ±1.5; a 35th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1116.9±1.0 and ±1.5; a 36th peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio within a range selected from a group consisting of 1154.7±1.0 and ±1.5; a 37th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1272.6±1.0 and ±1.5; a 38th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1285.6±1.0 and ±1.5; a 39th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1441.6±1.0 and ±1.5; a 40th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1206.3±1.0 and ±1.5; a 41st peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1179.1±1.0 and ±1.5; a 42nd peptide structure associated with the set of product ions that includes a product ion having a mass-to-charge (m/z) ratio that is within a range selected from a group consisting of 529.3±0.5, ±0.8, and ±1.0 and that is characterized as having a precursor ion having an m/z ratio within a range selected from a group consisting of 660.4±1.0 and ±1.5; a 43rd peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1310.6±1.0 and ±1.5; a 44th peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1021.4±1.0 and ±1.5; a 45th peptide structure associated with the set of product ions that includes a product ion having a mass-to-charge (m/z) ratio that is within a range selected from a group consisting of 234.1±0.5, ±0.8, and ±1.0 and that is characterized as having a precursor ion having an m/z ratio within a range selected from a group consisting of 764.9±1.0 and ±1.5; and (c) generating quantification data for the set of product ions using the reaction monitoring mass spectrometry system.

Embodiment 134. The method of embodiment 133, further comprising, prior to (a), obtaining the sample from the patient.

Embodiment 135. The method of embodiment 133 or 134, further comprising: generating a diagnosis output using the quantification data and a model that has been trained using at least one of supervised or unsupervised machine learning.

Embodiment 136. The method of embodiment 133 any of embodiments 133-135, wherein the reaction monitoring mass spectrometry system uses at least one of multiple reaction monitoring mass spectrometry (MRM-MS), or selected reaction monitoring mass spectrometry (SRM-MS) to detect the set of product ions and generate the quantification data.

Embodiment 137. The method of embodiment 133 any one of embodiments 133-136, wherein the sample comprises a plasma sample.

Embodiment 138. The method of embodiment 133 any one of embodiments 133-137, wherein the sample comprises a serum sample.

Embodiment 139. The method of any one of embodiments 133-138, wherein preparing the sample comprises at least one of: denaturing one or more proteins in the sample to form one or more denatured proteins; reducing the one or more denatured proteins in the sample to form one or more reduced proteins; alkylating the one or more proteins in the sample using an alkylating agent to prevent reformation of disulfide bonds in the one or more reduced proteins to form one or more alkylated proteins; or digesting the one or more alkylated proteins in the sample using a proteolysis catalyst to form the prepared sample comprising the set of peptide structures.

Embodiment 140. A composition comprising at least one of peptide structures PS-1 to PS-45 identified in Table 1-1.

Embodiment 141. A composition comprising a peptide structure or a product ion, wherein: the peptide structure or product ion comprises an amino acid sequence having at least 90% sequence identity to any one of SEQ ID NOS: 84-116, corresponding to peptide structures PS-1 to PS-45 in Table 1-1; and the product ion is selected as one from a group consisting of product ions identified in Table 4-1 including product ions falling within an identified m/z range.

Embodiment 142. A composition comprising a glycopeptide structure selected as one from a group of glycopeptide structures consisting of: a first glycopeptide structure having a monoisotopic mass of 4447.5 and comprising: the amino acid sequence of SEQ ID NO: 84; and glycan structure GL NO. 5401 linked to the 12^{th} residue of SEQ ID NO: 84; a second glycopeptide structure having a monoisotopic mass of 4440.74 and comprising: the amino acid sequence of SEQ ID NO. 85; and glycan structure GL NO. 5401 linked to the 15^{th} residue of SEQ ID NO. 85; a third glycopeptide structure having a monoisotopic mass of 4779.95 and comprising: the amino acid sequence of SEQ ID NO. 86; and glycan structure GL NO. 6503 linked to the 15^{th} residue of SEQ ID NO. 86; a fourth glycopeptide structure having a monoisotopic mass 4926.00 of and comprising: the amino acid sequence of SEQ ID NO. 86; glycan structure GL NO. 6513 linked to the 15^{th} residue of SEQ ID NO. 86; a fifth glycopeptide structure having a monoisotopic mass of 5829.49 and comprising: the amino acid sequence of SEQ ID NO. 87; and glycan structure GL NO. 7602 linked to the 15^{th} residue of SEQ ID NO. 87; a sixth glycopeptide structure having a monoisotopic mass of 3515.36 and comprising: the amino acid sequence of SEQ ID NO. 88; and glycan structure GL NO. 5402 linked to the 4^{th} residue of SEQ ID NO. 88; a seventh glycopeptide structure having a monoisotopic mass of 3979.65 and comprising: the amino acid sequence of SEQ ID NO. 89; and glycan structure GL NO. 5420 linked to the 10^{th} residue of SEQ ID NO. 89; an eighth glycopeptide structure having a monoisotopic mass of 4242.66 and comprising: the amino acid sequence of SEQ ID NO. 90; and glycan structure GL NO. 5412 linked to the 10^{th} residue of SEQ ID NO. 90; a ninth glycopeptide structure having a monoisotopic mass of 2621.06 and comprising: the amino acid sequence of SEQ ID NO. 91; and glycan structure GL NO. 5301 linked to the 1st residue of SEQ ID NO. 91; a tenth glycopeptide structure having a monoisotopic mass of 4900.17 and comprising: the amino acid sequence of SEQ ID NO. 92; and glycan structure GL NO. 6411 linked to the 6^{th} residue of SEQ ID NO. 92; an 11^{th} glycopeptide structure having a monoisotopic mass of 4729.04 and comprising: the amino acid sequence of SEQ ID NO. 93; and glycan structure GL NO. 7602 linked to the 6^{th} residue of SEQ ID NO. 93; a 12^{th} glycopeptide structure having a monoisotopic mass of 4450.92 and comprising: the amino acid sequence of SEQ ID NO. 94; and glycan structure GL NO. 5412 linked to the 4^{th} residue of SEQ ID NO. 94; a 13^{th} glycopeptide structure having a monoisotopic mass of 5107.14 and comprising: the amino acid sequence of SEQ ID NO. 94; and glycan structure GL NO. 6513 linked to the 4^{th} residue of SEQ ID NO. 94; a 14^{th} glycopeptide structure having a monoisotopic mass of 3163.24 and comprising: the amino acid sequence of SEQ ID NO. 95; and glycan structure GL NO. 5401 linked to the 3^{rd} residue of SEQ ID NO. 95; a 15^{th} glycopeptide structure having a monoisotopic mass of 5133.19 and comprising: the amino acid sequence of SEQ ID NO. 96; and glycan structure GL NO. 5402 linked to the 19^{th} residue of SEQ ID NO. 96; a 16^{th} glycopeptide structure having a monoisotopic mass of 5133.19 and comprising: the amino acid sequence of SEQ ID NO. 96; and glycan structure GL NO. 5421 linked to the 19^{th} residue of SEQ ID NO. 96; a 17^{th} glycopeptide structure having a monoisotopic mass of 3959.66 and comprising: the amino acid sequence of SEQ ID NO. 97; and glycan structure GL NO. 5402 linked to the 4^{th} residue of SEQ ID NO. 97; an 18^{th} glycopeptide structure having a monoisotopic mass of 4105.72 and comprising: the amino acid sequence of SEQ ID NO. 97; and glycan structure GL NO. 5412 linked to the 4^{th} residue of SEQ ID NO. 97; a 19^{th} glycopeptide structure having a monoisotopic mass of 4615.89 and comprising: the amino acid sequence of SEQ ID NO. 97; and glycan structure GL NO. 6503 linked to the 4^{th} residue of SEQ ID NO. 97; a 20^{th} glycopeptide structure having a monoisotopic mass of 4758.93 and comprising: the amino acid sequence of SEQ ID NO. 98; and glycan structure GL NO. 6513 linked to the 4^{th} residue of SEQ ID NO. 98; a 21^{st} glycopeptide structure having a monoisotopic mass of 2633.04 and comprising: the amino acid sequence of SEQ ID NO. 99; and glycan structure GL NO. 3410 linked to the 5^{th} residue of SEQ ID NO. 99; a 22^{nd} glycopeptide structure having a monoisotopic mass of 2925.15 and comprising: the amino acid sequence of SEQ ID NO. 99; and glycan structure GL NO. 5410 linked to the 5^{th} residue of SEQ ID NO. 99; a 23^{rd} glycopeptide structure having a monoisotopic mass of 3128.23 and comprising: the amino acid sequence of SEQ ID NO. 99; and glycan structure GL NO. 5510 linked to the 5^{th} residue of SEQ ID NO. 99; a 24^{th} glycopeptide structure having a monoisotopic mass of 4677.79 and comprising: the amino acid sequence of SEQ ID NO. 100; and glycan structure GL NO. 6503 linked to the 9^{th} residue of SEQ ID NO. 100; a 25^{th} glycopeptide structure having a monoisotopic mass of 6822.70 and comprising: the amino acid sequence of SEQ ID NO. 101; and glycan structure GL NO. 5402 linked to the 9^{th} residue of SEQ ID NO. 101; a 26^{th} glycopeptide structure having a monoisotopic mass of 2813.31 and comprising: the amino acid sequence of SEQ ID NO. 102; and glycan structure GL NO. 1101 linked to the 12^{th} residue of SEQ ID NO. 102; a 27^{th} glycopeptide structure having a monoisotopic mass of 5174.11 and comprising: the amino acid sequence of SEQ ID NO. 103; and glycan structure GL NO. 6523 linked to the 9^{th} residue of SEQ ID NO. 103; a 28^{th} glycopeptide structure having a monoisotopic mass of 3933.66 and comprising: the amino acid sequence of SEQ ID NO. 104; and glycan structure GL NO. 5401 linked to the 15^{th} residue of SEQ ID NO. 104; a 29^{th} glycopeptide structure having a monoisotopic mass of 5463.24 and comprising: the amino acid sequence of SEQ ID NO. 105; and glycan structure GL NO. 5401 linked to the 16^{th} residue of SEQ ID NO. 105; a 30^{th} glycopeptide structure having a monoisotopic mass of 4791.91 and comprising: the amino acid sequence of SEQ ID NO. 106; and glycan structure GL NO. 7420 linked to the 4^{th} residue of SEQ ID NO. 106; a 31^{st} glycopeptide structure having a monoisotopic mass of 5578.17 and comprising: the amino acid sequence of SEQ ID NO. 111; and glycan structure GL NO. 7614 linked to the 7^{th} residue of SEQ ID NO. 111; a 32th glycopeptide structure having a monoisotopic mass of 4612.87 and comprising: the amino acid sequence of SEQ ID NO. 98; and glycan structure GL NO. 6503 linked to the 4^{th} residue of SEQ ID NO. 98; a 33^{rd} glycopeptide structure having a monoisotopic mass of 5084.28 and comprising: the amino acid sequence of SEQ ID NO. 112; and glycan structure GL NO. 5401 linked to the 17^{th} residue of SEQ ID NO. 112; a 34^{th} glycopeptide structure having a monoisotopic mass of 3853.74 and comprising: the amino acid sequence of SEQ ID NO. 93; and glycan structure GL NO. 5411 linked to the 6^{th} residue of SEQ ID NO. 93; a 35^{th} glycopeptide structure having a monoisotopic mass of 4321.78 and comprising: the amino acid sequence of SEQ ID NO. 98; and glycan structure GL NO. 6502 linked to the 4^{th} residue of SEQ ID NO. 98; a 36^{th} glycopeptide structure having a monoisotopic mass of 8432.83 and comprising: the amino acid sequence of SEQ ID NO. 113; and glycan structure GL NO. 5402 linked to the 46^{th} residue of SEQ ID NO. 113; a 37^{th} glycopeptide structure having a monoisotopic mass of 5890.66 and comprising: the amino acid sequence of SEQ ID NO. 97; and glycan structure GL NO. 5412 linked to the 4^{th} residue of SEQ ID NO. 97; a 38^{th} glycopeptide structure having a monoisotopic mass of 3927.64 and comprising: the amino acid sequence of SEQ ID NO. 115; and glycan structure GL NO. 5401 linked to the 5^{th} residue of SEQ ID NO. 115; a 39th glycopeptide structure having a monoisotopic mass of 4079.71 and comprising: the amino acid sequence of SEQ ID NO. 104; and glycan structure GL NO. 5411 linked to the 15^{th} residue of SEQ ID NO. 104; and wherein:
the glycan structure GL NO. 1101 comprises:

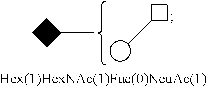

the glycan structure GL NO. 3410 comprises:

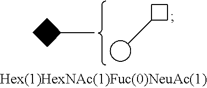

the glycan structure GL NO. 5401 comprises:

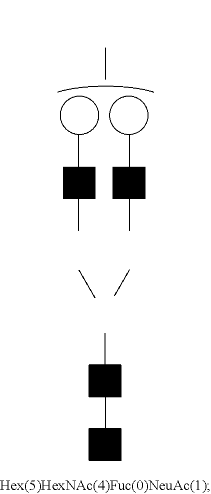

the glycan structure (GL NO. 5402) comprises:

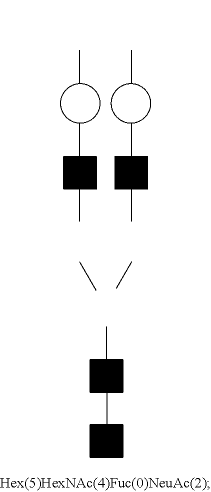

the glycan structure (GL NO. 5410) comprises:

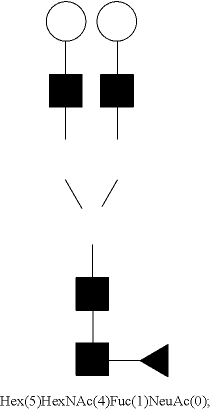

the glycan structure (GL NO. 5411) comprises:

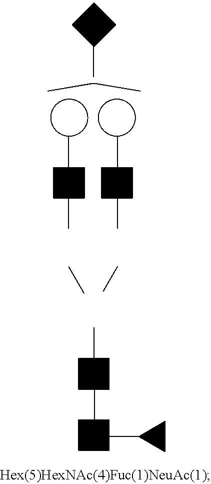

the glycan structure (GL NO. 5412) comprises:

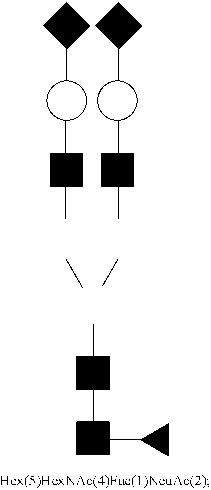

the glycan structure (GL NO. 5420) comprises:

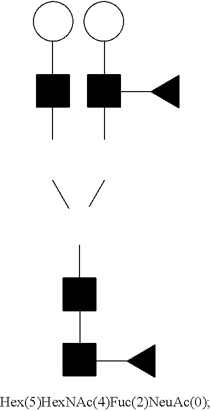

the glycan structure (GL NO. 5510) comprises:

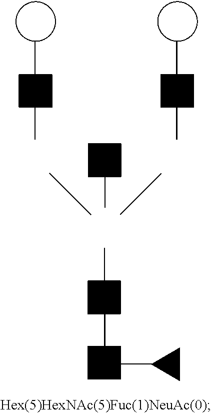

the glycan structure (GL NO. 6411) comprises:

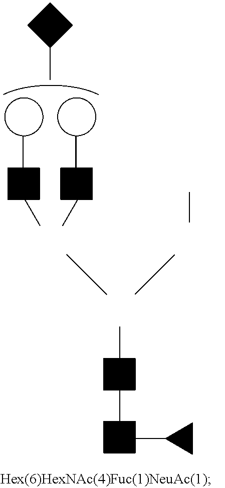

the glycan structure (GL NO. 6502) comprises:

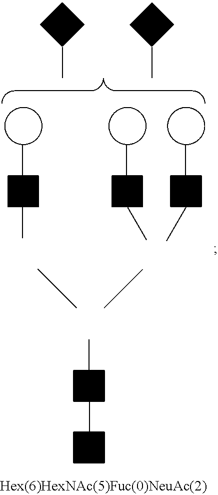

the glycan structure (GL NO. 6503) comprises:

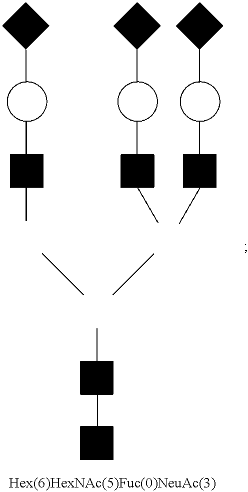

the glycan structure (GL NO. 6523) comprises:

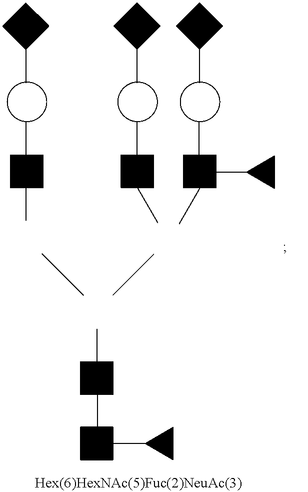

the glycan structure (GL NO. 7420) comprises:

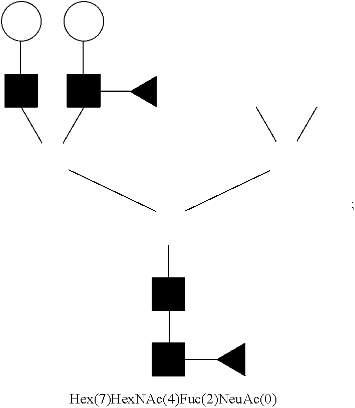

and the glycan structure (GL NO. 7602) comprises:

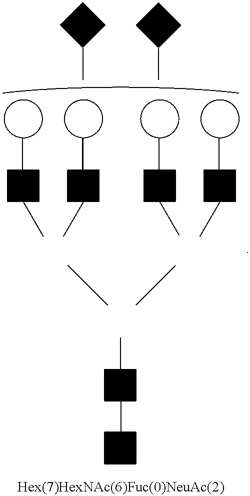

Embodiment 143. The composition of embodiment 142, wherein: the first glycopeptide structure has a precursor ion having a charge of 5; the second glycopeptide structure has a precursor ion having a charge of 4; the third glycopeptide structure has a precursor ion having a charge of 4; the fourth glycopeptide structure has a precursor ion having a charge of 4; the fifth glycopeptide structure has a precursor ion having a charge of 5; the sixth glycopeptide structure has a precursor ion having a charge of 3; the seventh glycopeptide structure has a precursor ion having a charge of 3; the eighth glycopeptide structure has a precursor ion having a charge of 4; the ninth glycopeptide structure has a precursor ion having a charge of 3; the tenth glycopeptide structure has a precursor ion having a charge of 4; the 11^{th} glycopeptide structure has a precursor ion having a charge of 4; the 12^{th} glycopeptide structure has a precursor ion having a charge of 4; the 13^{th} glycopeptide structure has a precursor ion having a charge of 4; the 14^{th} glycopeptide structure has a precursor ion having a charge of 3; the 15^{th} glycopeptide structure has a precursor ion having a charge of 4; the 16^{th} glycopeptide structure has a precursor ion having a charge of 4; the 17^{th} glycopeptide structure has a precursor ion having a charge of 4; the 18^{th} glycopeptide structure has a precursor ion having a charge of 4; the 19^{th} glycopeptide structure has a precursor ion having a charge of 4; the 20^{th} glycopeptide structure has a precursor ion having a charge of 4; the 21^{st} glycopeptide structure has a precursor ion having a charge of 3; the 22^{nd} glycopeptide structure has a precursor ion having a charge of 3; the 23^{rd} glycopeptide structure has a precursor ion having a charge of 3; the 24^{th} glycopeptide structure has a precursor ion having a charge of 4; the 25^{th} glycopeptide structure has a precursor ion having a charge of 5; the 26^{th} glycopeptide structure has a precursor ion having a charge of 3; the 27^{th} glycopeptide structure has a precursor ion having a charge of 4; the 28^{th} glycopeptide structure has a precursor ion having a charge of 4; the 29^{th} glycopeptide structure has a precursor ion having a charge of 4; the 30^{th} glycopeptide structure has a precursor ion having a charge of 4; the 31^{st} glycopeptide structure has a precursor ion having a charge of 5; the 32^{th} glycopeptide structure has a precursor ion having a charge of 4; the 33^{rd} glycopeptide structure has a precursor ion having a charge of 4; the 34^{th} glycopeptide structure has a precursor ion having a charge of 3; the 35^{th} glycopeptide structure has a precursor ion having a charge of 3; the 36^{th} glycopeptide structure has a precursor ion having a charge of 7; the 37^{th} glycopeptide structure has a precursor ion having a charge of 5; the 38^{th} glycopeptide structure has a precursor ion having a charge of 3; and the 39^{th} glycopeptide structure has a precursor ion having a charge of 4.

Embodiment 144. The composition of embodiment 142, wherein: the first glycopeptide structure has a product ion having a mass-to-charge (m/z) ratio within a range selected from a group consisting of 366.1±0.5, ±0.8, and ±1.0; the second glycopeptide structure has a product ion having a mass-to-charge (m/z) ratio that is within a range selected from a group consisting of 204.1±0.5, ±0.8, and ±1.0; the third glycopeptide structure associated with the corresponding set of product ions that includes a product ion having a mass-to-charge (m/z) ratio that is within a range selected from a group consisting of 1062.5±0.5, ±0.8, and ±1.0; the fourth glycopeptide structure has a product ion having a mass-to-charge (m/z) ratio that is within a range selected from a group consisting of 1062.5±0.5, ±0.8, and ±1.0; the fifth glycopeptide structure has a product ion having a mass-to-charge (m/z) ratio that is within a range selected from a group consisting of 366.1±0.5, ±0.8, and ±1.0; the sixth glycopeptide structure has a product ion having a mass-to-charge (m/z) ratio that is within a range selected from a group consisting of 366.1±0.5, ±0.8, and ±1.0; the seventh glycopeptide structure has a product ion having a mass-to-charge (m/z) ratio that is within a range selected from a group consisting of 366.1±0.5, ±0.8, and ±1.0; the eighth glycopeptide structure has a product ion having a mass-to-charge (m/z) ratio that is within a range selected from a group consisting of 366.1±0.5, ±0.8, and ±1.0; the ninth glycopeptide structure has a product ion having a mass-to-charge (m/z) ratio that is within a range selected from a group consisting of 366.1±0.5, ±0.8, and ±1.0; the tenth glycopeptide structure has a product ion having a mass-to-charge (m/z) ratio that is within a range selected from a group consisting of 366.1±0.5, ±0.8, and ±1.0; the 11^{th} glycopeptide structure has a product ion having a mass-to-charge (m/z) ratio that is within a range selected from a group consisting of 366.1±0.5, ±0.8, and ±1.0; the 12^{th} glycopeptide structure has a product ion having a mass-to-charge (m/z) ratio that is within a range selected from a group consisting of 204.1±0.5, ±0.8, and ±1.0; the 13^{th} glycopeptide structure has a product ion having a mass-to-charge (m/z) ratio that is within a range selected from a group consisting of 204.1±0.5, ±0.8, and ±1.0; the 14^{th} glycopeptide structure has a product ion having a mass-to-charge (m/z) ratio that is within a range selected from a group consisting of 1453.6±0.5, ±0.8, and ±1.0; the 15^{th} glycopeptide structure has a product ion having a mass-to-charge (m/z) ratio that is within a range selected from a group consisting of 366.1±0.5, ±0.8, and ±1.0; the 16^{th} glycopeptide structure has a product ion having a mass-to-charge (m/z) ratio that is within a range selected from a group consisting of 366.1±0.5, ±0.8, and ±1.0; the 17^{th} glycopeptide structure has a product ion having a mass-to-charge (m/z) ratio that is within a range selected from a group consisting of 366.1±0.5, ±0.8, and ±1.0; the 18^{th} glycopeptide structure has a product ion having a mass-to-charge (m/z) ratio that is within a range selected from a group consisting of 366.1±0.5, ±0.8, and ±1.0; the 19^{th} glycopeptide structure has a product ion having a mass-to-charge (m/z) ratio that is within a range selected from a group consisting of 274.1±0.5, ±0.8, and ±1.0; the 20^{th} glycopeptide structure has a product ion having a mass-to-charge (m/z) ratio that is within a range selected from a group consisting of 366.1±0.5, ±0.8, and ±1.0; the 21^{st} glycopeptide structure has a product ion having a mass-to-charge (m/z) ratio that is within a range selected from a group consisting of 204.1±0.5, ±0.8, and ±1.0; the 22^{nd} glycopeptide structure has a product ion having a mass-to-charge (m/z) ratio that is within a range selected from a group consisting of 366.1±0.5, ±0.8, and ±1.0; the 23^{rd} glycopeptide structure has a product ion having a mass-to-charge (m/z) ratio that is within a range selected from a group consisting of 366.1±0.5, ±0.8, and ±1.0; the 24^{th} glycopeptide structure has a product ion having a mass-to-charge (m/z) ratio that is within a range selected from a group consisting of 366.1±0.5, ±0.8, and ±1.0; the 25^{th} glycopeptide structure has a product ion having a mass-to-charge (m/z) ratio that is within a range selected from a group consisting of 366.1±0.5, ±0.8, and ±1.0; the 26^{th} glycopeptide structure has a product ion having a mass-to-charge (m/z) ratio that is within a range selected from a group consisting of 274.1±0.5, ±0.8, and ±1.0; the 27^{th} glycopeptide structure has a product ion having a mass-to-charge (m/z) ratio that is within a range selected from a group consisting of 274.1±0.5, ±0.8, and ±1.0; the 28^{th} glycopeptide structure has a product ion having a mass-to-charge (m/z) ratio that is within a range selected from a group consisting of 366.1±0.5, ±0.8, and ±1.0; the 29^{th} glycopeptide structure has a product ion having a mass-to-charge (m/z) ratio that is within a range selected from a group consisting of 366.1±0.5, ±0.8, and ±1.0; the 30^{th} glycopeptide structure has a product ion having a mass-to-charge (m/z) ratio that is within a range selected from a group consisting of 366.1±0.5, ±0.8, and ±1.0; the 31^{st} glycopeptide structure has a product ion having a mass-to-charge (m/z) ratio that is within a range selected from a group consisting of 366.1±0.5, ±0.8, and ±1.0; the 32^{nd} glycopeptide structure has a product ion having a mass-to-charge (m/z) ratio that is within a range selected from a group consisting of 274.1±0.5, ±0.8, and ±1.0; the 33^{rd} glycopeptide structure has a product ion having a mass-to-charge (m/z) ratio that is within a range selected from a group consisting of 366.1±0.5, ±0.8, and ±1.0; the 34^{th} glycopeptide structure has a product ion having a mass-to-charge (m/z) ratio that is within a range selected from a group consisting of 366.1±0.5, ±0.8, and ±1.0; the 35^{th} glycopeptide structure has a product ion having a mass-to-charge (m/z) ratio that is within a range selected from a group consisting of 366.1±0.5, ±0.8, and ±1.0; the 36^{th} glycopeptide structure has a product ion having a mass-to-charge (m/z) ratio that is within a range selected from a group consisting of 366.1±0.5, ±0.8, and ±1.0; the 37^{th} glycopeptide structure has a product ion having a mass-to-charge (m/z) ratio that is within a range selected from a group consisting of 366.1±0.5, ±0.8, and ±1.0; the 39^{th} glycopeptide structure has a product ion having a mass-to-charge (m/z) ratio that is within a range selected from a group consisting of 366.1±0.5, ±0.8, and ±1.0; and the 39^{th} glycopeptide structure has a product ion having a mass-to-charge (m/z) ratio that is within a range selected from a group consisting of 366.1±0.5, ±0.8, and ±1.0.

Embodiment 145. The composition of embodiment 142, wherein: the first glycopeptide structure has a precursor ion having an m/z ratio within a range selected from a group consisting of 891±1.0 and ±1.5; the second glycopeptide structure has a precursor ion having an m/z ratio within a range selected from a group consisting of 1111.7±1.0 and ±1.5; the third glycopeptide structure has a precursor ion having an m/z ratio within a range selected from a group consisting of 1196.5±1.0 and ±1.5; the fourth glycopeptide structure has a precursor ion having an m/z ratio within a range selected from a group consisting of 1233±1.0 and ±1.5; the fifth glycopeptide structure has a precursor ion having an m/z ratio within a range selected from a group consisting of 1167.3±1.0 and ±1.5; the sixth glycopeptide structure has a precursor ion having an m/z ratio within a range selected from a group consisting of 1173.1±1.0 and ±1.5; the seventh glycopeptide structure has a precursor ion having an m/z ratio within a range selected from a group consisting of 1327.6±1.0 and ±1.5; the eighth glycopeptide structure has a precursor ion having an m/z ratio within a range selected from a group consisting of 1061.9±1.0 and ±1.5; the ninth glycopeptide structure has a precursor ion having an m/z ratio within a range selected from a group consisting of 874.7±1.0 and ±1.5; the tenth glycopeptide structure has a precursor ion having an m/z ratio within a range selected from a group consisting of 1225.8±1.0 and ±1.5; the 11^{th} glycopeptide structure has a precursor ion having an m/z ratio within a range selected from a group consisting of 1183.5±1.0 and ±1.5; the 12^{th} glycopeptide structure has a precursor ion having an m/z ratio within a range selected from a group consisting of 1114.2±1.0 and ±1.5; the 13^{th} glycopeptide structure has a precursor ion having an m/z ratio within a range selected from a group consisting of 1278.3±1.0 and ±1.5; the 14^{th} glycopeptide structure has a precursor ion having an m/z ratio within a range selected from a group consisting of 1055.8±1.0 and ±1.5; the 15^{th} glycopeptide structure has a precursor ion having an m/z ratio within a range selected from a group consisting of 1284.6±1.0 and ±1.5; the 16^{th} glycopeptide structure has a precursor ion having an m/z ratio within a range selected from a group consisting of 1285.1±1.0 and ±1.5; the 17^{th} glycopeptide structure has a precursor ion having an m/z ratio within a range selected from a group consisting of 991.2±1.0 and ±1.5; the 18^{th} glycopeptide structure has a precursor ion having an m/z ratio within a range selected from a group consisting of 1027.7±1.0 and ±1.5; the 19^{th} glycopeptide structure has a precursor ion having an m/z ratio within a range selected from a group consisting of 1155±1.0 and ±1.5; the 20^{th} glycopeptide structure has a precursor ion having an m/z ratio within a range selected from a group consisting of 1191.2±1.0 and ±1.5; the 21^{st} glycopeptide structure has a precursor ion having an m/z ratio within a range selected from a group consisting of 878.8±1.0 and ±1.5; the 22^{nd} glycopeptide structure has a precursor ion having an m/z ratio within a range selected from a group consisting of 976.1±1.0 and ±1.5; the 23^{rd} glycopeptide structure has a precursor ion having an m/z ratio within a range selected from a group consisting of 1043.8±1.0 and ±1.5; the 24^{th} glycopeptide structure has a precursor ion having an m/z ratio within a range selected from a group consisting of 1199.5±1.0 and ±1.5; the 25^{th} glycopeptide structure has a precursor ion having an m/z ratio within a range selected from a group consisting of 1366.3±1.0 and ±1.5; the 26^{th} glycopeptide structure has a precursor ion having an m/z ratio within a range selected from a group consisting of 939.1±1.0 and ±1.5; the 27^{th} glycopeptide structure has a precursor ion having an m/z ratio within a range selected from a group consisting of 1295±1.0 and ±1.5; the 28^{th} glycopeptide structure has a precursor ion having an m/z ratio within a range selected from a group consisting of 984.7±1.0 and ±1.5; the 29^{th} glycopeptide structure has a precursor ion having an m/z ratio within a range selected from a group consisting of 1367.6±1.0 and ±1.5; the 30^{th} glycopeptide structure has a precursor ion having an m/z ratio within a range selected from a group consisting of 1199.2±1.0 and ±1.5; the 31^{st} glycopeptide structure has a precursor ion having an m/z ratio within a range selected from a group consisting of 1116.9±1.0 and ±1.5; the 32^{nd} glycopeptide structure has a precursor ion having an m/z ratio within a range selected from a group consisting of 1154.7±1.0 and ±1.5; the 33^{rd} glycopeptide structure has a precursor ion having an m/z ratio within a range selected from a group consisting of 1272.6±1.0 and ±1.5; the 34^{th} glycopeptide structure has a precursor ion having an m/z ratio within a range selected from a group consisting of 1285.6±1.0 and ±1.5; the 35^{th} glycopeptide structure has a precursor ion having an m/z ratio within a range selected from a group consisting of 1441.6±1.0 and ±1.5; the 36^{th} glycopeptide structure has a precursor ion having an m/z ratio within a range selected from a group consisting of 1206.3±1.0 and ±1.5; the 37^{th} glycopeptide structure has a precursor ion having an m/z ratio within a range selected from a group consisting of 1179.1±1.0 and ±1.5; the 39^{th} glycopeptide structure has a precursor ion having an m/z ratio within a range selected from a group consisting of 1310.6±1.0 and ±1.5; and the 40^{th} glycopeptide structure has a precursor ion having an m/z ratio within a range selected from a group consisting of 1021.4±1.0 and ±1.5.

Embodiment 146. A composition comprising a peptide structure selected as one from a group of aglycosylated peptide structures consisting of: a first peptide structure having a monoisotopic mass of 1234.68 and comprising the amino acid sequence of SEQ ID NO: 107;
a second peptide structure having a monoisotopic mass of 1178.67 comprising the amino acid sequence of SEQ ID NO: 108; a third peptide structure having a monoisotopic mass of 2454.14 comprising the amino acid sequence of SEQ ID NO: 109; a fourth peptide structure having a monoisotopic mass of 1029.53 comprising the amino acid sequence of SEQ ID NO: 110; a fifth peptide structure having a monoisotopic mass of 1318.73 comprising the amino acid sequence of SEQ ID NO: 114; and a sixth peptide structure having a monoisotopic mass of 1527.74 comprising the amino acid sequence of SEQ ID NO: 116.

Embodiment 147. The composition of embodiment 146, wherein: the first peptide structure has a precursor ion having a charge of 2; the second peptide structure has a precursor ion having a charge of 2; the third peptide structure has a precursor ion having a charge of 3; the fourth peptide structure has a precursor ion having a charge of 2; the fifth peptide structure has a precursor ion having a charge of 2; and the sixth peptide structure has a precursor ion having a charge of 2.

Embodiment 148. The composition of embodiment 146, wherein: the first peptide structure has a product ion having a mass-to-charge (m/z) ratio that is within a range selected from a group consisting of 736.4±0.5, ±0.8, and ±1.0; the second peptide structure has a product ion having a mass-to-charge (m/z) ratio that is within a range selected from a group consisting of 342.2±0.5, ±0.8, and ±1.0; the third peptide structure has a product ion having a mass-to-charge (m/z) ratio that is within a range selected from a group consisting of 609.3±0.5, ±0.8, and ±1.0; the fourth peptide structure has a product ion having a mass-to-charge (m/z) ratio that is within a range selected from a group consisting of 607.3±0.5, ±0.8, and ±1.0; the fifth peptide structure has a product ion having a mass-to-charge (m/z) ratio that is within a range selected from a group consisting of 529.3±0.5, ±0.8, and ±1.0; and the sixth peptide structure has a product ion having a mass-to-charge (m/z) ratio that is within a range selected from a group consisting of 234.1±0.5, ±0.8, and ±1.0.

Embodiment 149. The composition of embodiment 146, wherein: the first peptide structure has a precursor ion having an m/z ratio within a range selected from a group consisting of 618.3±1.0 and ±1.5; the second peptide structure has a precursor ion having an m/z ratio within a range selected from a group consisting of 590.3±1.0 and ±1.5; the third peptide structure has a precursor ion having an m/z ratio within a range selected from a group consisting of 819.1±1.0 and ±1.5; the fourth peptide structure has a precursor ion having an m/z ratio within a range selected from a group consisting of 515.8±1.0 and ±1.5; the fifth peptide structure has a precursor ion having an m/z ratio within a range selected from a group consisting of 660.4±1.0 and ±1.5; and the sixth peptide structure has a precursor ion having an m/z ratio within a range selected from a group consisting of 764.9±1.0 and ±1.5.

Embodiment 150. A kit comprising at least one agent for quantifying at least one peptide structure identified in Table 1-1 to carry out the method of any one of embodiments 63-139.

Embodiment 151. A kit comprising at least one of a glycopeptide standard, a buffer, or a set of peptide sequences to carry out the method of any one of embodiments 63-139, a peptide sequence of the set of peptide sequences identified by a corresponding one of SEQ ID NOS: 84-116, defined in Table 5-1.

Embodiment 152. A system comprising: one or more data processors; and a non-transitory computer readable storage medium containing instructions which, when executed on the one or more data processors, cause the one or more data processors to perform part or all of any one of embodiments 63-85, 87-92, 93-120, or 122-126.

Embodiment 153. A method of diagnosing a coronavirus disease (COVID) in a subject, comprising the step of identifying one or more peptide structures identified in Table 2-1 from a sample from the subject.

Embodiment 154. The method of embodiment 153, wherein the sample comprises blood, plasma, serum, urine, saliva, mucosal excretions, sputum, stool, nasal mucus, phlegm, and/or tears.

Embodiment 155. The method of embodiment 153 or 154, wherein the step of identifying occurs once.

Embodiment 156. The method of embodiment 153 or 154, wherein the step of identifying occurs multiple times.

Embodiment 157. The method of embodiment 156, wherein one or more symptoms becomes undetectable between multiple identifying steps.

Embodiment 158. A method of identifying or managing an at-risk subject for a coronavirus disease (COVID), the method comprising measuring whether a biological sample obtained from the subject evidences COVID using part or all of the method of any one of embodiments 63-85, 87-92, 93-120, 122-126, or 153-157, and subjecting the subject to one or more medical tests or procedures, and/or subjecting the subject to one or more preventatives or therapies in response to the identification of the symptomatic disease state.

Embodiment 159. The method of embodiment 158, wherein the subject has one or more COVID symptoms at the time of measuring and/or at the time of obtaining the sample.

Embodiment 160. The method of embodiment 158, wherein the subject is asymptomatic at the time of measuring and/or at the time of obtaining the sample.

Embodiment 161. A method of identifying a subject suitable for, or in need of, COVID prevention or treatment, the method comprising the step of measuring from a biological sample taken from the subject for the presence of one or a combination of peptide structures identified in Table 2-1, wherein their detection indicates that the subject should have COVID prevention or treatment.

Embodiment 162. The method of embodiment 161, wherein the subject has one or more COVID symptoms at the time of measuring and/or at the time of obtaining the sample.

Embodiment 163. The method of embodiment 161, wherein the subject is asymptomatic at the time of measuring and/or at the time of obtaining the sample.

Embodiment 164. A method of predicting whether a subject will be symptomatic upon coronavirus infection, comprising the step of measuring from a biological sample taken from the subject for the presence of one or a combination of peptide structures identified in Table 2-1.

Embodiment 165. A computer-program product tangibly embodied in a non-transitory machine-readable storage medium, including instructions configured to cause one or more data processors to perform part or all of any one of embodiments 63-85, 87-92, 93-120, 122-126, or 154-164.

### XV. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A method of determining whether a biological sample corresponds to a sepsis state, the method comprising:
inputting quantification data identified from peptide structure data for a set of peptide structures into a supervised machine learning model, wherein the set of peptide structures includes at least one peptide structure identified in Table 1;
analyzing the quantification data using the supervised machine learning model to generate a disease indicator that classifies the biological sample as corresponding to a cluster of a plurality of clusters that has a least distance to the biological sample, the plurality of clusters corresponding a plurality of states that includes the sepsis state; and
generating a diagnosis output based on the disease indicator.

2. The method of claim 1, further comprising receiving peptide structure data corresponding to the biological sample obtained from a subject.

3. The method of claim 1, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 24-49 as defined in Table 3.

4. The method of claim 1, wherein the at least one peptide structure comprises an aglycosylated peptide structure defined by a peptide sequence, as identified in Table 1, with the peptide sequence being one of SEQ ID NOS: 50-54 as defined in Table 3.

5. The method of claim 1, wherein the supervised machine learning model comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

6. The method of claim 1, further comprising:
training the supervised machine learning model using training data generated from an unsupervised machine learning model,
wherein the training data comprises a plurality of peptide structure profiles for a plurality of subjects and identifies a corresponding state of the plurality of states for each peptide structure profile of the plurality of peptide structure profiles; and
wherein the unsupervised machine learning model is trained to cluster the plurality of peptide structure profiles into the plurality of clusters.

7. The method of claim 1, wherein the unsupervised machine learning model is a k-means clustering model.

8. The method of claim 1, wherein a peptide structure profile of the plurality of peptide structure profiles for a corresponding subject of the plurality of subjects is selected based on a differential expression analysis of quantification metrics for a panel of peptide structures for the plurality of subjects.

9. The method of claim 8, further comprising:
comparing, using the differential expression analysis, the quantification metrics corresponding to the panel of peptide structures for a first portion of the plurality of subjects diagnosed with the sepsis state to that of each portion of a set of other portions of the plurality of subjects to generate a set of comparisons; and
selecting a portion of the panel of peptide structures having a false discovery rate below 0.05 across the set of comparisons as the set of peptide structures.

10. The method of claim 9, wherein the comparing comprises:
comparing the quantification metrics corresponding to the panel of peptide structures for the first portion of the plurality of subjects diagnosed with the sepsis state to that of a second portion of the plurality of subjects diagnosed with a symptomatic disease state of a coronavirus disease to generate a first comparison of the set of comparisons.

11. The method of claim 10, wherein the comparing further comprises:
comparing the quantification metrics corresponding to the panel of peptide structures for the first portion of the plurality of subjects diagnosed with the sepsis state to that of at least one of:
a third portion of the plurality of subjects diagnosed with a common cold state to generate a second comparison of the set of comparisons;
a fourth portion of the plurality of subjects diagnosed with a healthy state to generate a third comparison of the set of comparisons;
a fifth portion of the plurality of subjects diagnosed with an asymptomatic state of the coronavirus disease to generate a fourth comparison of the set of comparisons.

12. The method of claim 1, wherein the quantification data for a peptide structure of the set of peptide structures comprises at least one of a relative quantity, an adjusted quantity, a normalized quantity, a relative concentration, an adjusted concentration, or a normalized concentration.

13. The method of claim 1, wherein the peptide structure data is generated using multiple reaction monitoring mass spectrometry (MRM-MS).

14. The method of claim 1, wherein the sepsis state is selected from a group consisting of a mild sepsis state, a moderate sepsis state, or a severe sepsis state.

15. The method of claim 1, further comprising:
creating a sample from the biological sample; and
preparing the sample using reduction, alkylation, and enzymatic digestion to form a prepared sample that includes a set of peptide structures.

16. The method of claim 1, further comprising:
generating the peptide structure data from the prepared sample using multiple reaction monitoring mass spectrometry (MRM-MS).

17. The method of claim 1, wherein generating the diagnosis output comprises:
generating a report that includes a diagnosis that the subject is positive for the sepsis state in response to the disease indicator classifying the biological sample as corresponding to the sepsis state.

18. The method of claim 1, wherein the biological sample comprises at least one of a whole blood sample, a plasma sample, or a serum sample.

19. The method of claim 1, further comprising:
generating a treatment output based on at least one of the diagnosis output or the disease indicator.

20. The method of claim 19, wherein the treatment output comprises at least one of an identification of a treatment to treat the subject, a design for the treatment, a manufacturing plan for the treatment, or a treatment plan for administering the treatment.

21. The method of claim 19, wherein the treatment comprises at least one antibiotic.

22. The method of claim 19, wherein the treatment comprises at least one of a broad-spectrum antibiotic, a targeted antibiotic, or a vasopressor.

23. The method of claim 19, wherein generating the treatment output comprises:
determining a dosage for a therapeutic to treat the subject based on at least one of the diagnosis output or the disease indicator.

24. The method of claim 19, wherein the diagnosis output identifies that the biological sample is positive for the sepsis state and further comprising:
administering a therapeutic dosage of a therapeutic for the sepsis state to the subject, the therapeutic comprising one or more antibiotics.

25. A method of identifying a sepsis-specific set of peptide structures for use in diagnosing a sepsis state, the method comprising:
receiving quantification data for a panel of peptide structures for a plurality of subjects diagnosed with a plurality of states, the plurality of states including the sepsis state;
comparing quantification metrics corresponding to the panel of peptide structures for a first portion of the plurality of subjects diagnosed with the sepsis state to that of each portion of a set of other portions of the plurality of subjects to generate a set of comparisons using a differential expression analysis;
selecting a portion of the panel of peptide structures having a false discovery rate below 0.05 across the set of comparisons as a set of peptide structures to be associated with the sepsis state,
wherein the set of peptide structures includes at least one glycopeptide structure defined by a peptide sequence and a glycan structure linked to the peptide sequence at a linking site of the peptide sequence;
analyzing the quantification data for the set of peptide structures for the plurality of subjects using an unsupervised machine learning model to cluster the plurality of subjects; and
training a supervised machine learning model to determine whether a biological sample obtained from a subject corresponds to the sepsis state or another state of the plurality of states.

26. The method of claim 25, further comprising:
analyzing the biological sample obtained from the subject using the supervised machine learning model that has been trained to generate a disease indicator that indicates whether the biological subject is positive for the sepsis state.

27. The method of claim 25, wherein the plurality of states further includes at least one of a common cold state, a healthy state, a symptomatic disease state of a coronavirus disease (COVID), or an asymptomatic disease state of the coronavirus disease.

28. The method of claim 25, wherein the sepsis state is either a mild sepsis state or a severe sepsis state.

29. The method of claim 25, wherein the unsupervised machine learning model comprises a k-means clustering model and wherein the supervised machine learning model comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

30. The method of claim 25, wherein the set of peptide structures includes at least three peptide structures identified in Table 1.

31. A method of evaluating a biological sample obtained from a subject with respect to a sepsis state, the method comprising:
identifying a peptide structure profile for the biological sample using peptide structure data, the peptide structure profile comprising quantification data for a set of peptide structures associated with the sepsis state,
wherein the set of peptide structures includes at least two peptide structures from a selected group of peptide structures identified in Table 1; and
wherein at least two peptide structures in the selected group of peptide structures includes a glycopeptide structure defined by a peptide sequence and a glycan structure linked to a linking site of the peptide sequence;
computing a disease indicator using the peptide structure profile and a model, wherein the disease indicator indicates whether the biological sample is positive for the sepsis state; and
generating at least one of a diagnosis output or a treatment output based on the disease indicator.

32. The method of claim 31, further comprising receiving peptide structure data corresponding to the biological sample obtained from the subject.

33. The method of claim 31, wherein the model includes a supervised machine learning model that comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

34. The method of claim 31, wherein the model comprises a supervised machine learning model trained using an output of an unsupervised machine learning model that is trained to cluster a plurality of peptide structure profiles for a plurality of subjects according to a plurality of states, the plurality of states including the sepsis state.

35. The method of claim 31, wherein the treatment output comprises at least one of an identification of a treatment to treat the subject, a design for the treatment, a manufacturing plan for the treatment, or a treatment plan for administering the treatment.

36. The method of claim 31, further comprising:
administering a therapeutic dosage of a therapeutic for the sepsis state to the subject based on the at least one of the diagnosis output or the treatment output, the therapeutic comprising one or more antibiotics.

37. A method of designing a treatment for a sepsis state in a subject, the method comprising:
designing a therapeutic for treating the subject in response to identifying the subject as being positive for the sepsis state using the method of any one of claims 1-23, 25-30, or 31-35.

38. A method of planning a treatment for a sepsis state in a subject, the method comprising:
generating a treatment plan for treating the subject in response to identifying the subject as being positive for the sepsis state using the method of any one of claims 1-23, 25-30, or 31-35.

39. A method of manufacturing a treatment for a sepsis state in a subject, the method comprising:
manufacturing a therapeutic for treating the subject in response to identifying the subject as being positive for the sepsis state using the method of any one of claims 1-23, 25-30, or 31-35.

40. A method of treating a sepsis state in a subject, the method comprising:
administering to the subject a therapeutic to treat the subject based on identifying the subject as being positive for the sepsis state using the method of any one of claims 1-23, 25-30, or 31-35.

41. A method of treating a sepsis state in a subject, the method comprising:
selecting a therapeutic to treat the subject based on determining that the subject is responsive to the therapeutic using the method of any of claims 1-23, 25-30, or 31-35; and
administering the selected therapeutic to the subject.

42. A method for analyzing a set of peptide structures in a sample from a patient, the method comprising:
(a) preparing a sample from the patient to form a prepared sample comprising a set of peptide structures;
(b) inputting the prepared sample into a reaction monitoring mass spectrometry system to detect a set of product ions associated with each peptide structure of the set of peptide structures, the set of peptide structures comprising at least one of:
a first peptide structure associated with the set of product ions that includes a product ion having a mass-to-charge (m/z) ratio within a range selected from a group consisting of 366.1±0.5, ±0.8, and ±1.0 and that is characterized as having a precursor ion having an m/z ratio of 1386.1 within a range selected from a group consisting of ±1.0 and ±1.5;
a second peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1167.3 within a range selected from a group consisting of ±1.0 and ±1.5;
a third peptide structure associated with the corresponding set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1270.2 within a range selected from a group consisting of ±1.0 and ±1.5;
a fourth peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 1152.5 within a range selected from a group consisting of ±1.0 and ±1.5;
a fifth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 918.4 within a range selected from a group consisting of ±1.0 and ±1.5;
a sixth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1032.9 within a range selected from a group consisting of ±1.0 and ±1.5;
a seventh peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1197 within a range selected from a group consisting of ±1.0 and ±1.5;
an eighth peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 984.4 within a range selected from a group consisting of ±1.0 and ±1.5;
a ninth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 874.7 within a range selected from a group consisting of ±1.0 and ±1.5;
a tenth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1225.8 within a range selected from a group consisting of ±1.0 and ±1.5;
an 11^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1073.4 within a range selected from a group consisting of ±1.0 and ±1.5;
a 12^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1183.5 within a range selected from a group consisting of ±1.0 and ±1.5;
a 13^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1015.2 within a range selected from a group consisting of ±1.0 and ±1.5;
a 14^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1284.6 within a range selected from a group consisting of ±1.0 and ±1.5;
a 15^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1039.1 within a range selected from a group consisting of ±1.0 and ±1.5;
a 16^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1285.1 within a range selected from a group consisting of ±1.0 and ±1.5;
a 17^{th} peptide structure associated with the set of product ions that includes a product ion having a mass-to-charge (m/z) ratio that is within a range selected from a group consisting of 1249.3±0.5, ±0.8, and ±1.0 and that is characterized as having a precursor ion having an m/z ratio of 1150.3 within a range selected from a group consisting of ±1.0 and ±1.5;
an 18^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1191.2 within a range selected from a group consisting of ±1.0 and ±1.5;
a 19^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1050.4 within a range selected from a group consisting of ±1.0 and ±1.5;
a 20^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 788.3 within a range selected from a group consisting of ±1.0 and ±1.5;
a 21^{st} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 878.8 within a range selected from a group consisting of ±1.0 and ±1.5;
a 22^{nd} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 946.5 within a range selected from a group consisting of ±1.0 and ±1.5;
a 23^{rd} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1281.5 within a range selected from a group consisting of ±1.0 and ±1.5;
a 24^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 873.4 within a range selected from a group consisting of ±1.0 and ±1.5;
a 25^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 922.1 within a range selected from a group consisting of ±1.0 and ±1.5;
a 26^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 764.8 within a range selected from a group consisting of ±1.0 and ±1.5;
a 27^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 1087.1 within a range selected from a group consisting of ±1.0 and ±1.5;
a 28^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 976.1 within a range selected from a group consisting of ±1.0 and ±1.5;
a 29^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1073.1 within a range selected from a group consisting of ±1.0 and ±1.5;
a 30^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1043.8 within a range selected from a group consisting of ±1.0 and ±1.5;
a 31^{st} peptide structure associated with the set of product ions that includes a product ion having a mass-to-charge (m/z) ratio that is within a range selected from a group consisting of 1009±0.5, ±0.8, and ±1.0 and that is characterized as having a precursor ion having an m/z ratio of 891.8 within a range selected from a group consisting of ±1.0 and ±1.5;
a 32^{nd} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 988.8 within a range selected from a group consisting of ±1.0 and ±1.5;
a 33^{rd} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio of 1142 within a range selected from a group consisting of ±1.0 and ±1.5;
a 34^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 911.4 within a range selected from a group consisting of ±1.0 and ±1.5;
a 35^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1041 within a range selected from a group consisting of ±1.0 and ±1.5;
a 36^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1008.2 within a range selected from a group consisting of ±1.0 and ±1.5;
a 37^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1109.8 within a range selected from a group consisting of ±1.0 and ±1.5;
a 38^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 761.7 within a range selected from a group consisting of ±1.0 and ±1.5;
a 39^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio of 939.1 within a range selected from a group consisting of ±1.0 and ±1.5;
a 40^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1206.3 within a range selected from a group consisting of ±1.0 and ±1.5;
a 41^{st} structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio of 1199.2 within a range selected from a group consisting of ±1.0 and ±1.5;
a 42^{nd} peptide structure associated with the set of product ions that includes a product ion having a mass-to-charge (m/z) ratio that is within a range selected from a group consisting of 565.1±0.5, ±0.8, and ±1.0 and that is characterized as having a precursor ion having an m/z ratio of 697.4 within a range selected from a group consisting of ±1.0 and ±1.5;
a 43^{rd} peptide structure associated with the set of product ions that includes a product ion having a mass-to-charge (m/z) ratio that is within a range selected from a group consisting of 736.4±0.5, ±0.8, and ±1.0 and that is characterized as having a precursor ion having an m/z ratio of 618.3 within a range selected from a group consisting of ±1.0 and ±1.5;
a 44^{th} peptide structure associated with the set of product ions that includes a product ion having a mass-to-charge (m/z) ratio that is within a range selected from a group consisting of 342.2±0.5, ±0.8, and ±1.0 and that is characterized as having a precursor ion having an m/z ratio of 590.3 within a range selected from a group consisting of ±1.0 and ±1.5;
a 45^{th} peptide structure associated with the set of product ions that includes a product ion having a mass-to-charge (m/z) ratio that is within a range selected from a group consisting of 342.2±0.5, ±0.8, and ±1.0 and that is characterized as having a precursor ion having an m/z ratio of 590.3 within a range selected from a group consisting of ±1.0 and ±1.5; and
a 46^{th} peptide structure associated with the set of product ions that includes a product ion having a mass-to-charge (m/z) ratio that is within a range selected from a group consisting of 646.4±0.5, ±0.8, and ±1.0 and that is characterized as having a precursor ion having an m/z ratio of 416.7 within a range selected from a group consisting of ±1.0 and ±1.5; and
(c) generating quantification data for the set of product ions using the reaction monitoring mass spectrometry system.

43. The method of claim 42, further comprising, prior to (a), obtaining the sample from the patient.

44. The method of claim 42, further comprising:
generating a diagnosis output using the quantification data and a model that has been trained using at least one of supervised or unsupervised machine learning.

45. The method of claim 42, wherein the reaction monitoring mass spectrometry system uses or at least one of multiple reaction monitoring mass spectrometry (MRM-MS) or selected reaction monitoring mass spectrometry (SRM-MS) to detect the set of product ions and generate the quantification data.

46. The method of claim 42, wherein the sample comprises a plasma sample.

47. The method of claim 42, wherein the sample comprises a serum sample.

48. The method of claim 42, wherein preparing the sample comprises at least one of:
denaturing one or more proteins in the sample using heat to form one or more denatured proteins;
reducing the one or more denatured proteins in the sample using a reducing agent to form one or more reduced proteins;
alkylating the one or more proteins in the sample using an alkylating agent to prevent reformation of disulfide bonds in the one or more reduced proteins to form one or more alkylated proteins; or
digesting the one or more alkylated proteins in the sample using a proteolysis catalyst to form the prepared sample comprising the set of peptide structures.

49. A composition comprising at least one of peptide structures PS-1 to PS-46 identified in Table 1.

50. A composition comprising a peptide structure or a product ion, wherein:
the peptide structure or product ion comprises an amino acid sequence having at least 90% sequence identity to any one of SEQ ID NOS: 24-54, corresponding to peptide structures PS-1 to PS-46 in Table 1; and
the product ion is selected as one from a group consisting of product ions identified in Table 2 including product ions falling within an identified m/z range.

51. A composition comprising a glycopeptide structure selected as one from a group of glycopeptide structures consisting of:
wherein:
a first glycopeptide structure having a monoisotopic mass of 5538.39 and comprising:
the amino acid sequence of SEQ ID NO: 24; and
glycan structure GL NO. 7601 linked to the 15^{th} residue of SEQ ID NO: 24;
a second glycopeptide structure having a monoisotopic mass of 5829.49 and comprising:
the amino acid sequence of SEQ ID NO: 24; and
glycan structure GL NO. 7602 linked to the 15^{th} residue of SEQ ID NO: 24;
a third glycopeptide structure having a monoisotopic mass of 3807.44 and comprising:
the amino acid sequence of SEQ ID NO: 25; and
glycan structure GL NO. 6503 linked to the 5^{th} residue of SEQ ID NO: 25;
a fourth glycopeptide structure having a monoisotopic mass of 4603.94 and comprising: the amino acid sequence of SEQ ID NO: 26; and
glycan structure GL NO. 5412 linked to the 16^{th} residue of SEQ ID NO: 26;
a fifth glycopeptide structure having a monoisotopic mass of 4584.93 and comprising:
the amino acid sequence of SEQ ID NO: 27; and
glycan structure GL NO. 5421 linked to the 2^{nd} residue of SEQ ID NO: 27;
a sixth glycopeptide structure having a monoisotopic mass of 4125.73 and comprising:
the amino acid sequence of SEQ ID NO: 28; and
glycan structure GL NO. 5401 linked to the 3^{rd} residue of SEQ ID NO: 28;
a seventh glycopeptide structure having a monoisotopic mass of 4781.95 and comprising:
the amino acid sequence of SEQ ID NO: 28; and
glycan structure GL NO. 6502 linked to the 3^{rd} residue of SEQ ID NO: 28;
an eighth glycopeptide structure having a monoisotopic mass of 4915.03 and comprising:
the amino acid sequence of SEQ ID NO: 29; and
glycan structure GL NO. 5402 linked to the 8^{th} residue of SEQ ID NO: 29;
a ninth glycopeptide structure having a monoisotopic mass of 2621.06 and comprising:
the amino acid sequence of SEQ ID NO: 30; and
glycan structure GL NO. 5301 linked to the 1^{st} residue of SEQ ID NO: 30;
a tenth glycopeptide structure having a monoisotopic mass of 4900.17 and comprising:
the amino acid sequence of SEQ ID NO: 31; and
glycan structure GL NO. 6411 linked to the 6^{th} residue of SEQ ID NO: 31;
an 11^{th} glycopeptide structure having a monoisotopic mass of 4231.67 and comprising:
the amino acid sequence of SEQ ID NO: 32; and
glycan structure GL NO. 6610 linked to the 11^{th} residue of SEQ ID NO: 32;
a 12^{th} glycopeptide structure having a monoisotopic mass of 4729.04 and comprising:
the amino acid sequence of SEQ ID NO: 33; and
glycan structure GL NO. 7602 linked to the 6^{th} residue of SEQ ID NO: 33;
a 13^{th} glycopeptide structure having a monoisotopic mass of 4055.56 and comprising:
the amino acid sequence of SEQ ID NO: 34; and
glycan structure GL NO. 5402 linked to the 1^{st} or 7^{th} residue of SEQ ID NO: 34;
a 14^{th} glycopeptide structure having a monoisotopic mass of 5133.19 and comprising:
the amino acid sequence of SEQ ID NO: 34; and
glycan structure GL NO. 5402 linked to the 7^{th} residue of SEQ ID NO: 34;
a 15^{th} glycopeptide structure having a monoisotopic mass of 5191.23 and comprising:
comprising the amino acid sequence of SEQ ID NO: 35; and
glycan structure GL NO. 5402 linked to the 19^{th} residue of SEQ ID NO: 35;
a 16^{th} glycopeptide structure having a monoisotopic mass of 5133.19 and comprising:
comprising the amino acid sequence of SEQ ID NO: 35; and
glycan structure GL NO. 5511 linked to the 19^{th} residue of SEQ ID NO: 35;
a 17^{th} glycopeptide structure having a monoisotopic mass of 5744.60 and comprising:
the amino acid sequence of SEQ ID NO: 35; and
glycan structure GL NO. 5402 linked to the 19^{th} residue of SEQ ID NO: 35;
a 18^{th} glycopeptide structure having a monoisotopic mass of 4758.93 and comprising:
the amino acid sequence of SEQ ID NO: 36; and
glycan structure GL NO. 5402 linked to the 4^{th} residue of SEQ ID NO: 36;
a 19^{th} glycopeptide structure having a monoisotopic mass of 3148.20 and comprising:
the amino acid sequence of SEQ ID NO: 37; and
glycan structure GL NO. 6513 linked to the 4^{th} residue of SEQ ID NO: 37;
a 20^{th} glycopeptide structure having a monoisotopic mass of 3148.20 and comprising:
the amino acid sequence of SEQ ID NO: 38; and
glycan structure GL NO. 5402 linked to the 2^{nd} residue of SEQ ID NO: 38;
a 21^{st} glycopeptide structure having a monoisotopic mass of 2633.04 and comprising:
the amino acid sequence of SEQ ID NO: 39; and
glycan structure GL NO. 3410 linked to the 5^{th} residue of SEQ ID NO: 39;
a 22^{nd} glycopeptide structure having a monoisotopic mass of 2836.12 and comprising:
the amino acid sequence of SEQ ID NO: 39; and
glycan structure GL NO. 3510 linked to the 5^{th} residue of SEQ ID NO: 39;
a 23^{rd} glycopeptide structure having a monoisotopic mass of 2560.02 and comprising:
the amino acid sequence of SEQ ID NO: 39; and
glycan structure GL NO. 4310 linked to the 5^{th} residue of SEQ ID NO: 39;
a 24^{th} glycopeptide structure having a monoisotopic mass of 2617.04 and comprising:
the amino acid sequence of SEQ ID NO: 39; and
glycan structure GL NO. 4400 linked to the 5^{th} residue of SEQ ID NO: 39;
a 25^{th} glycopeptide structure having a monoisotopic mass of 2763.10 and comprising:
the amino acid sequence of SEQ ID NO: 39; and
glycan structure GL NO. 4410 linked to the 5^{th} residue of SEQ ID NO: 39;
a 26^{th} glycopeptide structure having a monoisotopic mass of 3054.20 and comprising:
the amino acid sequence of SEQ ID NO: 39; and
glycan structure GL NO. 4411 linked to the 5^{th} residue of SEQ ID NO: 39;
a 27^{th} glycopeptide structure having a monoisotopic mass of 3257.28 and comprising:
the amino acid sequence of SEQ ID NO: 39; and
glycan structure GL NO. 4511 linked to the 5^{th} residue of SEQ ID NO: 39;
a 28^{th} glycopeptide structure having a monoisotopic mass of 2925.15 and comprising:
the amino acid sequence of SEQ ID NO: 39; and
glycan structure GL NO. 5410 linked to the 5^{th} residue of SEQ ID NO: 39;
a 29^{th} glycopeptide structure having a monoisotopic mass of 3216.25 and comprising:
the amino acid sequence of SEQ ID NO: 39; and
glycan structure GL NO. 5411 linked to the 5^{th} residue of SEQ ID NO: 39;
a 30^{th} glycopeptide structure having a monoisotopic mass of 3128.23 and comprising:
the amino acid sequence of SEQ ID NO: 39; and
glycan structure GL NO. 5510 linked to the 5^{th} residue of SEQ ID NO: 39;
a 31^{st} glycopeptide structure having a monoisotopic mass of 2671.29 and comprising:
the amino acid sequence of SEQ ID NO: 40; and
glycan structure GL NO. 1101 linked to the 6^{th} residue of SEQ ID NO: 40;
a 32^{nd} glycopeptide structure having a monoisotopic mass of 2962.39 and comprising:
the amino acid sequence of SEQ ID NO: 40; and
glycan structure GL NO. 1102 linked to the 6^{th} residue of SEQ ID NO: 40;
a 33^{rd} glycopeptide structure having a monoisotopic mass of 4561.95 and comprising:
the amino acid sequence of SEQ ID NO: 41; and
glycan structure GL NO. 5402 linked to the 6^{th} residue of SEQ ID NO: 41;
a 34^{th} glycopeptide structure having a monoisotopic mass of 3640.56 and comprising:
the amino acid sequence of SEQ ID NO: 42; and
glycan structure GL NO. 5402 linked to the 7^{th} residue of SEQ ID NO: 42;
a 35^{th} glycopeptide structure having a monoisotopic mass of 4159.85 and comprising:
the amino acid sequence of SEQ ID NO: 43; and
glycan structure GL NO. 5401 linked to the 6^{th} residue of SEQ ID NO: 43;
a 36^{th} glycopeptide structure having a monoisotopic mass of 5034.18 and comprising:
the amino acid sequence of SEQ ID NO: 44; and
glycan structure GL NO. 5402 linked to the 2^{nd} residue of SEQ ID NO: 44;
a 37^{th} glycopeptide structure having a monoisotopic mass of 3326.42 and comprising:
the amino acid sequence of SEQ ID NO: 45; and
glycan structure GL NO. 6301 linked to the 10^{th} residue of SEQ ID NO: 45;
a 38^{th} glycopeptide structure having a monoisotopic mass of 2280.98 and comprising:
the amino acid sequence of SEQ ID NO: 46; and
glycan structure GL NO. 1102 linked to the 8^{th} residue of SEQ ID NO: 46;
a 39^{th} glycopeptide structure having a monoisotopic mass of 2813.31 and comprising:
the amino acid sequence of SEQ ID NO: 47; and
glycan structure GL NO. 1101 linked to the 12^{th} residue of SEQ ID NO: 47;
a 40^{th} glycopeptide structure having a monoisotopic mass of 8432.83 and comprising:
the amino acid sequence of SEQ ID NO: 48; and
glycan structure GL NO. 5402 linked to the 46^{th} residue of SEQ ID NO: 48;
a 41^{st} glycopeptide structure having a monoisotopic mass of 4791.91 and comprising:
the amino acid sequence of SEQ ID NO: 49; and
glycan structure GL NO. 7420 linked to the 4^{th} residue of SEQ ID NO: 49;
wherein:
the glycan structure GL NO. 1101 comprises: 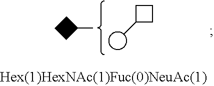
the glycan structure GL NO. 1102 comprises: 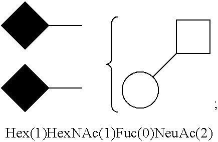
the glycan structure GL NO. 3410 comprises: 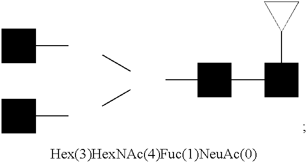
the glycan structure GL NO. 3510 comprises: 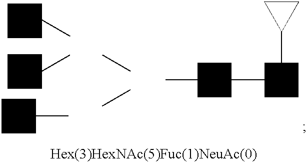
the glycan structure GL NO. 3510 comprises: 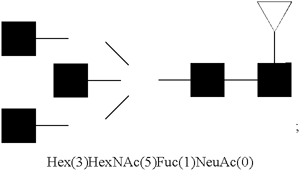
the glycan structure GL NO. 4310 comprises: 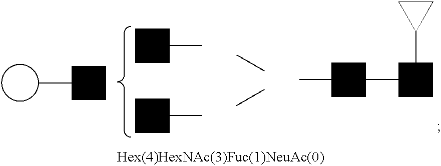
the glycan structure GL NO. 4400 comprises: 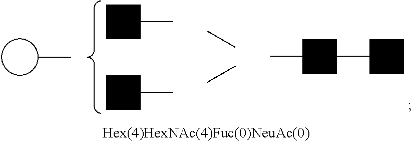
the glycan structure GL NO. 4410 comprises: 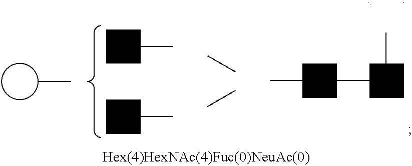
the glycan structure GL NO. 4411 comprises: 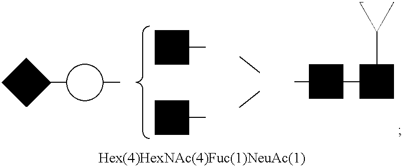
the glycan structure GL NO. 4511 comprises: 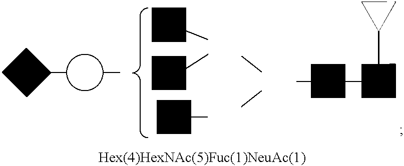
the glycan structure GL NO. 5301 comprises: 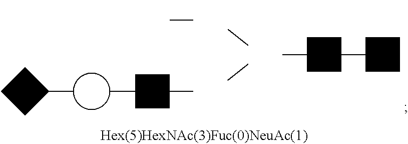
the glycan structure GL NO. 5401 comprises: 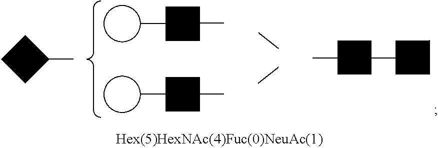
the glycan structure GL NO. 5402 comprises: 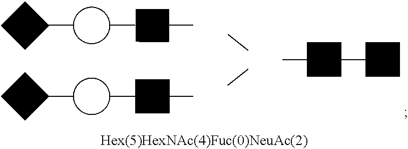
the glycan structure GL NO. 5410 comprises: 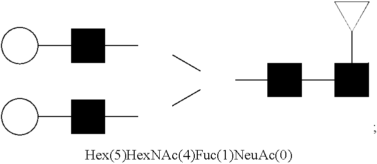
the glycan structure GL NO. 5411 comprises: 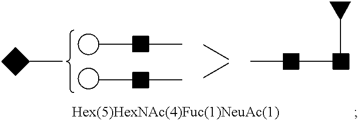
the glycan structure GL NO. 5412 comprises: 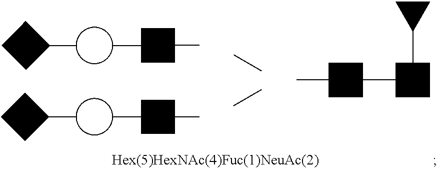
the glycan structure GL NO. 5421 comprises: 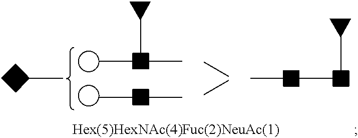
the glycan structure GL NO. 5510 comprises: 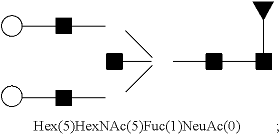
the glycan structure GL NO. 5511 comprises: 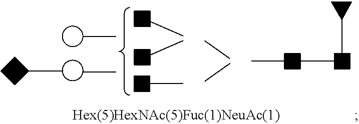
the glycan structure GL NO. 5511 comprises: 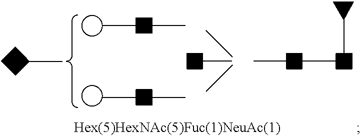
the glycan structure GL NO. 6301 comprises: 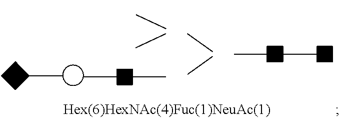
the glycan structure GL NO. 6411 comprises: 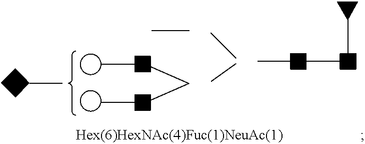
the glycan structure GL NO. 6502 comprises: 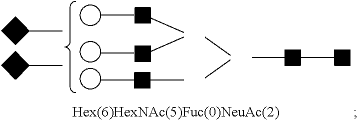
the glycan structure GL NO. 6503 comprises: 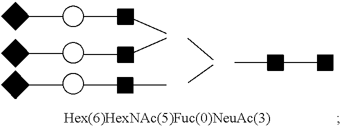
the glycan structure GL NO. 6513 comprises: 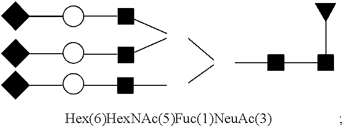
the glycan structure GL NO. 6610 comprises: 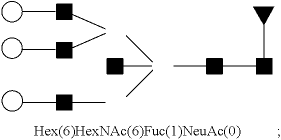
the glycan structure GL NO. 7420 comprises: 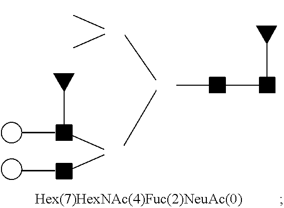
the glycan structure GL NO. 7602 comprises: 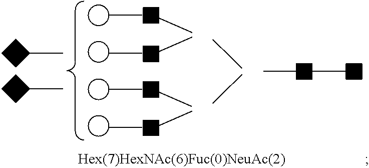
and
the glycan structure GL NO. 7601 comprises:

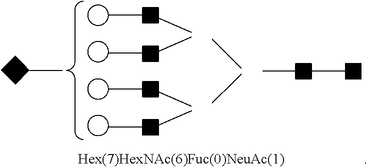

52. The composition of claim 51, wherein:
the first glycopeptide structure has a precursor ion having a charge of 4;
the second glycopeptide structure has a precursor ion having a charge of 5;
the third glycopeptide structure has a precursor ion having a charge of 3;
the fourth glycopeptide structure has a precursor ion having a charge of 4;
the fifth glycopeptide structure has a precursor ion having a charge of 5;
the sixth glycopeptide structure has a precursor ion having a charge of 4;
the seventh glycopeptide structure has a precursor ion having a charge of 4;
the eighth glycopeptide structure has a precursor ion having a charge of 5;
the ninth glycopeptide structure has a precursor ion having a charge of 3;
the tenth glycopeptide structure has a precursor ion having a charge of 4;
the 11^{th} glycopeptide structure has a precursor ion having a charge of 4;
the 12^{th} glycopeptide structure has a precursor ion having a charge of 4;
the 13^{th} glycopeptide structure has a precursor ion having a charge of 4;
the 14^{th} glycopeptide structure has a precursor ion having a charge of 4;
the 15^{th} glycopeptide structure has a precursor ion having a charge of 5;
the 16^{th} glycopeptide structure has a precursor ion having a charge of 4;
the 17^{th} glycopeptide structure has a precursor ion having a charge of 5;
the 18^{th} glycopeptide structure has a precursor ion having a charge of 4;
the 19^{th} glycopeptide structure has a precursor ion having a charge of 3;
the 20^{th} glycopeptide structure has a precursor ion having a charge of 4;
the 21^{st} glycopeptide structure has a precursor ion having a charge of 3;
the 22^{nd} glycopeptide structure has a precursor ion having a charge of 3;
the 23^{rd} glycopeptide structure has a precursor ion having a charge of 2;
the 24^{th} glycopeptide structure has a precursor ion having a charge of 3;
the 25^{th} glycopeptide structure has a precursor ion having a charge of 3;
the 26^{th} glycopeptide structure has a precursor ion having a charge of 4;
the 27^{th} glycopeptide structure has a precursor ion having a charge of 3;
the 28^{th} glycopeptide structure has a precursor ion having a charge of 3;
the 29^{th} glycopeptide structure has a precursor ion having a charge of 3;
the 30^{th} glycopeptide structure has a precursor ion having a charge of 3;
the 31^{st} glycopeptide structure has a precursor ion having a charge of 3;
the 32^{nd} glycopeptide structure has a precursor ion having a charge of 3;
the 33^{rd} glycopeptide structure has a precursor ion having a charge of 4;
the 34^{th} glycopeptide structure has a precursor ion having a charge of 4;
the 35^{th} glycopeptide structure has a precursor ion having a charge of 4;
the 36^{th} glycopeptide structure has a precursor ion having a charge of 5;
the 37^{th} glycopeptide structure has a precursor ion having a charge of 3;
the 38^{th} glycopeptide structure has a precursor ion having a charge of 3;
the 39^{th} glycopeptide structure has a precursor ion having a charge of 3;
the 40^{th} glycopeptide structure has a precursor ion having a charge of 7; and
the 41^{st} glycopeptide structure has a precursor ion having a charge of 4.

53. The composition of claim 51 or claim 52, wherein:
the first glycopeptide structure has a product ion having a mass-to-charge (m/z) ratio within a range selected from a group consisting of 366.1±0.5, ±0.8, and ±1.0;
the second glycopeptide structure has a product ion having a mass-to-charge (m/z) ratio that is within a range selected from a group consisting of 366.1±0.5, ±0.8, and ±1.0;
the third glycopeptide structure associated with the corresponding set of product ions that includes a product ion having a mass-to-charge (m/z) ratio that is within a range selected from a group consisting of 366.1±0.5, ±0.8, and ±1.0;
the fourth glycopeptide structure has a product ion having a mass-to-charge (m/z) ratio that is within a range selected from a group consisting of 274.1±0.5, ±0.8, and ±1.0;
the fifth glycopeptide structure has a product ion having a mass-to-charge (m/z) ratio that is within a range selected from a group consisting of 366.1±0.5, ±0.8, and ±1.0;
the sixth glycopeptide structure has a product ion having a mass-to-charge (m/z) ratio that is within a range selected from a group consisting of 366.1±0.5, ±0.8, and ±1.0;
the seventh glycopeptide structure has a product ion having a mass-to-charge (m/z) ratio that is within a range selected from a group consisting of 366.1±0.5, ±0.8, and ±1.0;
the eighth glycopeptide structure has a product ion having a mass-to-charge (m/z) ratio that is within a range selected from a group consisting of 274.1±0.5, ±0.8, and ±1.0;
the ninth glycopeptide structure has a product ion having a mass-to-charge (m/z) ratio that is within a range selected from a group consisting of 366.1±0.5, ±0.8, and ±1.0;
the tenth glycopeptide structure has a product ion having a mass-to-charge (m/z) ratio that is within a range selected from a group consisting of 366.1±0.5, ±0.8, and ±1.0;
the 11^{th} glycopeptide structure has a product ion having a mass-to-charge (m/z) ratio that is within a range selected from a group consisting of 366.1±0.5, ±0.8, and ±1.0;
the 12^{th} glycopeptide structure has a product ion having a mass-to-charge (m/z) ratio that is within a range selected from a group consisting of 366.1±0.5, ±0.8, and ±1.0;
the 13^{th} glycopeptide structure has a product ion having a mass-to-charge (m/z) ratio that is within a range selected from a group consisting of 366.1±0.5, ±0.8, and ±1.0;
the 14^{th} glycopeptide structure has a product ion having a mass-to-charge (m/z) ratio that is within a range selected from a group consisting of 366.1±0.5, ±0.8, and ±1.0;
the 15^{th} glycopeptide structure has a product ion having a mass-to-charge (m/z) ratio that is within a range selected from a group consisting of 366.1±0.5, ±0.8, and ±1.0;
the 16^{th} glycopeptide structure has a product ion having a mass-to-charge (m/z) ratio that is within a range selected from a group consisting of 366.1±0.5, ±0.8, and ±1.0;
the 17^{th} glycopeptide structure has a product ion having a mass-to-charge (m/z) ratio that is within a range selected from a group consisting of 1249.3±0.5, ±0.8, and ±1.0;
the 18^{th} glycopeptide structure has a product ion having a mass-to-charge (m/z) ratio that is within a range selected from a group consisting of 366.1±0.5, ±0.8, and ±1.0;
the 19^{th} glycopeptide structure has a product ion having a mass-to-charge (m/z) ratio that is within a range selected from a group consisting of 366.1±0.5, ±0.8, and ±1.0;
the 20^{th} glycopeptide structure has a product ion having a mass-to-charge (m/z) ratio that is within a range selected from a group consisting of 366.1±0.5, ±0.8, and ±1.0;
the 21^{st} glycopeptide structure has a product ion having a mass-to-charge (m/z) ratio that is within a range selected from a group consisting of 204.1±0.5, ±0.8, and ±1.0;
the 22^{nd} glycopeptide structure has a product ion having a mass-to-charge (m/z) ratio that is within a range selected from a group consisting of 204.1±0.5, ±0.8, and ±1.0;
the 23^{rd} glycopeptide structure has a product ion having a mass-to-charge (m/z) ratio that is within a range selected from a group consisting of 366.1±0.5, ±0.8, and ±1.0;
the 24^{th} glycopeptide structure has a product ion having a mass-to-charge (m/z) ratio that is within a range selected from a group consisting of 204.1±0.5, ±0.8, and ±1.0;
the 25^{th} glycopeptide structure has a product ion having a mass-to-charge (m/z) ratio that is within a range selected from a group consisting of 204.1±0.5, ±0.8, and ±1.0;
the 26^{th} glycopeptide structure has a product ion having a mass-to-charge (m/z) ratio that is within a range selected from a group consisting of 204.1±0.5, ±0.8, and ±1.0;
the 27^{th} glycopeptide structure has a product ion having a mass-to-charge (m/z) ratio that is within a range selected from a group consisting of 204.1±0.5, ±0.8, and ±1.0;
the 28^{th} glycopeptide structure has a product ion having a mass-to-charge (m/z) ratio that is within a range selected from a group consisting of 366.1±0.5, ±0.8, and ±1.0;
the 29^{th} glycopeptide structure has a product ion having a mass-to-charge (m/z) ratio that is within a range selected from a group consisting of 366.1±0.5, ±0.8, and ±1.0;
the 30^{th} glycopeptide structure has a product ion having a mass-to-charge (m/z) ratio that is within a range selected from a group consisting of 366.1±0.5, ±0.8, and ±1.0;
the 31^{st} glycopeptide structure has a product ion having a mass-to-charge (m/z) ratio that is within a range selected from a group consisting of 1009±0.5, ±0.8, and ±1.0;
the 32^{nd} glycopeptide structure has a product ion having a mass-to-charge (m/z) ratio that is within a range selected from a group consisting of 274.1±0.5, ±0.8, and ±1.0;
the 33^{rd} glycopeptide structure has a product ion having a mass-to-charge (m/z) ratio that is within a range selected from a group consisting of 204.1±0.5, ±0.8, and ±1.0;
the 34^{th} glycopeptide structure has a product ion having a mass-to-charge (m/z) ratio that is within a range selected from a group consisting of 274.1±0.5, ±0.8, and ±1.0;
the 35^{th} glycopeptide structure has a product ion having a mass-to-charge (m/z) ratio that is within a range selected from a group consisting of 366.1±0.5, ±0.8, and ±1.0;
the 36^{th} glycopeptide structure has a product ion having a mass-to-charge (m/z) ratio that is within a range selected from a group consisting of 366.1±0.5, ±0.8, and ±1.0;
the 37^{th} glycopeptide structure has a product ion having a mass-to-charge (m/z) ratio that is within a range selected from a group consisting of 366.1±0.5, ±0.8, and ±1.0;
the 38^{th} glycopeptide structure has a product ion having a mass-to-charge (m/z) ratio that is within a range selected from a group consisting of 274.1±0.5, ±0.8, and ±1.0;
the 39^{th} glycopeptide structure has a product ion having a mass-to-charge (m/z) ratio that is within a range selected from a group consisting of 274.1±0.5, ±0.8, and ±1.0;
the 40^{th} glycopeptide structure has a product ion having a mass-to-charge (m/z) ratio that is within a range selected from a group consisting of 366.1±0.5, ±0.8, and ±1.0; and
the 41^{st} glycopeptide structure has a product ion having a mass-to-charge (m/z) ratio that is within a range selected from a group consisting of 366.1±0.5, ±0.8, and ±1.0.

54. The composition of any one of claims 51-53, wherein:
the first glycopeptide structure has a precursor ion having an m/z ratio of 1386.1 within a range selected from a group consisting of ±1.0 and ±1.5;
the second glycopeptide structure has a precursor ion having an m/z ratio of 1167.3 within a range selected from a group consisting of ±1.0 and ±1.5;
the third glycopeptide structure has a precursor ion having an m/z ratio of 1270.2 within a range selected from a group consisting of ±1.0 and ±1.5;
the fourth glycopeptide structure has a precursor ion having an m/z ratio of 1152.5 within a range selected from a group consisting of ±1.0 and ±1.5;
the fifth glycopeptide structure has a precursor ion having an m/z ratio of 918.4 within a range selected from a group consisting of ±1.0 and ±1.5;
the sixth glycopeptide structure has a precursor ion having an m/z ratio of 1032.9 within a range selected from a group consisting of ±1.0 and ±1.5;
the seventh glycopeptide structure has a precursor ion having an m/z ratio of 1197 within a range selected from a group consisting of ±1.0 and ±1.5;
the eighth glycopeptide structure has a precursor ion having an m/z ratio of 984.4 within a range selected from a group consisting of ±1.0 and ±1.5;
the ninth glycopeptide structure has a precursor ion having an m/z ratio of 874.7 within a range selected from a group consisting of ±1.0 and ±1.5;
the tenth glycopeptide structure has a precursor ion having an m/z ratio of 1225.8 within a range selected from a group consisting of ±1.0 and ±1.5;
the 11^{th} glycopeptide structure has a precursor ion having an m/z ratio of 1073.4 within a range selected from a group consisting of ±1.0 and ±1.5;
the 12^{th} glycopeptide structure has a precursor ion having an m/z ratio of 1183.5 within a range selected from a group consisting of ±1.0 and ±1.5;
the 13^{th} glycopeptide structure has a precursor ion having an m/z ratio of 1015.2 within a range selected from a group consisting of ±1.0 and ±1.5;
the 14^{th} glycopeptide structure has a precursor ion having an m/z ratio of 1284.6 within a range selected from a group consisting of ±1.0 and ±1.5;
the 15^{th} glycopeptide structure has a precursor ion having an m/z ratio of 1039.1 within a range selected from a group consisting of ±1.0 and ±1.5;
the 16^{th} glycopeptide structure has a precursor ion having an m/z ratio of 1285.1 within a range selected from a group consisting of ±1.0 and ±1.5;
the 17^{th} glycopeptide structure has a precursor ion having an m/z ratio of 1150.3 within a range selected from a group consisting of ±1.0 and ±1.5;
the 18^{th} glycopeptide structure has a precursor ion having an m/z ratio of 1191.2 within a range selected from a group consisting of ±1.0 and ±1.5;
the 19^{th} glycopeptide structure has a precursor ion having an m/z ratio of 1050.4 within a range selected from a group consisting of ±1.0 and ±1.5;
the 20^{th} glycopeptide structure has a precursor ion having an m/z ratio of 788.3 within a range selected from a group consisting of ±1.0 and ±1.5;
the 21^{st} glycopeptide structure has a precursor ion having an m/z ratio of 878.8 within a range selected from a group consisting of ±1.0 and ±1.5;
the 22^{nd} glycopeptide structure has a precursor ion having an m/z ratio of 946.5 within a range selected from a group consisting of ±1.0 and ±1.5;
the 23^{rd} glycopeptide structure has a precursor ion having an m/z ratio of 1281.5 within a range selected from a group consisting of ±1.0 and ±1.5;
the 24^{th} glycopeptide structure has a precursor ion having an m/z ratio of 873.4 within a range selected from a group consisting of ±1.0 and ±1.5;
the 25^{th} glycopeptide structure has a precursor ion having an m/z ratio of 922.1 within a range selected from a group consisting of ±1.0 and ±1.5;
the 26^{th} glycopeptide structure has a precursor ion having an m/z ratio of 764.8 within a range selected from a group consisting of ±1.0 and ±1.5;
the 27^{th} glycopeptide structure has a precursor ion having an m/z ratio of 1087.1 within a range selected from a group consisting of ±1.0 and ±1.5;
the 28^{th} glycopeptide structure has a precursor ion having an m/z ratio of 976.1 within a range selected from a group consisting of ±1.0 and ±1.5;
the 29^{th} glycopeptide structure has a precursor ion having an m/z ratio of 1073.1 within a range selected from a group consisting of ±1.0 and ±1.5;
the 30^{th} glycopeptide structure has a precursor ion having an m/z ratio of 1043.8 within a range selected from a group consisting of ±1.0 and ±1.5;
the 31^{st} glycopeptide structure has a precursor ion having an m/z ratio of 891.8 within a range selected from a group consisting of ±1.0 and ±1.5;
the 32^{nd} glycopeptide structure has a precursor ion having an m/z ratio of 988.8 within a range selected from a group consisting of ±1.0 and ±1.5;
the 33^{rd} glycopeptide structure has a precursor ion having an m/z ratio of 1142 within a range selected from a group consisting of ±1.0 and ±1.5;
the 34^{th} glycopeptide structure has a precursor ion having an m/z ratio of 911.4 within a range selected from a group consisting of ±1.0 and ±1.5;
the 35^{th} glycopeptide structure has a precursor ion having an m/z ratio of 1041 within a range selected from a group consisting of ±1.0 and ±1.5;
the 36^{th} glycopeptide structure has a precursor ion having an m/z ratio of 1008.2 within a range selected from a group consisting of ±1.0 and ±1.5;
the 37^{th} glycopeptide structure has a precursor ion having an m/z ratio of 1109.8 within a range selected from a group consisting of ±1.0 and ±1.5;
the 38^{th} glycopeptide structure has a precursor ion having an m/z ratio of 761.7 within a range selected from a group consisting of ±1.0 and ±1.5;
the 39^{th} glycopeptide structure has a precursor ion having an m/z ratio of 939.1 within a range selected from a group consisting of ±1.0 and ±1.5;
the 40^{th} glycopeptide structure has a precursor ion having an m/z ratio of 1206.3 within a range selected from a group consisting of ±1.0 and ±1.5; and
the 41^{st} structure has a precursor ion having an m/z ratio of 1199.2 within a range selected from a group consisting of ±1.0 and ±1.5.

55. A composition comprising a peptide structure selected as one from a group of aglycosylated peptide structures consisting of:
a first peptide structure having a monoisotopic mass of 1392.69 and comprising the amino acid sequence of SEQ ID NO: 50;
a second peptide structure having a monoisotopic mass of 1234.68 and comprising the amino acid sequence of SEQ ID NO: 51;
a third peptide structure having a monoisotopic mass of 1178.67 and comprising the amino acid sequence of SEQ ID NO: 52;
a fourth peptide structure having a monoisotopic mass of 2454.14 and comprising the amino acid sequence of SEQ ID NO: 53; and
a fifth peptide structure having a monoisotopic mass of 831.47 and comprising the amino acid sequence of SEQ ID NO: 54.

56. The composition of claim 55, wherein:
the first peptide structure has a precursor ion having a charge of 2;
the second peptide structure has a precursor ion having a charge of 2;
the third peptide structure has a precursor ion having a charge of 2;
the fourth peptide structure has a precursor ion having a charge of 3; and
the fifth peptide structure has a precursor ion having a charge of 2.

57. The composition of claim 55 or 56, wherein:
the first peptide structure has a precursor ion m/z ratio within a range selected from a group consisting of 697.4+/−1.0, and +/−1.5;
the second peptide structure has a precursor ion m/z ratio within a range selected from a group consisting of 618.3+/−1.0, and +/−1.5;
the third peptide structure has a precursor ion m/z ratio within a range selected from a group consisting of 590.3+/−1.0, and +/−1.5;
the fourth peptide structure has a precursor ion m/z ratio within a range selected from a group consisting of 819.1+/−1.0, and +/−1.5; and
the fifth peptide structure has a precursor ion m/z ratio within a range selected from a group consisting of 416.7+/−1.0, and +/−1.5.

58. The composition of claim 55 or 56, wherein:
the first peptide structure has a product ion m/z ratio within a range selected from a group consisting of 565.3+/−0.5, +/−0.8, and +/−1.0;
the second peptide structure has a product ion m/z ratio within a range selected from a group consisting of 736.4+/−0.5, +/−0.8, and +/−1.0;
the third peptide structure has a product ion m/z ratio within a range selected from a group consisting of 342.2+/−0.5, +/−0.8, and +/−1.0;
the fourth peptide structure has a product ion m/z ratio within a range selected from a group consisting of 609.3+/−0.5, +/−0.8, and +/−1.0; and
the fifth peptide structure has a product ion m/z ratio within a range selected from a group consisting of 646.4+/−0.5, +/−0.8, and +/−1.0.

59. A kit comprising at least one agent for quantifying at least one peptide structure identified in Table 1 to carry out the method of any one of claims 1-48.

60. A kit comprising at least one of a glycopeptide standard, a buffer, or a set of peptide sequences to carry out the method of any one of claims 1-48, a peptide sequence of the set of peptide sequences identified by a corresponding one of SEQ ID NOS: 24-54, defined in Table 3.

61. A system comprising:
one or more data processors; and
a non-transitory computer readable storage medium containing instructions which, when executed on the one or more data processors, cause the one or more data processors to perform part or all of any one of claims 1-23, 25-30, or 31-35.

62. A computer-program product tangibly embodied in a non-transitory machine-readable storage medium, including instructions configured to cause one or more data processors to perform part or all of any one of claims 1-23, 25-30, or 31-35.

63. A method of determining whether a biological sample corresponds to a symptomatic disease state of a coronavirus disease (COVID), the method comprising:
inputting quantification data identified from peptide structure data for a set of peptide structures into a supervised machine learning model, wherein the set of peptide structures includes at least one peptide structure identified in Table 2-1;
analyzing the quantification data using the supervised machine learning model to generate a disease indicator that classifies the biological sample as corresponding to a cluster of a plurality of clusters that has a least distance to the biological sample, the plurality of clusters corresponding a plurality of states that includes the symptomatic disease state; and
generating a diagnosis output based on the disease indicator.

64. The method of claim 63, further comprising receiving peptide structure data corresponding to the biological sample obtained from a subject.

65. The method of claim 63, wherein the at least one peptide structure comprises a glycopeptide structure defined by a peptide sequence and a glycan structure linked to the peptide sequence at a linking site of the peptide sequence, as identified in Table 2-1, with the peptide sequence being one of SEQ ID NOS: 84-116 as defined in Table 5-1.

66. The method of claim 63, wherein the at least one peptide structure comprises an aglycosylated peptide structure defined by a peptide sequence, as identified in Table 2-1, with the peptide sequence being one of SEQ ID NOS: 107-110 as defined in Table 5-1.

67. The method of claim 63, wherein the supervised machine learning model comprises a Support Vector Machine (SVM) classifier.

68. The method of claim 63, wherein the supervised machine learning model comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

69. The method of claim 63, further comprising:
training the supervised machine learning model using training data generated from an unsupervised machine learning model,
wherein the training data comprises a plurality of peptide structure profiles for a plurality of subjects and identifies a corresponding state of the plurality of states for each peptide structure profile of the plurality of peptide structure profiles; and
wherein the unsupervised machine learning model is trained to cluster the plurality of peptide structure profiles into the plurality of clusters.

70. The method of claim 69, wherein the unsupervised machine learning model is a k-means clustering model.

71. The method of claim 69, wherein a peptide structure profile of the plurality of peptide structure profiles for a corresponding subject of the plurality of subjects is selected based on a differential expression analysis of quantification metrics for a panel of peptide structures for the plurality of subjects.

72. The method of claim 71, further comprising:
comparing, using the differential expression analysis, the quantification metrics corresponding to the panel of peptide structures for a first portion of the plurality of subjects diagnosed with the symptomatic disease state to that of each portion of a set of other portions of the plurality of subjects to generate a set of comparisons; and
selecting a portion of the panel of peptide structures having a false discovery rate below 0.05 across the set of comparisons as the set of peptide structures.

73. The method of claim 72, wherein the comparing comprises:
comparing the quantification metrics corresponding to the panel of peptide structures for the first portion of the plurality of subjects diagnosed with the symptomatic disease state to that of a second portion of the plurality of subjects diagnosed with a sepsis state to generate a first comparison of the set of comparisons.

74. The method of claim 73, wherein the comparing further comprises:
comparing the quantification metrics corresponding to the panel of peptide structures for the first portion of the plurality of subjects diagnosed with the symptomatic disease state to that of at least one of:
a third portion of the plurality of subjects diagnosed with a common cold state to generate a second comparison of the set of comparisons;
a third portion of the plurality of subjects diagnosed with an asymptomatic disease state of the coronavirus disease to generate a third comparison of the set of comparisons; or
a fourth portion of the plurality of subjects diagnosed with a healthy state to generate a fourth comparison of the set of comparisons.

75. The method of claim 63, wherein the quantification data for a peptide structure of the set of peptide structures comprises at least one of a relative quantity, an adjusted quantity, a normalized quantity, a relative concentration, an adjusted concentration, or a normalized concentration.

76. The method of claim 63, wherein the peptide structure data is generated using multiple reaction monitoring mass spectrometry (MRM-MS).

77. The method of claim 63, further comprising:
creating a sample from the biological sample; and
preparing the sample using reduction, alkylation, and enzymatic digestion to form a prepared sample that includes a set of peptide structures.

78. The method of claim 63, further comprising:
generating the peptide structure data from the prepared sample using multiple reaction monitoring mass spectrometry (MRM-MS).

79. The method of claim 63, wherein generating the diagnosis output comprises:
generating a report that includes a diagnosis that the subject is positive for the symptomatic disease state in response to the disease indicator classifying the biological sample as corresponding to the symptomatic disease state of the coronavirus disease.

80. The method of claim 63, wherein the coronavirus disease is caused by severe acute respiratory syndrome coronavirus 2 (SARS-CoV-2).

81. The method of claim 63, further comprising:
generating a treatment output based on at least one of the diagnosis output or the disease indicator.

82. The method of claim 81, wherein the treatment output comprises at least one of an identification of a treatment to treat the subject, a design for the treatment, a manufacturing plan for the treatment, or a treatment plan for administering the treatment.

83. The method of claim 81, wherein the treatment comprises at least one of an antiviral therapeutic, an anti-inflammatory therapeutic, or an immune-based therapeutic.

84. The method of claim 81, wherein the treatment comprises at least one of remdesivir, baricitinib, tocilizumab, favipiravir, merimepodib, a monoclonal antibody therapeutic, Nirmatrelvir with Ritonavi, Molnupiravir, and/or an immune checkpoint inhibitor.

85. The method of claim 81, wherein generating the treatment output comprises:
determining a dosage for a therapeutic to treat the subject based on at least one of the diagnosis output or the disease indicator.

86. The method of any one of claims 63-85, wherein the diagnosis output identifies that the biological sample is positive for the symptomatic disease state and further comprising:
administering a therapeutic dosage of a therapeutic for the coronavirus disease to the subject, the therapeutic being selected from the group consisting of remdesivir, baricitinib, tocilizumab, favipiravir, merimepodib, a monoclonal antibody treatment, Nirmatrelvir with Ritonavi, Molnupiravir, and/or an immune checkpoint inhibitor.

87. A method of identifying a coronavirus disease (COVID)-specific set of peptide structures for use in diagnosing a symptomatic disease state of the coronavirus disease, the method comprising:
receiving quantification data for a panel of peptide structures for a plurality of subjects diagnosed with a plurality of states, the plurality of states including the symptomatic disease state of the coronavirus disease;
comparing quantification metrics corresponding to the panel of peptide structures for a first portion of the plurality of subjects diagnosed with the symptomatic disease state to that of each portion of a set of other portions of the plurality of subjects to generate a set of comparisons using a differential expression analysis;
selecting a portion of the panel of peptide structures having a false discovery rate below 0.05 across the set of comparisons as a set of peptide structures to be associated with the coronavirus disease,
wherein the set of peptide structures includes at least one glycopeptide structure defined by a peptide sequence and a glycan structure linked to the peptide sequence at a linking site of the peptide sequence;
analyzing the quantification data for the set of peptide structures for the plurality of subjects using an unsupervised machine learning model to cluster the plurality of subjects; and
training a supervised machine learning model to determine whether a biological sample obtained from a subject corresponds to the symptomatic disease state or another state of the plurality of states.

88. The method of claim 87, further comprising:
analyzing the biological sample obtained from the subject using the supervised machine learning model that has been trained to generate a disease indicator that indicates whether the biological subject is positive for the symptomatic disease state.

89. The method of claim 87, wherein the plurality of states further includes at least one of a sepsis state, a common cold state, a healthy state, or an asymptomatic disease state of the coronavirus disease.

90. The method of claim 87, wherein the coronavirus disease is caused by severe acute respiratory syndrome coronavirus 2 (SARS-CoV-2).

91. The method of claim 87, wherein the unsupervised machine learning model comprises a k-means clustering model and wherein the supervised machine learning model comprises at least one of a Support Vector Machine (SVM) classifier, a Support Vector Classifier (SVC) model, a linear classifier, a decision tree, a random forest algorithm, a k-Nearest Neighbors algorithm, a Naive Bayes algorithm, or a gradient boosting algorithm.

92. The method of claim 87, wherein the set of peptide structures includes at least three peptide structures identified in Table 2-1.

93. A method of diagnosing a symptomatic disease state of a coronavirus disease (COVID), the method comprising:
analyzing peptide structure data using a supervised machine learning model to generate a disease indicator that indicates whether a biological sample is positive for the symptomatic disease state based on at least 3 peptide structures selected from a group of peptide structures identified in Table 3-1,
wherein the group of peptide structures in Table 3-1 comprises a group of peptide structures associated with the symptomatic disease state; and
wherein the group of peptide structures is listed in Table 3-1 with respect to relative significance to the disease indicator; and
generating a diagnosis output based on the disease indicator.

94. The method of claim 93, further comprising receiving peptide structure data corresponding to a biological sample obtained from a subject.

95. The method of claim 93, wherein a peptide structure of the at least 3 peptide structures comprises a glycopeptide structure defined by a peptide sequence and a glycan structure linked to the peptide sequence at a linking site of the peptide sequence, as identified in Table 3-1, with the peptide sequence, which is one of SEQ ID NOS: 84, 85, 92, 94, 96, 97, 104 and 111-116, being defined in Table 5-1.

96. The method of claim 93, wherein a peptide structure of the at least 3 peptide structures comprises an aglycosylated peptide structure defined by a peptide sequence, as identified in Table 3-1, with the peptide sequence, which is one of SEQ ID NOS: 107-110, being defined in Table 5-1.

97. The method of claim 93, wherein the at least 3 peptide structures includes 16 glycopeptide structures and 2 aglycosylated peptide structures.

98. The method of claim 93, wherein the supervised machine learning model comprises a regression model.

99. The method of claim 93, wherein the supervised machine learning model comprises a penalized multivariable regression model.

100. The method of claim 93, wherein the peptide structure data comprises quantification data for each peptide structure of a panel of peptide structures, the panel of peptide structures including the at least 3 peptide structures.

101. The method of claim 100, wherein the quantification data for a peptide structure of the plurality of peptide structures comprises at least one of a relative quantity, an adjusted quantity, a normalized quantity, a relative concentration, an adjusted concentration, or a normalized concentration.

102. The method of claim 93, wherein the disease indicator comprises at least one of a probability that the subject is positive for the symptomatic disease state, an odds that the subject is positive for the symptomatic disease state, a logarithm of the odds that the subject is positive for the symptomatic disease state, or a classification of the biological sample as either positive or negative for the symptomatic disease state.

103. The method of claim 93, wherein generating the diagnosis output comprises:
generating a report that includes a diagnosis that the subject is positive for the symptomatic disease state in response to a determination that the disease indicator is above a selected threshold.

104. The method of claim 103, wherein the selected threshold comprises at least one a probability threshold selected as a value between a range from 0.50 to 0.95 or a logit threshold selected as a value either equal to or above 0.0.

105. The method of claim 93, wherein analyzing the peptide structure data comprises:
computing the disease indicator using a weight coefficient associated with each peptide structure of the at least 3 peptide structures, the weight coefficient of a corresponding peptide structure of the at least 3 peptide structures indicating the relative significance of the corresponding peptide structure to the disease indicator.

106. The method of claim 93, wherein analyzing the peptide structure data comprises:
computing a peptide structure profile for the biological sample that identifies a weighted value for each peptide structure of the at least 3 peptide structures, wherein the weighted value for a peptide structure of the at least 3 peptide structures is a product of a quantification metric for the peptide structure identified from the peptide structure data and a weight coefficient for the peptide structure; and
computing the disease indicator using the peptide structure profile.

107. The method of claim 93, wherein the disease indicator comprises a probability that the biological sample is positive for the symptomatic disease state and wherein the supervised machine learning model is configured to generate an output that identifies the biological sample as either positive for the symptomatic disease state when the disease indicator is greater than a selected threshold or negative for the symptomatic disease state when the disease indicator is not greater than the selected threshold.

108. The method of claim 107, wherein the selected threshold is a value selected as either equal to or above 0.5.

109. The method of claim 107, wherein the selected threshold is a value within ±0.02 of 0.525.

110. The method of claim 93, wherein:
the supervised machine learning model is trained to determine weight coefficients for a panel of peptide structures such that a first portion of the weight coefficients for a first portion of the panel of peptide structures are non-zero and a second portion of the weight coefficients for a second portion of the panel of peptide structures are zero; and
the first portion of the panel of peptide structures forms the group of peptide structures identified in Table 3-1.

111. The method of claim 110, further comprising:
training the supervised machine learning model using training data that comprises a plurality of peptide structure profiles for a plurality of subjects and a corresponding state of a plurality of states for each peptide structure profile of the plurality of peptide structure profiles,
wherein the plurality of subjects includes a first portion diagnosed with the symptomatic disease state and at least one of:
a second portion having a healthy state;
a third portion diagnosed with a common cold state;
a fourth portion diagnosed with an asymptomatic disease state of the coronavirus disease (COVID); or
a fifth portion diagnosed with a sepsis state.

112. The method of claim 93, wherein the symptomatic disease state is one of a plurality of symptomatic disease states for the coronavirus (COVID), the plurality of symptomatic disease states corresponding to varying levels of severity.

113. The method of claim 93, further comprising:
creating a sample from the biological sample; and
preparing the sample using reduction, alkylation, and enzymatic digestion to form a prepared sample that includes a set of peptide structures.

114. The method of claim 113, further comprising:
generating the peptide structure data from the prepared sample using multiple reaction monitoring mass spectrometry (MRM-MS).

115. The method of claim 93, wherein the coronavirus disease (COVID) is caused by severe acute respiratory syndrome coronavirus 2 (SARS-CoV-2).

116. The method of claim 93, further comprising:
generating a treatment output based on at least one of the diagnosis output or the disease indicator.

117. The method of claim 116, wherein the treatment output comprises at least one of an identification of a treatment to treat the subject, a design for the treatment, a manufacturing plan for the treatment, or a treatment plan for administering the treatment.

118. The method of claim 116, wherein the treatment comprises at least one of an antiviral therapeutic, an anti-inflammatory therapeutic, or an immune-based therapeutic.

119. The method of claim 116, wherein the treatment comprises at least one of remdesivir, baricitinib, tocilizumab, favipiravir, merimepodib, a monoclonal antibody therapeutic, Nirmatrelvir with Ritonavi, Molnupiravir, and/or an immune checkpoint inhibitor.

120. The method of claim 116, wherein generating the treatment output comprises:
determining a dosage for a therapeutic to treat the subject based on at least one of the diagnosis output or the disease indicator.

121. The method of any one of claims 93-120, wherein the diagnosis output identifies that the biological sample is positive for the symptomatic disease state and further comprising:
administering a therapeutic dosage of a therapeutic for the coronavirus disease to the subject, the therapeutic being selected from the group consisting of remdesivir, baricitinib, tocilizumab, a monoclonal antibody treatment, Nirmatrelvir with Ritonavi, Molnupiravir, and/or an immune checkpoint inhibitor.

122. A method of evaluating a biological sample obtained from a subject with respect to a symptomatic disease state corresponding to a coronavirus disease (COVID), the method comprising:
identifying a peptide structure profile for the biological sample using peptide structure data, the peptide structure profile comprising quantification data for a set of peptide structures associated with the symptomatic disease state,
wherein the set of peptide structures includes at least two peptide structures from a selected group of peptide structures identified in Table 1-1; and
wherein at least two peptide structures in the selected group of peptide structures includes a glycopeptide structure defined by a peptide sequence and a glycan structure linked to a linking site of the peptide sequence;
computing a disease indicator using the peptide structure profile and a model, wherein the disease indicator indicates whether the biological sample is positive for the symptomatic disease state; and
generating at least one of a diagnosis output or a treatment output based on the disease indicator.

123. The method of claim 122, further comprising receiving peptide structure data corresponding to the biological sample obtained from the subject.

124. The method of claim 122, wherein the model is a machine learning model and computing the disease indicator comprises:
computing the disease indicator using the machine learning model, the machine learning model including a set of weight coefficients that corresponds to the set of peptide structures, respectively,
wherein the disease indicator comprises at least one of a probability that the subject is positive for the symptomatic disease state, an odds that the subject is positive for the symptomatic disease state, a logarithm of the odds that the subject is positive for the symptomatic disease state, or a classification of the biological sample as either positive or negative for the symptomatic disease state.

125. The method of claim 122, wherein the model comprises a supervised machine learning model trained using an output of an unsupervised machine learning model that is trained to cluster a plurality of peptide structure profiles for a plurality of subjects according to a plurality of states, the plurality of states including the symptomatic disease state.

126. The method of claim 122, wherein the treatment output comprises at least one of an identification of a therapeutic to treat the subject, a design for the therapeutic, or a treatment plan for administering the therapeutic.

127. The method of claim 122, further comprising:
administering a therapeutic dosage of a therapeutic for the coronavirus disease to the subject based on the at least one of the diagnosis output or the treatment output, the therapeutic being selected from the group consisting of remdesivir, baricitinib, tocilizumab, a monoclonal antibody treatment, an immune checkpoint inhibitor, Nirmatrelvir with Ritonavi, Molnupiravir, and a combination thereof.

128. A method of designing a treatment for a symptomatic disease state of a coronavirus disease (COVID) in a subject, the method comprising:
designing a therapeutic for treating the subject in response to identifying the subject as being positive for the symptomatic disease state using the method of any one of claims 63-85, 87-92, 93-120, or 122-126.

129. A method of planning a treatment for a symptomatic disease state of a coronavirus disease (COVID) in a subject, the method comprising:
generating a treatment plan for treating the subject in response to identifying the subject as being positive for the symptomatic disease state using the method of any one of claims 63-85, 87-92, 93-120, or 122-126.

130. A method of manufacturing a treatment for a symptomatic disease state of a coronavirus disease (COVID) in a subject, the method comprising:
manufacturing a therapeutic for treating the subject in response to identifying the subject as being positive for the symptomatic disease state using the method of any one of claims 63-85, 87-92, 93-120, or 122-126.

131. A method of treating a symptomatic disease state of a coronavirus disease (COVID) in a subject, the method comprising:
administering to the subject a therapeutic to treat the subject based on identifying the subject as being positive for the symptomatic disease state using the method of any one of claims 63-85, 87-92, 93-120, or 122-126.

132. A method of treating a symptomatic disease state of a coronavirus disease (COVID) in a subject, the method comprising:
selecting a therapeutic to treat the subject based on determining that the subject is responsive to the therapeutic using the method of any of claims 63-85, 87-92, 93-120, or 122-126; and
administering the selected therapeutic to the subject.

133. A method for analyzing a set of peptide structures in a sample from a patient, the method comprising:
(a) preparing a patient sample to form a prepared sample comprising a set of peptide structures;
(b) inputting the prepared sample into a reaction monitoring mass spectrometry system to detect a set of product ions associated with each peptide structure of the set of peptide structures, the set of peptide structures comprising at least one of:
a first peptide structure associated with the set of product ions that includes a product ion having a mass-to-charge (m/z) ratio within a range selected from a group consisting of 366.1±0.5, ±0.8, and ±1.0 and that is characterized as having a precursor ion having an m/z ratio within a range selected from a group consisting of 891±1.0 and ±1.5;
a second peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio within a range selected from a group consisting of 1111.7±1.0 and ±1.5;
a third peptide structure associated with the corresponding set of product ions that includes a product ion having a mass-to-charge (m/z) ratio that is within a range selected from a group consisting of 1062.5±0.5, ±0.8, and ±1.0 and that is characterized as having a precursor ion having an m/z ratio within a range selected from a group consisting of 1196.5±1.0 and ±1.5;
a fourth peptide structure associated with the set of product ions that includes a product ion having a mass-to-charge (m/z) ratio that is within a range selected from a group consisting of 1062.5±0.5, ±0.8, and ±1.0 and that is characterized as having a precursor ion having an m/z ratio within a range selected from a group consisting of 1233±1.0 and ±1.5;
a fifth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1167.3±1.0 and ±1.5;
a sixth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1173.1±1.0 and ±1.5;
a seventh peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1327.6±1.0 and ±1.5;
an eighth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1061.9±1.0 and ±1.5;
a ninth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 874.7±1.0 and ±1.5;
a tenth peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1225.8±1.0 and ±1.5;
an 11^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1183.5±1.0 and ±1.5;
a 12^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio within a range selected from a group consisting of 1114.2±1.0 and ±1.5;
a 13^{th} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio within a range selected from a group consisting of 1278.3±1.0 and ±1.5;
a 14^{th} peptide structure associated with the set of product ions that includes a product ion having a mass-to-charge (m/z) ratio that is within a range selected from a group consisting of 1453.6±0.5, ±0.8, and ±1.0 and that is characterized as having a precursor ion having an m/z ratio within a range selected from a group consisting of 1055.8±1.0 and ±1.5;
a 15^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1284.6±1.0 and ±1.5;
a 16^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1285.1±1.0 and ±1.5;
a 17^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 991.2±1.0 and ±1.5;
an 18^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1027.7±1.0 and ±1.5;
a 19^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio within a range selected from a group consisting of 1155±1.0 and ±1.5;
a 20^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1191.2±1.0 and ±1.5;
a 21^{st} peptide structure associated with the set of product ions that includes a product ion having a mass-to-charge (m/z) ratio that is within a range selected from a group consisting of 204.1±0.5, ±0.8, and ±1.0 and that is characterized as having a precursor ion having an m/z ratio within a range selected from a group consisting of 878.8±1.0 and ±1.5;
a 22^{nd} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 976.1±1.0 and ±1.5;
a 23^{rd} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1043.8±1.0 and ±1.5;
a 24^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1199.5±1.0 and ±1.5;
a 25^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1366.3±1.0 and ±1.5;
a 26^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio within a range selected from a group consisting of 939.1±1.0 and ±1.5;
a 27^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio within a range selected from a group consisting of 1295±1.0 and ±1.5;
a 28^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 984.7±1.0 and ±1.5;
a 29^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1367.6±1.0 and ±1.5;
a 30^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1199.2±1.0 and ±1.5;
a 31^{st} peptide structure associated with the set of product ions that includes a product ion having a mass-to-charge (m/z) ratio that is within a range selected from a group consisting of 736.4±0.5, ±0.8, and ±1.0 and that is characterized as having a precursor ion having an m/z ratio within a range selected from a group consisting of 618.3±1.0 and ±1.5;
a 32^{nd} peptide structure associated with the set of product ions that includes a product ion having a mass-to-charge (m/z) ratio that is within a range selected from a group consisting of 342.2±0.5, ±0.8, and ±1.0 and that is characterized as having a precursor ion having an m/z ratio within a range selected from a group consisting of 590.3±1.0 and ±1.5;
a 33^{rd} peptide structure associated with the set of product ions that includes a product ion having a mass-to-charge (m/z) ratio that is within a range selected from a group consisting of 609.3±0.5, ±0.8, and ±1.0 and that is characterized as having a precursor ion having an m/z ratio within a range selected from a group consisting of 819.1±1.0 and ±1.5;
a 34^{th} peptide structure associated with the set of product ions that includes a product ion having a mass-to-charge (m/z) ratio that is within a range selected from a group consisting of 607.3±0.5, ±0.8, and ±1.0 and that is characterized as having a precursor ion having an m/z ratio within a range selected from a group consisting of 515.8±1.0 and ±1.5;
a 35^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1116.9±1.0 and ±1.5;
a 36^{th} peptide structure associated with the set of product ions that includes a product ion having a mass-to-charge (m/z) ratio that is within a range selected from a group consisting of 274.1±0.5, ±0.8, and ±1.0 and that is characterized as having a precursor ion having an m/z ratio within a range selected from a group consisting of 1154.7±1.0 and ±1.5;
a 37^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1272.6±1.0 and ±1.5;
a 38^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1285.6±1.0 and ±1.5;
a 39^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1441.6±1.0 and ±1.5;
a 40^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1206.3±1.0 and ±1.5;
a 41^{st} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1179.1±1.0 and ±1.5;
a 42^{nd} peptide structure associated with the set of product ions that includes a product ion having a mass-to-charge (m/z) ratio that is within a range selected from a group consisting of 529.3±0.5, ±0.8, and ±1.0 and that is characterized as having a precursor ion having an m/z ratio within a range selected from a group consisting of 660.4±1.0 and ±1.5;
a 43^{rd} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1310.6±1.0 and ±1.5;
a 44^{th} peptide structure associated with the set of product ions that includes a product ion having a mass-to-charge (m/z) ratio that is within a range selected from a group consisting of 366.1±0.5, ±0.8, and ±1.0 and that is characterized as having a precursor ion having an m/z ratio within a range selected from a group consisting of 1021.4±1.0 and ±1.5;
a 45^{th} peptide structure associated with the set of product ions that includes a product ion having a mass-to-charge (m/z) ratio that is within a range selected from a group consisting of 234.1±0.5, ±0.8, and ±1.0 and that is characterized as having a precursor ion having an m/z ratio within a range selected from a group consisting of 764.9±1.0 and ±1.5; and
(c) generating quantification data for the set of product ions using the reaction monitoring mass spectrometry system.

134. The method of claim 133, further comprising, prior to (a), obtaining the sample from the patient.

135. The method of claim 133, further comprising:
generating a diagnosis output using the quantification data and a model that has been trained using at least one of supervised or unsupervised machine learning.

136. The method of claim 133, wherein the reaction monitoring mass spectrometry system uses at least one of multiple reaction monitoring mass spectrometry (MRM-MS), or selected reaction monitoring mass spectrometry (SRM-MS) to detect the set of product ions and generate the quantification data.

137. The method of claim 13336, wherein the sample comprises a plasma sample.

138. The method of claim 133, wherein the sample comprises a serum sample.

139. The method of claim 133, wherein preparing the sample comprises at least one of:
denaturing one or more proteins in the sample to form one or more denatured proteins;
reducing the one or more denatured proteins in the sample to form one or more reduced proteins;
alkylating the one or more proteins in the sample using an alkylating agent to prevent reformation of disulfide bonds in the one or more reduced proteins to form one or more alkylated proteins; or
digesting the one or more alkylated proteins in the sample using a proteolysis catalyst to form the prepared sample comprising the set of peptide structures.

140. A composition comprising at least one of peptide structures PS-1 to PS-45 identified in Table 1-1.

141. A composition comprising a peptide structure or a product ion, wherein:
the peptide structure or product ion comprises an amino acid sequence having at least 90% sequence identity to any one of SEQ ID NOS: 84-116, corresponding to peptide structures PS-1 to PS-45 in Table 1-1; and
the product ion is selected as one from a group consisting of product ions identified in Table 4-1 including product ions falling within an identified m/z range.

142. A composition comprising a glycopeptide structure selected as one from a group of glycopeptide structures consisting of:
a first glycopeptide structure having a monoisotopic mass of 4447.5 and comprising:
the amino acid sequence of SEQ ID NO: 84; and
glycan structure GL NO. 5401 linked to the 12^{th} residue of SEQ ID NO: 84;
a second glycopeptide structure having a monoisotopic mass of 4440.74 and comprising:
the amino acid sequence of SEQ ID NO. 85; and
glycan structure GL NO. 5401 linked to the 15^{th} residue of SEQ ID NO. 85;
a third glycopeptide structure having a monoisotopic mass of 4779.95 and comprising:
the amino acid sequence of SEQ ID NO. 86; and
glycan structure GL NO. 6503 linked to the 15^{th} residue of SEQ ID NO. 86;
a fourth glycopeptide structure having a monoisotopic mass 4926.00 of and comprising:
the amino acid sequence of SEQ ID NO. 86;
glycan structure GL NO. 6513 linked to the 15^{th} residue of SEQ ID NO. 86;
a fifth glycopeptide structure having a monoisotopic mass of 5829.49 and comprising:
the amino acid sequence of SEQ ID NO. 87; and
glycan structure GL NO. 7602 linked to the 15^{th} residue of SEQ ID NO. 87;
a sixth glycopeptide structure having a monoisotopic mass of 3515.36 and comprising:
the amino acid sequence of SEQ ID NO. 88; and
glycan structure GL NO. 5402 linked to the 4^{th} residue of SEQ ID NO. 88;
a seventh glycopeptide structure having a monoisotopic mass of 3979.65 and comprising:
the amino acid sequence of SEQ ID NO. 89; and
glycan structure GL NO. 5420 linked to the 10^{th} residue of SEQ ID NO. 89;
an eighth glycopeptide structure having a monoisotopic mass of 4242.66 and comprising:
the amino acid sequence of SEQ ID NO. 90; and
glycan structure GL NO. 5412 linked to the 10^{th} residue of SEQ ID NO. 90;
a ninth glycopeptide structure having a monoisotopic mass of 2621.06 and comprising:
the amino acid sequence of SEQ ID NO. 91; and
glycan structure GL NO. 5301 linked to the 1^{st} residue of SEQ ID NO. 91;
a tenth glycopeptide structure having a monoisotopic mass of 4900.17 and comprising:
the amino acid sequence of SEQ ID NO. 92; and
glycan structure GL NO. 6411 linked to the 6^{th} residue of SEQ ID NO. 92;
an 11^{th} glycopeptide structure having a monoisotopic mass of 4729.04 and comprising:
the amino acid sequence of SEQ ID NO. 93; and
glycan structure GL NO. 7602 linked to the 6^{th} residue of SEQ ID NO. 93;
a 12^{th} glycopeptide structure having a monoisotopic mass of 4450.92 and comprising:
the amino acid sequence of SEQ ID NO. 94; and
glycan structure GL NO. 5412 linked to the 4^{th} residue of SEQ ID NO. 94;
a 13^{th} glycopeptide structure having a monoisotopic mass of 5107.14 and comprising:
the amino acid sequence of SEQ ID NO. 94; and
glycan structure GL NO. 6513 linked to the 4^{th} residue of SEQ ID NO. 94;
a 14^{th} glycopeptide structure having a monoisotopic mass of 3163.24 and comprising:
the amino acid sequence of SEQ ID NO. 95; and
glycan structure GL NO. 5401 linked to the 3^{rd} residue of SEQ ID NO. 95;
a 15^{th} glycopeptide structure having a monoisotopic mass of 5133.19 and comprising:
the amino acid sequence of SEQ ID NO. 96; and
glycan structure GL NO. 5402 linked to the 19^{th} residue of SEQ ID NO. 96;
a 16^{th} glycopeptide structure having a monoisotopic mass of 5133.19 and comprising:
the amino acid sequence of SEQ ID NO. 96; and
glycan structure GL NO. 5421 linked to the 19^{th} residue of SEQ ID NO. 96;
a 17^{th} glycopeptide structure having a monoisotopic mass of 3959.66 and comprising:
the amino acid sequence of SEQ ID NO. 97; and
glycan structure GL NO. 5402 linked to the 4^{th} residue of SEQ ID NO. 97;
an 18^{th} glycopeptide structure having a monoisotopic mass of 4105.72 and comprising:
the amino acid sequence of SEQ ID NO. 97; and
glycan structure GL NO. 5412 linked to the 4^{th} residue of SEQ ID NO. 97;
a 19^{th} glycopeptide structure having a monoisotopic mass of 4615.89 and comprising:
the amino acid sequence of SEQ ID NO. 97; and
glycan structure GL NO. 6503 linked to the 4^{th} residue of SEQ ID NO. 97;
a 20^{th} glycopeptide structure having a monoisotopic mass of 4758.93 and comprising:
the amino acid sequence of SEQ ID NO. 98; and
glycan structure GL NO. 6513 linked to the 4^{th} residue of SEQ ID NO. 98;
a 21^{st} glycopeptide structure having a monoisotopic mass of 2633.04 and comprising:
the amino acid sequence of SEQ ID NO. 99; and
glycan structure GL NO. 3410 linked to the 5^{th} residue of SEQ ID NO. 99;
a 22^{nd} glycopeptide structure having a monoisotopic mass of 2925.15 and comprising:
the amino acid sequence of SEQ ID NO. 99; and
glycan structure GL NO. 5410 linked to the 5^{th} residue of SEQ ID NO. 99;
a 23^{rd} glycopeptide structure having a monoisotopic mass of 3128.23 and comprising:
the amino acid sequence of SEQ ID NO. 99; and
glycan structure GL NO. 5510 linked to the 5^{th} residue of SEQ ID NO. 99;
a 24^{th} glycopeptide structure having a monoisotopic mass of 4677.79 and comprising:
the amino acid sequence of SEQ ID NO. 100; and
glycan structure GL NO. 6503 linked to the 9^{th} residue of SEQ ID NO. 100;
a 25^{th} glycopeptide structure having a monoisotopic mass of 6822.70 and comprising:
the amino acid sequence of SEQ ID NO. 101; and
glycan structure GL NO. 5402 linked to the 9^{th} residue of SEQ ID NO. 101;
a 26^{th} glycopeptide structure having a monoisotopic mass of 2813.31 and comprising:
the amino acid sequence of SEQ ID NO. 102; and
glycan structure GL NO. 1101 linked to the 12^{th} residue of SEQ ID NO. 102;
a 27^{th} glycopeptide structure having a monoisotopic mass of 5174.11 and comprising:
the amino acid sequence of SEQ ID NO. 103; and
glycan structure GL NO. 6523 linked to the 9^{th} residue of SEQ ID NO. 103;
a 28^{th} glycopeptide structure having a monoisotopic mass of 3933.66 and comprising:
the amino acid sequence of SEQ ID NO. 104; and
glycan structure GL NO. 5401 linked to the 15^{th} residue of SEQ ID NO. 104;
a 29^{th} glycopeptide structure having a monoisotopic mass of 5463.24 and comprising:
the amino acid sequence of SEQ ID NO. 105; and
glycan structure GL NO. 5401 linked to the 16^{th} residue of SEQ ID NO. 105;
a 30^{th} glycopeptide structure having a monoisotopic mass of 4791.91 and comprising:
the amino acid sequence of SEQ ID NO. 106; and
glycan structure GL NO. 7420 linked to the 4^{th} residue of SEQ ID NO. 106;
a 31^{st} glycopeptide structure having a monoisotopic mass of 5578.17 and comprising:
the amino acid sequence of SEQ ID NO. 111; and
glycan structure GL NO. 7614 linked to the 7^{th} residue of SEQ ID NO. 111;
a 32^{th} glycopeptide structure having a monoisotopic mass of 4612.87 and comprising:
the amino acid sequence of SEQ ID NO. 98; and
glycan structure GL NO. 6503 linked to the 4^{th} residue of SEQ ID NO. 98;
a 33^{rd} glycopeptide structure having a monoisotopic mass of 5084.28 and comprising:
the amino acid sequence of SEQ ID NO. 112; and
glycan structure GL NO. 5401 linked to the 17^{th} residue of SEQ ID NO. 112;
a 34^{th} glycopeptide structure having a monoisotopic mass of 3853.74 and comprising:
the amino acid sequence of SEQ ID NO. 93; and
glycan structure GL NO. 5411 linked to the 6^{th} residue of SEQ ID NO. 93;
a 35^{th} glycopeptide structure having a monoisotopic mass of 4321.78 and comprising:
the amino acid sequence of SEQ ID NO. 98; and
glycan structure GL NO. 6502 linked to the 4^{th} residue of SEQ ID NO. 98;
a 36^{th} glycopeptide structure having a monoisotopic mass of 8432.83 and comprising:
the amino acid sequence of SEQ ID NO. 113; and
glycan structure GL NO. 5402 linked to the 46^{th} residue of SEQ ID NO. 113;
a 37^{th} glycopeptide structure having a monoisotopic mass of 5890.66 and comprising:
the amino acid sequence of SEQ ID NO. 97; and
glycan structure GL NO. 5412 linked to the 4^{th} residue of SEQ ID NO. 97;
a 38^{th} glycopeptide structure having a monoisotopic mass of 3927.64 and comprising:
the amino acid sequence of SEQ ID NO. 115; and
glycan structure GL NO. 5401 linked to the 5^{th} residue of SEQ ID NO. 115;
a 39^{th} glycopeptide structure having a monoisotopic mass of 4079.71 and comprising:
the amino acid sequence of SEQ ID NO. 104; and
glycan structure GL NO. 5411 linked to the 15^{th} residue of SEQ ID NO. 104; and
wherein:
the glycan structure GL NO. 1101 comprises: 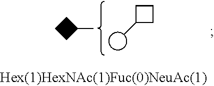
the glycan structure GL NO. 3410 comprises: 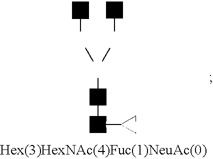
the glycan structure GL NO. 5401 comprises: 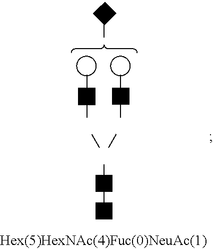
the glycan structure (GL NO. 5402) comprises: 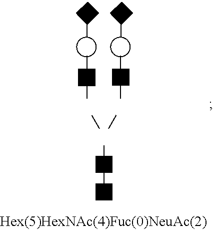
the glycan structure (GL NO. 5410) comprises: 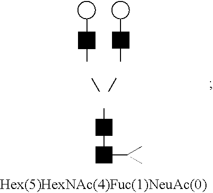
the glycan structure (GL NO. 5411) comprises: 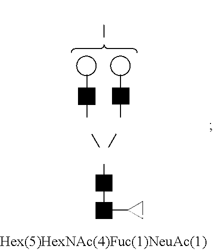
the glycan structure (GL NO. 5412) comprises: 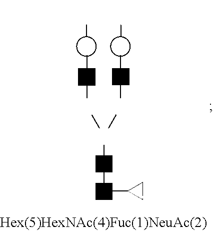
the glycan structure (GL NO. 5420) comprises: 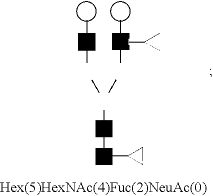
the glycan structure (GL NO. 5510) comprises: 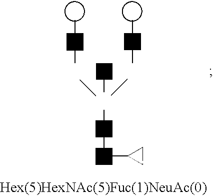
the glycan structure (GL NO. 6411) comprises: 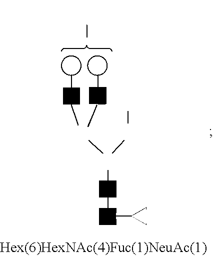
the glycan structure (GL NO. 6502) comprises: 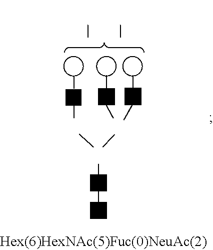
the glycan structure (GL NO. 6503) comprises: 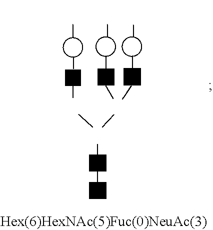
the glycan structure (GL NO. 6523) comprises: 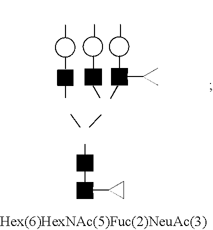
the glycan structure (GL NO. 7420) comprises: 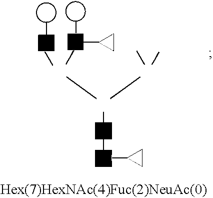
the glycan structure (GL NO. 7602) comprises:

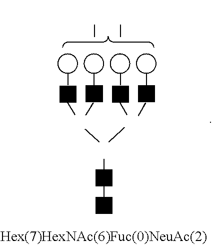

143. The composition of claim 142, wherein:
the first glycopeptide structure has a precursor ion having a charge of 5;
the second glycopeptide structure has a precursor ion having a charge of 4;
the third glycopeptide structure has a precursor ion having a charge of 4;
the fourth glycopeptide structure has a precursor ion having a charge of 4;
the fifth glycopeptide structure has a precursor ion having a charge of 5;
the sixth glycopeptide structure has a precursor ion having a charge of 3;
the seventh glycopeptide structure has a precursor ion having a charge of 3;
the eighth glycopeptide structure has a precursor ion having a charge of 4;
the ninth glycopeptide structure has a precursor ion having a charge of 3;
the tenth glycopeptide structure has a precursor ion having a charge of 4;
the 11^{th} glycopeptide structure has a precursor ion having a charge of 4;
the 12^{th} glycopeptide structure has a precursor ion having a charge of 4;
the 13^{th} glycopeptide structure has a precursor ion having a charge of 4;
the 14^{th} glycopeptide structure has a precursor ion having a charge of 3;
the 15^{th} glycopeptide structure has a precursor ion having a charge of 4;
the 16^{th} glycopeptide structure has a precursor ion having a charge of 4;
the 17^{th} glycopeptide structure has a precursor ion having a charge of 4;
the 18^{th} glycopeptide structure has a precursor ion having a charge of 4;
the 19^{th} glycopeptide structure has a precursor ion having a charge of 4;
the 20^{th} glycopeptide structure has a precursor ion having a charge of 4;
the 21^{st} glycopeptide structure has a precursor ion having a charge of 3;
the 22^{nd} glycopeptide structure has a precursor ion having a charge of 3;
the 23^{rd} glycopeptide structure has a precursor ion having a charge of 3;
the 24^{th} glycopeptide structure has a precursor ion having a charge of 4;
the 25^{th} glycopeptide structure has a precursor ion having a charge of 5;
the 26^{th} glycopeptide structure has a precursor ion having a charge of 3;
the 27^{th} glycopeptide structure has a precursor ion having a charge of 4;
the 28^{th} glycopeptide structure has a precursor ion having a charge of 4;
the 29^{th} glycopeptide structure has a precursor ion having a charge of 4;
the 30^{th} glycopeptide structure has a precursor ion having a charge of 4;
the 31^{st} glycopeptide structure has a precursor ion having a charge of 5;
the 32^{th} glycopeptide structure has a precursor ion having a charge of 4;
the 33^{rd} glycopeptide structure has a precursor ion having a charge of 4;
the 34^{th} glycopeptide structure has a precursor ion having a charge of 3;
the 35^{th} glycopeptide structure has a precursor ion having a charge of 3;
the 36^{th} glycopeptide structure has a precursor ion having a charge of 7;
the 37^{th} glycopeptide structure has a precursor ion having a charge of 5;
the 38^{th} glycopeptide structure has a precursor ion having a charge of 3; and
the 39^{th} glycopeptide structure has a precursor ion having a charge of 4.

144. The composition of claim 142, wherein:
the first glycopeptide structure has a product ion having a mass-to-charge (m/z) ratio within a range selected from a group consisting of 366.1±0.5, ±0.8, and ±1.0;
the second glycopeptide structure has a product ion having a mass-to-charge (m/z) ratio that is within a range selected from a group consisting of 204.1±0.5, ±0.8, and ±1.0;
the third glycopeptide structure associated with the corresponding set of product ions that includes a product ion having a mass-to-charge (m/z) ratio that is within a range selected from a group consisting of 1062.5±0.5, ±0.8, and ±1.0;
the fourth glycopeptide structure has a product ion having a mass-to-charge (m/z) ratio that is within a range selected from a group consisting of 1062.5±0.5, ±0.8, and ±1.0;
the fifth glycopeptide structure has a product ion having a mass-to-charge (m/z) ratio that is within a range selected from a group consisting of 366.1±0.5, ±0.8, and ±1.0;
the sixth glycopeptide structure has a product ion having a mass-to-charge (m/z) ratio that is within a range selected from a group consisting of 366.1±0.5, ±0.8, and ±1.0;
the seventh glycopeptide structure has a product ion having a mass-to-charge (m/z) ratio that is within a range selected from a group consisting of 366.1±0.5, ±0.8, and ±1.0;
the eighth glycopeptide structure has a product ion having a mass-to-charge (m/z) ratio that is within a range selected from a group consisting of 366.1±0.5, ±0.8, and ±1.0;
the ninth glycopeptide structure has a product ion having a mass-to-charge (m/z) ratio that is within a range selected from a group consisting of 366.1±0.5, ±0.8, and ±1.0;
the tenth glycopeptide structure has a product ion having a mass-to-charge (m/z) ratio that is within a range selected from a group consisting of 366.1±0.5, ±0.8, and ±1.0;
the 11^{th} glycopeptide structure has a product ion having a mass-to-charge (m/z) ratio that is within a range selected from a group consisting of 366.1±0.5, ±0.8, and ±1.0;
the 12^{th} glycopeptide structure has a product ion having a mass-to-charge (m/z) ratio that is within a range selected from a group consisting of 204.1±0.5, ±0.8, and ±1.0;
the 13^{th} glycopeptide structure has a product ion having a mass-to-charge (m/z) ratio that is within a range selected from a group consisting of 204.1±0.5, ±0.8, and ±1.0;
the 14^{th} glycopeptide structure has a product ion having a mass-to-charge (m/z) ratio that is within a range selected from a group consisting of 1453.6±0.5, ±0.8, and ±1.0;
the 15^{th} glycopeptide structure has a product ion having a mass-to-charge (m/z) ratio that is within a range selected from a group consisting of 366.1±0.5, ±0.8, and ±1.0;
the 16^{th} glycopeptide structure has a product ion having a mass-to-charge (m/z) ratio that is within a range selected from a group consisting of 366.1±0.5, ±0.8, and ±1.0;
the 17^{th} glycopeptide structure has a product ion having a mass-to-charge (m/z) ratio that is within a range selected from a group consisting of 366.1±0.5, ±0.8, and ±1.0;
the 18^{th} glycopeptide structure has a product ion having a mass-to-charge (m/z) ratio that is within a range selected from a group consisting of 366.1±0.5, ±0.8, and ±1.0;
the 19^{th} glycopeptide structure has a product ion having a mass-to-charge (m/z) ratio that is within a range selected from a group consisting of 274.1±0.5, ±0.8, and ±1.0;
the 20^{th} glycopeptide structure has a product ion having a mass-to-charge (m/z) ratio that is within a range selected from a group consisting of 366.1±0.5, ±0.8, and ±1.0;
the 21^{st} glycopeptide structure has a product ion having a mass-to-charge (m/z) ratio that is within a range selected from a group consisting of 204.1±0.5, ±0.8, and ±1.0;
the 22^{nd} glycopeptide structure has a product ion having a mass-to-charge (m/z) ratio that is within a range selected from a group consisting of 366.1±0.5, ±0.8, and ±1.0;
the 23^{rd} glycopeptide structure has a product ion having a mass-to-charge (m/z) ratio that is within a range selected from a group consisting of 366.1±0.5, ±0.8, and ±1.0;
the 24^{th} glycopeptide structure has a product ion having a mass-to-charge (m/z) ratio that is within a range selected from a group consisting of 366.1±0.5, ±0.8, and ±1.0;
the 25^{th} glycopeptide structure has a product ion having a mass-to-charge (m/z) ratio that is within a range selected from a group consisting of 366.1±0.5, ±0.8, and ±1.0;
the 26^{th} glycopeptide structure has a product ion having a mass-to-charge (m/z) ratio that is within a range selected from a group consisting of 274.1±0.5, ±0.8, and ±1.0;
the 27^{th} glycopeptide structure has a product ion having a mass-to-charge (m/z) ratio that is within a range selected from a group consisting of 274.1±0.5, ±0.8, and ±1.0;
the 28^{th} glycopeptide structure has a product ion having a mass-to-charge (m/z) ratio that is within a range selected from a group consisting of 366.1±0.5, ±0.8, and ±1.0;
the 29^{th} glycopeptide structure has a product ion having a mass-to-charge (m/z) ratio that is within a range selected from a group consisting of 366.1±0.5, ±0.8, and ±1.0;
the 30^{th} glycopeptide structure has a product ion having a mass-to-charge (m/z) ratio that is within a range selected from a group consisting of 366.1±0.5, ±0.8, and ±1.0;
the 31^{st} glycopeptide structure has a product ion having a mass-to-charge (m/z) ratio that is within a range selected from a group consisting of 366.1±0.5, ±0.8, and ±1.0;
the 32^{nd} glycopeptide structure has a product ion having a mass-to-charge (m/z) ratio that is within a range selected from a group consisting of 274.1±0.5, ±0.8, and ±1.0;
the 33^{rd} glycopeptide structure has a product ion having a mass-to-charge (m/z) ratio that is within a range selected from a group consisting of 366.1±0.5, ±0.8, and ±1.0;
the 34^{th} glycopeptide structure has a product ion having a mass-to-charge (m/z) ratio that is within a range selected from a group consisting of 366.1±0.5, ±0.8, and ±1.0;
the 35^{th} glycopeptide structure has a product ion having a mass-to-charge (m/z) ratio that is within a range selected from a group consisting of 366.1±0.5, ±0.8, and ±1.0;
the 36^{th} glycopeptide structure has a product ion having a mass-to-charge (m/z) ratio that is within a range selected from a group consisting of 366.1±0.5, ±0.8, and ±1.0;
the 37^{th} glycopeptide structure has a product ion having a mass-to-charge (m/z) ratio that is within a range selected from a group consisting of 366.1±0.5, ±0.8, and ±1.0;
the 39^{th} glycopeptide structure has a product ion having a mass-to-charge (m/z) ratio that is within a range selected from a group consisting of 366.1±0.5, ±0.8, and ±1.0; and
the 39^{th} glycopeptide structure has a product ion having a mass-to-charge (m/z) ratio that is within a range selected from a group consisting of 366.1±0.5, ±0.8, and ±1.0.

145. The composition of claim 142, wherein:
the first glycopeptide structure has a precursor ion having an m/z ratio within a range selected from a group consisting of 891±1.0 and ±1.5;
the second glycopeptide structure has a precursor ion having an m/z ratio within a range selected from a group consisting of 1111.7±1.0 and ±1.5;
the third glycopeptide structure has a precursor ion having an m/z ratio within a range selected from a group consisting of 1196.5±1.0 and ±1.5;
the fourth glycopeptide structure has a precursor ion having an m/z ratio within a range selected from a group consisting of 1233±1.0 and ±1.5;
the fifth glycopeptide structure has a precursor ion having an m/z ratio within a range selected from a group consisting of 1167.3±1.0 and ±1.5;
the sixth glycopeptide structure has a precursor ion having an m/z ratio within a range selected from a group consisting of 1173.1±1.0 and ±1.5;
the seventh glycopeptide structure has a precursor ion having an m/z ratio within a range selected from a group consisting of 1327.6±1.0 and ±1.5;
the eighth glycopeptide structure has a precursor ion having an m/z ratio within a range selected from a group consisting of 1061.9±1.0 and ±1.5;
the ninth glycopeptide structure has a precursor ion having an m/z ratio within a range selected from a group consisting of 874.7±1.0 and ±1.5;
the tenth glycopeptide structure has a precursor ion having an m/z ratio within a range selected from a group consisting of 1225.8±1.0 and ±1.5;
the 11^{th} glycopeptide structure has a precursor ion having an m/z ratio within a range selected from a group consisting of 1183.5±1.0 and ±1.5;
the 12^{th} glycopeptide structure has a precursor ion having an m/z ratio within a range selected from a group consisting of 1114.2±1.0 and ±1.5;
the 13^{th} glycopeptide structure has a precursor ion having an m/z ratio within a range selected from a group consisting of 1278.3±1.0 and ±1.5;
the 14^{th} glycopeptide structure has a precursor ion having an m/z ratio within a range selected from a group consisting of 1055.8±1.0 and ±1.5;
the 15^{th} glycopeptide structure has a precursor ion having an m/z ratio within a range selected from a group consisting of 1284.6±1.0 and ±1.5;
the 16^{th} glycopeptide structure has a precursor ion having an m/z ratio within a range selected from a group consisting of 1285.1±1.0 and ±1.5;
the 17^{th} glycopeptide structure has a precursor ion having an m/z ratio within a range selected from a group consisting of 991.2±1.0 and ±1.5;
the 18^{th} glycopeptide structure has a precursor ion having an m/z ratio within a range selected from a group consisting of 1027.7±1.0 and ±1.5;
the 19^{th} glycopeptide structure has a precursor ion having an m/z ratio within a range selected from a group consisting of 1155±1.0 and ±1.5;
the 20^{th} glycopeptide structure has a precursor ion having an m/z ratio within a range selected from a group consisting of 1191.2±1.0 and ±1.5;
the 21^{st} glycopeptide structure has a precursor ion having an m/z ratio within a range selected from a group consisting of 878.8±1.0 and ±1.5;
the 22^{nd} glycopeptide structure has a precursor ion having an m/z ratio within a range selected from a group consisting of 976.1±1.0 and ±1.5;
the 23^{rd} glycopeptide structure has a precursor ion having an m/z ratio within a range selected from a group consisting of 1043.8±1.0 and ±1.5;
the 24^{th} glycopeptide structure has a precursor ion having an m/z ratio within a range selected from a group consisting of 1199.5±1.0 and ±1.5;
the 25^{th} glycopeptide structure has a precursor ion having an m/z ratio within a range selected from a group consisting of 1366.3±1.0 and ±1.5;
the 26^{th} glycopeptide structure has a precursor ion having an m/z ratio within a range selected from a group consisting of 939.1±1.0 and ±1.5;
the 27^{th} glycopeptide structure has a precursor ion having an m/z ratio within a range selected from a group consisting of 1295±1.0 and ±1.5;
the 28^{th} glycopeptide structure has a precursor ion having an m/z ratio within a range selected from a group consisting of 984.7±1.0 and ±1.5;
the 29^{th} glycopeptide structure has a precursor ion having an m/z ratio within a range selected from a group consisting of 1367.6±1.0 and ±1.5;
the 30^{th} glycopeptide structure has a precursor ion having an m/z ratio within a range selected from a group consisting of 1199.2±1.0 and ±1.5;
the 31^{st} glycopeptide structure has a precursor ion having an m/z ratio within a range selected from a group consisting of 1116.9±1.0 and ±1.5;
the 32^{nd} glycopeptide structure has a precursor ion having an m/z ratio within a range selected from a group consisting of 1154.7±1.0 and ±1.5;
the 33^{rd} glycopeptide structure has a precursor ion having an m/z ratio within a range selected from a group consisting of 1272.6±1.0 and ±1.5;
the 34^{th} glycopeptide structure has a precursor ion having an m/z ratio within a range selected from a group consisting of 1285.6±1.0 and ±1.5;
the 35^{th} glycopeptide structure has a precursor ion having an m/z ratio within a range selected from a group consisting of 1441.6±1.0 and ±1.5;
the 36^{th} glycopeptide structure has a precursor ion having an m/z ratio within a range selected from a group consisting of 1206.3±1.0 and ±1.5;
the 37^{th} glycopeptide structure has a precursor ion having an m/z ratio within a range selected from a group consisting of 1179.1±1.0 and ±1.5;
the 39^{th} glycopeptide structure has a precursor ion having an m/z ratio within a range selected from a group consisting of 1310.6±1.0 and ±1.5; and
the 40^{th} glycopeptide structure has a precursor ion having an m/z ratio within a range selected from a group consisting of 1021.4±1.0 and ±1.5.

146. A composition comprising a peptide structure selected as one from a group of aglycosylated peptide structures consisting of:
a first peptide structure having a monoisotopic mass of 1234.68 and comprising the amino acid sequence of SEQ ID NO: 107;
a second peptide structure having a monoisotopic mass of 1178.67 comprising the amino acid sequence of SEQ ID NO: 108;
a third peptide structure having a monoisotopic mass of 2454.14 comprising the amino acid sequence of SEQ ID NO: 109;
a fourth peptide structure having a monoisotopic mass of 1029.53 comprising the amino acid sequence of SEQ ID NO: 110;
a fifth peptide structure having a monoisotopic mass of 1318.73 comprising the amino acid sequence of SEQ ID NO: 114; and
a sixth peptide structure having a monoisotopic mass of 1527.74 comprising the amino acid sequence of SEQ ID NO: 116.

147. The composition of claim 146, wherein:
the first peptide structure has a precursor ion having a charge of 2;
the second peptide structure has a precursor ion having a charge of 2;
the third peptide structure has a precursor ion having a charge of 3;
the fourth peptide structure has a precursor ion having a charge of 2;
the fifth peptide structure has a precursor ion having a charge of 2; and
the sixth peptide structure has a precursor ion having a charge of 2.

148. The composition of claim 146, wherein:
the first peptide structure has a product ion having a mass-to-charge (m/z) ratio that is within a range selected from a group consisting of 736.4±0.5, ±0.8, and ±1.0;
the second peptide structure has a product ion having a mass-to-charge (m/z) ratio that is within a range selected from a group consisting of 342.2±0.5, ±0.8, and ±1.0;
the third peptide structure has a product ion having a mass-to-charge (m/z) ratio that is within a range selected from a group consisting of 609.3±0.5, ±0.8, and ±1.0;
the fourth peptide structure has a product ion having a mass-to-charge (m/z) ratio that is within a range selected from a group consisting of 607.3±0.5, ±0.8, and ±1.0;
the fifth peptide structure has a product ion having a mass-to-charge (m/z) ratio that is within a range selected from a group consisting of 529.3±0.5, ±0.8, and ±1.0; and
the sixth peptide structure has a product ion having a mass-to-charge (m/z) ratio that is within a range selected from a group consisting of 234.1±0.5, ±0.8, and ±1.0.

149. The composition of claim 146, wherein:
the first peptide structure has a precursor ion having an m/z ratio within a range selected from a group consisting of 618.3±1.0 and ±1.5;
the second peptide structure has a precursor ion having an m/z ratio within a range selected from a group consisting of 590.3±1.0 and ±1.5;
the third peptide structure has a precursor ion having an m/z ratio within a range selected from a group consisting of 819.1±1.0 and ±1.5;
the fourth peptide structure has a precursor ion having an m/z ratio within a range selected from a group consisting of 515.8±1.0 and ±1.5;
the fifth peptide structure has a precursor ion having an m/z ratio within a range selected from a group consisting of 660.4±1.0 and ±1.5; and
the sixth peptide structure has a precursor ion having an m/z ratio within a range selected from a group consisting of 764.9±1.0 and ±1.5.

150. A kit comprising at least one agent for quantifying at least one peptide structure identified in Table 1-1 to carry out the method of any one of claims 63-139.

151. A kit comprising at least one of a glycopeptide standard, a buffer, or a set of peptide sequences to carry out the method of any one of claims 63-139, a peptide sequence of the set of peptide sequences identified by a corresponding one of SEQ ID NOS: 84-116, defined in Table 5-1.

152. A system comprising:
one or more data processors; and
a non-transitory computer readable storage medium containing instructions which, when executed on the one or more data processors, cause the one or more data processors to perform part or all of any one of claims 63-85, 87-92, 93-120, or 122-126.

153. A method of diagnosing a coronavirus disease (COVID) in a subject, comprising the step of identifying one or more peptide structures identified in Table 2-1 from a sample from the subject.

154. The method of claim 153, wherein the sample comprises blood, plasma, serum, urine, saliva, mucosal excretions, sputum, stool, nasal mucus, phlegm, and/or tears.

155. The method of claim 153 or 154, wherein the step of identifying occurs once.

156. The method of claim 153 or 154, wherein the step of identifying occurs multiple times.

157. The method of claim 156, wherein one or more symptoms becomes undetectable between multiple identifying steps.

158. A method of identifying or managing an at-risk subject for a coronavirus disease (COVID), the method comprising measuring whether a biological sample obtained from the subject evidences COVID using part or all of the method of any one of claims 63-85, 87-92, 93-120, 122-126, or 153-157, and
subjecting the subject to one or more medical tests or procedures, and/or
subjecting the subject to one or more preventatives or therapies in response to the identification of the symptomatic disease state.

159. The method of claim 158, wherein the subject has one or more COVID symptoms at the time of measuring and/or at the time of obtaining the sample.

160. The method of claim 158, wherein the subject is asymptomatic at the time of measuring and/or at the time of obtaining the sample.

161. A method of identifying a subject suitable for, or in need of, COVID prevention or treatment, the method comprising the step of measuring from a biological sample taken from the subject for the presence of one or a combination of peptide structures identified in Table 2-1, wherein their detection indicates that the subject should have COVID prevention or treatment.

162. The method of claim 161, wherein the subject has one or more COVID symptoms at the time of measuring and/or at the time of obtaining the sample.

163. The method of claim 161, wherein the subject is asymptomatic at the time of measuring and/or at the time of obtaining the sample.

164. A method of predicting whether a subject will be symptomatic upon coronavirus infection, comprising the step of measuring from a biological sample taken from the subject for the presence of one or a combination of peptide structures identified in Table 2-1.

165. A computer-program product tangibly embodied in a non-transitory machine-readable storage medium, including instructions configured to cause one or more data processors to perform part or all of any one of claims 63-85, 87-92, 93-120, 122-126, or 154-164.


SEQUENCE LISTING (116 sequences)

SEQ ID NO: 1 - protein, 201 aa, Homo sapiens
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 2 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 3 - protein, 189 aa, Homo sapiens
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 4 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 5 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 6 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 7 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 8 - protein, 591 aa, Homo sapiens
MKNSRTWAWRAPVELFLLCAALGCLSLPGSRGERPHSFGSNAVNKSFAKSRQMRSVDVTLMPIDCELSSWSSWTTCDPCQKKRYRYAYLLQPSQFHGEPCNFSDKEVEDCVTNRPCRSQVRCEGFVCAQTGRCVNRRLLCNGDNDCGDQSDEANCRRIYKKCQHEMDQYWGIGSLASGINLFTNSFEGPVLDHRYYAGGCSPHYILNTRFRKPYNVESYTPQTQGKYEFILKEYESYSDFERNVTEKMASKSGFSFGFKIPGIFELGISSQSDRGKHYIRRTKRFSHTKSVFLHARSDLEVAHYKLKPRSLMLHYEFLQRVKRLPLEYSYGEYRDLFRDFGTHYITEAVLGGIYEYTLVMNKEAMERGDYTLNNVHACAKNDFKIGGAIEEVYVSLGVSVGKCRGILNEIKDRNKRDTMVEDLVVLVRGGASEHITTLAYQELPTADLMQEWGDAVQYNPAIIKVKVEPLYELVTATDFAYSSTVRQNMKQALEEFQKEVSSCHCAPCQGNGVPVLKGSRCDCICPVGSQGLACEVSYRKNTPIDGKWNCWSNWSSCSGRRKTRQRQCNNPPPQNGGSPCSGPASETLDCS

SEQ ID NO: 9 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 10 - protein, 911 aa, Homo sapiens
MDGAMGPRGLLLCMYLVSLLILQAMPALGSATGRSKSSEKRQAVDTAVDGVFIRSLKVNCKVTSRFAHYVVTSQVVNTANEAREVAFDLEIPKTAFISDFAVTADGNAFIGDIKDKVTAWKQYRKAAISGENAGLVRASGRTMEQFTIHLTVNPQSKVTFQLTYEEVLKRNHMQYEIVIKVKPKQLVHHFEIDVDIFEPQGISKLDAQASFLPKELAAQTIKKSFSGKKGHVLFRPTVSQQQSCPTCSTSLLNGHFKVTYDVSRDKICDLLVANNHFAHFFAPQNLTNMNKNVVFVIDISGSMRGQKVKQTKEALLKILGDMQPGDYFDLVLFGTRVQSWKGSLVQASEANLQAAQDFVRGFSLDEATNLNGGLLRGIEILNQVQESLPELSNHASILIMLTDGDPTEGVTDRSQILKNVRNAIRGRFPLYNLGFGHNVDFNFLEVMSMENNGRAQRIYEDHDATQQLQGFYSQVAKPLLVDVDLQYPQDAVLALTQNHHKQYYEGSEIVVAGRIADNKQSSFKADVQAHGEGQEFSITCLVDEEEMKKLLRERGHMLENHVERLWAYLTIQELLAKRMKVDREERANLSSQALQMSLDYGFVTPLTSMSIRGMADQDGLKPTIDKPSEDSPPLEMLGPRRTFVLSALQPSPTHSSSNTQRLPDRVTGVDTDPHFIIHVPQKEDTLCFNINEEPGVILSLVQDPNTGFSVNGQLIGNKARSPGQHDGTYFGRLGIANPATDFQLEVTPQNITLNPGFGGPVFSWRDQAVLRQDGVVVTINKKRNLVVSVDDGGTFEVVLHRVWKGSSVHQDFLGFYVLDSHRMSARTHGLLGQFFHPIGFEVSDIHPGSDPTKPDATMVVRNRRLTVTRGLQKDYSKDPWHGAEVSCWFIHNNGAGLIDGAYTDYIVPDIF

SEQ ID NO: 11 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 12 - protein, 330 aa, Homo sapiens [sequence omitted]

SEQ ID NO: 13 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 14 - protein, 367 aa, Homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 15 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 16 - protein, 752 aa, Homo sapiens
MGPLMVLFCLLFLYPGLADSAPSCPQNVNISGGTFTLSHGWAPGSLLTYSCPQGLYPSPASRLCKSSGQWQTPGATRSLSKAVCKPVRCPAPVSFENGIYTPRLGSYPVGGNVSFECEDGFILRGSPVRQCRPNGMWDGETAVCDNGAGHCPNPGISLGAVRTGFRFGHGDKVRYRCSSNLVLTGSSERECQGNGVWSGTEPICRQPYSYDFPEDVAPALGTSFSHMLGATNPTQKTKESLGRKIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 17 - protein, 644 aa, Homo sapiens
MKLITILFLCSRLLLSLTQESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 18 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 19 - protein, 1703 aa, Homo sapiens
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHENLFHFVTDVQNNGVVKIPDAKGDDAWKVYYDETAQEIVDEFAMRYGVESIYQAMTHFACLSSKYMCPGVPAVMSTLLANINAYYAHTTASTNVSASDRFAASNFGKERFVKLLDQLHNSLRIDLSMYRNNFPASSPERLQDLKSTVDLLTSITFFRMKVQELQSPPRASQVVKDCVKACLNSTYEYIFNNCHELYSREYQTDPAKKGEVLPEEQGPSIKNLDFWSKLITLIVSIIEEDKNSYTPCLNQFPQELNVGKISAEVMWNLFAQDMKYAMEEHDKHRLCKSADYMNLHFKVKWLYNEYVTELPAFKDRVPEYPAWFEPFVIQWLDENEEVSRDFLHGALERDKKDGFQQTSEHALFSCSVVDVFSQLNQSFEIIKKLECPDPQIVGHYMRRFAKTISNVLLQYADIISKDFASYCSKEKEKVPCILMNNTQQLRVQLEKMFEAMGGKELDAEASDILKELQVKLNNVLDELSRVFATSFQPHIEECVKQMGDILSQVKGTGNVPASACSSVAQDADNVLQPIMDLLDSNLTLFAKICEKTVLKRVLKELWKLVMNTMEKTIVLPPLTDQTMIGNLLRKHGKGLEKGRVKLPSHSDGTQMIFNAAKELGQLSKLKDHMVREEAKSLTPKQCAVVELALDTIKQYFHAGGVGLKKTFLEKSPDLQSLRYALSLYTQATDLLIKTFVQTQSAQGLGVEDPVGEVSVHVELFTHPGTGEHKVTVKVVAANDLKWQTSGIFRPFIEVNIIGPQLSDKKRKFATKSKNNSWAPKYNESFQFTLSADAGPECYELQVCVKDYCFAREDRTVGLAVLQLRELAQRGSAACWLPLGRRIHMDDTGLTVLRILSQRSNDEVAKEFVKLKSDTRSAEEGGAAPAP

SEQ ID NO: 20 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 21 - protein, 347 aa, Homo sapiens
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 22 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 23 - protein, 381 aa, Homo sapiens
MWRSLGLALALCLLPSGGTESQDQSSLCKQPPAWSIRDQDPMLNSNGSVTVVALLQASUYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSUCCHCRHLIFEKTGSAITUQCKENLPSLCSUQGLRAEENITESCQURLPPAAUQISQQLIPTEASASURUKNQAKKUEUPSN

SEQ ID NO: 24 - protein, 24 aa, Homo sapiens
SVQEIQATFFYFTPNKTEDTIFLR

SEQ ID NO: 25 - protein, 8 aa, Homo sapiens
EDALNETR

SEQ ID NO: 26 - protein, 22 aa, Homo sapiens
ADGTVNQIEGEATPVNLTEPAK

SEQ ID NO: 27 - protein, 23 aa, Homo sapiens
NNATVHEQVGGPSLTSDLQAQSK

SEQ ID NO: 28 - protein, 20 aa, Homo sapiens
TLNQSSDELQLSMGNAMFVK

SEQ ID NO: 29 - protein, 23 aa, Homo sapiens
YPHKPEINSTTHPGADLQENFCR

SEQ ID NO: 30 - protein, 8 aa, Homo sapiens
NGSLFAFR

SEQ ID NO: 31 - protein, 24 aa, Homo sapiens
MVSHHNLTTGATLINEQWLLTTAK

SEQ ID NO: 32 - protein, 15 aa, Homo sapiens
EYESYSDFERNVTEK

SEQ ID NO: 33 - protein, 16 aa, Homo sapiens
VVLHPNYSQVDIGLIK

SEQ ID NO: 34 - protein, 17 aa, Homo sapiens
NGTGHGNSTHHGPEYMR

SEQ ID NO: 35 - protein, 25 aa, Homo sapiens
ICDLLVANNHFAHFFAPQNLTNMNK

SEQ ID NO: 36 - protein, 31 aa, Homo sapiens
YLGNATAIFFLPDEGKLQHLENELTHDIITK

SEQ ID NO: 37 - protein, 16 aa, Homo sapiens
YTGNASALFILPDQDK

SEQ ID NO: 38 - protein, 8 aa, Homo sapiens
HNSTGCLR

SEQ ID NO: 39 - protein, 9 aa, Homo sapiens
EEQYNSTYR

SEQ ID NO: 40 - protein, 21 aa, Homo sapiens
TVVQPSVGAAAGPVVPPCPGR

SEQ ID NO: 41 - protein, 21 aa, Homo sapiens
IVGGTNSSWGEWPWQVSLQVK

SEQ ID NO: 42 - protein, 13 aa, Homo sapiens
IYSGILNLSDITK

SEQ ID NO: 43 - protein, 19 aa, Homo sapiens
LQAPLNYTEFQKPICLPSK

SEQ ID NO: 44 - protein, 26 aa, Homo sapiens
ANLSSQALQMSLDYGFVTPLTSMSIR

SEQ ID NO: 45 - protein, 14 aa, Homo sapiens
QSVPAHFVALNGSK

SEQ ID NO: 46 - protein, 12 aa, Homo sapiens
SVSEINPTTQMK

SEQ ID NO: 47 - protein, 20 aa, Homo sapiens
TFVLSALQPSPTHSSSNTQR

SEQ ID NO: 48 - protein, 56 aa, Homo sapiens
ASVSVLGDILGSAMQNTQNLLQMPYGCGEQNMVLFAPNIYVLDYLNETQQLTPEIK

SEQ ID NO: 49 - protein, 20 aa, Homo sapiens
ACLNSTYEYIFNNCHELYSR

SEQ ID NO: 50 - protein, 12 aa, Homo sapiens
ASSIIDELFQDR

SEQ ID NO: 51 - protein, 11 aa, Homo sapiens
DLATVYVDVLK

SEQ ID NO: 52 - protein, 10 aa, Homo sapiens
DLLLPQPDLR

SEQ ID NO: 53 - protein, 22 aa, Homo sapiens
TSESGELHGLTTEEEFVEGIYK

SEQ ID NO: 54 - protein, 8 aa, Homo sapiens
VSLATVDK

SEQ ID NO: 55 - protein, 476 aa, Homo sapiens
MAPAGVSLRATILCLLAWAGLAAGDRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 56 - protein, 462 aa, Homo sapiens; identical to SEQ ID NO: 9

SEQ ID NO: 57 - protein, 201 aa, Homo sapiens; identical to SEQ ID NO: 1

SEQ ID NO: 58 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 59 - protein, 298 aa, Homo sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 60 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 61 - protein, 478 aa, Homo sapiens; identical to SEQ ID NO: 4

SEQ ID NO: 62 - protein, 406 aa, Homo sapiens; identical to SEQ ID NO: 7

SEQ ID NO: 63 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 64 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 65 - protein, 911 aa, Homo sapiens; identical to SEQ ID NO: 10

SEQ ID NO: 66 - protein, 418 aa, Homo sapiens; identical to SEQ ID NO: 11

SEQ ID NO: 67 - protein, 423 aa, Homo sapiens; identical to SEQ ID NO: 5

SEQ ID NO: 68 - protein, 330 aa, Homo sapiens [sequence omitted]

SEQ ID NO: 69 - protein, 326 aa, Homo sapiens; identical to SEQ ID NO: 13

SEQ ID NO: 70 - protein, 821 aa, Homo sapiens
MPTVISASVAPRTAAEPRSPGPVPHPAQSKATEAGGGNPSGIYSAIISRNFPIIGVKEKTFEQLHKKCLEKKVLYVDPEFPPDETSLFYSQKFPIQFVWKRPPEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDDGTNMTYGTSPSGLNMGELIARMVRNMDNSLLQDSDLDPRGSDERPTRTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEMHGNKQHLQKDFFLYNASKARSKTYINMREVSQRFRLPPSEYVIVPSTYEPHQEGEFILRVFSEKRNLSEEVENTISVDRPVKKKKTKPIIFVSDRANSNKELGVDQESEEGKGKTSPDKQKQSPQPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 71 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 72 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 73 - protein, 1703 aa, Homo sapiens; identical to SEQ ID NO: 19

SEQ ID NO: 74 - protein, 267 aa, Homo sapiens; identical to SEQ ID NO: 20

SEQ ID NO: 75 - protein, 347 aa, Homo sapiens; identical to SEQ ID NO: 21

SEQ ID NO: 76 - protein, 147 aa, Homo sapiens; identical to SEQ ID NO: 22

SEQ ID NO: 77 - protein, 644 aa, Homo sapiens; identical to SEQ ID NO: 17

SEQ ID NO: 78 - protein, 201 aa, Homo sapiens; identical to SEQ ID NO: 1

SEQ ID NO: 79 - protein, 622 aa, Homo sapiens; identical to SEQ ID NO: 6

SEQ ID NO: 80 - protein, 1474 aa, Homo sapiens; identical to SEQ ID NO: 18

SEQ ID NO: 81 - protein, 355 aa, Homo sapiens [sequence omitted]

SEQ ID NO: 82 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 83 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 84 - protein, 21 aa, Homo sapiens
VYIHPFHLVIHNESTCEQLAK

SEQ ID NO: 85 - protein, 22 aa, Homo sapiens
CSDGWSFDATTLDDNGTMLFFK

SEQ ID NO: 86 - protein, 16 aa, Homo sapiens
SVQEIQATFFYFTPNK

SEQ ID NO: 87 - protein, 24 aa, Homo sapiens
SVQEIQATFFYFTPNKTEDTIFLR

SEQ ID NO: 88 - protein, 12 aa, Homo sapiens
FLNNGTCTAEGK

SEQ ID NO: 89 - protein, 18 aa, Homo sapiens
DIVEYYNDSNGSHVLQGR

SEQ ID NO: 90 - protein, 16 aa, Homo sapiens
EHEGAIYPDNTTDFQR

SEQ ID NO: 91 - protein, 8 aa, Homo sapiens
NGSLFAFR

SEQ ID NO: 92 - protein, 24 aa, Homo sapiens
MVSHHNLTTGATLINEQWLLTTAK

SEQ ID NO: 93 - protein, 16 aa, Homo sapiens
VVLHPNYSQVDIGLIK

SEQ ID NO: 94 - protein, 19 aa, Homo sapiens
VLSNNSDANLELINTWVAK

SEQ ID NO: 95 - protein, 11 aa, Homo sapiens
LGNWSAMPSCK

SEQ ID NO: 96 - protein, 25 aa, Homo sapiens
ICDLLVANNHFAHFFAPQNLTNMNK

SEQ ID NO: 97 - protein, 16 aa, Homo sapiens
YLGNATAIFFLPDEGK

SEQ ID NO: 98 - protein, 16 aa, Homo sapiens
YTGNASALFILPDQDK

SEQ ID NO: 99 - protein, 9 aa, Homo sapiens
EEQYNSTYR

SEQ ID NO: 100 - protein, 15 aa, Homo sapiens
NPWGQVEWNGSWSDR

SEQ ID NO: 101 - protein, 40 aa, Homo sapiens
LNDTLDYECHDGYESNTGSTTGSIVCGYNGWSDLPICYER

SEQ ID NO: 102 - protein, 20 aa, Homo sapiens
TFVLSALQPSPTHSSSNTQR

SEQ ID NO: 103 - protein, 17 aa, Homo sapiens
ELHHLQEQNVSNAFLDK

SEQ ID NO: 104 - protein, 18 aa, Homo sapiens
IPCSQPPQIEHGTINSSR

SEQ ID NO: 105 - protein, 33 aa, Homo sapiens
QVFPGLNYCTSGAYSNASSTDSASYYPLTGDTR

SEQ ID NO: 106 - protein, 20 aa, Homo sapiens
ACLNSTYEYIFNNCHELYSR

SEQ ID NO: 107 - protein, 11 aa, Homo sapiens
DLATVYVDVLK

SEQ ID NO: 108 - protein, 10 aa, Homo sapiens
DLLLPQPDLR

SEQ ID NO: 109 - protein, 22 aa, Homo sapiens
TSESGELHGLTTEEEFVEGIYK

SEQ ID NO: 110 - protein, 8 aa, Homo sapiens
YFIDFVAR

SEQ ID NO: 111 - protein, 15 aa, Homo sapiens
QDQCIYNTTYLNVQR

SEQ ID NO: 112 - protein, 26 aa, Homo sapiens
WVLTAAHCLLYPPWDKNFTENDLLVR

SEQ ID NO: 113 - protein, 56 aa, Homo sapiens; identical to SEQ ID NO: 48

SEQ ID NO: 114 - protein, 11 aa, Homo sapiens
YVYIAELLAHK

SEQ ID NO: 115 - protein, 19 aa, Homo sapiens
VIDFNCTTSSVSSALANTK

SEQ ID NO: 116 - protein, 13 aa, Homo sapiens
FATTFYQHLADSK